(19) United States
(12) Patent Application Publication
(10) Pub. No.: US 2026/0337292 A1
(43) Pub. Date: Oct. 29, 2026

(54) ANTI-ADAM8 ANTIBODIES AND USES OF THE SAME

(71) Applicant: TRUSTEES OF TUFTS COLLEGE, Medford, MA (US)

(72) Inventors: Gail Sonenshein, Brookline, MA (US); Nora Mineva, Brighton, MA (US)

(73) Assignee: TRUSTEES OF TUFTS COLLEGE, Medford, MA (US)

(21) Appl. No.: 19/467,544

(22) Filed: Feb. 2, 2026

Related U.S. Application Data
(63) Continuation of application No. 17/615,312, filed on May 29, 2020, now Pat. No. 12,540,193.
(60) Provisional application No. 62/855,575, filed on May 31, 2019.

(51) Int. Cl. C07K 16/28; A61K 39/00; G01N 33/575

(52) U.S. Cl. CPC C07K 16/2896; G01N 33/5758; A61K 2039/505; C07K 2317/24; C07K 2317/33; C07K 2317/34; C07K 2317/73; C07K 2317/76; C07K 2317/92

(57) ABSTRACT

Provided herein are proteins that inhibit both the metalloprotease activity and disintegrin activity of human ADAM8, wherein the protein comprises an antigen-binding domain that: (i) binds specifically to human ADAM8; and (ii) binds to an epitope within human ADAM8 that includes at least one amino acid within the sequence of SEQ ID NO: 1, nucleic acids, vectors, compositions, and methods of use thereof (e.g., methods of treatment and methods of diagnosing).

### CROSS-REFERENCE TO RELATED APPLICATIONS

This application is a continuation of U.S. patent application Ser. No. 17/615,312, filed on Nov. 30, 2021, which claims priority to International Patent Application No. PCT/US2020/035280, filed on May 29, 2020, which claims priority to U.S. Provisional Patent Application Ser. No. 62/855,575, filed May 31, 2019; the entire contents of each of which are herein incorporated by reference.

### STATEMENT AS TO FEDERALLY SPONSORED RESEARCH

This invention was made with government support under grants CA194955, CA200161, and CA239942 awarded by the National Institutes of Health. The government has certain rights in the invention.

### SEQUENCE LISTING

This application contains a Sequence Listing that has been submitted electronically as an XML file named “46700-0002002_SL_ST26.XML.” The XML file, created on Feb. 2, 2026, is 110,037 bytes in size. The material in the XML file is hereby incorporated by reference in its entirety.

### TECHNICAL FIELD

The present disclosure relates generally to the fields of immunology and cancer biology. More specifically, the present disclosure relates to the use of proteins to treat cancer and to identify those patients that can benefit from this treatment.

### BACKGROUND

ADAM8 was found to be non-essential under physiological conditions, as evidenced by normal development, lack of pathological defects, and a normal life span of ADAM8-deficient mice. Under normal conditions in most human tissues and cells, ADAM8 mRNA is either undetectable or low, and protein expression is either limited to low levels or to an inactive cytoplasmic state. However, elevated ADAM8 expression has been detected in breast cancer and many other solid tumors, including adrenal, bone, brain, colorectal, esophageal, gastric, head and neck, hepatocellular, lung, pancreatic, prostate, renal, and thyroid cancers, as well as in lymphomas and leukemias. Overexpression of ADAM8 in solid tumors has been correlated with either a higher tumor grade, a more metastatic phenotype and/or poorer patient prognosis. Overall, the ADAM8 cell surface protein constitutes a crucial player in multiple steps of tumorigenesis and is a promising target for a large number of patients with aggressive ADAM8-driven cancers. Development of an anti-ADAM8 antagonist antibody could revolutionize treatment of patients affected by these cancers by providing an effective and tolerable therapeutic option, and reducing the mortality associated with metastatic disease.

### SUMMARY

The present invention relates to the discovery of a new class of proteins that target the disintegrin (DI) domain of ADAM8 and inhibit the activity of both the metalloproteinase (MP) and disintegrin (DI) domains of ADAM8. The effectiveness of these proteins indicates that the DI domain of ADAM8 has its own independent function and plays a role in maintaining the overall protein structure and in bridging/aligning the catalytic and substrate recognition activities necessary for proper MP domain functionality.

Provided herein are proteins that inhibit both the metalloprotease activity and disintegrin activity of human ADAM8, wherein the protein includes an antigen-binding domain that: (i) binds specifically to human ADAM8; and (ii) binds to an epitope within human ADAM8 that includes at least one amino acid within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

In some embodiments of any of the proteins described herein, the protein binds to human ADAM8 with a K_{D} of about 0.1 nM to about 100 nM. In some embodiments of any of the proteins described herein, the protein binds to human ADAM8 with a K_{D} of about 0.1 nM to about 10 nM.

In some embodiments of any of the proteins described herein, the protein includes a single polypeptide. In some embodiments of any of the proteins described herein, the antigen-binding domain is a VHH domain, a VNAR domain, or a scFv. In some embodiments of any of the proteins described herein, the protein is selected from the group consisting of: a BiTe, a (scFv) 2, a nanobody, a nanobody-HSA, a DART, a TandAb, a scDiabody, a scDiabody-CH3, scFv-CH-CL-scFv, a HSAbody, scDiabody-HAS, and a tandem-scFv.

In some embodiments of any of the proteins described herein, the protein includes two or more polypeptides. In some embodiments of any of the proteins described herein, the protein is selected from the group consisting of: an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′) 2-scFv2, a scFv-KIH, a Fab-scFv-Fc, a tetravalent HCAb, a scDiabody-Fc, a Diabody-Fc, a tandem scFv-Fc, an Intrabody, a dock and lock, an lmmTAC, an IgG-IgG conjugate, a Cov-X-Body, and a scFv1-PEG-scFV₂.

In some embodiments of any of the proteins described herein, the protein is an antibody that is an IgG antibody. In some embodiments of any of the proteins described herein, the IgG antibody is an IgG1, IgG2, IgG3, or IgG4 antibody. In some embodiments of any of the proteins described herein, the IgG antibody can comprise a lamba light chain or a kappa light chain. In some embodiments of any of the proteins described herein, the antibody is a monospecific antibody. In some embodiments of any of the proteins described herein, the antibody is a bispecific antibody. In some embodiments of any of the proteins described herein, the antigen-binding domain includes heavy chain variable domain CDRs of GFSFPDYY (SEQ ID NO: 2), IRDSANGYTT (SEQ ID NO: 3), and ARYSRYYGMDY (SEQ ID NO: 4), and light chain variable domain CDRs of QTVNYD (SEQ ID NO: 5), FAS (SEQ ID NO: 6), and QQDYSAPWT (SEQ ID NO: 7). In some embodiments of any of the proteins described herein, the antigen-binding domain includes a light chain variable domain sequence of: SIVMTQTPKILLVSAGDRVTITCKASQTVNYDVAWYQQKPGQSPKPVIYFASNRYTG VPDRFTGSGFGTDFTFTISTVQAEDLAVYFCQQDYSAPWTFGGGTKLEIK (SEQ ID NO: 8). In some embodiments of any of the proteins described herein, the antigen-binding domain includes a heavy chain variable domain sequence of:

(SEQ ID NO: 10)
EVKLVESGGGLVQPGGSLSLSCAASGFSFPDYYMSWVRQPPGKALEWLG
FIRDSANGYTTEYIASVKGRFTFSRDNSQSILYLQMNALRAEDSATYYC
ARYSRYYGMDYWGQGTSVTVSS.

In some embodiments of any of the proteins described herein, the antigen-binding domain includes heavy chain variable domain CDRs of GYTFTDYY (SEQ ID NO: 12), ISPNIGGA (SEQ ID NO: 13), and TRGGSSYPYFYAMDY (SEQ ID NO: 14), and light chain variable domain CDRs of QSLLYSSNQKKY (SEQ ID NO: 15), WAS (SEQ ID NO: 16), and QQFYSYPYT (SEQ ID NO: 17). In some embodiments of any of the proteins described herein, the antigen-binding domain includes a light chain variable domain sequence of: DIVMSQSPSSLAVSVGEKVTMSCKSSQSLLYSSNQKKYLAWYQQKPGQSPKLLIYW ASTRESGVPDRFTGSGSGTD FTLTISSVKAEDLAVYYCQQFYSYPYTFGGGTKLEINR (SEQ ID NO: 18). In some embodiments of any of the proteins described herein, the antigen-binding domain includes a heavy chain variable domain sequence of:

(SEQ ID NO: 20)
EVQLQQSGPEMVKPGTSVKISCKASGYTFTDYYINWVKQSHGKSLEWIG
DISPNIGGATYNPKFKGKAILTVDKSARTAYMELRSLTSEDSAVYCCTR
GGSSYPYFYAMDYWGQGTSVTVSS.

In some embodiments of any of the proteins described herein, the antigen-binding domain includes heavy chain variable domain CDRs of GFTFSDAW (SEQ ID NO: 22), IRGKVNNLAT (SEQ ID NO: 23), and LGRYDATYAMDY (SEQ ID NO: 24), and light chain variable domain CDRs of QSLVHSDGNTY (SEQ ID NO: 25), KLS (SEQ ID NO: 26), and SQSTHVPWT (SEQ ID NO: 27). In some embodiments of any of the proteins described herein, the antigen-binding domain includes a light chain variable domain sequence of: DVVMTQTPLSLPVSLGDQASISCRSSQSL VHSDGNTYLHWYLQKPGQSPKLLIYKLS NRFSGVPDRFSGSGSGTDF TLKISRVEAEDLGVYFCSQSTHVPWTFGGGTKLEIK (SEQ ID NO: 28). In some embodiments of any of the proteins described herein, the antigen-binding domain includes a heavy chain variable domain sequence of:

(SEQ ID NO: 30)
EVKLEESGGGLVQPGGSMKLSCAASGFTFSDAWMDWVRQSPEKGLEWVA
EIRGKVNNLATYYVESVKGRFTISRDDSKSSVYLQMNSLRAEDTGIYYC
LGRYDATYAMDYWGQGTSVTVSS.

In some embodiments of any of the proteins described herein, the antigen-binding domain includes heavy chain variable domain CDRs of GFSFTDYY (SEQ ID NO: 32), IRDSANGYTA (SEQ ID NO: 33), and ARYSRYYAMDY (SEQ ID NO: 34), and light chain variable domain CDRs of QSVNYD (SEQ ID NO: 35), FAS (SEQ ID NO: 36), and QQDYSSPWT (SEQ ID NO: 37). In some embodiments of any of the proteins described herein, the antigen-binding domain includes a light chain variable domain sequence of: FIVMTQTPKILLVSAGDRITITCKASQSVNYDVAWYQQKPGQSPKPVIYFASNRYTGV PDRFTGSGFGTDFTFTISTVQAEDLAVYFCQQDYSSPWTFGGGTKLEIK (SEQ ID NO: 38). In some embodiments of any of the proteins described herein, the antigen-binding domain includes a heavy chain variable domain sequence of:

(SEQ ID NO: 40)
EVKLVESGGGLVQPGGSLSLSCETSGFSFTDYYMIWVRQPPGKALEWLGF
IRDSANGYTAEYIASVKGRFTFSRDNSQSILYLQMNALRAEDSATYYCAR
YSRYYAMDYWGQGTSVTVAP.

In some embodiments of any of the proteins described herein, the antigen-binding domain includes heavy chain variable domain CDRs of GYTFTDYN (SEQ ID NO: 42), INPNNGGT (SEQ ID NO: 43), and ARKRGLGQAWLAY (SEQ ID NO: 44), and light chain variable domain CDRs of QSLLYSGNQKNY (SEQ ID NO: 45), GAS (SEQ ID NO: 46), and QNDHSYPLT (SEQ ID NO: 47). In some embodiments of any of the proteins described herein, the antigen-binding domain includes a light chain variable domain sequence of: DIVMTQSPSSRSVSAGEKVTMSCKSSQSLLYSGNQKNYLAWYQQKPGQPPKLLIYG ASTRESGVPDRFTGSGSGTDFTLTISSVQAEDLAVYYCQNDHSYPLTFGAGTKLELK (SEQ ID NO: 48). In some embodiments of any of the proteins described herein, the antigen-binding domain includes a heavy chain variable domain sequence of:

(SEQ ID NO: 50)
EVQLQQSGPELVKPGASVKIPCKASGYTFTDYNMDWVKQSHGKSLDWIGD
INPNNGGTIYNQKFKGKATLTVDKSSSTAYMELRSLTSEDTAVYYCARKR
GLGQAWLAYWGQGTLVTVSA.

In some embodiments of any of the proteins described herein, the antigen-binding domain includes heavy chain variable domain CDRs of GFTFSYAW (SEQ ID NO: 52), IRSKANNYAT (SEQ ID NO: 53), and MGRYDAAYGMDY (SEQ ID NO: 54), and light chain variable domain CDRs of QSLVHSNGITY (SEQ ID NO: 55), KVS (SEQ ID NO: 56), and SQSTHVPWT (SEQ ID NO: 57). In some embodiments of any of the proteins described herein, the antigen-binding domain includes a light chain variable domain sequence of: DVVMTQTPLSLPVSLGYQASISCRSSQSLVHSNGITYLHWYLQKPGQSPKLLIYKVSN RFSGVPDRFSGSGSGTDF TLKISRVEAEDLGVYFCSQSTHVPWTFGGGTKLEIK (SEQ ID NO: 58). In some embodiments of any of the proteins described herein, the antigen-binding domain includes a heavy chain variable domain sequence of:

(SEQ ID NO: 60)
EVKLEESGGGLVQPGGSMKLSCAASGFTFSYAWMDWVRQSPEKGLEWVAE
IRSKANNYATYYAESVKGRFTISRNDSKSSVYLQMNSLRIEDTGIYYCMG
RYDAAYGMDYWGQGTSVTVSS.

In some embodiments of any of the proteins described herein, the protein competitively binds to human ADAM8 with an antigen-binding domain including: (i) a light chain variable domain of SEQ ID NO: 8 and a heavy chain variable domain of SEQ ID NO: 10; (ii) a light chain variable domain of SEQ ID NO: 18 and a heavy chain variable domain of SEQ ID NO: 20; (iii) a light chain variable domain of SEQ ID NO: 28 and a heavy chain variable domain of SEQ ID NO: 30; (iv) a light chain variable domain of SEQ ID NO: 38 and a heavy chain variable domain of SEQ ID NO: 40; (v) a light chain variable domain of SEQ ID NO: 48 and a heavy chain variable domain of SEQ ID NO: 50; or (vi) a light chain variable domain of SEQ ID NO: 58 and a heavy chain variable domain of SEQ ID NO: 60.

In some embodiments of any of the proteins described herein, the antigen-binding domain binds to an epitope within human ADAM8 that includes at least one amino acid within the sequence of: CCNSTTCQLAEGAQCAHGTCCQECK (SEQ ID NO: 86) or RNRCCNSTTCQLAEGAQCAHGTCCQECK (SEQ ID NO: 104).

In some embodiments of any of the proteins described herein, the antigen-binding domain includes heavy chain variable domain CDRs of GFSFPDYY (SEQ ID NO: 2), IRDSANGYTT (SEQ ID NO: 3), and ARYSRYYGMDY (SEQ ID NO: 4), and light chain variable domain CDRs of QTVNYD (SEQ ID NO: 5), FAS (SEQ ID NO: 6), and QQDYSAPWT (SEQ ID NO: 7).

In some embodiments of any of the proteins described herein, the antigen-binding domain includes a light chain variable domain sequence of:

(SEQ ID NO: 8)
SIVMTQTPKILLVSAGDRVTITCKASQTVNYDVAWYQQKPGQSPKPVIY
FASNRYTGVPDRFTGSGFGTDFTFTISTVQAEDLAVYFCQQDYSAPWTF
GGGTKLEIK.

In some embodiments of any of the proteins described herein, the antigen-binding domain includes a heavy chain variable domain sequence of:

(SEQ ID NO: 10)
EVKLVESGGGLVQPGGSLSLSCAASGFSFPDYYMSWVRQPPGKALEWLG
FIRDSANGYTTEYIASVKGRFTFSRDNSQSILYLQMNALRAEDSATYYC
ARYSRYYGMDYWGQGTSVTVSS.

In some embodiments of any of the proteins described herein, the protein competitively binds to human ADAM8 with an antigen-binding domain including: (i) a light chain variable domain of SEQ ID NO: 18 and a heavy chain variable domain of SEQ ID NO: 20; (ii) a light chain variable domain of SEQ ID NO: 28 and a heavy chain variable domain of SEQ ID NO: 30; (iii) a light chain variable domain of SEQ ID NO: 38 and a heavy chain variable domain of SEQ ID NO: 40; (iv) a light chain variable domain of SEQ ID NO: 48 and a heavy chain variable domain of SEQ ID NO: 50; or (v) a light chain variable domain of SEQ ID NO: 58 and a heavy chain variable domain of SEQ ID NO: 60.

In some embodiments of any of the proteins described herein, the antigen-binding domain binds to an epitope within human ADAM8 that includes at least one amino acid within the sequence of:

(SEQ ID NO: 87)
LAEGAQCAHGTCCQECKVKPAGELCRPKKDMCDLEEFCDGRHPECPE
DAF.

In some embodiments of any of the proteins described herein, the antigen-binding domain includes heavy chain variable domain CDRs of GYTFTDYY (SEQ ID NO: 12), ISPNIGGA (SEQ ID NO: 13), and TRGGSSYPYFYAMDY (SEQ ID NO: 14), and light chain variable domain CDRs of QSLLYSSNQKKY (SEQ ID NO: 15), WAS (SEQ ID NO: 16), and QQFYSYPYT (SEQ ID NO: 17).

In some embodiments of any of the proteins described herein, the antigen-binding domain includes a light chain variable domain sequence of:

(SEQ ID NO: 18)
DIVMSQSPSSLAVSVGEKVTMSCKSSQSLLYSSNQKKYLAWYQQKPGQS
PKLLIYWASTRESGVPDRFTGSGSGTDFTLTISSVKAEDLAVYYCQQFY
SYPYTFGGGTKLEINR.

In some embodiments of any of the proteins described herein, the antigen-binding domain includes a heavy chain variable domain sequence of:

(SEQ ID NO: 20)
EVQLQQSGPEMVKPGTSVKISCKASGYTFTDYYINWVKQSHGKSLEWIG
DISPNIGGATYNPKFKGKAILTVDKSARTAYMELRSLTSEDSAVYCCTR
GGSSYPYFYAMDYWGQGTSVTVSS.

In some embodiments of any of the proteins described herein, the protein competitively binds to human ADAM8 with an antigen-binding domain including: (i) a light chain variable domain of SEQ ID NO:8 and a heavy chain variable domain of SEQ ID NO: 10; (ii) a light chain variable domain of SEQ ID NO: 38 and a heavy chain variable domain of SEQ ID NO: 40; or (iii) a light chain variable domain of SEQ ID NO: 48 and a heavy chain variable domain of SEQ ID NO: 50.

In some embodiments of any of the proteins described herein, the antigen-binding domain binds to an epitope within human ADAM8 that includes at least one amino acid within the sequence of: DCGPPEDCRNRCCNSTTCQ (SEQ ID NO: 88).

In some embodiments of any of the proteins described herein, the antigen-binding domain includes heavy chain variable domain CDRs of GFTFSDAW (SEQ ID NO: 22), IRGKVNNLAT (SEQ ID NO: 23), and LGRYDATYAMDY (SEQ ID NO: 24), and light chain variable domain CDRs of QSLVHSDGNTY (SEQ ID NO: 25), KLS (SEQ ID NO: 26), and SQSTHVPWT (SEQ ID NO: 27).

In some embodiments of any of the proteins described herein, the antigen-binding domain includes a light chain variable domain sequence of:

(SEQ ID NO: 28)
DVVMTQTPLSLPVSLGDQASISCRSSQSLVHSDGNTYLHWYLQKPGQSP
KLLIYKLSNRFSGVPDRFSGSGSGTDFTLKISRVEAEDLGVYFCSQSTH
VPWTFGGGTKLEIK.

In some embodiments of any of the proteins described herein, the antigen-binding domain includes a heavy chain variable domain sequence of:

(SEQ ID NO: 30)
EVKLEESGGGLVQPGGSMKLSCAASGFTFSDAWMDWVRQSPEKGLEWVA
EIRGKVNNLATYYVESVKGRFTISRDDSKSSVYLQMNSLRAEDTGIYYC
LGRYDATYAMDYWGQGTSVTVSS.

In some embodiments of any of the proteins described herein, the protein competitively binds to human ADAM8 with an antigen-binding domain including: (i) a light chain variable domain of SEQ ID NO: 8 and a heavy chain variable domain of SEQ ID NO: 10; (ii) a light chain variable domain of SEQ ID NO: 38 and a heavy chain variable domain of SEQ ID NO: 40; (iii) a light chain variable domain of SEQ ID NO: 48 and a heavy chain variable domain of SEQ ID NO: 50; or (iv) a light chain variable domain of SEQ ID NO: 58 and a heavy chain variable domain of SEQ ID NO: 60.

In some embodiments of any of the proteins described herein, the protein includes a human Fc domain.

In some embodiments of any of the proteins described herein, the protein further includes a conjugated toxin or a therapeutic agent.

Provided herein are nucleic acids encoding any of the proteins described herein, vectors including any of the nucleic acids described herein, and mammalian cells including any of the nucleic acids described herein or any of the vectors described herein.

Provided herein are methods of producing a protein that includes: (a) culturing a mammalian cell (e.g., any of the mammalian cells described herein) in a liquid culture medium under conditions sufficient to produce the protein; and (b) recovering the protein from the mammalian cell or the liquid culture medium. In some embodiments of any of the methods described herein, the method further includes: (c) isolating the protein recovered in step (b). In some embodiments of any of the methods described herein, the method further includes: (d) formulating the protein isolated in step (c) into a pharmaceutical composition.

Also provided herein are pharmaceutical compositions produced by any of the methods described herein.

Also provided herein are pharmaceutical compositions including a therapeutically effective amount of any of the proteins described herein.

Also provided herein are kits that include any of the proteins described herein or any of the pharmaceutical compositions described herein.

Also provided herein are methods for inhibiting migration and/or invasion of an ADAM8 expressing cell in a subject that include administering to the subject a therapeutically effective amount of any of the proteins described herein or any of the pharmaceutical compositions described herein.

In some embodiments of any of the methods described herein, the cell is an ADAM8-associated cancer cell. In some embodiments of any of the methods described herein, the ADAM8-associated cancer cell is from a cancer selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments of any of the methods described herein, the cancer cell is a triple negative breast cancer cell.

Provided herein are methods of decreasing the risk of developing a metastasis or developing an additional metastasis over a period of time in a subject identified as having an ADAM8-associated cancer that include administering to the subject a therapeutically effective amount of any of the proteins described herein or any of the pharmaceutical compositions described herein. In some embodiments of any of the methods described herein, the ADAM8-associated cancer is selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments of any of the methods described herein, the ADAM8-associated cancer is triple negative breast cancer. In some embodiments of any of the methods described herein, the metastasis or additional metastasis is one or more to a bone, lymph nodes, brain, lung, liver, skin, chest wall including bone, cartilage and soft tissue, abdominal cavity, contralateral breast, soft tissue, muscle, bone marrow, ovaries, adrenal glands, and pancreas. In some embodiments of any of the methods described herein, the period of time is about 1 month to about 5 years.

Provided herein are methods of inhibiting the growth of a solid tumor in a subject identified as having an ADAM8-associated cancer that include administering to the subject a therapeutically effective amount of any of the proteins described herein or any of the pharmaceutical compositions described herein.

In some embodiments of any of the methods described herein, the growth of a solid tumor is primary growth of a solid tumor. In some embodiments of any of the methods described herein, the growth of a solid tumor is recurrent growth of a solid tumor. In some embodiments of any of the methods described herein, the growth of a solid tumor is metastatic growth of a solid tumor. In some embodiments of any of the methods described herein, the ADAM8-associated cancer is selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, and bone cancer. In some embodiments of any of the methods described herein, the ADAM8-associated cancer is triple negative breast cancer.

Provided herein are methods of inhibiting the growth or proliferation of a hematological cancer in a subject identified as having an ADAM8-associated cancer that include administering to the subject a therapeutically effective amount of any of the proteins described herein or any of the pharmaceutical compositions described herein.

In some embodiments of any of the methods described herein, the hematological cancer is a leukemia. In some embodiments of any of the methods described herein, the hematological cancer is a lymphoma.

Also provided herein are methods of killing an ADAM8-associated cancer cell in a subject that include: administering to the subject a therapeutically effective amount of any of the proteins described herein or any of the pharmaceutical compositions described herein.

In some embodiments of any of the methods described herein, the ADAM8-associated cancer cell is from a cancer selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments of any of the methods described herein, the cancer cell is a triple negative breast cancer cell.

Provided herein are methods of treating an ADAM8-associated cancer in a subject that include: administering to a subject identified as having an ADAM8-associated cancer a therapeutically effective amount of any of the proteins described herein or any of the pharmaceutical compositions described herein.

In some embodiments of any of the methods described herein, the ADAM8-associated cancer is selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia.

In some embodiments of any of the methods described herein, the ADAM8-associated cancer is triple negative breast cancer.

In some embodiments of any of the methods described herein, the method further includes administering to the subject a therapeutically effective amount of a chemotherapeutic agent, a targeted therapy, or an immunotherapy. In some embodiments of any of the methods described herein, the chemotherapeutic agent is an antimetabolite, a plant alkaloid, a microtubule inhibitor, an anthracycline, a taxol, a platinum agent, or an alkylating agent. In some embodiments of any of the methods described herein, the targeted therapy is an angiogenesis or a kinase inhibitor. In some embodiments of any of the methods described herein, the immunotherapy is an inhibitor of PD-1, PD-L1, CTLA-4, LAG-3, CD70, CD80, ICOS, TIGIT, or IDO. In some embodiments of any of the methods described herein, the immunotherapy is a chimeric antigen receptor (CAR) T-cell therapy.

Provided herein are methods of identifying a protein including an antigen-binding domain that binds specifically to human ADAM8 and has the ability to inhibit both the metalloprotease activity and disintegrin activity of human ADAM8 that include: (a) identifying a protein including an antigen-binding domain that binds to an epitope within human ADAM8 that includes at least one amino acid within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

In some embodiments of any of the methods described herein, the method further includes confirming the ability of the identified protein to inhibit the metalloprotease activity and disintegrin activity of human ADAM8. In some embodiments of any of the methods described herein, step (a) includes identifying a protein including an antigen-binding domain that binds to an epitope within human ADAM8 that includes at least one amino acid within the sequence of: CCNSTTCQLAEGAQCAHGTCCQECK (SEQ ID NO: 86) or RNRCCNSTTCQLAEGAQCAHGTCCQECK (SEQ ID NO: 104).

In some embodiments of any of the methods described herein, step (a) includes identifying a protein including an antigen-binding domain that binds to an epitope within human ADAM8 that includes at least one amino acid within the sequence of:

(SEQ ID NO: 87)
LAEGAQCAHGTCCQECKVKPAGELCRPKKDMCDLEEFCDGRHPECPEDA
F.

In some embodiments of any of the methods described herein, step (a) includes identifying a protein including an antigen-binding domain that binds to an epitope within human ADAM8 that includes at least one amino acid within the sequence of:

(SEQ ID NO: 88)
DCGPPEDCRNRCCNSTTCQ.

Also provided herein are methods of diagnosing an ADAM8-associated cancer in a subject that include: (a) contacting a biological sample from the subject with any of the proteins described herein; (b) determining a level of the protein specifically bound to the biological sample; and (c) identifying the subject as having an ADAM8-associated cancer if the level of the protein specifically bound to the biological sample is elevated as compared to a control level. In some embodiments of any of the methods described herein, the biological sample is a biopsy tissue sample. In some embodiments of any of the methods described herein, the biological sample is not a fixed tissue sample. In some embodiments of any of the methods described herein, the biological sample is a fresh, frozen tissue sample.

Some embodiments of any of the methods described herein further include, prior to step (a), trypsinizing the biological sample. In some embodiments of any of the methods described herein, step (b) comprises the use of fluorescence-activated cell sorting.

In some embodiments of any of the methods described herein, the biological sample is a fixed tissue sample. In some embodiments of any of the methods described herein, the fixed tissue sample is a formalin-fixed paraffin-embedded (FFPE) tissue sample. Some embodiments of any of the methods described herein further include, before step (a), fixing the tissue sample. Some embodiments of any of the methods described herein further include, before step (a), decrosslinking the fixed tissue sample. In some embodiments of any of the methods described herein, the decrosslinking of the fixed tissue sample is performed using a Tris-EDTA-based, basic buffer. In some embodiments of any of the methods described herein, the decrosslinking is performed for 40 to 80 minutes at a temperature of about 65° C. to about 95° C. In some embodiments of any of the methods described herein, the decrosslinking of the fixed tissue sample is performed using an alkaline endopeptidase.

In some embodiments of any of the methods described herein, the alkaline endopeptidase is a serine protease.

In some embodiments of any of the methods described herein, the protein comprises a detectable label. In some embodiments of any of the methods described herein, step (b) comprises detecting the detectable label. In some embodiments of any of the methods described herein, the detectable label is a heavy metal, a fluorophore, or an enzyme.

In some embodiments of any of the methods described herein, the protein does not comprise a detectable label, and step (b) comprises the use of an agent that binds specifically to the protein specifically bound to the biological sample. In some embodiments of any of the methods described herein, the agent comprises an antibody. In some embodiments of any of the methods described herein, the agent comprises a detectable label. In some embodiments of any of the methods described herein, step (b) comprises detecting the detectable label. In some embodiments of any of the methods described herein, the detectable label comprises a heavy metal, a fluorophore, or an enzyme.

In some embodiments of any of the methods described herein, step (b) comprises imaging the biological sample. In some embodiments of any of the methods described herein, step (b) comprises performing immunohistochemistry or immunofluorescence.

In some embodiments of any of the methods described herein, the biological sample is a liquid biopsy sample. In some embodiments of any of the methods described herein, the liquid biopsy sample is a blood sample, a cerebrospinal fluid sample, a pleural effusion sample or an ascites sample. Some embodiments of any of the methods described herein further include obtaining the liquid biopsy sample from the subject. Some embodiments of the methods described herein further include, before step (a), concentrating the cells in the liquid biopsy sample. Some embodiments of any of the methods described herein further include, before step (a), fixing the liquid biopsy sample. In some embodiments of any of the methods described herein, step (b) comprises performing fluorescence-activated cell sorting. Some embodiments of any of the methods described herein further include, before step (a), lysing cells in the liquid biopsy sample. In some embodiments of any of the methods described herein, step (b) comprises performing an enzyme-linked immunosorbent assay. In some embodiments of any of the methods described herein, the protein comprises a detectable label. In some embodiments of any of the methods described herein, step (b) comprises detecting the detectable label. In some embodiments of any of the methods described herein, the detectable label is a heavy metal, a fluorophore, or an enzyme. In some embodiments of any of the methods described herein, the protein does not comprise a detectable label, and step (b) comprises the use of an agent that binds specifically to the protein specifically bound to the biological sample. In some embodiments of any of the methods described herein, the agent comprises an antibody. In some embodiments of any of the methods described herein, the agent comprises a detectable label.

Some embodiments of any of the methods described herein further include, after step (c), (d) selecting a therapeutically effective amount of the protein used in step (a) for treatment of the subject identified as having an ADAM8-associated cancer. Some embodiments of any of the methods described herein further include, after step (c), (d) administering a therapeutically effective amount of the protein used in step (a) to the subject identified as having an ADAM8-associated cancer.

Some embodiments of any of the methods described herein further include, after step (c), (d) administering a therapeutically effective amount of a chemotherapeutic agent, a targeted therapy, or an immunotherapy. In some embodiments of any of the methods described herein, the chemotherapeutic agent is an antimetabolite, a plant alkaloid, a microtubule inhibitor, an anthracycline, a taxol, a platinum agent, or an alkylating agent. In some embodiments of any of the methods described herein, the targeted therapy is an angiogenesis or a kinase inhibitor. In some embodiments of any of the methods described herein, the immunotherapy is an inhibitor of PD-1, PD-L1, CTLA-4, LAG-3, CD70, CD80, ICOS, TIGIT, or IDO. In some embodiments of any of the methods described herein, the immunotherapy is a chimeric antigen receptor (CAR) T-cell therapy. Some embodiments of any of the methods described herein further include, after step (c), determining the stage of the ADAM8-associated cancer in the subject based on the level of the protein specifically bound to the biological sample.

In some embodiments of any of the methods described herein, the subject is suspected of having an ADAM8-associated cancer. In some embodiments of any of the methods described herein, the subject is presenting with one or more symptoms of an ADAM8-associated cancer. In some embodiments of any of the methods described herein, the ADAM8-associated cancer is a cancer selected from the group of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments of any of the methods described herein, the ADAM8-associated cancer is triple negative breast cancer. In some embodiments of any of the methods described herein, the ADAM8-associated cancer is a hematological cancer. In some embodiments of any of the methods described herein, the hematological cancer is a leukemia. In some embodiments of any of the methods described herein, the hematological cancer is lymphoma.

In some embodiments of any of the methods described herein, the biological sample is obtained from a metastasis. In some embodiments of any of the methods described herein, the metastasis is obtained from bone, lymph node, brain, lung, liver, skin, chest wall (including bone, cartilage and soft tissue), abdominal cavity, contralateral breast, soft tissue, muscle, bone marrow, ovaries, adrenal glands, and pancreas.

Also provided herein are methods of determining the efficacy of treatment of an ADAM8-associated cancer in a subject that include: (a) contacting a first biological sample obtained from a subject having an ADAM8-associated cancer at first time point with any of the proteins described herein; (b) determining a first level of the protein specifically bound to the first biological sample; (c) contacting a second biological sample obtained from the same subject at a second time point with the protein, wherein the subject has been administered a treatment against an ADAM8-associated cancer between the first and second time points; (d) determining a second level of the protein specifically bound to the second biological sample; and (e) determining the treatment as being effective in a subject having a decreased second level as compared to the first level, or determining the treatment as not being effective in a subject having about the same or an increased second level as compared to the first level.

In some embodiments of any of the methods described herein, the subject has previously been diagnosed as having an ADAM8-associated cancer. Some embodiments of any of the methods described herein further include recording the determination in step (e) in the subject's medical record.

In some embodiments of any of the methods described herein, step (e) comprises determining the treatment as being effective in the subject. Some embodiments of any of the methods described herein further include, after step (e), selecting one or more additional doses of the treatment for the subject. Some embodiments of any of the methods described herein further include, after step (e), administering one or more additional doses of the treatment to the subject.

In some embodiments of any of the methods described herein, step (e) comprises determining the treatment was not effective in the subject. Some embodiments of any of the methods described herein further include, after step (e), selecting an alternative treatment for the subject. Some embodiments of any of the methods described herein further include, after step (e), administering an alternative treatment to the subject.

Some embodiments of any of the methods described herein further include administering the treatment to the subject between the first and second time points.

In some embodiments of any of the methods described herein, the treatment comprises the protein used in steps (a) and (c). In some embodiments of any of the methods described herein, the treatment comprises the protein conjugated to a cytotoxin or therapeutic agent. In some embodiments of any of the methods described herein, treatment comprises a chemotherapeutic agent, a targeted therapy, or an immunotherapy. In some embodiments of any of the methods described herein, the chemotherapeutic agent is an antimetabolite, a plant alkaloid, a microtubule inhibitor, an anthracycline, a taxol, a platinum agent, or an alkylating agent. In some embodiments of any of the methods described herein, the targeted therapy is an angiogenesis or a kinase inhibitor. In some embodiments of any of the methods described herein, the immunotherapy is an inhibitor of PD-1, PD-L1, CTLA-4, LAG-3, CD70, CD80, ICOS, TIGIT, or IDO. In some embodiments of any of the methods described herein, the immunotherapy is a chimeric antigen receptor (CAR) T-cell therapy.

In some embodiments of any of the methods described herein, the first and second biological samples are tissue samples. In some embodimens of any of the methods described herein, the tissue samples are biopsy tissue samples. In some embodiments of any of the methods described herein, the tissue samples are not fixed tissue samples. In some embodiments of any of the methods described herein, the tissue sample is a fresh, frozen tissue sample. Some embodiments of any of the methods described herein further include, prior to step (a), trypsinizing the tissue samples. In some embodiments of any of the methods described herein, step (b) comprises the use of fluorescence-activated cell sorting.

In some embodiments of any of the methods described herein, the tissue samples are fixed tissue samples. In some embodiments of any of the methods described herein, the fixed tissue samples are formalin-fixed paraffin-embedded (FFPE) tissue samples. Some embodiments of any of the methods described herein further include, before step (a), fixing the tissue samples. Some embodiments of any of the methods described herein further include, before step (a), decrosslinking the fixed tissue samples. In some embodiments of any of the methods described herein, the decrosslinking of the fixed tissue samples is performed using a Tris-EDTA-based, basic buffer. In some embodiments of any of the methods described herein, the decrosslinking is performed for 40 to 80 minutes at a temperature of about 65° C. to about 95° C. In some embodiments of any of the methods described herein, the decrosslinking of the fixed tissue sample is performed using an alkaline endopeptidase. In some embodiments of any of the methods described herein, the alkaline endopeptidase is a serine protease.

In some embodiments of any of the methods described herein, the protein comprises a detectable label. In some embodiments of any of the methods described herein, step (b) comprises detecting the detectable label. In some embodiments of any of the methods described herein, the detectable label is a heavy metal, a fluorophore, or an enzyme.

In some embodiments of any of the methods described herein, the protein does not comprise a detectable label, and steps (b) and (d) comprises the use of an agent that binds specifically to the protein specifically bound to the first and second biological samples, respectively. In some embodiments of any of the methods described herein, the agent comprises an antibody. In some embodiments of any of the methods described herein, the agent comprises a detectable label. In some embodiments of any of the methods described herein, steps (b) and (d) comprise detecting the detectable label. In some embodiments of any of the methods described herein, the detectable label comprises a heavy metal, a fluorophore, or an enzyme.

In some embodiments of any of the methods described herein, steps (b) and (d) comprise imaging the first and second biological samples. In some embodiments of any of the methods described herein, the determining in steps (b) and (d) comprise performing immunohistochemistry or immunofluorescence.

In some embodiments of any of the methods described herein, the first and second biological samples are liquid biopsy samples. In some embodiments of any of the methods described herein, the liquid biopsy samples are blood samples, cerebrospinal fluid samples, pleural effusion samples or ascites samples.

Some embodiments of any of the methods described herein further include concentrating cells in the biological sample(s). Some embodiments of any of the methods described herein further include, before steps (a) and (c), fixing the biological samples. In some embodiments of any of the methods described herein, steps (b) and (d) comprise performing fluorescence-activated cell sorting. Some embodiments of any of the methods described herein further include, before steps (a) and (c), lysing cells in the liquid biopsy sample. In some embodiments of any of the methods described herein, steps (b) and (d) comprise performing an enzyme-linked immunosorbent assay. In some embodiments of any of the methods described herein, the protein comprises a detectable label. In some embodiments of any of the methods described herein, step (b) comprises detecting the detectable label. In some embodiments of any of the methods described herein, the detectable label is a heavy metal, a fluorophore, or an enzyme.

In some embodiments of any of the methods described herein, the protein does not comprise a detectable label, and steps (b) and (d) comprise the use of an agent that binds specifically to the protein specifically bound to the biological sample. In some embodiments of any of the methods described herein, the agent comprises an antibody. In some embodiments of any of the methods described herein, the agent comprises a detectable label.

Also provided herein are kits that include any of the proteins described herein or any of the pharmaceutical compositions described herein. Also provided herein are kits that include any of the proteins described herein and instructions for performing any of the methods described herein. Some embodiments of any of the kits described herein further include one or more of: a positive and/or a negative control sample(s); a negative control antibody; an agent that binds specifically to the protein; and a reagent necessary for detection of a tag or enzyme activity.

The term “a” and “an” refers to one or to more than one (i.e., at least one) of the grammatical object of the article. By way of example, “a protein” encompasses one protein and more than one protein.

The term “conservative mutation” refers to a mutation that does not change the amino acid encoded at the site of the mutation (due to codon degeneracy).

Modifications can be introduced into a nucleotide sequence by standard techniques known in the art, such as site-directed mutagenesis, PCR-mediated mutagenesis, and CRISPR technology. Conservative amino acid substitutions are ones in which the amino acid residue is replaced with an amino acid residue having a similar side chain. Families of amino acid residues having similar side chains have been defined in the art. These families include amino acids with basic side chains (e.g., histidine, lysine and arginine), acidic side chains (e.g., glutamic acid and aspartic acid), uncharged polar side chains (e.g., asparagine, glycine, glutamine, serine, threonine, tyrosine, cysteine, and tryptophan), nonpolar side chains (e.g., alanine, valine, leucine, isoleucine, proline, phenylalanine, and methionine), beta-branched side chains (e.g., threonine, valine and isoleucine), and aromatic side chains (e.g., tyrosine, phenylalanine, tryptophan, and histidine).

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Unless otherwise specified, a “nucleotide sequence encoding an amino acid sequence” includes all nucleotide sequences that are degenerate versions of each other and thus encode the same amino acid sequence.

The terms “protease” and “proteinase” are used interchangeably and refer to an enzyme that cleaves proteins into smaller peptides. Non-limiting examples of proteases and proteinases include ADAM8 metalloprotease or ADAM8 metalloproteinase. A metalloproteinase can be abbreivated as MP.

The term “transfect”, “transformed” or “transduced” refers to a process by which exogenous nucleic acid is transferred or introduced into a cell. A “transfected”, “transformed”, or “transduced” mammalian cell is one that has been transfected, transformed, or transduced with exogenous nucleic acid and can lead to ectopic or exogenous expression of protein.

The term “endogenous” expression refers to proteins that are expressed naturally from the mammalian cell genome.

The term “expression” refers to the transcription and/or translation of a particular nucleotide sequence encoding a protein.

The term “subject” refers to any mammal. In some embodiments, the subject is a rabbit, a sheep, a goat, a pig, a canine (e.g., a dog), a feline (e.g., a cat), a rodent (e.g., a mouse, a guinea pig, a hamster, or a rat), an equine (e.g., a horse), a bovine, simian (e.g., a monkey (e.g., a rhesus monkey, a cynomolgus monkey, a marmoset, or a baboon), or an ape (e.g., a gorilla, a chimpanzee, an orangutan, or a gibbon), or a non-human primate), or a human. In some embodiments, the subject has or is at risk of developing cancer. In some embodiments, the subject or “subject suitable for treatment” may be a non-human mammal, especially mammals that are conventionally used as models for demonstrating therapeutic efficacy in humans may be used, such as mice, rabbits, dogs, primates, or rats.

A treatment is “therapeutically effective” when it results in a reduction in one or more of the number, severity, and frequency of one or more symptoms of a disease state (e.g., cancer) in a subject (e.g., a human). In some embodiments, a therapeutically effective amount of a protein or a pharmaceutical composition can inhibit the growth of cancer, e.g., tumors and/or tumor cells, improve overall survival of a patient suffering from or at risk for cancer, and/or improve the outcome of other cancer treatments. In some embodiments, a treatment can reduce cancer progression, reduce the histopathological severity of a cancer, and/or reduce the risk of re-occurrence of a cancer.

The term “cancer” includes a variety of cancerous growths, e.g., primary tumors, recurrent tumors, metastatic tumors, and malignantly transformed cells, tissues, or organs, irrespective of histopathologic type or stage of invasiveness.

An “ADAM8-associated cancer” refers to a cancer characterized by a population of cancer cells that expressed increased levels and/or activity of ADAM8, e.g., compared to a control cell. For example, an ADAM8-associated cancer can be selected from the group of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma, and leukemia. In some examples, an ADAM8-associated cancer is a triple negative breast cancer.

An “ADAM8-associated cancer cell” refers to a cancer cell of an ADAM8-associated cancer. For example, an ADAM8 associated cancer cell can be from an ADAM8 associated cancer selected from the group of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma, and leukemia. In some examples, an ADAM8-associated cancer cell is a triple negative breast cancer cell.

A metastatic tumor can arise from a multitude of primary tumor types, including but not limited to those of breast, lung, colon, bone, prostate, and liver origin. Metastases develop, e.g., when tumor cells shed from a primary tumor, adhere to vascular endothelium, invade the vasculature, penetrate into surrounding tissues, and grow to form independent tumors at sites separate from a primary tumor. In some examples, a metastatic tumor may form after a period of latency and/or dormancy (e.g., months, or years).

The term “triple negative breast cancer” or “TNBC” refers to a breast cancer characterized by a population of breast cancer cells that are estrogen receptor (ER)-negative, progesterone receptor (PR)-negative and human epidermal growth factor receptor 2 (HER2)-negative. In some examples, a TNBC is also characterized by a population of breast cancer cells that have a mutation in the breast cancer gene 1 (BRCA1) and/or breast cancer gene 2 (BRCA2) gene. In some examples, a breast cancer is determined to be a TNBC based on immunohistochemistry staining of a breast tissue biopsy sample.

The term “population” when used before a noun means two or more of the specific noun. For example, the phrase “a population of cancer cells” means “two or more cancer cells”. Non-limiting examples of cancer cells are described herein.

A “chemotherapeutic agent” refers to a chemical compound useful in the treatment of cancer. Chemotherapeutic agents include, e.g., “anti-hormonal agents” or “endocrine therapeutics,” which act to regulate, reduce, block, or inhibit the effects of hormones that can promote the growth of cancer. Non-limiting examples of chemotherapeutic agents include: alkylating agents, plant alkaloid, microtubule inhibitors, anthracyclines (e.g., doxorubicin), taxols (e.g., Paclitaxel), platinum agents, antimetabolites, e.g., purine antagonists, pyrimidine antagonists, and/or folate antagonists; antibiotics, e.g., bleomycin, and/or mitomycin; inorganic ions, e.g, cisplatin; and nitrosureas. Additional classes, subclasses, and examples of chemotherapeutic agents are known in the art.

Individuals considered at risk for developing metastasis may benefit from the present disclosure, e.g., because prophylactic treatment can begin before there is any evidence and/or diagnosis of metastasis. In some examples, the subject of any of the methods described herein may have previously received cancer treatment (e.g., any of the cancer treatments described herein).

Skilled practitioners will appreciate that a patient can be diagnosed, e.g., by a medical professional, e.g., a physician or a nurse (or a veterinarian, as appropriate for the subject being diagnosed), using any method known in the art, e.g., by assessing a patient's medical history, employing imaging techniques, or performing diagnostic tests.

Skilled practitioners will also appreciate that treatment need not be administered to a subject by the same individual who diagnosed the subject (or the same individual who prescribed the treatment for the subject). Treatment can be administered (and/or administration can be supervised), e.g., by the diagnosing and/or prescribing individual, and/or any other individual (e.g., infusion nurse), including the subject her/him/themselves (e.g., where the subject is capable of self-administration).

Also contemplated by the present disclosure is administration of a protein (e.g., any of the proteins described herein), a pharmaceutical composition (e.g., any of the pharmaceutical compositions described herein) to a subject in combination with a chemotherapeutic agent (e.g., any of the chemotherapeutic agents described herein), a targeted therapy (e.g., any of the targeted therapies described herein), an immunotherapy (e.g., any of the immunotherapies described herein), or a radiation therapy (e.g., using γ-radiation, electron beams, neutron beams, and/or radioactive isotopes). In some embodiments of any of the methods described herein, the chemotherapeutic agent is an antimetabolite, a plant alkaloid, a microtubule inhibitor, an anthracycline, a taxol, a platinum agent, or an alkylating agent. In some embodiments of any of the methods described herein, the targeted therapy is an angiogenesis or a kinase inhibitor. In some embodiments of any of the methods described herein, the immunotherapy is an inhibitor of PD-1, PD-L1, CTLA-4, LAG-3, CD70, CD80, ICOS, TIGIT, or IDO. In some embodiments of any of the methods described herein, the immunotherapy is a chimeric antigen receptor (CAR) T-cell therapy.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features, and advantages of the disclosure will be apparent from the following detailed description and figures, and from the claims.

### BRIEF DESCRIPTION OF DRAWINGS

FIG. 1A is a schematic representation of human ADAM8, its various domains, forms and activities. Synthesized as an inactive “Proform”, the transmembrane ADAM8 protein can dimerize or multimerize and autocatalytically clip off its Prodomain. This processing generates a 90 kDa “Active” membrane-anchored form with Metalloproteinase (MP) and Disintegrin (DI) activities. Active ADAM8 can further be clipped to a 60 kDa “Remnant” form, which lacks the MP domain and therefore has only DI activity. The molecular weight and activity status of each form is indicated.

FIG. 1B is a schematic representation of the MP and DI functions of “Active ADAM8” using example substrates CD23 and Integrin. MP function: proteolysis of cell surface substrates including receptors (e.g., CD23) and precursors of angiogenic factors, cytokines and immunoglobulins, cell adhesion molecules and extracellular matrix (ECM) components. Substrate recognition is mediated via sequences within the MP domain and the hypervariable region (HVR) of the Cysteine-rich domain (CRD). DI function: binding to ECM components and cell surface molecules (e.g., integrins). The binding of ADAM8 monomer to an integrin has been attributed to a hairpin loop within the DI domain, containing the sequence CRPKKDMCD (aa466-474) (SEQ ID NO: 89), which leads to integrin activation. The Remnant form also contains this sequence and displays DI activity (not shown).

FIG. 2 is a diagram showing the steps used to generate highly specific human ADAM8 dual MP and DI domain inhibitory mouse monoclonal ADP antibodies and to select ADP2 and ADP13 as lead candidate therapeutics. Anti-ADAM8 antibodies were generated using the standard hybridoma method (indicated in top black box) followed by a complex novel screening strategy (bottom black box). The three phases of screening, performed to isolated antibodies with the desired functional characteristics, are indicated. Dotted boxes show specific methods and features described in U.S. Patent Publication No. 2016/0130365 as necessary for the generation of a successful Triple-Negative Breast Cancer (TNBC) therapeutic antibody.

FIG. 3 is a table showing the isotype, subclass and type of light chain for each ADP antibody.

FIG. 4 is a table that demonstrates the ability of each ADP to bind to native ADAM8 ectopically expressed on the cell surface. Fluorescent Activated Cell Sorting (FACS) analysis was performed using HEK293 cells stably overexpressing full-length ADAM8 (HEK293-ADAM8) or empty vector DNA (HEK293-Empty Vector), as a control for binding specificity, as a function of decreasing antibody concentration. Mean Fluorescent Intensity (MFI) indicates the extent of binding of each antibody. As negative and positive controls normal mouse IgG (mIgG) and a test bleed sample from a mouse injected with recombinant human ADAM8 (rHuADAM8) were used, respectively.

FIG. 5 is a table showing the binding affinity of each ADP antibody to rHuADAM8 determined through Enzyme-linked immunosorbent assays (ELISA) and surface plasmon resonance (Biacore™) assays. The half maximal effective concentration (EC₅₀) [nM] for each ADP obtained in ELISA assays is presented. Furthermore, binding kinetics, such as association rate constant (kₐ), dissociation rate constant (k_{d}) and equilibrium dissociation constant (KD) obtained through Biacore surface plasmon resonance studies are shown.

FIG. 6 is a table showing the epitope binning analysis of ADP antibodies. The binding of each ADP [indicated in the “Antibody” column] to rHuADAM8 was challenged with excess of a second competitor ADP (indicated in the top row of the table). Percentages indicate extent of cross-competition for ADAM8. High levels of cross-competition, defined as equal to or greater than 75% (marked in black), delineate five ADP epitope clusters, which have been labelled Epitope 1 through 5. White boxes are the values obtained for competition with self.

FIG. 7 is a diagram showing how the 18 ADPs can be separated into 5 epitope groups based on the epitope binning analysis in FIG. 6, four of which partially overlap.

FIG. 8 is a table demonstrating that all ADP antibodies have specific binding to ADAM8 and do not cross-react with related ADAM proteins ADAM9, ADAM12 or ADAM15. ELISA assays were performed with recombinant ADAM proteins. Normal mouse IgG was used as a negative control (NC) and indicates the level of general non-specific binding. A test bleed sample from a rHuADAM8 injected mouse was used as a positive control (PC).

FIG. 9 is a bar graph representation of the results presented in FIG. 8, demonstrating ADPs bind ADAM8 specifically. That is, all ADP antibodies bind to ADAM8, but not to related ADAM proteins ADAM9, ADAM12 or ADAM15 in ELISA assays performed with recombinant ADAM proteins. Normal mouse IgG was used as a NC and indicates the level of general non-specific binding. A test bleed sample from a rHuADAM8 injected mouse was used as a PC.

FIG. 10 is a table showing the dual antagonist MP and DI inhibitory activity of ADP antibodies in cell-based functional assays. MP activity was assessed using a CD23 cleavage assay. Percent inhibition of MP activity was calculated as a decrease in the cleavage of the ADAM8 target protein CD23 in the supernatant of HEK293 cells co-expressing CD23 and full-length ADAM8, following ADP treatment vs treatment with isotype-matched control IgG (set to 100%). DI activity was assessed in two assays: binding of α9β1 integrin to ADAM8 and transendothelial migration (TEM), both processes mediated through an active DI domain. Percent inhibition of DI activity was calculated as a decrease in the adhesion of Chinese Hamster Ovary (CHO) cells expressing α9β1 integrin to rHuADAM8 and a decrease in the ability of MDA-MB-231 ADAM8-expressing TNBC cells to move through a layer of human umbilical vein endothelial cells (HUVEC), following treatment with ADP vs control IgG (set to 100%). Inhibitory activity of the prototype dual antagonist MAB1031 antibody, that was used in U.S. Patent Publication No. 2016/0130365, was also determined. Mean±standard deviation (S.D.) from 3 independent experiments is given in each case.

FIG. 11 is a bar graph representation of the results seen in FIG. 10 showing the MP inhibitory activity of ADP antibodies in CD23 cleavage cell-based functional assays vs their isotype-matched control IgGs. Values are Mean±S.D. from 3 independent experiments. Dashed line represents the level of activity seen with the prototype MAB1031.

FIG. 12A is a bar graph representation of the results shown in FIG. 10 of the DI inhibitory activity of ADP antibodies in assays measuring α9β1 integrin binding to ADAM8. Mean±S.D. from 3 independent experiments is given. Dashed line represents the level of activity seen with MAB1031.

FIG. 12B is a bar graph representation of the results shown in FIG. 10 of the DI inhibitory activity of ADP antibodies in assays measuring TEM. Mean±S.D. from 3 independent experiments is given. Dashed line represents the level of activity seen with MAB1031.

FIG. 13 is a graphic representation of the ability of ADP13 to inhibit growth of pre-existing ADAM8-positive MDA-MB-231 TNBC cell line-derived primary orthotopic tumors in a dose-dependent manner when compared to its isotype-matched control IgG1. Tumor volume (Mean±S.E.M.) over time is presented. n=number of animals/group. **, P-value<0.05 for IgG1 (10 mg/kg) vs ADP13 (3 mg/kg); #, P-value<0.05 for IgG1 (10 mg/kg) vs ADP13 (10 mg/kg) using a Student's t-test. Treatment with 10 mg/kg ADP13 resulted in a significant 40% reduction in pre-existing primary tumor growth. A higher dose of 30 mg/kg ADP13 did not provide any additional benefit (not shown).

FIG. 14A is a graphic representation showing the single dose in vivo comparison of the ability of ADP2 to inhibit pre-existing MDA-MB-231 cell line-derived TNBC orthotopic tumor growth in mice, which identified ADP2 as a lead inhibitory antibody. Tumor volume (mean±S.E.M. for ADP2 vs its matched control) over time is shown. n=number of animals/group. *P-value<0.05 using a Student's t-test. Treatment with ADP2 resulted in significant reductions in tumor volume of 47%.

FIG. 14B is a graphic representation showing the single dose in vivo comparison of the ability of ADP3 to inhibit pre-existing MDA-MB-231 cell line-derived TNBC orthotopic tumor growth in mice. Tumor volume (mean±S.E.M. for ADP3 vs its matched control) over time is shown. n=number of animals/group. *P-value<0.05 using a Student's t-test. Treatment with ADP3 resulted in significant reductions in tumor volume of 28%.

FIG. 14C is a graphic representation showing the single dose in vivo comparison of the ability of ADP13 to inhibit pre-existing MDA-MB-231 cell line-derived TNBC orthotopic tumor growth in mice, which identified ADP13 as a lead inhibitory antibody. Tumor volume (mean±S.E.M. for ADP13 vs its matched control) over time is shown. n=number of animals/group. *P-value<0.05 using a Student's t-test. Treatment with ADP13 resulted in significant reductions in tumor volume of 52%.

FIG. 14D is a graphic representation showing the single dose in vivo comparison of the ability of ADP19 to inhibit pre-existing MDA-MB-231 cell line-derived TNBC orthotopic tumor growth in mice. Tumor volume (mean±S.E.M. for ADP19 vs its matched control) over time is shown. n=number of animals/group.

FIG. 15 is a graphic representation of the ability of ADP2 to inhibit pre-existing MDA-MB-231 TNBC cell line-derived primary orthotopic tumor growth in a dose-dependent manner when compared to its isotype-matched control IgG2b. Tumor volume (Mean±S.E.M.) over time is presented. n=number of animals/group. *, P-value<0.05 for IgG2b (10 mg/kg) vs ADP2 (1 mg/kg); **, P-value<0.05 for IgG2b (10 mg/kg) vs ADP2 (3 mg/kg); #, P-value<0.05 for IgG2b (10 mg/kg) vs ADP2 (10 mg/kg) using a Student's t-test. Treatment with 10 mg/kg ADP2 resulted in a significant 50% reduction in pre-existing primary tumor growth. A higher dose of 30 mg/kg ADP2 did not provide any additional benefit (not shown).

FIG. 16A is an image of a Western blot showing effective knockdown of ADAM8 protein expression in SUM149 TNBC cells treated with ADAM8-specific siRNAs vs a scrambled control siRNA (siCtrl). Position of marker proteins and ADAM8 forms are indicated. siA8-1: siADAM8 RNA-1 (SEQ ID NO: 101); siA8-2: siADAM8 RNA-2 (SEQ ID NO: 102).

FIG. 16B is a bar graph showing inhibition of anchorage independent growth in an agarose colony formation assay of SUM149 cells transfected with siA8-1 and siA8-2 vs siCtrl. Bar graph is representative of two independent experiments with similar results. siA8-1: siADAM8 RNA-1 (SEQ ID NO: 101); siA8-2: siADAM8 RNA-2 (SEQ ID NO: 102); *, P-value<0.05 by Student's t-test.

FIG. 16C is a set of images showing invasion of SUM149 cells transfected with siA8-1 and siA8-2 vs siCtrl through Matrigel.

FIG. 16D is a bar graph showing formation of spheroids by SUM149 cells transfected with siA8-1 and siA8-2 vs siCtrl grown in suspension for 5 or 7 days. Bar graph is representative of two independent experiments with similar results. siA8-1: siADAM8 RNA-1 (SEQ ID NO: 101); siA8-2: siADAM8 RNA-2 (SEQ ID NO: 102).

FIG. 17A is an image of a Western blot showing cleavage of CD23 in the presence of ADP13 or control isotype matched IgG1 (top panel) in the cell supernatant of SUM149 cells co-expressing CD23 and ADAM8. Bottom panel is a bar graph showing band intensity quantified. Bar graph and image are representative of two independent experiments in each case.

FIG. 17B is a bar graph showing DI activity using a SUM149 cancer cell-to-HUVEC endothelial cell adhesion assay. Percent relative adhesion of SUM149 cells in the absence of HUVEC cells or in the presence of HUVEC cells and either ADP13, control isotype matched IgG1 or no treatment (untreated) were assessed. Bar graph is representative of two independent experiments. **, P-value<0.05 by Student's t-test.

FIG. 18 is a graph showing tumor volume (cm³; Mean±S.E.M.) over time (in days) of SUM149 primary orthotopic tumors implanted in the female NOD/SCID mice (n=number of animals/group). Mice were treated with the indicated doses (1.5 mg/kg, 4.5 mg/kg) of ADP13 or control IgG1 in intraperitoneal (i.p.) injection 2×/week from the time of implantation. *, P-value<0.05 by Student's t-test.

FIG. 19 is a graph showing tumor volume (cm³; Mean±S.E.M.) over time (in days) of SUM149 primary orthotopic tumors implanted in the female NOD/SCID mice (n=number of animals/group). Mice with 50-75 mm³ tumors were treated with the indicated doses (5 mg/kg, 10 mg/kg, 20 mg/kg) of ADP13 or control IgG1 in i.p. injection 3×/week (n=number of animals/group). *, P-value<0.05 for IgG1 Control (Ctrl) vs ADP13 (5 mg/kg); **, P-value<0.05 for IgG1 Ctrl vs ADP13 (10 mg/kg); #, P-value<0.05 for IgG1 Ctrl vs ADP13 (20 mg/kg) using a Student's t-test.

FIG. 20A is a graph showing the number of cells demonstrating fluorescence using FACS analysis in HEK293-ADAM33 (A33) and control HEK293-Empty Vector (EV) after exposure to ADP2 antibody. An ADAM33 antibody was used as a positive control. Both antibodies were matched with their respective control isotype IgG.

FIG. 20B is a graph showing the number of cells demonstrating fluorescence using FACS analysis in HEK293-ADAM33 (A33) and control HEK293-Empty Vector (EV) after exposure to ADP13 antibody. An ADAM33 antibody was used as a positive control. Both antibodies were matched with their respective control isotype IgG.

FIG. 21A is a Kaplan-Meier curve of the ability of ADP2 to increase disease-free survival of mice with pre-existing MDA-MB-231 TNBC cell line-derived orthotopic tumors when administered in a neoadjuvant (tumor resection) treatment protocol vs its isotype-matched control IgG. n=number of animals/group. P-values were calculated using a Log-rank test.

FIG. 21B is a Kaplan-Meier curve of the ability of ADP13 to increase disease-free survival of mice with pre-existing MDA-MB-231 TNBC cell line-derived orthotopic tumors when administered in a neoadjuvant (tumor resection) treatment protocol vs its isotype-matched control IgG. n=number of animals/group. P-values were calculated using a Log-rank test.

FIG. 21C is a Kaplan-Meier curve of the ability of ADP2 to increase overall survival of mice with pre-existing MDA-MB-231 TNBC cell line-derived orthotopic tumors when administered in a neoadjuvant (tumor resection) treatment protocol vs its isotype-matched control IgG. n=number of animals/group. P-values were calculated using a Log-rank test.

FIG. 21D is a Kaplan-Meier curve of the ability of ADP13 to increase overall survival of mice with pre-existing MDA-MB-231 TNBC cell line-derived orthotopic tumors when administered in a neoadjuvant (tumor resection) treatment protocol vs its isotype-matched control IgG. n=number of animals/group. P-values were calculated using a Log-rank test.

FIG. 22A is a bar graph showing the presence and extent of bone metastases determined using biophotonic imaging of dissected bones for detection of activity from the luciferase tag expressed in MDA-MB-231 cells in mice that had pre-existing MDA-MB-231 TNBC cell line-derived orthotopic tumors and were administered isotype control IgG2b.

FIG. 22B is a bar graph showing the presence and extent of bone metastases determined using biophotonic imaging of dissected bones for detection of activity from the luciferase tag expressed in MDA-MB-231 cells in mice that had pre-existing MDA-MB-231 TNBC cell line-derived orthotopic tumors and were administered ADP2 antibody in a neoadjuvant (tumor resection) treatment protocol. Representative images of isolated bones from individual mice (M) are shown in top panel. Top left panel representing bones from mice administered with isotype control IgG2b. Top right panel representing bones from mice administered with ADP2 antibody. A grey color on the bone indicates a small to moderate size metastasis. A black color on the bone corresponds to a large metastatic lesion. A white color on the bone indicates no metastasis.

FIG. 22C is a bar graph showing the presence and extent of bone metastases determined using biophotonic imaging of dissected bones for detection of activity from the luciferase tag expressed in MDA-MB-231 cells in mice that had pre-existing MDA-MB-231 TNBC cell line-derived orthotopic tumors and were administered isotype control IgG1.

FIG. 22D is a bar graph showing the presence and extent of bone metastases determined using biophotonic imaging of dissected bones for detection of activity from the luciferase tag expressed in MDA-MB-231 cells in mice that had pre-existing MDA-MB-231 TNBC cell line-derived orthotopic tumors and were administered ADP13 antibody in a neoadjuvant (tumor resection) treatment protocol. Representative images of isolated bones from individual mice (M) are shown in top panel. Top left panel representing bones from mice administered with isotype control IgG1. Top right panel representing bones from mice administered with ADP13 antibody. A grey color on the bone indicates a small to moderate size metastasis. A black color on the bone corresponds to a large metastatic lesion. A white color on the bone indicates no metastasis.

FIG. 23A is a graph showing the pharmacokinetic profile of ADP2 48 hours post i.p. injection of a single 10 mg/kg dose of ADP2 into NOD/SCID mice. The concentration of ADP2 protein in plasma, isolated from the blood of the injected mice at various time points (0 hours, 2 hours, 4 hours, 6 hours, 8 hours, 24 hours, and 48 hours) was determined in three independent ELISA runs, in which rHuADAM8 was used for ADP2 capture and an anti-Mouse IgG-horseradish peroxidase (HRP) secondary for signal detection. number of mice, n=3.

FIG. 23B is a graph showing the pharmacokinetic profile of ADP2 over a 21-day period post i.p. injection of a single 10 mg/kg dose of ADP2 into NOD/SCID mice. The concentration of ADP2 protein in plasma, isolated from the blood of the injected mice at various time points (0 hours, 2 hours, 4 hours, 6 hours, 8 hours, 24 hours, 48 hours, 96 hours, 168 hours, 336 hours, and 504 hours) was determined in three independent ELISA runs, in which rHuADAM8 was used for ADP2 capture and an anti-Mouse IgG-HRP secondary for signal detection.

FIG. 23C is a table showing the specific ADP2 concentration values (nM)+/−standard deviation (S.D.) at the individual time points of mice used in FIG. 23B.

FIG. 24A is a graph showing the pharmacokinetic profile of ADP13 48 hours post i.p. injection of a single 10 mg/kg dose of ADP13 into NOD/SCID mice. ADP13 plasma concentration from blood collected at each time point (0 hours, 2 hours, 4 hours, 6 hours, 8 hours, 24 hours, and 48 hours) was determined in three independent ELISA runs.

FIG. 24B is a graph showing the pharmacokinetic profile of ADP13 over a 21-day period post i.p. injection of a single 10 mg/kg dose of ADP13 into NOD/SCID mice. ADP13 plasma concentration from blood collected at each time point (0 hours, 2 hours, 4 hours, 6 hours, 8 hours, 24 hours, 48 hours, 96 hours, 168 hours, 336 hours, and 504 hours) was determined in three independent ELISA runs.

FIG. 24C is a table showing the specific ADP13 concentration values (nM)+/−standard deviation (S.D.) at the individual time points of mice used in FIG. 24B.

FIG. 25A is a graphic representation of the experimental decay for ADP2. The ADP2 semi-log plasma concentration curves were plotted and found to be composed each of a distribution phase alpha-phase (a) and an elimination phase beta-phase (B).

FIG. 25B is a table showing specific PK parameters (area under the curve (AUC), elimination rate constant (Ke), half-time (T½) and clearance) for ADP2.

FIG. 25C is a graphic representation of the experimental decay for ADP13. The ADP13 semi-log plasma concentration curves were plotted and found to be composed each of a distribution phase alpha-phase (a) and an elimination phase beta-phase (B).

FIG. 25D is a table showing specific PK parameters (area under the curve (AUC), elimination rate constant (Ke), half-time (T½) and clearance) for ADP13.

FIG. 26A is a graph showing plasma ADP2 and ADP13 concentration over time (in hours) following a treatment regimen composed of a loading dose of 20 mg/kg antibody followed by maintenance doses of 10 mg/kg (3×/week) in NOD/SCID mice. Dotted box indicates achievement of steady state concentration. Values are obtained from three independent ELISA runs of plasma isolated from the blood of mice (n=3/time point) treated with ADP2 or ADP13 based on the proposed loading/maintenance dose regimen.

FIG. 26B is a table showing the specific ADP2 concentration values (nM)+/−standard deviation (S.D.) at each time point of FIG. 26A.

FIG. 26C is a table showing the specific ADP13 concentration values (nM)+/−standard deviation (S.D.) at each individual time point of FIG. 26A.

FIG. 27 is a graph showing tumor volume (Mean±standard error of mean (S.E.M.)) over time in a tumor regrowth model. Mice carrying pre-existing MDA-MB-231-luciferase-tagged cell-derived orthotopic tumors were treated with control IgG2b+Saline, ADP2+Saline, IgG2b+Nanoparticle Albumin-Bound Paclitaxel (NPAC) or the combination of ADP2+NPAC (n=number of animals/group). NPAC (in saline) was administered in 2 cycles of 5 consecutive intravenous (i.v.) treatments with one week of rest in between; an equivalent volume of vehicle saline was also given. ADP2 or IgG2b were administered i.p. 3×/week. Percentages indicate level of inhibition of tumor growth vs corresponding control group. P-values were determined using a Student's t-test.

FIG. 28A is a bar graph showing the presence and extent of bone metastasis from TNBC tumors of mice used in FIG. 27 (IgG2b+Saline vs ADP2+Saline) using biphotonic imaging of dissected bones for detection of activity from the luciferase tag expressed in MDA-MB-231 cells.

FIG. 28B is a bar graph showing the presence and extent of total bone metastasis from TNBC tumors of mice used in FIG. 27 (IgG2b+NPAC vs ADP2+NPAC) using biphotonic imaging of dissected bones for detection of activity from the luciferase tag expressed in MDA-MB-231 cells.

FIG. 28C is a collection of images showing all hind leg bone metastases from individual mice (M) treated with IgG2b+Saline or ADP2+Saline as in FIG. 28A. A grey color on the bone indicates a small to moderate-sized metastasis. A black color on the bone corresponds to a large metastatic lesion. A white color on the bone indicates no metastasis.

FIG. 28D is a collection of images showing all hind leg bone metastases from individual mice (M) treated with IgG2b+NPAC or ADP2+NPAC as in FIG. 28B. A grey color on the bone indicates a small to moderate-sized metastasis. A black color on the bone corresponds to a large metastatic lesion. A white color on the bone indicates no metastasis.

FIG. 29A is a Kaplan-Meier curve of the ability of ADP2 monotherapy (IgG2b+Saline vs ADP2+Saline) to increase overall survival in the mice used in FIG. 27. Statistical significance was determined using a Log-rank test; P-values=0.0111.

FIG. 29B is a Kaplan-Meier curve of the ability of combination therapy with NPAC (IgG2b+NPAC vs ADP2+NPAC) to increase overall survival in the mice used in FIG. 27. Statistical significance was determined using a Log-rank test; P-values=0.0366.

FIG. 30 is a graph showing tumor volume (Mean±S.E.M.) over time in a tumor regrowth model. Mice carrying pre-existing MDA-MB-231-luciferase-tagged cell-derived orthotopic tumors were treated with control IgG1+Saline, ADP13+Saline, IgG1+NPAC or ADP13+NPAC (n=number of animals/group). NPAC (in saline) was administered in 2 cycles of 5 consecutive i.v. treatments with one week of rest in between; an equivalent volume of vehicle saline was also given. ADP13 or IgGI were administered i.p. 3×/week. Percentages indicate level of inhibition of tumor growth vs corresponding control group. P-values were determined using a Student's t-test.

FIG. 31A is a bar graph showing the presence and extent of total bone metastasis from TNBC tumors of mice used in FIG. 30 (IgG1+Saline vs ADP13+Saline) using biphotonic imaging of dissected bones for detection of activity from the luciferase tag expressed in MDA-MB-231 cells.

FIG. 31B is a bar graph showing the presence and extent of bone metastasis from TNBC tumors of mice used in FIG. 30 (IgG1+NPAC vs ADP13+NPAC) using biphotonic imaging of dissected bones for detection of activity from the luciferase tag expressed in MDA-MB-231 cells.

FIG. 31C is a collection of images showing all hind leg bone metastases from individual mice (M) treated with IgG1+Saline or ADP13+Saline as in FIG. 31A. A grey color on the bone indicates a small to moderate-sized metastasis. A black color on the bone corresponds to a large metastatic lesion. A white color on the bone indicates no metastasis.

FIG. 31D is a collection of images showing all hind leg bone metastases from individual mice (M) treated with IgG1+NPAC or ADP13+NPAC as in FIG. 31B. A grey color on the bone indicates a small to moderate-sized metastasis. A black color on the bone corresponds to a large metastatic lesion. A white color on the bone indicates no metastasis.

FIG. 32A is a Kaplan-Meier curve of the ability of ADP13 monotherapy (IgG1+Saline vs ADP13+Saline) to increase overall survival in the mice used in FIG. 30. Statistical significance was determined using a Log-rank test; P-values=0.0035.

FIG. 32B is a Kaplan-Meier curve of the ability of combination therapy with NPAC (IgG1+NPAC vs ADP13+NPAC) to increase overall survival in the mice used in FIG. 30. Statistical significance was determined using a Log-rank test; P-values=0.0072.

FIG. 33A is a fluorescent-activated cell sorting (FACS) analysis histogram of HEK293 cells stably expressing either empty vector control DNA (EV), full-length ADAM8 or remnant ADAM8 (which lacks the promodomain and MP domain) showing ADP2 binding to the disintegrin (DI) region of ADAM8.

FIG. 33B is a FACS analysis histogram of HEK293 cells stably expressing either EV, full-length ADAM8 or remnant ADAM8 showing ADP3 binding to the DI region of ADAM8.

FIG. 33C is a FACS analysis histogram of HEK293 cells stably expressing either EV, full-length ADAM8 or remnant ADAM8 showing ADP13 binding to the DI region of ADAM8.

FIG. 33D is a schematic representation of the ADAM8 constructs used in FIGS. 33A-C, with domain information and amino acid numbers, as well as the immunogen injected into mice for generation of the ADP antibodies. The identified ADP2, ADP3 and ADP13 epitope binding region is indicated (dotted grey box). ADAM8 domains: Pro-prodomain; MP-metalloproteinase; DI-disintegrin; CDR-cysteine-rich; ELD-EGF-like; TM-transmembrane; CTD-cytoplasmic domain.

FIG. 34 is a diagram indicating the epitopes at the peptide level within the DI domain of human ADAM8 to which ADP2, ADP3 or ADP13 bind, as identified using hydrogen deuterium exchange (HDX) mass spectrometry analysis. HDX mass spectrometry analysis with rHuADAM8 in the presence or absence of ADP2, ADP3 or ADP13 identified the indicated protected peptide sequences as judged by a decrease in deuterium exchange level upon antibody binding. These peptides are thus identified as containing the epitope amino acid sequences of ADAM8 for specific ADP2, ADP3 and ADP13 antibody binding. Calcium ion binding site I (involving the 4 starred amino acids in the MP domain), and sites II and III in the DI domain are indicated. The integrin binding region (DMCD, open box) within the “disintegrin mobile hairpin loop” is stabilized by disulfide bonds and calcium binding to the adjacent site III. The first 3 amino acids for each ADAM8 domain are given in the inset box. The GenBank number for human ADAM8 is AAI15405.1

FIG. 35 is an image of a 3D model of the extracellular structure of ADAM8 as predicted using Swiss-model software and the crystal structure of ADAM22 as a template. The ADAM8 ectodomain structure (residues 195-647, which include the MP, DI, CRD and ELD domains) was predicted. Peptides of ADP2, ADP3 and ADP13 binding, identified from the HDX mass spectrometry analysis in FIG. 34 are indicated, including common regions. The MP domain with its active catalytic site, the DI domain with its integrin binding region and the hypervariable region (HVR) of the CDR domain are also shown.

FIG. 36A is a graph showing binding of chimeric ADP2 and ADP13 antibodies composed of mouse ADP V regions and human IgG1 C regions (chADP2 and chADP13) to human ADAM8 (rHuADAM8) using ELISA assays over a concentration range from 0 to 2.5 nM. KD_{(chADP2)} is 0.03664 nM. KD_{(chADP13)} is 0.07948 nM.

FIG. 36B is a FACS analysis histogram of HEK293 cells stably expressing either empty vector control DNA (HEK293-EV) or full-length ADAM8 (HEK293-A8) showing chADP2 binding to native ADAM8. Human IgG1 (hlgG1) was used as an antibody isotype-matched control.

FIG. 36C is a FACS analysis histogram of HEK293 cells stably expressing either empty vector control DNA (HEK293-EV) or full-length ADAM8 (HEK293-A8) showing chADP13 binding to native ADAM8. Human IgG1 (hlgG1) was used as an antibody isotype-matched control.

FIG. 37A is a bar graph showing the ability of chimeric ADP2 (chADP2) and mouse ADP2 (mADP2) treatment to inhibit MP activity as assessed using CD23 cleavage as in FIG. 10. Human IgG1 (hlgG1) and mouse IgG2b (mIgG2b) were used as controls, as appropriate.

FIG. 37B is a bar graph showing the ability of chimeric ADP13 (chADP13) and mouse ADP13 (mADP13) treatment to inhibit MP activity as assessed using CD23 cleavage, as in FIG. 10. Human IgG1 (hlgG1) or mouse IgG1 (mIgG1) were used as controls, as appropriate.

FIG. 37C is a bar graph showing the ability of chADP2 vs mADP2 to inhibit DI activity using transendothelial migration (TEM) assays as in FIG. 10. Levels of activity seen with control IgGs (mIgG2b, hlgG1) were set to 100%. Values are Mean±S.D. from three independent experiments.

FIG. 37D is a bar graph showing the ability of chADP13 vs mADP13 to inhibit DI activity using transendothelial migration (TEM) assays as in FIG. 10. Levels of activity seen with control IgGs (mIgG1, hlgG1) were set to 100%. Values are Mean=S.D. from three independent experiments.

FIG. 38 is a table showing the amino acid residues important for ADP2 and ADP13 binding to ADAM8 identified using shotgun mutagenesis. Mean binding reactivity (in duplicate samples) of the test ADP2 and ADP13 antigen-binding fragments (Fabs), under high stringency (HS) conditions, and of the positive control ADAM8 antibody (Control Ab), to the mutated ADAM8 protein residues at the indicated positions (Mutation) is listed as a percentage of binding to the corresponding wild type (WT) residue. The range of binding reactivity (maximum-minimum) in each case is indicated in parentheses. Amino acids that reached the threshold guidelines for critical binding residues (i.e., control antibody binding >70% of WT and test antibody binding <20% of WT binding) are shown in closed boxes. Mutations in these amino acid positions resulted in a significant reduction of ADP2 and ADP13 Fab binding, but no reduction of control antibody binding. Dotted boxes show residues of secondary importance, i.e., that did not reach the <20% of WT binding criterion for critical residues but still led to a substantial reduction in ADP2 and ADP13 Fab binding activity. This in combination with their proximity to critical residues indicates that they are part of the antibody epitope.

FIG. 39A is a graphic representation of the extracellular structure of ADAM8 with epitope amino acid residues for ADP2 Fab binding, identified through shotgun mutagenesis, indicated. Residues were visualized on a crystal structure model of ADAM8 based on the structure of vascular apoptosis-inducing protein-1 (PDB ID #2ERP, Takeda et al., EMBO J. 25:2388-2396, 2006). E444 is an ADP2 Fab critical binding residue. R431, G445, and K458 are secondary binding residues for ADP2 Fab.

FIG. 39B is a graphic representation of the extracellular structure of ADAM8 with epitope amino acid residues for ADP13 Fab binding, identified through shotgun mutagenesis, indicated. Residues were visualized on a crystal structure model of ADAM8 based on the structure of vascular apoptosis-inducing protein-1 (PDB ID #2ERP, Takeda et al., EMBO J. 25:2388-2396, 2006). G445, Q447, K458, and R482 are ADP13 Fab critical binding residues. V459 and A462 are secondary binding residues for ADP13 Fab.

FIG. 40 is a diagram of the strategy with steps used to identify ADP2 as a diagnostic antibody for use in immunohistochemistry (IHC)-based detection of ADAM8 expression on cancer cells and patient-derived xenograft (PDX) tumor tissue samples and to establish a breast control cell line microarray (CCM) with a gradient of low, medium, and high ADAM8 levels for quantitation of tissue sample staining.

FIG. 41A are FACS analysis histograms demonstrating that the ADP antibodies retain their ability to recognize ADAM8 on the cell surface under fixed conditions. ADP2, ADP3, ADP4 and ADP6 were tested in FACS analysis using 2D cultured HEK293-ADAM8 cells, which ectopically (exogenously) overexpress full-length ADAM8 (HEK-A8-2D), or HEK293-Empty vector DNA cells (HEK-EV-2D) under native/unfixed conditions (left panels) or following fixation (right panels) versus appropriate IgG controls.

FIG. 41B are FACS analysis histograms demonstrating that the ADP antibodies retain their ability to recognize ADAM8 on the cell surface under fixed conditions. ADP7, ADP9, ADP10 and ADP11 were tested in FACS analysis using 2D cultured HEK293-ADAM8 cells, which ectopically (exogenously) overexpress full-length ADAM8 (HEK-A8-2D), or HEK293-Empty vector DNA cells (HEK-EV-2D) under native/unfixed conditions (left panels) or following fixation (right panels) versus appropriate IgG controls.

FIG. 41C are FACS analysis histograms demonstrating that the ADP antibodies retain their ability to recognize ADAM8 on the cell surface under fixed conditions. ADP13, ADP17, ADP18 and ADP19 were tested in FACS analysis using 2D cultured HEK293-ADAM8 cells, which ectopically (exogenously) overexpress full-length ADAM8 (HEK-A8-2D), or HEK293-Empty vector DNA cells (HEK-EV-2D) under native/unfixed conditions (left panels) or following fixation (right panels) versus appropriate IgG controls.

FIG. 42 are images showing that ADP2, ADP13 and ADP17 can detect ectopically (exogenously) expressed ADAM8 in HEK293-ADAM8 using IHC conditions optimized for the LS-B4068 ADAM8 antibody. Images are at 40× magnification.

FIG. 43 are images demonstrating that ADP2 and ADP17 are comparable to LS-B4068 in detecting exogenously expressed ADAM8 in HEK293-ADAM8 cells. IHC was performed as above in FIG. 42 with 2D cultured HEK293-ADAM8 (HEK-A8-2D) and HEK293-Empty Vector (HEK-EV-2D) cells and 1:100 dilutions of LS-B4068, ADP2 and ADP17. Images are at 40× magnification.

FIG. 44 is a Western blot image comparing the levels of endogenously expressed ADAM8 in untransformed breast cells and breast cancer cells versus exogenously expressed ADAM8 in HEK293-ADAM8 cells, while β-actin was used as a loading control. MCF10A-2D, MDA-MB-231-2D and MDA-MB-231-3D cells were selected to create a breast CCM with a gradient of low, medium, and high ADAM8 levels. HEK-EV-2D and HEK-A8-2D cells were selected as negative and positive controls, respectively. MDA-MB-231 (MB-231) cells; 2D and 3D at the end of each cell line name indicate specific growth conditions.

FIG. 45 are images showing that ADP2 and ADP17 detect exogenously expressed ADAM8 in HEK293 cells, but not endogenously expressed ADAM8 in breast cells under the IHC conditions optimized for the LS-B4068 antibody. IHC was performed with CCM slides and 1:100 dilutions of LS-B4068, ADP2 and ADP17 versus their isotype matched controls, rabbit polyclonal IgG, mouse IgG2b and mouse IgG1, respectively. HEK293-Empty Vector (HEK-EV), HEK293-ADAM8 (HEK-A8) and MDA-MB-231 (MB-231) cells; 2D and 3D at the end of each cell line name indicate specific growth conditions. Images are at 40× magnification.

FIG. 46 are images demonstrating ADAM8 staining in the breast CCM under the optimized conditions for use of ADP antibodies in IHC. IHC was performed with ADP2 as a prototype ADP antibody (at 1:100 dilution) and with CCM slides. HEK293-Empty Vector (HEK-EV), HEK293-ADAM8 (HEK-A8) and MDA-MB-231 (MB-231) cells; 2D and 3D at the end of each cell line name indicate specific growth conditions. Images are at 40× magnification.

FIG. 47A are images comparing the IHC activity of ADP2 versus other antibodies within the ADP panel. The activity of ADP2 versus ADP3, ADP4, ADP9 and ADP12, at 1:100 dilutions, was compared using fresh slides of the breast CCM (described in FIG. 46) and the optimal staining conditions for use of ADP antibodies in IHC. Images are at 40× magnification.

FIG. 47B are images comparing the IHC activity of ADP2 versus other antibodies within the ADP panel. The activity of ADP2 versus ADP13, ADP18, and ADP19, at 1:100 dilutions, was compared using fresh slides of the breast CCM (described in FIG. 46) and the new optimal staining conditions for use of ADP antibodies in IHC. Images are at 40× magnification.

FIG. 48 are images comparing the performance of ADP2, ADP17 and LS-B4068 versus their control IgGs, at 1:100 dilutions, using slides of the CCM (described in FIG. 46) and the new optimal IHC staining conditions for ADP antibodies. Images are at 40× magnification.

FIG. 49 are images showing the IHC scoring system established using the breast specific CCM. Cells, grown in 2D or 3D conditions, were either processed in a single block to generate the breast specific CCM (described in FIG. 46) and slides analyzed by IHC using ADP2 (with ADP optimal staining conditions and 1:100 dilution) or protein extracted and subjected to Western blotting using the LS-C20181 anti-ADAM8 antibody. A stepwise ~5-7-fold increase in relative active ADAM8 levels (indicated below images), and a low, medium, and high percent cell culture staining positivity was seen between the three cell lines of breast origin in Western blotting and IHC, respectively. Based on active ADAM8 levels and culture positivity, these cells were defined as having a simple 1+, 2+ and 3+ADAM8 IHC staining score (indicated below images in parentheses). Images are at 40× magnification.

FIG. 50 are images demonstrating the range and linearity of ADP2 ADAM8 IHC staining. IHC was performed using slides of the CCM (described in FIG. 46) and a range of ADP2 dilutions from 1:50 to 1:120,000 under the optimized ADP staining conditions versus control IgG2b at 1:50. Images are at 40× magnification.

FIG. 51 are images demonstrating the specificity of ADP2 IHC staining for ADAM8. ADP2 at 1:1000 dilution was pre-incubated overnight at 4° C. in the absence or presence of 1×, 10× or 100× molar equivalents of purified recombinant human ADAM8 protein and used in HEK293-ADAM8 (HEK-A8)-2D and MDA-MB-231 (MB-231)-3D IHC. Images are at 40× magnification.

FIG. 52 are images demonstrating the ability of ADP2 to detect ADAM8 in TNBC patient-derived xenograft (PDX) tumor samples. IHC was performed using ADP2 at dilutions of 1:50, 1:100 and 1:500 versus its isotype-matched control IgG2b at 1:50 and TNBC PDX samples 5998, 3561, and 4849, which were found previously to have high ADAM8 expression based on a preliminary IHC screen of 30 PDX samples with LS-B4068 and ADP2. Images are at 40× magnification.

FIG. 53A are images demonstrating ADP2 IHC staining is reproducible in TNBC PDX samples. Two sets of single section slides of PDX 5998 tumors were subjected on different days to IHC using ADP2 and its isotype-matched control IgG2b at 1:50 dilutions. Images are at 10× magnification.

FIG. 53B are images demonstrating ADP2 IHC staining is reproducible in TNBC PDX samples. Two sets of single section slides of PDX 3561 tumors were subjected on different days to IHC using ADP2 and its isotype-matched control IgG2b at 1:50 dilutions. Images are at 10× magnification.

FIG. 53C are images demonstrating ADP2 IHC staining is reproducible in TNBC PDX samples. Two sets of single section slides of PDX 4849 tumors were subjected on different days to IHC using ADP2 and its isotype-matched control IgG2b at 1:50 dilutions. Images are at 10× magnification.

FIG. 54 are images showing the ADAM8 IHC scores for TNBC PDX samples are within the range of the CCM. ADP2 or its isotype-matched control IgG2b was used at a 1:50 dilution in IHC of the breast CCM (top panels) and the TNBC PDX 5998, PDX 3561, and PDX 4849 samples (lower panels). PDX staining scores were determined by direct visual comparison to the breast lines within the CCM (described in FIG. 46) and their IHC scores as established in FIG. 49. Sample scores are indicated at the bottom left corner of each image. Images are at 40× magnification.

### DETAILED DESCRIPTION

Provided herein are proteins that inhibit both the metalloprotease activity and disintegrin activity of human ADAM8, wherein the protein includes an antigen-binding domain that: (i) binds specifically to human ADAM8; and (ii) binds to an epitope within human ADAM8 that includes at least one (e.g., at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68 amino acids) amino acid within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

Provided herein are nucleic acid sequences encoding any of the proteins described herein, vectors including any of the nucleic acids described herein, and mammalian cells including any of the nucleic acids described herein or any of the vectors described herein.

Provided herein are methods of producing a protein that includes: (a) culturing a mammalian cell (e.g., any of the mammalian cells described herein) in a liquid culture medium under conditions sufficient to produce the protein; and (b) recovering the protein from the mammalian cell or the liquid culture medium. In some embodiments of any of the methods described herein, the method further includes: (c) isolating the protein recovered in step (b). In some embodiments of any of the methods described herein, the method further includes: (d) formulating the protein isolated in step (c) into a pharmaceutical composition.

Also provided herein are pharmaceutical compositions produced by any of the methods described herein.

Also provided herein are pharmaceutical compositions including a therapeutically effective amount of any of the proteins described herein.

Also provided herein are kits that include any of the proteins described herein or any of the pharmaceutical compositions described herein.

Also provided herein are methods for inhibiting migration and/or invasion of an ADAM8 expressing cell in a subject that include administering to the subject a therapeutically effective amount of any of the proteins described herein or any of the pharmaceutical compositions described herein.

Provided herein are methods of decreasing the risk of developing a metastasis or developing an additional metastasis over a period of time in a subject identified as having an ADAM8-associated cancer that include administering to the subject a therapeutically effective amount of any of the proteins described herein or any of the pharmaceutical compositions described herein.

Provided herein are methods of inhibiting the growth of a solid tumor in a subject identified as having an ADAM8-associated cancer that include administering to the subject a therapeutically effective amount of any of the proteins described herein or any of the pharmaceutical compositions described herein.

Provided herein are methods of inhibiting the growth or proliferation of a hematological cancer in a subject identified as having an ADAM8-associated cancer that include administering to the subject a therapeutically effective amount of any of the proteins described herein or any of the pharmaceutical compositions described herein.

Also provided herein are methods of killing an ADAM8-associated cancer cell in a subject that include: administering to the subject a therapeutically effective amount of any of the proteins described herein or any of the pharmaceutical compositions described herein.

Provided herein are methods of treating an ADAM8-associated cancer in a subject that include: administering to a subject identified as having an ADAM8-associated cancer a therapeutically effective amount of any of the proteins described herein or any of the pharmaceutical compositions described herein.

Provided herein are methods of identifying a protein including an antigen-binding domain that binds specifically to human ADAM8 and has the ability to inhibit both the metalloprotease activity and disintegrin activity of human ADAM8 that include: (a) identifying a protein including an antigen-binding domain that binds to an epitope within human ADAM8 that includes at least one amino acid (e.g., at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68 amino acids) within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

### ADAM8

ADAM8 is a type I transmembrane protein that belongs to the ADAM (A Disintegrin And Metalloprotease) family. ADAM8 mediates cell adhesion, cell migration, and proteolysis of various substrates, including receptors and ligands for cytokines and immunoglobulins (Ig), cell adhesion molecules and extracellular matrix components.

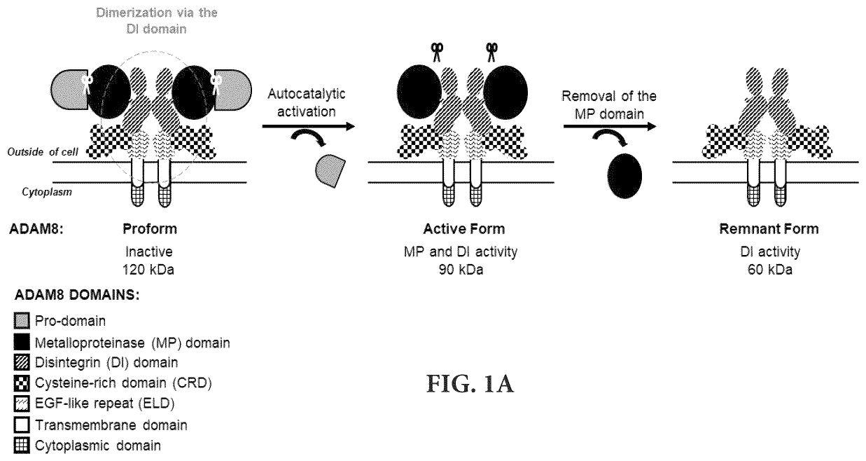

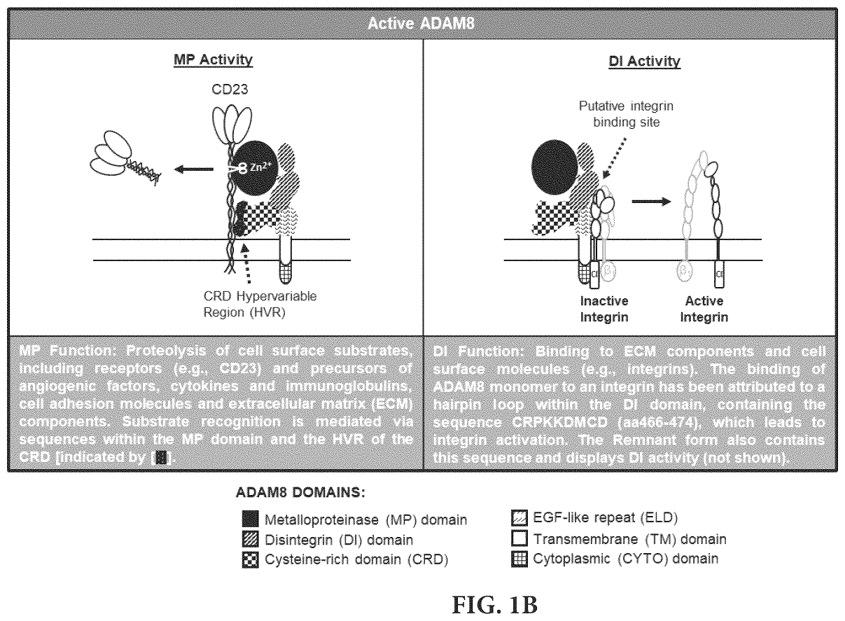

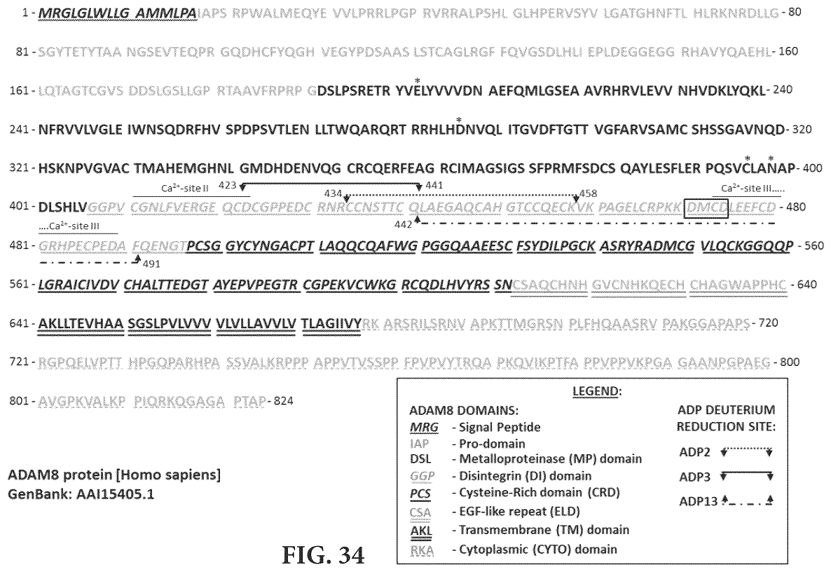

Human ADAM8 is synthesized as an inactive 120 kDa (824 amino acid) proform with a signal peptide (amino acids 1-16), which is clipped off upon entry into the rough endoplasmic reticulum on its way to the cell surface, and an inhibitory amino terminal prodomain (amino acids 17-191). Upon dimerization or multimerization, ADAM8 autocatalytically removes its prodomain, leading to the formation of a 90 kDa membrane-anchored “active” form, which has four functional extracellular domains: the metalloproteinase (MP, amino acids 192-406), disintegrin (DI, amino acids 407-496), cysteine-rich domain (CRD, amino acids 497-612), and EGF-like domain (ELD, amino acids 613-640) (FIG. 1A and FIG. 34). These are followed by a transmembrane region (TM, amino acids 641-678) and a cytoplasmic tail (CYTO, amino acids 679-824). Active ADAM8 has both MP and DI activities, but can be further processed by removal of the MP domain to a transmembrane 60 kDa Remnant form (amino acids 407-824). ADAM8 Remnant consists of the DI, CRD, and ELD domains, and the TM and CYTO (FIG. 1A), and retains DI activity (FIG. 1B).

The crystal structure of the human ADAM8 MP domain with batimastat, a broad-spectrum inhibitor of metalloproteinases, revealed an overall similarity to other members of the ADAM family, including a characteristic central five-stranded β-sheet, four long α-helices and one short N-terminal α-helix (Hall et al., Acta Crystallogr Sect F Struc Biol Cryst Commun, 68 (Pt 6): 616-621, 2012). The MP catalytic active site has a characteristic Zn²⁺ ion binding consensus sequence: HEXXHXXGXXH (amino acids 334-344) (SEQ ID NO: 90). The three histidines (H, underlined) coordinate the binding of Zn²⁺ while the glutamic acid (E, bold) functions as part of a catalytic base within the active site cleft; consistently, an E335Q mutation inhibits protease activity (Srinivasan et al., J Biol Chem, 289 (48): 33676-22688, 2014). The MP domain of Active ADAM8 modulates cellular signals through its sheddase activity by cleaving proteins on the cancer cell surface (FIG. 1B), including receptors such as the CD23 receptor for IgE, pro-angiogenic cytokines (see below), growth factors, as well as components of the extracellular matrix. The 90 amino acid-DI domain in ADAM8 Active and Remnant forms mediates cell adhesion, notably by direct binding to and activation of integrins, including β1-integrin (as depicted for the Active form in FIG. 1B). The DI and CRD domains of ADAM8 are rich in cysteine residues, which play critical roles in maintaining a tight 3D structure. Specifically, highly conserved cysteine residues form disulfide linkages between the two halves of the DI domain and with the amino terminal portion of the CRD domain that result in the formation of a rigid C-shaped structure. This structure is further stabilized by binding of calcium ions to two distinct sites at either end of the DI domain (Ca²⁺-binding site II and Ca²⁺-binding site III, respectively). At the base of this C-shape structure is a loop of variable length and amino acid sequence within the CRD domain, which has been termed the hypervariable region (HVR). The HVR aligns the MP active site and has been implicated in binding/recognition of substrates and their correct processing by the MP domain (FIG. 1B). An additional calcium ion site (Ca²⁺-binding site I), located within the MP domain and in proximity to the DI domain, is also essential for MP activity. Finally, while the ADAM8 DI domain lacks a typical RGD integrin binding sequence, it contains a consensus integrin binding (D/S) XCD sequence, e.g., DMCD (aa471-474), which in other ADAM proteins is located at the tip of a highly mobile hairpin loop (FIG. 1B). The present invention is based, at least in part, on the discovery that the binding of antibodies to the DI domain of ADAM8 inhibits the activities of both its DI and MP domains, which is believed to be necessary for a significant therapeutic effect in ADAM8-associated cancer patients.

An exemplary human wildtype ADAM8 is or includes the sequence of SEQ ID NO: 91, 93, 95, 97, or 99. Non-limiting examples of nucleotide sequences encoding a wildtype ADAM8 protein are or include SEQ ID NO: 92, 94, 96, 98, or 100.

Human Wildtype ADAM8 Protein Transcript Variant 1
(SEQ ID NO: 91)
MRGLGLWLLGAMMLPAIAPSRPWALMEQYEVVLPWRLPGPRVRRALPSHLGLHPERVSYVLGATGHNFTLHLRKN
RDLLGSGYTETYTAANGSEVTEQPRGQDHCFYQGHVEGYPDSAASLSTCAGLRGFFQVGSDLHLIEPLDEGGEGG
RHAVYQAEHLLQTAGTCGVSDDSLGSLLGPRTAAVFRPRPGDSLPSRETRYVELYVVVDNAEFQMLGSEAAVRHR
VLEVVNHVDKLYQKLNFRVVLVGLEIWNSQDRFHVSPDPSVTLENLLTWQARQRTRRHLHDNVQLITGVDFTGTT
VGFARVSAMCSHSSGAVNQDHSKNPVGVACTMAHEMGHNLGMDHDENVQGCRCQERFEAGRCIMAGSIGSSFPRM
FSDCSQAYLESFLERPQSVCLANAPDLSHLVGGPVCGNLFVERGEQCDCGPPEDCRNRCCNSTTCQLAEGAQCAH
GTCCQECKVKPAGELCRPKKDMCDLEEFCDGRHPECPEDAFQENGTPCSGGYCYNGACPTLAQQCQAFWGPGGQA
AEESCFSYDILPGCKASRYRADMCGVLQCKGGQQPLGRAICIVDVCHALTTEDGTAYEPVPEGTRCGPEKVCWKG
RCQDLHVYRSSNCSAQCHNHGVCNHKQECHCHAGWAPPHCAKLLTEVHAASGSLPVFVVVVLVLLAVVLVTLAGI
IVYRKARSRILSRNVAPKTTMGRSNPLFHQAASRVPAKGGAPAPSRGPQELVPTTHPGQPARHPASSVALKRPPP
APPVTVSSPPFPVPVYTRQAPKQVIKPTFAPPVPPVKPGAGAANPGPAEGAVGPKVALKPPIQRKQGAGAPTAP
(NCBI Accession No. NM_001109.5)
Human Wildtype ADAM8 Transcript Variant 1 cDNA
(SEQ ID NO: 92)
atgcgcggcctcgggctctggctgctgggcgcgatgatgctgcctgcgattgcccccagccggccctgggccctc
atggagcagtatgaggtcgtgttgccgtggcgtctgccaggcccccgagtccgccgagctctgccctcccacttg
ggcctgcacccagagagggtgagctacgtccttggggccacagggcacaacttcaccctccacctgcggaagaac
agggacctgctgggctccggctacacagagacctatacggctgccaatggctccgaggtgacggagcagcctcgc
gggcaggaccactgcttctaccagggccacgtagaggggtacccggactcagccgccagcctcagcacctgtgcc
ggcctcaggggtttcttccaggtggggtcagacctgcacctgatcgagcccctggatgaaggtggcgagggcgga
cggcacgccgtgtaccaggctgagcacctgctgcagacggccgggacctgcggggtcagcgacgacagcctgggc
agcctcctgggaccccggacggcagccgtcttcaggcctcggcccggggactctctgccatcccgagagacccgc
tacgtggagctgtatgtggtcgtggacaatgcagagttccagatgctggggagcgaagcagccgtgcgtcatcgg
gtgctggaggtggtgaatcacgtggacaagctatatcagaaactcaacttccgtgtggtcctggtgggcctggag
atttggaatagtcaggacaggttccacgtcagccccgaccccagtgtcacactggagaacctcctgacctggcag
gcacggcaacggacacggcggcacctgcatgacaacgtacagctcatcacgggtgtcgacttcaccgggactacc
gtggggtttgccagggtgtccgccatgtgctcccacagctcaggggctgtgaaccaggaccacagcaagaacccc
gtgggcgtggcctgtaccatggcccatgagatgggccacaacctgggcatggaccatgatgagaacgtccagggc
tgccgctgccaggaacgcttcgaggccggccgctgcatcatggcgggcagcattggctccagtttccccaggatg
ttcagtgactgcagccaggcctacctggagagctttttggagcggccgcagtcggtgtgcctcgccaacgcccct
gacctcagccacctggtgggcggccccgtgtgtgggaacctgtttgtggagcgtggggagcagtgcgactgcggc
ccccccgaggactgccggaaccgctgctgcaactctaccacctgccagctggctgagggggcccagtgtgcgcac
ggtacctgctgccaggagtgcaaggtgaagccggctggtgagctgtgccgtcccaagaaggacatgtgtgacctc
gaggagttctgtgacggccggcaccctgagtgcccggaagacgccttccaggagaacggcacgccctgctccggg
ggctactgctacaacggggcctgtcccacactggcccagcagtgccaggccttctgggggccaggtgggcaggct
gccgaggagtcctgcttctcctatgacatcctaccaggctgcaaggccagccggtacagggctgacatgtgtggc
gttctgcagtgcaagggtgggcagcagcccctggggcgtgccatctgcatcgtggatgtgtgccacgcgctcacc
acagaggatggcactgcgtatgaaccagtgcccgagggcacccggtgtggaccagagaaggtttgctggaaagga
cgttgccaggacttacacgtttacagatccagcaactgctctgcccagtgccacaaccatggggtgtgcaaccac
aagcaggagtgccactgccacgcgggctgggccccgccccactgcgcgaagctgctgactgaggtgcacgcagcg
tccgggagcctccccgtcttcgtggtggtggttctggtgctcctggcagttgtgctggtcaccctggcaggcatc
atcgtctaccgcaaagcccggagccgcatcctgagcaggaacgtggctcccaagaccacaatggggcgctccaac
cccctgttccaccaggctgccagccgcgtgccggccaagggcggggctccagccccatccaggggcccccaagag
ctggtccccaccacccacccgggccagcccgcccgacacccggcctcctcggtggctctgaagaggccgccccct
gctcctccggtcactgtgtccagcccacccttcccagttcctgtctacacccggcaggcaccaaagcaggtcatc
aagccaacgttcgcacccccagtgcccccagtcaaacccggggctggtgcggccaaccctggtccagctgagggt
gctgttggcccaaaggttgccctgaagccccccatccagaggaagcaaggagccggagctcccacagcaccctag
Human Wildtype ADAM8 Protein Transcript Variant 2
(SEQ ID NO: 93)
MRGLGLWLLGAMMLPAIAPSRPWALMEQYEVVLPWRLPGPRVRRALPSHLGLHPERVSYVLGATGHNFTLHLRKN
RDLLGSGYTETYTAANGSEVTEQPRGQDHCFYQGHVEGYPDSAASLSTCAGLRGFFQVGSDLHLIEPLDEGGEGG
RHAVYQAEHLLQTAGTCGVSDDSLGSLLGPRTAAVFRPRPGDSLPSRETRYVELYVVVDNAEFQMLGSEAAVRHR
VLEVVNHVDKLYQKLNFRVVLVGLEIWNSQDRFHVSPDPSVTLENLLTWQARQRTRRHLHDNVQLITGVDFTGTT
VGFARVSAMCSHSSGAVNQDHSKNPVGVACTMAHEMGHNLGMDHDENVQGCRCQERFEAGRCIMAGSIGSSFPRM
FSDCSQAYLESFLERPQSVCLANAPDLSHLVGGPVCGNLFVERGEQCDCGPPEDCRNRCCNSTTCQLAEGAQCAH
GTCCQECKVKPAGELCRPKKDMCDLEEFCDGRHPECPEDAFQENGTPCSGGYCYNGACPTLAQQCQAFWGPGGQA
AEESCFSYDILPGCKASRYRADMCGVLQCKGGQQPLGRAICIVDVCHALTTEDGTAYEPVPEGTRCGPEKVCWKG
RCQDLHVYRSSNCSAQCHNHGVCNHKQECHCHAGWAPPHCAKLLTEVHAGCQPRAGQGRGSSPIQGPPRAGPHHP
PGPARPTPGLLGGSEEAAPCSSGHCVQPTLPSSCLHPAGTKAGHQANVRTPSAPSQTRGWCGQPWSS
(NCBI Accession No. NM_001164489.1)
Human Wildtype ADAM8 Transcript Variant 2 cDNA
(SEQ ID NO: 94)
atgcgcggcctcgggctctggctgctgggcgcgatgatgctgcctgcgattgcccccagccggccctgggccct
catggagcagtatgaggtcgtgttgccgtggcgtctgccaggcccccgagtccgccgagctctgccctcccactt
gggcctgcacccagagagggtgagctacgtccttggggccacagggcacaacttcaccctccacctgcggaagaa
cagggacctgctgggctccggctacacagagacctatacggctgccaatggctccgaggtgacggagcagcctcg
cgggcaggaccactgcttctaccagggccacgtagaggggtacccggactcagccgccagcctcagcacctgtgc
cggcctcaggggtttcttccaggtggggtcagacctgcacctgatcgagcccctggatgaaggtggcgagggcgg
acggcacgccgtgtaccaggctgagcacctgctgcagacggccgggacctgcggggtcagcgacgacagcctggg
cagcctcctgggaccccggacggcagccgtcttcaggcctcggcccggggactctctgccatcccgagagacccg
ctacgtggagctgtatgtggtcgtggacaatgcagagttccagatgctggggagcgaagcagccgtgcgtcatcg
ggtgctggaggtggtgaatcacgtggacaagctatatcagaaactcaacttccgtgtggtcctggtgggcctgga
gatttggaatagtcaggacaggttccacgtcagccccgaccccagtgtcacactggagaacctcctgacctggca
ggcacggcaacggacacggcggcacctgcatgacaacgtacagctcatcacgggtgtcgacttcaccgggactac
cgtggggtttgccagggtgtccgccatgtgctcccacagctcaggggctgtgaaccaggaccacagcaagaaccc
cgtgggcgtggcctgtaccatggcccatgagatgggccacaacctgggcatggaccatgatgagaacgtccaggg
ctgccgctgccaggaacgcttcgaggccggccgctgcatcatggcgggcagcattggctccagtttccccaggat
gttcagtgactgcagccaggcctacctggagagctttttggagcggccgcagtcggtgtgcctcgccaacgcccc
tgacctcagccacctggtgggcggccccgtgtgtgggaacctgtttgtggagcgtggggagcagtgcgactgcgg
cccccccgaggactgccggaaccgctgctgcaactctaccacctgccagctggctgagggggcccagtgtgcgca
cggtacctgctgccaggagtgcaaggtgaagccggctggtgagctgtgccgtcccaagaaggacatgtgtgacct
cgaggagttctgtgacggccggcaccctgagtgcccggaagacgccttccaggagaacggcacgccctgctccgg
gggctactgctacaacggggcctgtcccacactggcccagcagtgccaggccttctgggggccaggtgggcaggc
tgccgaggagtcctgcttctcctatgacatcctaccaggctgcaaggccagccggtacagggctgacatgtgtgg
cgttctgcagtgcaagggtgggcagcagcccctggggcgtgccatctgcatcgtggatgtgtgccacgcgctcac
cacagaggatggcactgcgtatgaaccagtgcccgagggcacccggtgtggaccagagaaggtttgctggaaagg
acgttgccaggacttacacgtttacagatccagcaactgctctgcccagtgccacaaccatggggtgtgcaacca
caagcaggagtgccactgccacgcgggctgggccccgccccactgcgcgaagctgctgactgaggtgcacgcagg
ctgccagccgcgtgccggccaagggcggggctccagccccatccaggggcccccaagagctggtccccaccaccc
acccgggccagcccgcccgacacccggcctcctcggtggctctgaagaggccgccccctgctcctccggtcactg
tgtccagcccacccttcccagttcctgtctacacccggcaggcaccaaagcaggtcatcaagccaacgttcgcac
ccccagtgcccccagtcaaacccggggctggtgcggccaaccctggtccagctga
Human Wildtype ADAM8 Protein Transcript Variant 3
(SEQ ID NO: 95)
MRGLGLWLLGAMMLPGPAPREGELRPWGHRAQLHPPPAEEQGPAGLRLHRDLYGCQWLRGDGAASRAGPLLLPGP
RRGVPGLSRQPQHLCRPQVGSDLHLIEPLDEGGEGGRHAVYQAEHLLQTAGTCGVSDDSLGSLLGPRTAAVFRPR
PGDSLPSRETRYVELYVVVDNAEFQMLGSEAAVRHRVLEVVNHVDKLYQKLNFRVVLVGLEIWNSQDRFHVSPDP
SVTLENLLTWQARQRTRRHLHDNVQLITGVDFTGTTVGFARVSAMCSHSSGAVNQDHSKNPVGVACTMAHEMGHN
LGMDHDENVQGCRCQERFEAGRCIMAGSIGSSFPRMFSDCSQAYLESFLERPQSVCLANAPDLSHLVGGPVCGNL
FVERGEQCDCGPPEDCRNRCCNSTTCQLAEGAQCAHGTCCQECKVKPAGELCRPKKDMCDLEEFCDGRHPECPED
AFQENGTPCSGGYCYNGACPTLAQQCQAFWGPGGQAAEESCFSYDILPGCKASRYRADMCGVLQCKGGQQPLGRA
ICIVDVCHALTTEDGTAYEPVPEGTRCGPEKVCNHKQECHCHAGWAPPHCAKLLTEVHAASGSLPVFVVVVLVLL
AVVLVTLAGIIVYRKARSRILSRNVAPKTTMGRSNPLFHQAASRVPAKGGAPAPSRGPQELVPTTHPGQPARHPA
SSVALKRPPPAPPVTVSSPPFPVPVYTRQAPKQGAVGPKVALKPPIQRKQGAGAPTAP
(NCBI Accession No. NM_001164490.1)
Human Wildtype ADAM8 Transcript Variant 3 cDNA
(SEQ ID NO: 96)
atgcgcggcctcgggctctggctgctgggcgcgatgatgctgcctgggcctgcacccagagagggtgagctacg
tccttggggccacagggcacaacttcaccctccacctgcggaagaacagggacctgctgggctccggctacacag
agacctatacggctgccaatggctccgaggtgacggagcagcctcgcgggcaggaccactgcttctaccagggcc
acgtagaggggtacccggactcagccgccagcctcagcacctgtgccggcctcaggtggggtcagacctgcacct
gatcgagcccctggatgaaggtggcgagggcggacggcacgccgtgtaccaggctgagcacctgctgcagacggc
cgggacctgcggggtcagcgacgacagcctgggcagcctcctgggaccccggacggcagccgtcttcaggcctcg
gcccggggactctctgccatcccgagagacccgctacgtggagctgtatgtggtcgtggacaatgcagagttcca
gatgctggggagcgaagcagccgtgcgtcatcgggtgctggaggtggtgaatcacgtggacaagctatatcagaa
actcaacttccgtgtggtcctggtgggcctggagatttggaatagtcaggacaggttccacgtcagccccgaccc
cagtgtcacactggagaacctcctgacctggcaggcacggcaacggacacggcggcacctgcatgacaacgtaca
gctcatcacgggtgtcgacttcaccgggactaccgtggggtttgccagggtgtccgccatgtgctcccacagctc
aggggctgtgaaccaggaccacagcaagaaccccgtgggcgtggcctgtaccatggcccatgagatgggccacaa
cctgggcatggaccatgatgagaacgtccagggctgccgctgccaggaacgcttcgaggccggccgctgcatcat
ggcgggcagcattggctccagtttccccaggatgttcagtgactgcagccaggcctacctggagagctttttgga
gcggccgcagtcggtgtgcctcgccaacgcccctgacctcagccacctggtgggcggccccgtgtgtgggaacct
gtttgtggagcgtggggagcagtgcgactgcggcccccccgaggactgccggaaccgctgctgcaactctaccac
ctgccagctggctgagggggcccagtgtgcgcacggtacctgctgccaggagtgcaaggtgaagccggctggtga
gctgtgccgtcccaagaaggacatgtgtgacctcgaggagttctgtgacggccggcaccctgagtgcccggaaga
cgccttccaggagaacggcacgccctgctccgggggctactgctacaacggggcctgtcccacactggcccagca
gtgccaggccttctgggggccaggtgggcaggctgccgaggagtcctgcttctcctatgacatcctaccaggctg
caaggccagccggtacagggctgacatgtgtggcgttctgcagtgcaagggtgggcagcagcccctggggcgtgc
catctgcatcgtggatgtgtgccacgcgctcaccacagaggatggcactgcgtatgaaccagtgcccgagggcac
ccggtgtggaccagagaaggtgtgcaaccacaagcaggagtgccactgccacgcgggctgggccccgccccactg
cgcgaagctgctgactgaggtgcacgcagcgtccgggagcctccccgtcttcgtggtggtggttctggtgctcct
ggcagttgtgctggtcaccctggcaggcatcatcgtctaccgcaaagcccggagccgcatcctgagcaggaacgt
ggctcccaagaccacaatggggcgctccaaccccctgttccaccaggctgccagccgcgtgccggccaagggcgg
ggctccagccccatccaggggcccccaagagctggtccccaccacccacccgggccagcccgcccgacacccggc
ctcctcggtggctctgaagaggccgccccctgctcctccggtcactgtgtccagcccacccttcccagttcctgt
ctacacccggcaggcaccaaagcagggtgctgttggcccaaaggttgccctgaagccccccatccagaggaagca
aggagccggagctcccacagcaccctag
Mouse Wildtype ADAM8 Protein Transcript Variant 1
(SEQ ID NO: 97)
MLGLWLLSVLWTPAVAPGPPLPHVKQYEVVWPRRLAASRSRRALPSHWGQYPESLSYALGTSGHVFTLHLRKNRD
LLGSSYTETYSAANGSEVTEQLQEQDHCLYQGHVEGYEGSAASISTCAGLRGFFRVGSTVHLIEPLDADEEGQHA
MYQAKHLQQKAGTCGVKDTNLNDLGPRALEIYRAQPRNWLIPRETRYVELYVVADSQEFQKLGSREAVRQRVLEV
VNHVDKLYQELSFRVVLVGLEIWNKDKFYISRYANVTLENFLSWREQNLQGQHPHDNVQLITGVDFIGSTVGLAK
VSALCSRHSGAVNQDHSKNSIGVASTMAHELGHNLGMSHDEDIPGCYCPEPREGGGCIMTESIGSKFPRIFSRCS
KIDLESFVTKPQTGCLTNVPDVNRFVGGPVCGNLFVEHGEQCDCGTPQDCQNPCCNATTCQLVKGAECASGTCCH
ECKVKPAGEVCRLSKDKCDLEEFCDGRKPTCPEDAFQQNGTPCPGGYCFDGSCPTLAQQCRDLWGPGARVAADSC
YTFSIPPGCNGRMYSGRINRCGALYCEGGQKPLERSFCTFSSNHGVCHALGTGSNIDTFELVLQGTKCEEGKVCM
DGSCQDLRVYRSENCSAKCNNHGVCNHKRECHCHKGWAPPNCVQRLADVSDEQAASTSLPVSVVVVLVILVAAMV
IVAGIVIYRKAPRQIQRRSVAPKPISGLSNPLFYTRDSSLPAKNRPPDPSETVSTNQPPRPIVKPKRPPPAPPGA
VSSSPLPVPVYAPKIPNQFRPDPPTKPLPELKPKQVKPTFAPPTPPVKPGTGGTVPGATQGAGEPKVALKVPIQK
R (NCBI Accession No. NM_007403.3)
Mouse Wildtype ADAM8 Transcript Variant 1 cDNA
(SEQ ID NO: 98)
atgcttggcctctggctgctcagcgtcttatggacaccagcagtagcccctggacctcctttgccccatgtgaaa
cagtatgaagtggtttggcctcggcgcctagctgcatcccgctcccgcagagccctgccctcccactggggccag
tacccagagagtctgagctatgctcttgggaccagcgggcacgttttcaccctgcaccttcgaaagaacagggac
ctgctgggctcaagctacacagagacctactcagctgccaatggctctgaggtgacagagcaactgcaggagcag
gaccattgcctctaccaaggccatgtggaagggtacgagggctcagctgccagtattagcacctgtgctggcctc
aggggctttttccgagttgggtccactgtccacttgattgagcctctggatgctgatgaagaggggcaacatgcg
atgtatcaggcaaagcatctgcaacagaaggctgggacctgtggggtcaaagataccaacctgaatgacctaggg
cctcgggcattagaaatatacagggctcagccacggaactggctgatacccagagaaacccgctatgtggagttg
tatgtggttgcagacagccaagagttccagaagttggggagcagagaggccgtgcgccagcgagtgctggaggtt
gtaaaccacgtggacaagctttatcaggaactcagttttcgagttgtcctggtgggcctggagatctggaacaag
gacaaattctacatcagccgctatgccaacgtgacactggagaacttcttgtcctggagggaacagaacttgcaa
gggcagcacccacatgacaacgtgcaacttatcacgggggttgatttcattgggagcactgttggactggctaag
gtgtctgccctgtgttcccgtcactccggagctgtgaatcaggaccactccaagaactccattggtgtagcctcc
accatggcccatgagctgggccacaacctgggcatgagccatgatgaggacattccaggatgctactgtcctgaa
ccacgggagggtggtggctgcatcatgaccgaaagcatcggctccaagttccccaggatattcagcaggtgtagc
aagattgacctagagtcattcgtgacaaaaccccagacaggctgcctgaccaatgttccagatgtcaaccggttc
gtgggtggccctgtgtgtggaaacctgtttgtggagcatggagagcagtgtgactgtggcacacctcaggactgt
caaaacccctgctgcaatgccaccacttgccagctggtcaagggggcagagtgtgccagtggtacctgttgtcat
gaatgcaaggtgaagccagctggagaggtgtgtcgtctcagtaaggacaaatgtgacctggaggagttctgtgat
ggccggaagccaacatgtcccgaagatgccttccaacagaatggcactccctgcccagggggctactgctttgat
gggagctgtcccaccctggcacagcagtgccgggatctgtgggggccaggtgctcgggtagcagccgactcctgc
tatacctttagcatccctccgggctgcaatgggaggatgtactctggcaggatcaaccggtgtggagcgctgtac
tgtgagggaggccagaagccccttgaacgctccttctgcactttctcctccaaccatggagtctgccatgctctt
ggcacaggcagcaacattgacacctttgagctggtattgcagggcaccaagtgcgaggagggaaaggtttgcatg
gatggaagctgccaggacctccgtgtatacagatctgaaaactgctctgctaaatgcaacaaccatggggtatgc
aaccacaagagggagtgccactgtcacaagggctgggcaccacccaactgtgtacagcggctggcagatgtatca
gatgaacaagcagcgtctacgagcctcccagtcagtgtggttgtggtcttggtgatcctggtggctgcgatggtc
atcgtggcaggcatcgtcatctaccgaaaggctccgagacaaatccagaggaggagtgtggcacccaagcctatc
tcggggctctccaaccccctattctacacaagggacagcagcctgccagctaagaacaggcctccagacccttct
gagacagtttctaccaaccagcccccaagacccatagtgaaaccaaagaggcctccccctgcacctccaggtgct
gtgtccagttcaccactcccagttcctgtttatgccccaaagataccaaatcagtttagacctgatcctcccacc
aagcccctcccagagctgaaacccaagcaggtcaagccaacctttgcacccccgacaccaccagtcaagcccggg
actggagggacggtgcctggagcaactcagggagctggtgagccaaaggttgctctgaaggtccccatccagaag
aggtga
Mouse Wildtype ADAM8 Protein Transcript Variant 2
(SEQ ID NO: 99)
MLGLWLLSVLWTPVAPGPPLPHVKQYEVVWPRRLAASRSRRALPSHWGQYPESLSYALGTSGHVFTLHLRKNRDL
LGSSYTETYSAANGSEVTEQLQEQDHCLYQGHVEGYEGSAASISTCAGLRGFFRVGSTVHLIEPLDADEEGQHAM
YQAKHLQQKAGTCGVKDTNLNDLGPRALEIYRAQPRNWLIPRETRYVELYVVADSQEFQKLGSREAVRQRVLEVV
NHVDKLYQELSFRVVLVGLEIWNKDKFYISRYANVTLENFLSWREQNLQGQHPHDNVQLITGVDFIGSTVGLAKV
SALCSRHSGAVNQDHSKNSIGVASTMAHELGHNLGMSHDEDIPGCYCPEPREGGGCIMTESIGSKFPRIFSRCSK
IDLESFVTKPQTGCLTNVPDVNRFVGGPVCGNLFVEHGEQCDCGTPQDCQNPCCNATTCQLVKGAECASGTCCHE
CKVKPAGEVCRLSKDKCDLEEFCDGRKPTCPEDAFQQNGTPCPGGYCFDGSCPTLAQQCRDLWGPGARVAADSCY
TFSIPPGCNGRMYSGRINRCGALYCEGGQKPLERSFCTFSSNHGVCHALGTGSNIDTFELVLQGTKCEEGKVCMD
GSCQDLRVYRSENCSAKCNNHGVCNHKRECHCHKGWAPPNCVQRLADVSDEQAASTSLPVSVVVVLVILVAAMVI
VAGIVIYRKAPRQIQRRSVAPKPISGLSNPLFYTRDSSLPAKNRPPDPSETVSTNQPPRPIVKPKRPPPAPPGAV
SSSPLPVPVYAPKIPNQFRPDPPTKPLPELKPKQVKPTFAPPTPPVKPGTGGTVPGATQGAGEPKVALKVPIQKR
(NCBI Accession No. NM_001291066.2)
Mouse Wildtype ADAM8 Transcript Variant 2 cDNA
(SEQ ID NO: 100)
atgcttggcctctggctgctcagcgtcttatggacaccagtagcccctggacctcctttgccccatgtgaaacag
tatgaagtggtttggcctcggcgcctagctgcatcccgctcccgcagagccctgccctcccactggggccagtac
ccagagagtctgagctatgctcttgggaccagcgggcacgttttcaccctgcaccttcgaaagaacagggacctg
ctgggctcaagctacacagagacctactcagctgccaatggctctgaggtgacagagcaactgcaggagcaggac
cattgcctctaccaaggccatgtggaagggtacgagggctcagctgccagtattagcacctgtgctggcctcagg
ggctttttccgagttgggtccactgtccacttgattgagcctctggatgctgatgaagaggggcaacatgcgatg
tatcaggcaaagcatctgcaacagaaggctgggacctgtggggtcaaagataccaacctgaatgacctagggcct
cgggcattagaaatatacagggctcagccacggaactggctgatacccagagaaacccgctatgtggagttgtat
gtggttgcagacagccaagagttccagaagttggggagcagagaggccgtgcgccagcgagtgctggaggttgta
aaccacgtggacaagctttatcaggaactcagttttcgagttgtcctggtgggcctggagatctggaacaaggac
aaattctacatcagccgctatgccaacgtgacactggagaacttcttgtcctggagggaacagaacttgcaaggg
cagcacccacatgacaacgtgcaacttatcacgggggttgatttcattgggagcactgttggactggctaaggtg
tctgccctgtgttcccgtcactccggagctgtgaatcaggaccactccaagaactccattggtgtagcctccacc
atggcccatgagctgggccacaacctgggcatgagccatgatgaggacattccaggatgctactgtcctgaacca
cgggagggtggtggctgcatcatgaccgaaagcatcggctccaagttccccaggatattcagcaggtgtagcaag
attgacctagagtcattcgtgacaaaaccccagacaggctgcctgaccaatgttccagatgtcaaccggttcgtg
ggtggccctgtgtgtggaaacctgtttgtggagcatggagagcagtgtgactgtggcacacctcaggactgtcaa
aacccctgctgcaatgccaccacttgccagctggtcaagggggcagagtgtgccagtggtacctgttgtcatgaa
tgcaaggtgaagccagctggagaggtgtgtcgtctcagtaaggacaaatgtgacctggaggagttctgtgatggc
cggaagccaacatgtcccgaagatgccttccaacagaatggcactccctgcccagggggctactgctttgatggg
agctgtcccaccctggcacagcagtgccgggatctgtgggggccaggtgctcgggtagcagccgactcctgctat
acctttagcatccctccgggctgcaatgggaggatgtactctggcaggatcaaccggtgtggagcgctgtactgt
gagggaggccagaagccccttgaacgctccttctgcactttctcctccaaccatggagtctgccatgctcttggc
acaggcagcaacattgacacctttgagctggtattgcagggcaccaagtgcgaggagggaaaggtttgcatggat
ggaagctgccaggacctccgtgtatacagatctgaaaactgctctgctaaatgcaacaaccatggggtatgcaac
cacaagagggagtgccactgtcacaagggctgggcaccacccaactgtgtacagcggctggcagatgtatcagat
gaacaagcagcgtctacgagcctcccagtcagtgtggttgtggtcttggtgatcctggtggctgcgatggtcatc
gtggcaggcatcgtcatctaccgaaaggctccgagacaaatccagaggaggagtgtggcacccaagcctatctcg
gggctctccaaccccctattctacacaagggacagcagcctgccagctaagaacaggcctccagacccttctgag
acagtttctaccaaccagcccccaagacccatagtgaaaccaaagaggcctccccctgcacctccaggtgctgtg
tccagttcaccactcccagttcctgtttatgccccaaagataccaaatcagtttagacctgatcctcccaccaag
cccctcccagagctgaaacccaagcaggtcaagccaacctttgcacccccgacaccaccagtcaagcccgggact
ggagggacggtgcctggagcaactcagggagctggtgagccaaaggttgctctgaaggtccccatccagaagagg
tga

### ADAM8 in Breast Cancer Patients

Breast cancer is the most frequently diagnosed cancer and the leading cause of cancer-related deaths worldwide among women (World Health Organization) with 600,000 breast cancer deaths yearly, mainly from metastatic disease. Large-scale transcriptional analyses identified ADAM8 as one of the most overexpressed genes in breast cancer compared to normal breast tissue (Romagnoli et al., EMBO Mol Med, 6 (2): 278-294, 2014). High ADAM8 levels were an independent predictor of both poor disease-free and overall survival. Breast cancers are heterogeneous with different tumor drivers identified in distinct patient subpopulations. In many cases, breast tumors are driven by aberrant receptor signaling, e.g., the estrogen receptor a (ER), or the epidermal growth factor receptor 2 (HER2).

In the past two decades, tremendous advances have been made with the introduction of targeted therapies against these receptors: hormone modulators (e.g. tamoxifen) for ER-positive tumors, and the humanized anti-HER2 antibody (trastuzumab) for HER2-positive tumors. However, approximately 15% of breast tumors, termed triple-negative breast cancers (TNBCs), are devoid of elevated levels of ER, HER2, or progesterone receptor expression, and have thus not benefitted from these recent treatment advances. ADAM8 mRNA levels were significantly higher in Grade 3 vs. Grade 1 and 2 breast cancers and especially in basal-like tumors, known to be mostly TNBCs. In immunohistochemistry (IHC) studies, 34.0% of primary patient TNBC tumor samples, and 48.2% of all breast cancer-derived metastases displayed high ADAM8 levels whereas normal mammary tissue was negative (n=50) (Romagnoli et al., EMBO Mol Med, 6 (2): 278-294, 2014). TNBCs are highly aggressive and occur preferentially in women who are younger or of African-American descent.

Due to the lack of elevated expression of hormone and HER2 receptors, treatment options for TNBCs are restricted to chemotherapy and radiation, which are insufficient to block tumor progression or metastatic dissemination, and have severe side effects. Patients frequently recur with locoregional disease or distant metastasis. The main sites of metastasis include the bones, lymph nodes, brain, lungs, and liver, but skin, chest wall (including bone, cartilage, and soft tissue), contralateral breast, soft tissue, bone marrow, ovary, adrenal gland, and even pancreatic spread have been observed. Furthermore, IHC of samples from patients with a particularly aggressive TNBC subclass termed Triple-Negative Inflammatory Breast Cancer were ADAM8-positive. Specifically, 45.5% of primary and 50.0% of axillary lymph node metastases expressed high levels of ADAM8. In addition, elevated ADAM8 mRNA or protein levels were also detected in HER2+ breast cancers and in 13.5% of premalignant Ductal Carcinoma in Situ (DCIS) samples from patients who later progressed to malignant breast cancer (Romagnoli et al., EMBO Mol Med, 6 (2): 278-294, 2014). Due to their enhanced ability to spread compared to other breast tumor types and the poor response rate to Standard of Care (SoC) treatments, TNBCs account for more than 25% of breast cancer deaths, despite having a lower incidence rate.

### Mouse and Cell-Based Mechanistic Studies of ADAM8 Role in Breast Cancer

Stable ADAM8 knockdown (KD) in MDA-MB-231 TNBC cells resulted in profound tumor growth inhibition (TGI, percent reduction in mean tumor volume) in an orthotopic mammary fat pad (MFP) model (Romagnoli et al., EMBO Mol Med, 6 (2): 278-294, 2014). Notably, female NOD/SCID mice injected with Control MDA-MB-231 TNBC cells [containing a control shRNA and thus retaining high levels of ADAM8] started to develop tumors within 2 weeks, which progressed rapidly to 1 cm³ (the experimental endpoint). In contrast, tumors derived from ADAM8 KD MDA-MB-231 TNBC cells [stably expressing shADAM8 RNA and thus with substantially reduced ADAM8 expression] failed to grow beyond a palpable size (0.05 cm³) even after more than 4 weeks. Tumors derived from ADAM8 KD MDA-MB-231 TNBC cells displayed reduced angiogenesis, shed fewer circulating tumor cells (CTCs) and CTC clusters (CTCCs) into the bloodstream, and displayed a substantial reduction in metastasis to the brain (Romagnoli et al., EMBO Mol Med, 6 (2): 278-294, 2014; Lyons et al., Biomed Opt Express, 7 (3): 1042-1050, 2016).

In a follow-up experiment, in which cells were injected directly into the mouse blood stream through the heart, ADAM8 KD TNBC cells were unable to colonize distant organs while mice with ADAM8-expressing TNBC cells formed large metastases in a variety of organs, including bones, brain, and lungs. Consistently, in TNBC cells in culture, a reduction in ADAM8 levels strongly diminished secretion of pro-angiogenic factors, in vitro angiogenesis, and migratory and invasive properties (Romagnoli et al., EMBO Mol Med, 6 (2): 278-294, 2014). Furthermore, hypoxic areas in control MDA-MB-231 tumors were often associated with elevated ADAM8 staining, which was consistent with cell culture models where growth under hypoxic conditions (1% O₂ for 24 h) led to increased ADAM8 levels (Romagnoli et al., EMBO Mol Med, 6 (2): 278-294, 2014). Overall, studies conducted in cell-based and orthotopic mouse models revealed that ADAM8 promotes tumor growth and dissemination by stimulating: (i) angiogenesis via the cleavage and release of cell-bound precursor proteins into active angiogenic factors, such as VEGF-A, PDGF-AA, angiogenin, and placenta growth factors, through its MP domain activity, and (ii) tumor cell intra/extravasation and metastasis via activation of β1-integrin and enhancement of interactions between cancer cells and endothelial cells lining blood vessel walls, through its DI domain activity (Romagnoli et al., EMBO Mol Med, 6 (2): 278-294, 2014) (FIG. 1B).

The proteins provided herein show significant specificity, e.g., the proteins provided herein strongly inhibit both the ADAM8 MP and DI activities, but fail to interact with closely related ADAM9, ADAM12, ADAM15 or ADAM33. The data herein also demonstrate that the proteins provided herein very effectively inhibit the MP domain of ADAM8 responsible for tumor growth, as well as, the DI domain of ADAM8, which is critical to stop tumor dissemination, the ultimate cause of patient mortality.

### Proteins

Provided herein are proteins that inhibit both the metalloprotease activity and disintegrin activity of human ADAM8, wherein the protein includes an antigen-binding domain that: (i) binds specifically to human ADAM8; and (ii) binds to an epitope within human ADAM8 that includes at least one (e.g., at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, or at least 25, at least 26, at least 27, at least 28, at least 29, at least 30, at least 31, at least 32, at least, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68 amino acids) amino acid within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

In some embodiments of any of the proteins described herein, the protein binds to an epitope within human ADAM8 that includes about 1 amino acid to about 60 amino acids (e.g., about 1 amino acid to about 55 amino acids, about 1 amino acid to about 50 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, about 1 amino acid to about 15 amino acids, about 1 amino acid to about 10 amino acids, about 1 amino acid to about 5 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 to about 25 amino acids, about 5 amino acids to about 20 amino acids, about 5 amino acids to about 15 amino acid, about 5 amino acids to about 10 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acid, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 60 amino acids, about 40 amino acids to about 55 amino acids, about 40 amino acids to about 50 amino acids, about 40 amino acids to about 45 amino acids, about 45 amino acids to about 60 amino acids, about 45 amino acids to about 55 amino acids, about 45 amino acids to about 50 amino acids, about 50 amino acids to about 60 amino acids, about 50 amino acids to about 55 amino acids, or about 55 amino acids to about 60 amino acids) within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

In some embodiments of any of the proteins described herein, the protein binds to human ADAM8 with a K_{D} of about 0.1 nM to about 250 nM (e.g., about 0.1 nM to about 200 nM, about 0.1 nM to about 150 nM, about 0.1 nM to about 100 nM, about 0.1 nM to about 80 nM, about 0.1 nM to about 60 nM, about 0.1 nM to about 40 nM, about 0.1 nM to about 20 nM, about 0.1 nM to about 10 nM, about 0.1 nM to about 5 nM, about 0.1 nM to about 1 nM, about 1 nM to about 250 nM, about 1 nM to about 200 nM, about 1 nM to about 150 nM, about 1 nM to about 100 nM, about 1 nM to about 80 nM, about 1 nM to about 60 nM, about 1 nM to about 40 nM, about 1 nM to about 20 nM, about 1 nM to about 10 nM, about 1 nM to about 5 nM, about 5 nM to about 250 nM, about 5 nM to about 200 nM, about 5 nM to about 150 nM, about 5 nM to about 100 nM, about 5 nM to about 80 nM, about 5 nM to about 60 nM, about 5 nM to about 40 nM, about 5 nM to about 20 nM, about 5 nM to about 10 nM, about 10 nM to about 250 nM, about 10 nM to about 200 nM, about 10 nM to about 150 nM, about 10 nM to about 100 nM, about 10 nM to about 80 nM, about 10 nM to about 60 nM, about 10 nM to about 40 nM, about 10 nM to about 20 nM, about 20 nM to about 250 nM, about 20 nM to about 200 nM, about 20 nM to about 150 nM, about 20 nM to about 100 nM, about 20 nM to about 80 nM, about 20 nM to about 60 nM, about 20 nM to about 40 nM, about 40 nM to about 250 nM, about 40 nM to about 200 nM, about 40 nM to about 150 nM, about 40 nM to about 100 nM, about 40 nM to about 80 nM, about 40 nM to about 60 nM, about 60 nM to about 250 nM, about 60 nM to about 200 nM, about 60 nM to about 150 nM, about 60 nM to about 100 nM, about 60 nM to about 80 nM, about 80 nM to about 250 nM, about 80 nM to about 200 nM, about 80 nM to about 150 nM, about 80 nM to about 100 nM, about 100 nM to about 250 nM, about 100 nM to about 200 nM, about 100 nM to about 150 nM, about 150 nM to about 250 nM, about 150 nM to about 200 nM, or about 200 nM to about 250 nM).

In some embodiments of any of the proteins described herein, the protein binds to human ADAM8 with a K_{D} of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments of any of the proteins described herein, the protein binds to human ADAM8 with a K_{D} of about 1×10⁻³ M to about 1×10⁻¹³ M, about 1×10⁻³ M to about 1×10⁻¹² M, about 1×10⁻³ M to about 1×10⁻¹¹ M, about 1×10⁻³ M to about 1×10⁻¹⁰ M, about 1×10⁻³ M to about 1×10⁻⁹ M, about 1×10⁻³ M to about 1×10⁻⁸ M, about 1×10⁻³ M to about 1×10⁻⁷ M, about 1×10⁻³ M to about 1×10⁻⁶ M, about 1×10⁻³ M to about 1×10⁻⁵ M, about 1×10⁻⁴ M to about 1×10⁻¹³ M, about 1×10⁻⁴ M to about 1×10⁻¹² M, about 1×10⁻⁴ M to about 1×10⁻¹¹ M, about 1×10⁻⁴ M to about 1×10⁻¹⁰ M, about 1×10⁻⁴ M to about 1×10⁻⁹ M, about 1×10⁻⁴ M to about 1×10⁻⁸ M, about 1×10⁻⁴ M to about 1×10⁻⁷ M, about 1×10⁻⁴ M to about 1×10⁻⁶ M, about 1×10⁻⁴ M to about 1×10⁻⁵ M, about 1×10⁻⁵ M to about 1×10⁻¹³ M, about 1×10⁻⁵ M to about 1×10⁻¹² M, about 1×10⁻⁵ M to about 1×10⁻¹¹ M, about 1×10⁻⁵ M to about 1×10⁻¹⁰ M, about 1×10⁻⁵ M to about 1×10⁻⁹ M, about 1×10⁻⁵ M to about 1×10⁻⁸ M, about 1×10⁻⁵ M to about 1×10⁻⁷ M, about 1×10⁻⁵ M to about 1×10⁻⁵ M, about 1×10⁻⁶ M to about 1×10⁻⁸ M, about 1×10⁻⁶ M to about 1×10⁻¹³ M, about 1×10⁻⁶ M to about 1×10⁻¹² M, about 1×10⁻⁶ M to about 1×10⁻¹¹ M, about 1×10⁻⁶ M to about 1×10⁻¹⁰ M, about 1×10⁻⁶ M to about 1×10⁻⁹ M, about 1×10⁻⁶ M to about 1×10⁻⁸ M, about 1×10⁻⁶ M to about 1×10⁻⁷ M, about 1×10⁻⁷ M to about 1×10⁻¹³ M, about 1×10⁻⁷ M to about 1×10⁻¹² M, about 1×10⁻⁷ M to about 1×10⁻¹¹ M, about 1×10⁻⁷ M to about 1×10⁻¹⁰ M, about 1×10⁻⁷ M to about 1×10⁻⁹ M, about 1×10⁻⁷ M to about 1×10⁻⁸ M, about 1×10⁻⁸ M to about 1×10⁻¹³ M, about 1×10⁻⁸ M to about 1×10⁻¹² M, about 1×10⁻⁸ M to about 1×10-11 M, about 1×10⁻⁸ M to about 1×10⁻¹⁰ M, about 1×10⁻⁸ M to about 1×10⁻⁹ M, about 1×10⁻⁹ M to about 1×10⁻¹³ M, about 1×10⁻⁹ M to about 1×10⁻¹² M, about 1×10⁻⁹ M to about 1×10⁻¹¹ M, about 1×10⁻⁹ M to about 1×10⁻¹⁰ M, about 1×10⁻¹⁰ M to about 1×10⁻¹³ M, about 1×10⁻¹⁰ M to about 1×10⁻¹² M, about 1×10⁻¹⁰ M to about 1×10⁻¹¹ M, about 1×10⁻¹¹ M to about 1×10⁻¹³ M, about 1×10⁻¹¹ M to about 1×10⁻¹² M, or about 1×10⁻¹² M to about 1×10⁻¹³ M (inclusive).

In some embodiments of any of the proteins described herein, the protein includes a single polypeptide. In some embodiments of any of the proteins described herein, the antigen-binding domain is a VHH domain, a VNAR domain, or a scFv. In some embodiments of any of the proteins described herein, the protein is selected from the group consisting of: a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, and a tandem-scFv.

In some embodiments of any of the proteins described herein, the protein includes two or more polypeptides. In some embodiments of any of the proteins described herein, the protein is selected from the group consisting of: an antibody, a VHH-scAb, a VHH-Fab, a Dual scFab, a F(ab′) 2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KA-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′) 2-scFv2, a scFv-KIH, a Fab-scFv-Fc, a tetravalent HCAb, a scDiabody-Fc, a Diabody-Fc, a tandem scFv-Fc, an Intrabody, a dock and lock, an lmmTAC, an IgG-IgG conjugate, a Cov-X-Body, and a scFv1-PEG-scFV2.

In some embodiments of any of the proteins described herein, the protein is an antibody that is an IgG antibody. In some embodiments of any of the proteins described herein, the IgG antibody is an IgG1, IgG2, IgG3, or IgG4 antibody. In some embodiments of any of the proteins described herein, the antibody is a monospecific antibody. In some embodiments of any of the proteins described herein, the antibody is a multi-specific (e.g., bispecific antibody, e.g., a knobs-in-hole bispecific antibody). In some embodiments of any of the proteins described herein, the antibody is a bispecific antibody.

In some embodiments of any of the proteins described herein, the antigen-binding domain includes heavy chain variable domain CDRs of GFSFPDYY (SEQ ID NO: 2), IRDSANGYTT (SEQ ID NO: 3), and ARYSRYYGMDY (SEQ ID NO: 4), and light chain variable domain CDRs of QTVNYD (SEQ ID NO: 5), FAS (SEQ ID NO: 6), and QQDYSAPWT (SEQ ID NO: 7). In some embodiments of any of the proteins described herein, the antigen-binding domain includes a light chain variable domain sequence of: SIVMTQTPKILLVSAGDRVTITCKASQTVNYDVAWYQQKPGQSPKPVIYFASNRYTG VPDRFTGSGFGTDFTFTISTVQAEDLAVYFCQQDYSAPWTFGGGTKLEIK (SEQ ID NO: 8). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 8. In some embodiments of any of the proteins described herein, the antigen-binding domain includes a light chain variable domain sequence of SEQ ID NO: 8, except that it includes about 1 to about 6 amino acid substitutions (e.g., 1, 2, 3, 4, 5, or 6 amino acid substitutions) in SEQ ID NO: 8. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 9. In some embodiments of any of the proteins described herein, the antigen-binding domain includes a heavy chain variable domain sequence of: EVKLVESGGGLVQPGGSLSLSCAASGFSFPDYYMSWVRQPPGKALEWLGFIRDSAN GYTTEYIASVKGRFTFSRDNSQSILYLQMNALRAEDSATYYCARYSRYYGMDYWGQ GTSVTVSS (SEQ ID NO: 10). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 10. In some embodiments of any of the proteins described herein, the antigen-binding domain includes a heavy chain variable domain sequence of SEQ ID NO: 10, except that it includes about 1 to about 6 amino acid substitutions (e.g., 1, 2, 3, 4, 5, or 6 amino acid substitutions) in SEQ ID NO: 10. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 11.

In some embodiments of any of the proteins described herein, the antigen-binding domain includes heavy chain variable domain CDRs of GYTFTDYY (SEQ ID NO: 12), ISPNIGGA (SEQ ID NO: 13), and TRGGSSYPYFYAMDY (SEQ ID NO: 14), and light chain variable domain CDRs of QSLLYSSNQKKY (SEQ ID NO: 15), WAS (SEQ ID NO: 16), and QQFYSYPYT (SEQ ID NO: 17). In some embodiments of any of the proteins described herein, the antigen-binding domain includes a light chain variable domain sequence of: DIVMSQSPSSLAVSVGEKVTMSCKSSQSLLYSSNQKKYLAWYQQKPGQSPKLLIYW ASTRESGVPDRFTGSGSGTD FTLTISSVKAEDLAVYYCQQFYSYPYTFGGGTKLEINR (SEQ ID NO: 18). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 18. In some embodiments of any of the proteins described herein, the antigen-binding domain includes a light chain variable domain sequence of SEQ ID NO: 18, except that it includes about 1 to about 6 amino acid substitutions (e.g., 1, 2, 3, 4, 5, or 6 amino acid substitutions) in SEQ ID NO: 18. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 19. In some embodiments of any of the proteins described herein, the antigen-binding domain includes a heavy chain variable domain sequence of: EVQLQQSGPEMVKPGTSVKISCKASGYTFTDYYINWVKQSHGKSLEWIGDISPNIGG ATYNPKFKGKAILTVDKSARTAYMELRSLTSEDSAVYCCTRGGSSYPYFYAMDYWG QGTSVTVSS (SEQ ID NO: 20). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 20. In some embodiments of any of the proteins described herein, the antigen-binding domain includes a heavy chain variable domain sequence of SEQ ID NO: 20, except that it includes about 1 to about 6 amino acid substitutions (e.g., 1, 2, 3, 4, 5, or 6 amino acid substitutions) in SEQ ID NO: 20. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 21.

In some embodiments of any of the proteins described herein, the antigen-binding domain includes heavy chain variable domain CDRs of GFTFSDAW (SEQ ID NO: 22), IRGKVNNLAT (SEQ ID NO: 23), and LGRYDATYAMDY (SEQ ID NO: 24), and light chain variable domain CDRs of QSLVHSDGNTY (SEQ ID NO: 25), KLS (SEQ ID NO: 26), and SQSTHVPWT (SEQ ID NO: 27). In some embodiments of any of the proteins described herein, the antigen-binding domain includes a light chain variable domain sequence of: DVVMTQTPLSLPVSLGDQASISCRSSQSLVHSDGNTYLHWYLQKPGQSPKLLIYKLS NRFSGVPDRFSGSGSGTDF TLKISR VEAEDLGVYFCSQSTHVPWTFGGGTKLEIK (SEQ ID NO: 28). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 28. In some embodiments of any of the proteins described herein, the antigen-binding domain includes a light chain variable domain sequence of SEQ ID NO: 28, except that it includes about 1 to about 6 amino acid substitutions (e.g., 1, 2, 3, 4, 5, or 6 amino acid substitutions) in SEQ ID NO: 28. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 29. In some embodiments of any of the proteins described herein, the antigen-binding domain includes a heavy chain variable domain sequence of: EVKLEESGGGLVQPGGSMKLSCAASGFTFSDAWMDWVRQSPEKGLEWVAEIRGKV NNLATYYVESVKGRFTISRDDSKSSVYLQMNSLRAEDTGIYYCLGRYDATYAMDY WGQGTSVTVSS (SEQ ID NO: 30). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 30. In some embodiments of any of the proteins described herein, the antigen-binding domain includes a heavy chain variable domain sequence of SEQ ID NO: 30, except that it includes about 1 to about 6 amino acid substitutions (e.g., 1, 2, 3, 4, 5, or 6 amino acid substitutions) in SEQ ID NO: 30. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 31.

In some embodiments of any of the proteins described herein, the antigen-binding domain includes heavy chain variable domain CDRs of GFSFTDYY (SEQ ID NO: 32), IRDSANGYTA (SEQ ID NO: 33), and ARYSRYYAMDY (SEQ ID NO: 34), and light chain variable domain CDRs of QSVNYD (SEQ ID NO: 35), FAS (SEQ ID NO: 36), and QQDYSSPWT (SEQ ID NO: 37). In some embodiments of any of the proteins described herein, the antigen-binding domain includes a light chain variable domain sequence of: FIVMTQTPKILLVSAGDRITITCKASQSVNYDVAWYQQKPGQSPKPVIYFASNRYTGV PDRFTGSGFGTDFTFTISTVQAEDLAVYFCQQDYSSPWTFGGGTKLEIK (SEQ ID NO: 38). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 38. In some embodiments of any of the proteins described herein, the antigen-binding domain includes a light chain variable domain sequence of SEQ ID NO: 38, except that it includes about 1 to about 6 amino acid substitutions (e.g., 1, 2, 3, 4, 5, or 6 amino acid substitutions) in SEQ ID NO: 38. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 39. In some embodiments of any of the proteins described herein, the antigen-binding domain includes a heavy chain variable domain sequence of: EVKLVESGGGLVQPGGSLSLSCETSGFSFTDYYMIWVRQPPGKALEWLGFIRDSANG YTAEYIASVKGRFTFSRDNSQSILYLQMNALRAEDSATYYCARYSRYYAMDYWGQ GTSVTVAP (SEQ ID NO: 40). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 40. In some embodiments of any of the proteins described herein, the antigen-binding domain includes a heavy chain variable domain sequence of SEQ ID NO: 40, except that it includes about 1 to about 6 amino acid substitutions (e.g., 1, 2, 3, 4, 5, or 6 amino acid substitutions) in SEQ ID NO: 40. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 41.

In some embodiments of any of the proteins described herein, the antigen-binding domain includes heavy chain variable domain CDRs of GYTFTDYN (SEQ ID NO: 42), INPNNGGT (SEQ ID NO: 43), and ARKRGLGQAWLAY (SEQ ID NO: 44), and light chain variable domain CDRs of QSLLYSGNQKNY (SEQ ID NO: 45), GAS (SEQ ID NO: 46), and QNDHSYPLT (SEQ ID NO: 47). In some embodiments of any of the proteins described herein, the antigen-binding domain includes a light chain variable domain sequence of: DIVMTQSPSSRSVSAGEKVTMSCKSSQSLLYSGNQKNYLAWYQQKPGQPPKLLIYG ASTRESGVPDRFTGSGSGTDFTLTISSVQAEDLAVYYCQNDHSYPLTFGAGTKLELK (SEQ ID NO: 48). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 48. In some embodiments of any of the proteins described herein, the antigen-binding domain includes a light chain variable domain sequence of SEQ ID NO: 48, except that it includes about 1 to about 6 amino acid substitutions (e.g., 1, 2, 3, 4, 5, or 6 amino acid substitutions) in SEQ ID NO: 48. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 49. In some embodiments of any of the proteins described herein, the antigen-binding domain includes a heavy chain variable domain sequence of: EVQLQQSGPELVKPGASVKIPCKASGYTFTDYNMDWVKQSHGKSLDWIGDINPNNG GTIYNQKFKGKATLTVDKSSSTAYMELRSLTSEDTAVYYCARKRGLGQAWLAYWG QGTLVTVSA (SEQ ID NO: 50). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 50. In some embodiments of any of the proteins described herein, the antigen-binding domain includes a heavy chain variable domain sequence of SEQ ID NO: 50, except that it includes about 1 to about 6 amino acid substitutions (e.g., 1, 2, 3, 4, 5, or 6 amino acid substitutions) in SEQ ID NO: 50. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 51.

In some embodiments of any of the proteins described herein, the antigen-binding domain includes heavy chain variable domain CDRs of GFTFSYAW (SEQ ID NO: 52), IRSKANNYAT (SEQ ID NO: 53), and MGRYDAAYGMDY (SEQ ID NO: 54), and light chain variable domain CDRs of QSLVHSNGITY (SEQ ID NO: 55), KVS (SEQ ID NO: 56), and SQSTHVPWT (SEQ ID NO: 57). In some embodiments of any of the proteins described herein, the antigen-binding domain includes a light chain variable domain sequence of: DVVMTQTPLSLPVSLGYQASISCRSSQSLVHSNGITYLHWYLQKPGQSPKLLIYKVSN RFSGVPDRFSGSGSGTDF TLKISRVEAEDLGVYFCSQSTHVPWTFGGGTKLEIK (SEQ ID NO: 58). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 58. In some embodiments of any of the proteins described herein, the antigen-binding domain includes a light chain variable domain sequence of SEQ ID NO: 58, except that it includes about 1 to about 6 amino acid substitutions (e.g., 1, 2, 3, 4, 5, or 6 amino acid substitutions) in SEQ ID NO: 58. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 59. In some embodiments of any of the proteins described herein, the antigen-binding domain includes a heavy chain variable domain sequence of: EVKLEESGGGLVQPGGSMKLSCAASGFTFSYAWMDWVRQSPEKGLEWVAEIRSKA NNYATYYAESVKGRFTISRNDSKSSVYLQMNSLRIEDTGIYYCMGRYDAAYGMDY WGQGTSVTVSS (SEQ ID NO: 60). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 60. In some embodiments of any of the proteins described herein, the antigen-binding domain includes a heavy chain variable domain sequence of SEQ ID NO: 60, except that it includes about 1 to about 6 amino acid substitutions (e.g., 1, 2, 3, 4, 5, or 6 amino acid substitutions) in SEQ ID NO: 60. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 61.

In some embodiments of any of the proteins described herein, the protein competitively binds to human ADAM8 with an antigen-binding domain including: (i) a light chain variable domain of SEQ ID NO: 8 and a heavy chain variable domain of SEQ ID NO: 10; (ii) a light chain variable domain of SEQ ID NO: 18 and a heavy chain variable domain of SEQ ID NO: 20; (iii) a light chain variable domain of SEQ ID NO: 28 and a heavy chain variable domain of SEQ ID NO: 30; (iv) a light chain variable domain of SEQ ID NO: 38 and a heavy chain variable domain of SEQ ID NO: 40; (v) a light chain variable domain of SEQ ID NO: 48 and a heavy chain variable domain of SEQ ID NO: 50; or (vi) a light chain variable domain of SEQ ID NO: 58 and a heavy chain variable domain of SEQ ID NO: 60.

In some embodiments of any of the proteins described herein, the antigen-binding domain includes heavy chain variable domain CDRs of GFTFSDAW (SEQ ID NO: 62), IRNKANSHAT (SEQ ID NO: 63), and TRDGGYYAWFAY (SEQ ID NO: 64), and light chain variable domain CDRs of QSIVHSDGNTY (SEQ ID NO: 65), RVS (SEQ ID NO: 66), and FHGSHIPLT (SEQ ID NO: 67). In some embodiments of any of the proteins described herein, the antigen-binding domain includes a light chain variable domain sequence of: GALLTQTPLSLPVSLGDQVSISCRSSQSIVHSDGNTYLEWYLQKPGQSPKLLIYRVSN RFSGVPDRFSGSGSGTDF TLKISRVEAEDLGIYYCFHGSHIPLTFGAGTKLELK (SEQ ID NO: 68). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 68. In some embodiments of any of the proteins described herein, the antigen-binding domain includes a light chain variable domain sequence of SEQ ID NO: 68, except that it includes about 1 to about 6 amino acid substitutions (e.g., 1, 2, 3, 4, 5, or 6 amino acid substitutions) in SEQ ID NO: 68. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 69. In some embodiments of any of the proteins described herein, the antigen-binding domain includes a heavy chain variable domain sequence of: EVKFEESGGGLVQPGGSMKLSCAASGFTFSDAWMDWVRQSPEKGLEWVAEIRNKA NSHATYYAESVKGRFTISRDDSKSTVYLQMNSLRAEDTGIYYCTRDGGYYAWFAY WGQGTLVTVSA (SEQ ID NO: 70). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 70. In some embodiments of any of the proteins described herein, the antigen-binding domain includes a heavy chain variable domain sequence of SEQ ID NO: 70, except that it includes about 1 to about 6 amino acid substitutions (e.g., 1, 2, 3, 4, 5, or 6 amino acid substitutions) in SEQ ID NO: 70. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 71.

In some embodiments of any of the proteins described herein, the antigen-binding domain includes heavy chain variable domain CDRs of GFTFSDAW (SEQ ID NO: 72), IRNKANNHAT (SEQ ID NO: 73), and TRDGGYYAWFAY (SEQ ID NO: 74), and light chain variable domain CDRs of QSIVHSDGNTY (SEQ ID NO: 75), KVS (SEQ ID NO: 76), and FHGSHIPLT (SEQ ID NO: 77). In some embodiments of any of the proteins described herein, the antigen-binding domain includes a light chain variable domain sequence of: GVLMTQTPLSLPVSLGDQASISCRSSQSIVHSDGNTYLEWYLQKPGQSPKLLLYKVS NRFSGVPDRFSGSGSGTDF TLKISR VEAEDLGVYYCFHGSHIPLTFGAGTKLELK (SEQ ID NO: 78). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 78. In some embodiments of any of the proteins described herein, the antigen-binding domain includes a light chain variable domain sequence of SEQ ID NO: 78, except that it includes about 1 to about 6 amino acid substitutions (e.g., 1, 2, 3, 4, 5, or 6 amino acid substitutions) in SEQ ID NO: 78. In some embodiments of any of the proteins described herein, the light chain variable domain sequence is encoded by a nucleic acid including a sequence that is at least 95% (e.g., at least 96%, at least 97%, at least 98%, at least 99%, or 100%) identical to SEQ ID NO: 79. In some embodiments of any of the proteins described herein, the antigen-binding domain includes a heavy chain variable domain sequence of: EVKFEESGGGLVQPGGSMKLSCAASGFTFSDAWMDWVRQSPEKGLEWVAEIRNKA NNHATYYAESVKGRFTISRDDSKSSVYLQMNSLRAEDTGIYYCTRDGGYYAWFAY WGQGTLVTVSA (SEQ ID NO: 80). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 80. In some embodiments of any of the proteins described herein, the antigen-binding domain includes a heavy chain variable domain sequence of SEQ ID NO: 80, except that it includes about 1 to about 6 amino acid substitutions (e.g., 1, 2, 3, 4, 5, or 6 amino acid substitutions) in SEQ ID NO: 80. In some embodiments of any of the proteins described herein, the heavy chain variable domain sequence is encoded by a nucleic acid including a sequence that is at least 95% (e.g., at least 96%, at least 97%, at least 98%, at least 99%, or 100%) identical to SEQ ID NO: 81.

In some embodiments of any of the proteins described herein, the antigen-binding domain binds to an epitope within human ADAM8 that includes at least one amino acid (e.g., at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, or at least 24 amino acids) within the sequence of: CCNSTTCQLAEGAQCAHGTCCQECK (SEQ ID NO: 86).

In some embodiments of any of the proteins described herein, the antigen-binding domain binds to an epitope within human ADAM8 that includes at least one amino acid (e.g., at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 26, or at least 27 amino acids) within the sequence of: RNRCCNSTTCQLAEGAQCAHGTCCQECK (SEQ ID NO: 104). In some embodiments of any of the proteins described herein, the antigen-binding domain includes heavy chain variable domain CDRs of GFSFPDYY (SEQ ID NO: 2), IRDSANGYTT (SEQ ID NO: 3), and ARYSRYYGMDY (SEQ ID NO: 4), and light chain variable domain CDRs of QTVNYD (SEQ ID NO: 5), FAS (SEQ ID NO: 6), and QQDYSAPWT (SEQ ID NO: 7). In some embodiments of any of the proteins described herein, the antigen-binding domain includes a light chain variable sequence of: SIVMTQTPKILLVSAGDRVTITCKASQTVNYDVAWYQQKPGQSPKPVIYFASNRYTG VPDRFTGSGFGTDFTFTISTVQAEDLAVYFCQQDYSAPWTFGGGTKLEIK (SEQ ID NO: 8). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 8. In some embodiments of any of the proteins described herein, the antigen-binding domain includes a light chain variable domain sequence of SEQ ID NO: 8, except that it includes about 1 to about 6 amino acid substitutions (e.g., 1, 2, 3, 4, 5, or 6 amino acid substitutions) in SEQ ID NO: 8. In some embodiments of any of the proteins described herein, the antigen-binding domain includes a heavy chain variable domain sequence of: EVKLVESGGGLVQPGGSLSLSCAASGFSFPDYYMSWVRQPPGKALEWLGFIRDSAN GYTTEYIASVKGRFTFSRDNSQSILYLQMNALRAEDSATYYCARYSRYYGMDYWGQ GTSVTVSS (SEQ ID NO: 10). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 10. In some embodiments of any of the proteins described herein, the antigen-binding domain includes a heavy chain variable domain sequence of SEQ ID NO: 10, except that it includes about 1 to about 6 amino acid substitutions (e.g., 1, 2, 3, 4, 5, or 6 amino acid substitutions) in SEQ ID NO: 10. In some embodiments of any of the proteins described herein, the protein competitively binds to human ADAM8 with an antigen-binding domain that includes: (i) a light chain variable domain of SEQ ID NO: 18 and a heavy chain variable domain of SEQ ID NO: 20; (ii) a light chain variable domain of SEQ ID NO: 28 and a heavy chain variable domain of SEQ ID NO: 30; (iii) a light chain variable domain of SEQ ID NO: 38 and a heavy chain variable domain of SEQ ID NO: 40; (iv) a light chain variable domain of SEQ ID NO: 48 and a heavy chain variable domain of SEQ ID NO: 50; or (v) a light chain variable domain of SEQ ID NO: 58 and a heavy chain variable domain of SEQ ID NO: 60.

In some embodiments of any of the proteins described herein, the antigen-binding domain binds to an epitope within human ADAM8 that includes at least one amino acid (e.g., at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least, at least 33, at least 34, at least 35, at least 36, at least 37, at least 38, at least 39, at least 40, at least 41, at least 42, at least 43, at least 44, at least 45, at least 46, at least 47, at least 48, or at least 49 amino acids) within the sequence of:

(SEQ ID NO: 87)
LAEGAQCAHGTCCQECKVKPAGELCRPKKDMCDLEEFCDGRHPECPE
DAF.

In some embodiments of any of the proteins described herein, the antigen-binding domain includes heavy chain variable domain CDRs of GYTFTDYY (SEQ ID NO: 12), ISPNIGGA (SEQ ID NO: 13), and TRGGSSYPYFYAMDY (SEQ ID NO: 14), and light chain variable domain CDRs of QSLLYSSNQKKY (SEQ ID NO: 15), WAS (SEQ ID NO: 16), and QQFYSYPYT (SEQ ID NO: 17). In some embodiments of any of the proteins described herein, the antigen-binding domain includes a light chain variable domain sequence of: DIVMSQSPSSLAVSVGEKVTMSCKSSQSLLYSSNQKKYLAWYQQKPGQSPKLLIYW ASTRESGVPDRFTGSGSGTD FTLTISSVKAEDLAVYYCQQFYSYPYTFGGGTKLEINR (SEQ ID NO: 18). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 18. In some embodiments of any of the proteins described herein, the antigen-binding domain includes a light chain variable domain sequence of SEQ ID NO: 18, except that it includes about 1 to about 6 amino acid substitutions (e.g., 1, 2, 3, 4, 5, or 6 amino acid substitutions) in SEQ ID NO: 18. In some embodiments of any of the proteins described herein, the antigen-binding domain includes a heavy chain variable domain sequence of: EVQLQQSGPEMVKPGTSVKISCKASGYTFTDYYINWVKQSHGKSLEWIGDISPNIGG ATYNPKFKGKAILTVDKSARTAYMELRSLTSEDSAVYCCTRGGSSYPYFYAMDYWG QGTSVTVSS (SEQ ID NO: 20). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 20. In some embodiments of any of the proteins described herein, the antigen-binding domain includes a heavy chain variable domain sequence of SEQ ID NO: 20, except that it includes about 1 to about 6 amino acid substitutions (e.g., 1, 2, 3, 4, 5, or 6 amino acid substitutions) in SEQ ID NO: 20. In some embodiments of any of the proteins described herein, the protein competitively binds to human ADAM8 with an antigen-binding domain including: (i) a light chain variable domain of SEQ ID NO:8 and a heavy chain variable domain of SEQ ID NO: 10; (ii) a light chain variable domain of SEQ ID NO: 38 and a heavy chain variable domain of SEQ ID NO: 40; or (iii) a light chain variable domain of SEQ ID NO: 48 and a heavy chain variable domain of SEQ ID NO: 50.

In some embodiments of any of the proteins described herein, the antigen-binding domain binds to an epitope within human ADAM8 that includes at least one amino acid (e.g., at least two, at least three, at least four, at least five, at least six, at least seven, at least eight, at least nine, at least ten, at least 11, at least 12, at least 13, at least 14, at least 15, at least 16, at least 17, or at least 18 amino acids) within the sequence of: DCGPPEDCRNRCCNSTTCQ (SEQ ID NO: 88). In some embodiments of any of the proteins described herein, the antigen-binding domain includes heavy chain variable domain CDRs of GFTFSDAW (SEQ ID NO: 22), IRGKVNNLAT (SEQ ID NO: 23), and LGRYDATYAMDY (SEQ ID NO: 24), and light chain variable domain CDRs of QSLVHSDGNTY (SEQ ID NO: 25), KLS (SEQ ID NO: 26), and SQSTHVPWT (SEQ ID NO: 27). In some embodiments of any of the proteins described herein, the antigen-binding domain includes a light chain variable domain sequence of: DVVMTQTPLSLPVSLGDQASISCRSSQSLVHSDGNTYLHWYLQKPGQSPKLLIYKLS NRFSGVPDRFSGSGSGTDF TLKISR VEAEDLGVYFCSQSTHVPWTFGGGTKLEIK (SEQ ID NO: 28). In some embodiments of any of the proteins described herein, the antigen-binding domain includes a light chain variable domain sequence that is at least 95% (e.g., at least 96%, at least 97%, at least 98%, or at least 99%) identical to SEQ ID NO: 28. In some embodiments of any of the proteins described herein, the antigen-binding domain includes a light chain variable domain sequence of SEQ ID NO: 28, except that it includes about 1 to about 6 amino acid substitutions (e.g., 1, 2, 3, 4, 5, or 6 amino acid substitutions) in SEQ ID NO: 28. In some embodiments of any of the proteins described herein, the antigen-binding domain includes a heavy chain variable domain sequence of: EVKLEESGGGLVQPGGSMKLSCAASGFTFSDAWMDWVRQSPEKGLEWVAEIRGKV NNLATYYVESVKGRFTISRDDSKSSVYLQMNSLRAEDTGIYYCLGRYDATYAMDY WGQGTSVTVSS (SEQ ID NO: 30). In some embodiments of any of the proteins described herein, the antigen-binding domain includes a heavy chain variable domain sequence that is at least 95% (e.g., at least 96%, at least 97%, at least 98%, or at least 99%) identical to SEQ ID NO: 30. In some embodiments of any of the proteins described herein, the antigen-binding domain includes a heavy chain variable domain sequence of SEQ ID NO: 30, except that it includes about 1 to about 6 amino acid substitutions (e.g., 1, 2, 3, 4, 5, or 6 amino acid substitutions) in SEQ ID NO: 30. In some embodiments of any of the proteins described herein, the protein competitively binds to human ADAM8 with an antigen-binding domain including: (i) a light chain variable domain of SEQ ID NO: 8 and a heavy chain variable domain of SEQ ID NO: 10; (ii) a light chain variable domain of SEQ ID NO: 38 and a heavy chain variable domain of SEQ ID NO: 40; (iii) a light chain variable domain of SEQ ID NO: 48 and a heavy chain variable domain of SEQ ID NO: 50; or (iv) a light chain variable domain of SEQ ID NO: 58 and a heavy chain variable domain of SEQ ID NO: 60.

In some embodiments of any of the proteins described herein, the protein includes a human Fc domain (e.g., a human IgG1 Fc domain, a human IgG2 Fc domain, a human IgG3 Fc domain, or a human IgG4 Fc domain).

In some embodiments of any of the proteins described herein, the protein further includes a conjugated toxin (e.g., ozogamicin, emtansine, vedotin) or therapeutic agent. Non-limiting examples of toxins and therapeutic agents (e.g., known to be useful for the treatment of cancer) are known in the art. In some embodiments of any of the proteins described herein, the protein is conjugated to the toxin or the therapeutic agent via a cleavable linker (e.g., a disulfide bond, a hydrazone, or a peptide). In some embodiments, the cleavable linker is a protease cleavage site (e.g., a peptide linker).

Methods for determining competitive binding of two different proteins to an ADAM8 protein sequence are known in the art and include, e.g., enzyme-linked immunosorbent assays (ELISA) or surface plasmon resonance. Methods for determining the binding affinity of any of the proteins described herein are known in the art and include, e.g., surface plasmon resonance or ELISA.

### Nucleic Acids, Vectors, and Mammalian Cells

Also provided herein are nucleic acids encoding any of the proteins described herein, vectors including any of the nucleic acids described herein, and mammalian cells (e.g., a CHO cell, a HEK cell or a hybridoma cell) including any of the nucleic acids described herein or any of the vectors described herein.

The term “vector” refers to a polynucleotide capable of carrying at least one exogenous nucleic acid fragment, and includes sufficient elements for expression.

In some embodiments, the vector is a plasmid, an adeno-associated viral (AAV) vector, an adenovirus, a retrovirus, a cosmid, or an artificial chromosome. Also provided are a pair of vectors that together encode a first polypeptide (e.g., an antibody light chain) and a second polypeptide (e.g., an antibody heavy chain) that together form any of the proteins described herein.

Methods of introducing vectors (e.g., expression vectors) into a mammalian cell (e.g., any of the mammalian cells described herein) are known in the art. Non-limiting examples of methods that can be used to introduce a vector (e.g., an expression vector, any of the vectors described herein) into a mammalian cell include: transfection, lipofection, electroporation, microinjection, calcium phosphate transfection, sonoporation, cell squeezing, cationic polymer transfection, optical transfection, dendrimer-based transfection, hydrodynamic delivery, magnetofection, nanoparticle transfection, or viral transduction (e.g., adeno-associated viral transduction, retroviral transduction, and lentiviral transduction).

### Methods of Producing a Protein

Also provided herein are methods of producing a protein (e.g., any of the proteins described herein) that includes: (a) culturing a mammalian cell (e.g., any of the mammalian cells described herein that include nucleic acid encoding the protein or include any of the vectors described herein) in a liquid culture medium under conditions sufficient to produce the protein; and (b) recovering the protein from the mammalian cell or the liquid culture medium. In some embodiments of any of the methods described herein, the method further includes: (c) isolating the protein recovered in step (b). In some embodiments of any of the methods described herein, the method further includes: (d) formulating the protein isolated in step (c) into a pharmaceutical composition.

Also provided herein is a cell (e.g., any of the exemplary mammalian cells described herein) that includes any of the nucleic acids, vectors, or proteins described herein. Skilled practitioners will appreciate that the nucleic acids and vectors described herein can be introduced into any mammalian cell. Non-limiting examples of vectors and methods for introducing vectors and proteins into mammalian cells are described herein.

In some embodiments, the mammalian cell is a human cell, a mouse cell, a rat cell, a rabbit cell, a dog cell, a cat cell, a porcine cell, a hamster cell, or a non-human primate cell.

In some embodiments, the mammalian cell is in vitro. In some embodiments, the mammalian cell is present in a mammal.

Methods of culturing mammalian cells are well-known in the art. Mammalian cells can be maintained in vitro under conditions that favor proliferation and growth. For example, a mammalian cell can be cultured by contacting a mammalian cell (e.g., any of the mammalian cells described herein) with a cell culture medium that includes the necessary growth factors and supplements to support cell viability and growth.

Exemplary methods of introducing nucleic acids into a mammalian cell (e.g., any of the mammalian cells described herein) are described herein. Additional methods of introducing nucleic acids into a mammalian cell are known in the art.

Provided herein are methods that further include isolation of a protein (e.g., any of the proteins described herein) from a mammalian cell (e.g., any of the mammalian cells described herein) using techniques well-known in the art (e.g., size exclusion chromatography, metal-affinity chromatography, ligand-affinity chromatography, ion exchange chromatography (anion or cation), ammonium sulfate precipitation, polyethylene glycol precipitation).

Methods of detecting expression and/or activity of any of the proteins described herein are known in the art. In some embodiments, the level of expression of a protein (e.g., any of the proteins described herein) can be detected directly (e.g., detecting protein expression). Non-limiting examples of techniques that can be used to detect expression and/or activity of a protein include: ELISA, Western blotting, immunohistochemistry, or immunofluorescence.

### Methods of Treatment

Provided herein are methods for inhibiting migration and/or invasion of an ADAM8 expressing cell in a subject (e.g., any of the exemplary subjects described herein) that include administering to the subject a therapeutically effective amount of any of the proteins described herein or any of the pharmaceutical compositions described herein.

In some embodiments of any of the methods described herein, the ADAM8 expressing cell is an ADAM8-associated cancer cell. In some embodiments of any of the methods described herein, the ADAM8-associated cancer cell is from a cancer selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma, and leukemia. In some embodiments of any of the methods described herein, the cancer cell is a triple negative breast cancer cell.

Also provided herein are methods of decreasing the risk of developing a metastasis or developing an additional metastasis over a period of time in a subject identified as having an ADAM8-associated cancer (e.g., any of the exemplary ADAM8-associated cancers described herein) that include administering to the subject a therapeutically effective amount of any of the proteins described herein or any of the pharmaceutical compositions described herein (e.g., as compared to a subject having a similar cancer and receiving a different treatment or receiving no treatment). In some embodiments of any of the methods described herein, the ADAM8-associated cancer is selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia.

In some embodiments of any of the methods described herein, the ADAM8-associated cancer is triple negative breast cancer. In some embodiments of any of the methods described herein, the metastasis or additional metastasis is one or more to a bone, lymph nodes, brain, lung, liver, skin, chest wall including bone, cartilage and soft tissue, abdominal cavity, contralateral breast, soft tissue, muscle, bone marrow, ovaries, adrenal glands, and pancreas.

In some embodiments of any of the methods described herein, the period of time is about 1 month to about 5 years (e.g., about 1 month to about 4 years, about 1 month to about 3.5 years, about 1 month to about 3 years, about 1 month to about 2.5 years, about 1 month to about 2 years, about 2 months to about 5 years, about 2 months to about 4 years, about 2 months to about 3.5 years, about 2 months to about 3 years, about 2 months to about 2.5 years, about 2 months to about 2 years, about 2 months to about 1.5 years, about 1 month to about 1 year, about 1 month to about 6 months, about 1 month to about 5 months, about 1 month to about 4 months, about 2 months to about 5 years, about 2 months to about 2 years, about 2 months to about 1 year, about 2 months to about 6 months, about 4 months to about 5 years, about 4 months to about 2 years, about 4 months to about 1 year, about 4 months to about 6 months, about 6 months to about 5 years, about 6 months to about 2 years, about 6 months to about 1 year, about 1 year to about 5 years, about 1 year to about 2 years, about 2 years to about 5 years, or about 4 years to about 5 years).

In some embodiments, the risk of developing a metastasis or developing an additional metastasis over a period of time in a subject identified as having an ADAM8-associated cancer is decreased by about 1% to about 99% (e.g., about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 1% to about 5%, about 5% to about 99%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 20% to about 99%, about 20% to about 90%, about 20% to about 80%, about 20% to about 70%, about 20% to about 60%, about 20% to about 50%, about 20% to about 40%, about 20% to about 30%, about 30% to about 99%, about 30% to about 90%, about 30% to about 80%, about 30% to about 70%, about 30% to about 60%, about 30% to about 50%, about 30% to about 40%, about 40% to about 99%, about 40% to about 90%, about 40% to about 80%, about 40% to about 70%, about 40% to about 60%, about 40% to about 50%, about 50% to about 99%, about 50% to about 90%, about 50% to about 80%, about 50% to about 70%, about 50% to about 60%, about 60% to about 99%, about 60% to about 90%, about 60% to about 80%, about 60% to about 70%, about 70% to about 99%, about 70% to about 90%, about 70% to about 80%, about 80% to about 99%, about 80% to about 90%, or about 90% to about 99%), e.g., as compared to the risk in a subject having a similar cancer receiving a different treatment or receiving no treatment.

Provided herein are methods of inhibiting the growth of a solid tumor in a subject (e.g., any of the subjects described herein) identified as having an ADAM8-associated cancer that include administering to the subject a therapeutically effective amount of any of the proteins described herein or any of the pharmaceutical compositions described herein (e.g., as compared to the growth of the solid tumor in the subject prior to treatment or the growth of a similar solid tumor in a different subject receiving a different treatment or receiving no treatment).

In some embodiments of any of the methods described herein, the growth of a solid tumor is primary growth of a solid tumor. In some embodiments of any of the methods described herein, the growth of a solid tumor is recurrent growth of a solid tumor. In some embodiments of any of the methods described herein, the growth of a solid tumor is metastatic growth of a solid tumor. In some embodiments of any of the methods described herein, the ADAM8-associated cancer is selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, and bone cancer. In some embodiments of any of the methods described herein, the ADAM8-associated cancer is triple negative breast cancer. In some embodiments, treatment results in about a 1% decrease to about 99% decrease (or any of the subranges of this range described herein) in the growth of a solid tumor in the subject (e.g., as compared to the growth of the solid tumor in the subject prior to treatment or the growth of a similar solid tumor in a different subject receiving a different treatment or receiving no treatment). The growth of a solid tumor in a subject can be assessed by a variety of different imaging methods, e.g., positron emission tomograph, X-ray computed tomography, computed axial tomography, and magnetic resonance imaging.

Provided herein are methods of inhibiting the growth or proliferation of a hematological cancer in a subject identified as having an ADAM8-associated cancer that include administering to the subject a therapeutically effective amount of any of the proteins described herein or any of the pharmaceutical compositions described herein (e.g., as compared to the growth or proliferation of the hematological cancer in the subject prior to treatment or the growth of a similar hematological cancer in a different subject receiving a different treatment or receiving no treatment). In some embodiments of any of the methods described herein, the hematological cancer is a leukemia. In some embodiments of any of the methods described herein, the hematological cancer is a lymphoma. In some embodiments, treatment results in about a 1% decrease to about 99% decrease (or any of the subranges of this range described herein) in the growth or proliferation of a hematological cancer in the subject (e.g., as compared to the growth or proliferation of the hematological cancer in the subject prior to treatment or the growth of a similar hematological cancer in a different subject receiving a different treatment or receiving no treatment). The growth or proliferation of a hematological cancer in a subject can be assessed by a variety of hematological tests.

Also provided herein are methods of killing an ADAM8-associated cancer cell in a subject that include: administering to the subject a therapeutically effective amount of any of the proteins described herein or any of the pharmaceutical compositions described herein. In some embodiments of any of the methods described herein, the killing of an ADAM8-associated cancer cell (e.g., cell death of an ADAM8-associated cancer cell) is apoptosis. In some embodiments of any of the methods described herein, the ADAM8-associated cancer cell is from a cancer selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments of any of the methods described herein, the cancer cell is a triple negative breast cancer cell.

Also provided herein are methods of treating an ADAM8-associated cancer in a subject that include: administering to a subject identified as having an ADAM8-associated cancer a therapeutically effective amount of any of the proteins described herein or any of the pharmaceutical compositions described herein. In some embodiments of any of the methods described herein, the ADAM8-associated cancer is selected from the group consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments of any of the methods described herein, the ADAM8-associated cancer is triple negative breast cancer.

In some embodiments of any of the methods described herein, the method further includes administering to the subject a therapeutically effective amount of a chemotherapeutic agent, a targeted therapy, or an immunotherapy. In some embodiments of any of the methods described herein, the chemotherapeutic agent is an antimetabolite, a plant alkaloid, a microtubule inhibitor, an anthracycline, a taxol, a platinum agent, or an alkylating agent. In some embodiments of any of the methods described herein, the targeted therapy is an angiogenesis or a kinase inhibitor. In some embodiments of any of the methods described herein, the immunotherapy is an inhibitor of PD-1, PD-L1, CTLA-4, LAG-3, CD70, CD80, ICOS, TIGIT, or IDO. In some embodiments of any of the methods described herein, the immunotherapy is a chimeric antigen receptor (CAR) T-cell therapy.

An immunotherapy can be administered to the patient in any of the methods described herein. The term “immunotherapy” refers to a therapeutic treatment that involves administering to a patient an agent that modulates the immune system. For example, an immunotherapy can decrease the expression and/or activity of a regulator of the immune system. In other instances, an immunotherapy can increase the expression and/or activity of a regulator of the immune system. In some instances, an immunotherapy can enhance or recruit the activity of an immune cell. In some embodiments of any of the methods described herein, the immunotherapy is an inhibitor of PD-1, PD-L1, CTLA-4, LAG-3, CD70, CD80, ICOS, TIGIT, or IDO. In some embodiments of any of the methods described herein, the immunotherapy is an antibody therapy (e.g., a conjugated therapy, a monoclonal antibody). Non-limiting examples of antibody therapies include: alemtuzumab (Campath®), bevacizumab (Mvasti™, Avastin®), dinutuximab (Unituxin®), avelumab (Bavencio®), rituximab (MabThera™, Rituxan®), elotuzumab (Empliciti™), edrecolomab (Panorex), daratumumab (Dazalex®), panitumumab (Vectibix®), pembrolizumab (Keytruda®), ramucirumab (Cyramza®), olaratumab (Lartruvo™), ofatumumab (Arzerra®), oregovomab, tremelimumab, ibritumomab (Zevalin®), mogamulizumab (Poteligeo®), denosumab (Xgeva®).

In some embodiments of any of the methods described herein, the immunotherapy is an immune checkpoint inhibitor, e.g., a CTLA-4 inhibitor, a PD-1 inhibitor, or PD-L1 inhibitor, or combinations thereof. In other embodiments of any of the methods described herein, the immunotherapy can be a cellular immunotherapy (e.g., dendritic cell therapy, natural killer cell therapy, adoptive T-cell therapy). For example, the cellular immunotherapy can be sipuleucel-T (Provenge™). In some instances, the cellular immunotherapy includes cells that express a chimeric antigen receptor (CAR). In some instances, the cellular immunotherapy can be a CAR-T cell therapy, e.g., tisagenlecleucel (Kyrmriah™).

In some embodiments of any of the methods described herein, the immunotherapy is a cytokine therapy (e.g., an interleukin 2 (IL-2) therapy, a granulocyte colony stimulating factor (G-CSF) therapy, an erythropoietin-alpha (EPO) therapy). A targeted therapy can be administered to the patient in any of the methods described herein. The term “targeted therapy” refers to a therapeutic agent that acts by interacting and/or binding with a specific molecular target. In some embodiments of any of the methods described herein, the targeted therapy is an angiogenesis inhibitor or a kinase inhibitor. In some embodiments of any of the methods described herein, the targeted therapy is an angiogenesis inhibitor (e.g., axitinib (Inlyta®, bevacizumab (Avastin®), cabozantinib (Cometriq®), everolimus (Afinitor®), lenalidomide (Revlimid®), lenvatinib mesylate (Lenvima®), pazopanib (Votrient®), ramucirumab (Cyramza®), regorafenib (Stivarga®), sorafenib (Nexavar®), vandetanib (Caprelsa®), ziv-aflibercept (Zaltrap®), sunitinib (Sutent®), thalidomide (Synovir, Thalomid®)). In some embodiments of any of the methods described herein, the targeted therapy is a kinase inhibitor. Non-limiting examples of kinase inhibitors include inhibitors that target EGFR, kit, ROS1, AKT, PDGFR, ABL, SRC, and mTOR. In some embodiments of any of the methods described herein, the kinase inhibitor is a tyrosine kinase inhibitor or a serine/threonine kinase inhibitor, or a combination thereof. Non-limiting examples of kinase inhibitors include, e.g., crizotinib (Xalkori®), ceritinib (Zykadia®), alectinib (Alecensa®), brigatinib (Alunbrig®), bosutinib (Bosulif®), dasatinib (Sprycel®), imatinib (Gleevec®), nilotinib (Tasigna®), ponatinib (Iclusig®), vemurafenib (Zelboraf®), dabrafenib (Tafinlar®), ibrutinib (Imbruvica®), palbociclib (Ibrance®), sorafenib (Nexavar®), ribociclib (Kisqali®), cabozantinib (Cometriq®), gefitinib (Iressa®), erlotinib (Tarceva®), lapatinib (Tykerb®), vandetanib (Caprelsa®), afatinib (Gilotrif®), osimertinib (Tagrisso®), ruxolitinib (Jakafi®), tofacitinib (Xeljanz®), trametinib (Mekinist®), axitinib (Inlyta®), lenvatinib (Lenvima®), nintedanib (OFEV®), regorafenib (Stivarga®), sunitinib (Sutent®).

In some embodiments of any of the methods described herein, the immunotherapy is a chimeric antigen receptor (CAR) T-cell therapy.

The administering, in any of the methods described herein, may be performed, e.g., at least once (e.g., at least 2-times, at least 3-times, at least 4-times, at least 5-times, at least 6-times, at least 7-times, at least 8-times, at least 9-times, at least 10-times, at least 11-times, at least 12-times, at least 13-times, at least 14-times, or at least 15-times) a week. Also contemplated are monthly administrations, e.g., administering at least once per month for at least 1 month (e.g., at least two, three, four, five, six, seven, or eight or more months, e.g., 12 or more months), and yearly (e.g., administering once a year for one or more years).

Administration can be via any art-known means, e.g., intravenous, subcutaneous, intraperitoneal, oral and/or rectal administration, or any combination of known administration methods.

Administration can include administering pharmaceutical compositions formulated in any useful form. One useful pharmaceutical composition may be a combination pharmaceutical composition comprising any of the proteins described herein and an angiogenesis inhibitor, a checkpoint inhibitor, a kinase inhibitor, and/or a chemotherapeutic agent(s).

Methods of Identifying a Protein that Binds Specifically to Human ADAM8 and Inhibits Both its MP and DI Activity

Also provided herein are methods of identifying a protein including an antigen-binding domain that binds specifically to human ADAM8 and has the ability to inhibit both the metalloprotease activity and disintegrin activity of human ADAM8 that include: (a) identifying a protein including an antigen-binding domain that binds to an epitope within human ADAM8 that includes at least one amino acid within the sequence of:

(SEQ ID NO: 1)
DCGPPEDCRNRCCNSTTCQLAEGAQCAHGTCCQECKVKPAGELCRPKKD
MCDLEEFCDGRHPECPEDAF.

In some embodiments of any of the methods described herein, the method further includes confirming the ability of the identified protein to inhibit the metalloprotease activity and disintegrin activity of human ADAM8.

In some embodiments of any of the methods described herein, step (a) includes identifying a protein including an antigen-binding domain that binds to an epitope within human ADAM8 that includes at least one amino acid within the sequence of:

(SEQ ID NO: 86)
CCNSTTCQLAEGAQCAHGTCCQECK
or
(SEQ ID NO: 104)
RNRCCNSTTCQLAEGAQCAHGTCCQECK.

In some embodiments of any of the methods described herein, step (a) includes identifying a protein including an antigen-binding domain that binds to an epitope within human ADAM8 that includes at least one amino acid within the sequence of:

(SEQ ID NO: 87)
LAEGAQCAHGTCCQECKVKPAGELCRPKKDMCDLEEFCDGRHPECPE
DAF.

In some embodiments of any of the methods described herein, step (a) includes identifying a protein including an antigen-binding domain that binds to an epitope within human ADAM8 that includes at least one amino acid within the sequence of:

(SEQ ID NO: 88)
DCGPPEDCRNRCCNSTTCQ.

Some embodiments further include performing an animal model study of an ADAM8-associated cancer using the protein identified using any of the methods described herein.

### Methods of Diagnosing

Preliminary immunohistochemistry (IHC) studies with research use only (RUO) anti-ADAM8 antibodies, indicate that a significant percentage but not all patients affected by a diverse group of aggressive cancers (e.g., those of the breast, colon, stomach, liver, pancreas, lungs, head and neck, and bones) have elevated ADAM8 levels. See Romagnoli et al., EMBO Mol Med, 6 (2): 278-294, 2014; Yang et al., BMC Cancer, 14:568-579, 2014; Huang et al., Transl Res, 166 (6): 602-613, 2015; Zhang et al., Pathol Oncol Res, 19 (1): 79-88, 2013; Valkovskaya et al., J Cell Mol Med, 11 (5): 1162-1174, 2007; Ishikawa et al., Clin Cancer Res, 10 (24): 8363-8370, 2004; Zielinski et al., BMC Cancer, 12:76, 2012; and Abd El-Rehim et al., J Egypt Natl Canc Inst, 27 (1): 1-9, 2015. Thus, a diagnostic assay is critical for the identification of patients with ADAM8-positive tumors who can benefit from an ADAM8-targeted therapy.

The strategy used herein to identify patients that could benefit from an ADAM8-targeted therapy includes an IHC-based assay of formalin-fixed paraffin embedded (FFPE) biopsy samples. Following cancer detection, patient tumor samples (biopsies) are taken for histologic examination and disease staging, as well as IHC analysis for detection of any known molecular markers, to guide appropriate diagnosis and treatment selection. For example, for patients with breast, gastric and gastroesophogeal cancers, biopsies are routinely analyzed by the HercepTest IHC assay (Dako), which is FDA-approved for evaluation of HER2 status and selection of candidates for Herceptin (Genentech) treatment. Similarly, for patients with lung cancer, the anaplastic lymphoma kinase (ALK) D5F3 [Ventana] IHC assay is used to assess ALK status and identify patients who can benefit from Zykadia [Novartis] treatment. IHC is a well-accepted diagnostic technology by the FDA, does not require collection of any additional patient samples, and can be performed on automated platforms already deployed worldwide in diagnostic laboratories. These characteristics are particularly advantageous as they ensure rapid introduction of diagnostic products to the market, enabling timely patient access to new therapies. Currently, there is no FDA-approved diagnostic for detection of ADAM8-positive cancers. Herein, the identification of ADP antibodies capable of IHC-based detection of ADAM8 is described as well as IHC diagnostic antibodies that specifically recognize the target of interest under IHC conditions, that is, following fixation and retrieval of the tissue antigen from paraffin embedding.

Targeted therapies have profoundly extended and improved the quality of life for cancer patients whose tumors express specific driver genes. However, these advances would not be possible without the use of companion diagnostics that characterize the patient's tumor, providing oncologists critical information that allows them to select the most appropriate treatment regimen for a specific patient. An ADAM8 diagnostic product, based on the ADP antibodies, for detection of patients with ADAM8-positive disease can provide patients with access to a targeted therapy that can significantly improve their outcome. As ADAM8 is highly expressed on multiple aggressive tumors, such a diagnostic product has the potential to impact a large section of the overall cancer population.

Provided herein are methods of diagnosing an ADAM8-associated cancer in a subject that include: (a) contacting a biological sample from the subject with any of the proteins described herein; (b) determining a level of the protein specifically bound to the biological sample; and (c) identifying the subject as having an ADAM8-associated cancer if the level of the protein specifically bound to the biological sample is elevated as compared to a control level (e.g., a level of the protein bound to a control sample, e.g., a biological sample obtained from a subject not having or suspected of having an ADAM8-associated cancer, or a biological sample from a healthy subject with a low risk of developing an ADAM8-associated cancer).

In some examples, the biological sample is a liquid biopsy sample (e.g., blood, cerebrospinal fluid, pleural effusion, ascites). In some embodiments, the methods can further include obtaining the liquid biopsy sample from the subject.

In some embodiments, the methods can further include, prior to step (a), concentrating the cells in the liquid biopsy sample.

In some embodiments, the method can further include, prior to step (a), lysing the cells in the liquid biopsy sample. In some embodiments, step (b) can include performing an enzyme-linked immunosorbent assay (ELISA).

In some embodiments, step (b) can include the use of fluorescence-activated cell sorting. In some embodiments, step (b) can include fixing and permeabilizing the cells in the liquid biopsy sample.

In some examples, the biological sample is a tissue sample. In some examples, the tissue sample is a biopsy tissue sample. In some embodiments, the methods can further include obtaining the biopsy tissue sample from the subject.

In some examples, the tissue sample is not a fixed tissue sample (e.g., a fresh, frozen tissue sample). In some embodiments, the method can further include, prior to step (a), trypsinizing the tissue sample. In some embodiments, step (b) can include the use of fluorescence-activated cell sorting.

In some embodiments, the tissue sample can be a fixed tissue sample (e.g., a formalin-fixed paraffin-embedded (FFPE) tissue sample). In some embodiments, the methods can further include, before step (a), fixing the tissue sample. In some embodiments, the method can further include before step (a), decrosslinking the fixed tissue sample. Non-limiting examples of methods and conditions that can be used to decrosslink a tissue sample are described in the Examples. For example, decrosslinking of the fixed tissue sample can be performed using a Tris-EDTA-based, basic buffer. In some embodiments, the decrosslinking can be performed for about 40 minutes to about 80 minutes (e.g., about 40 minutes to about 75 minutes, about 40 minutes to about 70 minutes, about 40 minutes to about 65 minutes, about 40 minutes to about 60 minutes, about 40 minutes to about 55 minutes, about 40 minutes to about 50 minutes, about 40 minutes to about 45 minutes, about 45 minutes to about 80 minutes, about 45 minutes to about 75 minutes, about 45 minutes to about 70 minutes, about 45 minutes to about 65 minutes, about 45 minutes to about 60 minutes, about 45 minutes to about 55 minutes, about 45 minutes to about 50 minutes, about 50 minutes to about 80 minutes, about 50 minutes to about 75 minutes, about 50 minutes to about 70 minutes, about 50 minutes to about 65 minutes, about 50 minutes to about 60 minutes, about 50 minutes to about 55 minutes, about 55 minutes to about 80 minutes, about 55 minutes to about 75 minutes, about 55 minutes to about 70 minutes, about 55 minutes to about 65 minutes, about 55 minutes to about 60 minutes, about 60 minutes to about 80 minutes, about 60 minutes to about 75 minutes, about 60 minutes to about 70 minutes, about 60 minutes to about 65 minutes, about 65 minutes to about 80 minutes, about 65 minutes to about 75 minutes, about 65 minutes to about 70 minutes, about 70 minutes to about 80 minutes, about 70 minutes to about 75 minutes, or about 75 minutes to about 80 minutes) at a temperature of about 65° C. to about 95° C. (e.g., about 65° C. to about 94° C., about 65° C. to about 92° C., about 65° C. to about 90° C., about 65° C. to about 88° C., about 65° C. to about 86° C., about 65° C. to about 85° C., about 65° C. to about 84° C., about 65° C. to about 82° C., about 65° C. to about 80° C., about 65° C. to about 78° C., about 65° C. to about 76° C., about 65° C. to about 75° C., about 65° C. to about 74° C., about 65° C. to about 72° C., about 65° C. to about 70° C., about 65° C. to about 68° C., about 66° C. to about 95° C., about 66° C. to about 94° C., about 66° C. to about 92° C., about 66° C. to about 90° C., about 66° C. to about 88° C., about 66° C. to about 86° C., about 66° C. to about 85° C., about 66° C. to about 84° C., about 66° C. to about 82° C., about 66° C. to about 80° C., about 66° C. to about 78° C., about 66° C. to about 76° C., about 66° C. to about 75° C., about 66° C. to about 74° C., about 66° C. to about 72° C., about 66° C. to about 70° C., about 66° C. to about 68° C., about 68° C. to about 95° C., about 68° C. to about 94° C., about 68° C. to about 92° C., about 68° C. to about 90° C., about 68° C. to about 88° C., about 68° C. to about 86° C., about 68° C. to about 85° C., about 68° C. to about 84° C., about 68° C. to about 82° C., about 68° C. to about 80° C., about 68° C. to about 78° C., about 68° C. to about 76° C., about 68° C. to about 75° C., about 68° C. to about 74° C., about 68° C. to about 72° C., about 68° C. to about 70° C., about 70° C. to about 95° C., about 70° C. to about 94° C., about 70° C. to about 92° C., about 70° C. to about 90° C., about 70° C. to about 88° C., about 70° C. to about 86° C., about 70° C. to about 85° C., about 70° C. to about 84° C., about 70° C. to about 82° C., about 70° C. to about 80° C., about 70° C. to about 78° C., about 70° C. to about 76° C., about 70° C. to about 75° C., about 70° C. to about 74° C., about 70° C. to about 72° C., about 72° C. to about 95° C., about 72° C. to about 94° C., about 72° C. to about 92° C., about 72° C. to about 90° C., about 72° C. to about 88° C., about 72° C. to about 86° C., about 72° C. to about 85° C., about 72° C. to about 84° C., about 72° C. to about 82° C., about 72° C. to about 80° C., about 72° C. to about 78° C., about 72° C. to about 76° C., about 72° C. to about 75° C., about 72° C. to about 74° C., about 74° C. to about 95° C., about 74° C. to about 94° C., about 74° C. to about 92° C., about 74° C. to about 90° C., about 74° C. to about 88° C., about 74° C. to about 86° C., about 74° C. to about 85° C., about 74° C. to about 84° C., about 74° C. to about 82° C., about 74° C. to about 80° C., about 74° C. to about 78° C., about 74° C. to about 76° C., about 74° C. to about 75° C., about 75° C. to about 95° C., about 75° C. to about 94° C., about 75° C. to about 92° C., about 75° C. to about 90° C., about 75° C. to about 88° C., about 75° C. to about 86° C., about 75° C. to about 85° C., about 75° C. to about 84° C., about 75° C. to about 82° C., about 75° C. to about 80° C., about 75° C. to about 78° C., about 76° C. to about 95° C., about 76° C. to about 94° C., about 76° C. to about 92° C., about 76° C. to about 90° C., about 76° C. to about 88° C., about 76° C. to about 86° C., about 76° C. to about 85° C., about 76° C. to about 84° C., about 76° C. to about 82° C., about 76° C. to about 80° C., about 78° C. to about 95° C., about 78° C. to about 94° C., about 78° C. to about 92° C., about 78° C. to about 90° C., about 78° C. to about 88° C., about 78° C. to about 86° C., about 78° C. to about 85° C., about 78° C. to about 84° C., about 78° C. to about 82° C., about 78° C. to about 80° C., about 80° C. to about 95° C., about 80° C. to about 94° C., about 80° C. to about 92° C., about 80° C. to about 90° C., about 80° C. to about 88° C., about 80° C. to about 86° C., about 80° C. to about 85° C., about 80° C. to about 84° C., about 80° C. to about 82° C., about 82° C. to about 95° C., about 82° C. to about 94° C., about 82° C. to about 92° C., about 82° C. to about 90° C., about 82° C. to about 88° C., about 82° C. to about 86° C., about 82° C. to about 85° C., about 82° C. to about 84° C., about 84° C. to about 95° C., about 84° C. to about 94° C., about 84° C. to about 92° C., about 84° C. to about 90° C., about 84° C. to about 88° C., about 84° C. to about 86° C., about 84° C. to about 85° C., about 85° C. to about 95° C., about 85° C. to about 94° C., about 85° C. to about 92° C., about 85° C. to about 90° C., about 85° C. to about 88° C., about 85° C. to about 86° C., about 86° C. to about 95° C., about 86° C. to about 94° C., about 86° C. to about 92° C., about 86° C. to about 90° C., about 86° C. to about 88° C., about 88° C. to about 95° C., about 88° C. to about 94° C., about 88° C. to about 92° C., about 88° C. to about 90° C., about 90° C. to about 95° C., about 90° C. to about 94° C., about 90° C. to about 92° C., about 92° C. to about 95° C., about 92° C. to about 94° C., or about 94° C. to about 95° C.).

In some embodiments, the decrosslinking of the fixed tissue sample is performed using an alkaline endopeptidase (e.g., a serine protease).

In some embodiments of any of the methods described herein, the protein comprises a detectable label. In some embodiments, step (b) comprises detecting the detectable label. In some embodiments, the detectable label is a heavy metal, a fluorophore, a chromophore, or an enzyme.

In some embodiments, the protein does not comprise a detectable label, and step (b) comprises the use of an agent that binds specifically to the protein specifically bound to the biological sample. In some embodiments, the agent comprises an antibody. In some embodiments, the agent comprises a detectable label. In some embodiments, step (b) comprises detecting the detectable label. In some embodiments, the detectable label comprises a heavy metal, a fluorophore, a chromophore, or an enzyme.

In some embodiments, step (b) comprises imaging the biological sample. In some embodiments, the determining in step (b) comprises performing immunohistochemistry or immunofluorescence.

Some embodiments of these methods further include, after step (c), (d) selecting a therapeutically effective amount of the protein used in step (a) for treatment of the subject identified as having an ADAM8-associated cancer. Some embodiments of these methods further include, after step (c), (d) administering a therapeutically effective amount of the protein used in step (a) to the subject identified as having an ADAM8-associated cancer. In some embodiments, the protein used in step (d) further comprises the protein conjugated to a toxin or a therapeutic agent.

Some embodiments of these methods further include, after step (c), (d) selecting a therapeutically effective amount of any of the proteins described herein (e.g., the same protein used in step (a) or a different protein from any of the exemplary proteins described herein) for treatment of the subject identified as having an ADAM8-associated cancer. Some embodiments of these methods further include, after step (c), (d) administering a therapeutically effective amount of any of the proteins described herein (e.g., the same protein used in step (a) or a different protein from any of the exemplary proteins described herein) to the subject identified as having an ADAM8-associated cancer. In some embodiments, the protein used in step (d) further comprises the protein conjugated to a toxin or a therapeutic agent.

Some embodiments of these methods further include, after step (c), (d) administering a therapeutically effective amount of a chemotherapeutic agent, a targeted therapy, or an immunotherapy to the subject identified as having the ADAM8-associated cancer. In some embodiments, the chemotherapeutic agent is an antimetabolite, a plant alkaloid, a microtubule inhibitor, an anthracycline, a taxol, a platinum agent, or an alkylating agent. In some embodiments, the targeted therapy is an angiogenesis or a kinase inhibitor. In some embodiments, the immunotherapy is an inhibitor of PD-1, PD-L1, CTLA-4, LAG-3, CD70, CD80, ICOS, TIGIT, or IDO. In some embodiments, the immunotherapy is a chimeric antigen receptor (CAR) T-cell therapy.

Some embodiments of these methods further include, after step (c), determining the stage of the ADAM8-associated cancer in the subject based on the level of the protein specifically bound to the biological sample.

In some embodiments, the subject is suspected of having an ADAM8-associated cancer. In some embodiments, the subject is presenting with one or more symptoms of an ADAM8-associated cancer.

In some embodiments, wherein the ADAM8-associated cancer is a cancer selected from the consisting of: breast cancer, brain cancer, head and neck cancer, thyroid cancer, esophageal cancer, lung cancer, adrenal cancer, renal cancer, liver cancer, pancreatic cancer, gastric cancer, neuroendocrine cancer, colorectal cancer, small intestine cancer, bladder cancer, ovarian cancer, cervical cancer, uterine cancer, prostate cancer, testicular cancer, skin cancer, bone cancer, lymphoma and leukemia. In some embodiments, the ADAM8-associated cancer is triple negative breast cancer. In some embodiments, the ADAM8-associated cancer is a hematological cancer. In some embodiments, the hematological cancer is a leukemia. In some embodiments, the hematological cancer is lymphoma.

In some embodiments, wherein the biological sample is obtained from a metastasis (e.g., a metastasis obtained from bone, lymph node, brain, lung, liver, skin, chest wall (including bone, cartilage and soft tissue), abdominal cavity, contralateral breast, soft tissue, muscle, bone marrow, ovaries, adrenal glands, or pancreas).

### Methods of Determining the Efficacy of Treatment

Also provided herein are methods of determining the efficacy of treatment of an ADAM8-associated cancer (e.g., any of the exemplary ADAM8-associated cancers described herein) in a subject that include: (a) contacting a first biological sample obtained from a subject having an ADAM8-associated cancer at first time point with any of the proteins described herein; (b) determining a first level of the protein specifically bound to the first biological sample; (c) contacting a second biological sample obtained from the same subject at a second time point with the protein, where the subject has been administered a treatment against an ADAM8-associated cancer between the first and second time points; (d) determining a second level of the protein specifically bound to the second biological sample; and (e) determining the treatment as being effective in a subject having a decreased second level as compared to the first level, or determining the treatment as not being effective in a subject having about the same or an increased second level as compared to the first level. In some embodiments, the subject has previously been diagnosed as having an ADAM8-associated cancer. Some embodiments of these methods further include recording the determination in step (e) in the subject's medical record.

In some embodiments, step (e) comprises determining the treatment as being effective in the subject. Some embodiments of these methods further include, after step (e), selecting one or more additional doses of the treatment for the subject. Some embodiments of these methods further include, after step (e), administering one or more additional doses of the treatment to the subject.

In some embodiments, step (e) comprises determining the treatment was not effective in the subject. Some embodiments of these methods further include, after step (e), selecting an alternative treatment for the subject. Some embodiments of these methods further include, after step (e), administering an alternative treatment to the subject.

Some embodiments of these methods further include administering the treatment to the subject between the first and second time points.

In some embodiments, the treatment comprises the protein used in steps (a) and (c). In some embodiments, the treatment comprises the protein conjugated to a cytotoxin or therapeutic agent.

In some embodiments, the treatment comprises any of the proteins described herein (e.g., the same protein used in steps (a) and (c), or a different protein from any of the exemplary proteins described herein). In some embodiments, the treatment comprises the protein conjugated to a cytotoxin or therapeutic agent.

In some embodiments, the treatment comprises a chemotherapeutic agent, a targeted therapy, or an immunotherapy. In some embodiments, the chemotherapeutic agent is an antimetabolite, a plant alkaloid, a microtubule inhibitor, an anthracycline, a taxol, a platinum agent, or an alkylating agent. In some embodiments, the targeted therapy is an angiogenesis or a kinase inhibitor. In some embodiments, the immunotherapy is an inhibitor of PD-1, PD-L1, CTLA-4, LAG-3, CD70, CD80, ICOS, TIGIT, or IDO. In some embodiments, the immunotherapy is a chimeric antigen receptor (CAR) T-cell therapy.

In some embodiments, the first and second biological samples are liquid biopsy samples (e.g., blood, cerebrospinal fluid, pleural effusion, ascites). In some embodiments, the methods can further include obtaining the first and second liquid biopsy samples from the subject.

In some embodiments, the methods can further include, prior to step (a), concentrating the cells in the first and second liquid biopsy samples.

In some embodiments, step (b) can include lysing the cells in the first and second liquid biopsy samples. In some embodiments, step (b) can include performing an enzyme-linked immunosorbent assay (ELISA).

In some embodiments, step (b) can include the use of fluorescence-activated cell sorting. In some embodiments, step (b) can include fixing and permeabilizing the cells in the first and second liquid biopsy samples.

In some embodiments, steps (b) and (d) comprise imaging the first and second biological samples. In some embodiments, the determining in steps (b) and (d) comprise performing immunohistochemistry or immunofluorescence.

In some embodiments, the first and second biological samples are tissue samples (e.g., biopsy tissue samples).

In some embodiments, the tissue samples are not fixed tissue samples. In some embodiments, the tissue samples are fresh, frozen tissue samples. In some embodiments, the method further comprises, prior to step (a), trypsinizing the tissue samples. In some embodiments, step (b) comprises the use of fluorescence-activated cell sorting.

In some embodiments, the tissue samples are fixed tissue samples (e.g., formalin-fixed paraffin-embedded (FFPE) tissue samples). Some embodiments of these methods further include, before step (a), fixing the tissue samples. Some embodiments of these methods further include, before step (a), decrosslinking the fixed tissue samples. In some embodiments, the decrosslinking of the fixed tissue samples is performed using a Tris-EDTA-based, basic buffer. In some embodiments, the decrosslinking is performed for about 40 minutes to about 80 minutes (or any of the exemplary subranges of this range described herein) at a temperature of about 65° C. to about 95° C. (or any of the exemplary subranges of this range described herein).

In some embodiments, the decrosslinking of the fixed tissue samples is performed using an alkaline endopeptidase (e.g., serine protease).

In some embodiments of any of the methods described herein, the protein comprises a detectable label. In some embodiments, step (b) comprises detecting the detectable label. In some embodiments, the detectable label is a heavy metal, a fluorophore, a chromophore, or an enzyme.

In some embodiments of any of the methods described herein, the protein does not comprise a detectable label, and steps (b) and (d) comprise the use of an agent that binds specifically to the protein specifically bound to the first and second biological samples, respectively. In some embodiments, the agent comprises an antibody. In some embodiments, the agent comprises a detectable label. In some embodiments, steps (b) and (d) comprise detecting the detectable label. In some embodiments, the detectable label comprises a heavy metal, a fluorophore, a chromophore, or an enzyme.

In some embodiments, steps (b) and (d) comprise imaging the first and second biological samples. In some embodiments, the determining in steps (b) and (d) comprises performing immunohistochemistry or immunofluorescence.

In some embodiments of any of the methods described herein, the second time point is about 1 month to about 5 years (e.g., about 1 month to about 4 years, about 1 month to about 3.5 years, about 1 month to about 3 years, about 1 month to about 2.5 years, about 1 month to about 2 years, about 2 months to about 5 years, about 2 months to about 4 years, about 2 months to about 3.5 years, about 2 months to about 3 years, about 2 months to about 2.5 years, about 2 months to about 2 years, about 2 months to about 1.5 years, about 1 month to about 1 year, about 1 month to about 6 months, about 1 month to about 5 months, about 1 month to about 4 months, about 2 months to about 5 years, about 2 months to about 2 years, about 2 months to about 1 year, about 2 months to about 6 months, about 2 months to about 4 months, about 2 months to about 3 months, about 4 months to about 5 years, about 4 months to about 2 years, about 4 months to about 1 year, about 4 months to about 6 months, about 5 months to about 5 years, about 5 months to about 2 years, about 5 months to about 1 year, about 6 months to about 5 years, about 6 months to about 2 years, about 6 months to about 1 year, about 1 year to about 5 years, about 1 year to about 2 years, about 2 years to about 5 years, or about 4 years to about 5 years; or about 1 month, about 2 months, about 3 months, about 4 months, about 5 months, about 6 months, about 8 months, about 9 months, about 10 months, about 1 year, about 2 years, about 3 years, about 4 years, or about 5 years) after the first time point.

### Pharmaceutical Compositions and Kits

Also provided herein are pharmaceutical compositions that include a therapeutically effective amount of any of the proteins described herein and one or more pharmaceutically or physiologically acceptable carriers, diluents, or excipients. Such pharmaceutical compositions may comprise one or more buffers, such as neutral-buffered saline, phosphate-buffered saline, and the like; one or more carbohydrates, such as glucose, mannose, dextran, and sucrose; mannitol; one or more proteins, polypeptides, or amino acids, such as glycine; one or more antioxidants; one or more chelating agents, such as EDTA or glutathione; and/or one or more preservatives.

In some embodiments, the pharmaceutical composition includes a pharmaceutically acceptable carrier (e.g., phosphate buffered saline, bacteriostatic water, or saline). Upon formulation, solutions will be administered in a manner compatible with the dosage formulation and in such amount as is therapeutically effective. The formulations are easily administered in a variety of dosage forms such as injectable solutions, injectable gels, infusions, drug-release capsules, and the like.

As used herein the term “pharmaceutically acceptable carrier” includes solvents, dispersion media, coatings, antibacterial agents, antifungal agents, and the like that are compatible with pharmaceutical administration. Supplementary active compounds can also be incorporated into any of the pharmaceutical compositions described herein.

In some embodiments, a single dose of any of the pharmaceutical compositions described herein can include a total sum amount of the protein of at least 1 mg, at least 2 mg, at least 4 mg, at least 5 mg, about 6 mg, about 8 mg, about 10 mg, about 12 mg, about 20 mg, about 30 mg, about 35 mg, about 40 mg, about 45 mg, about 50 mg, about 60 mg, about 80 mg, about 100 mg, about 120 mg, about 140 mg, about 150 mg, about 160 mg, about 180 mg, about 200 mg, about 220 mg, about 240 mg, about 250 mg, about 260 mg, about 280 mg, about 300 mg, about 350 mg, about 400 mg, about 450 mg, about 500 mg, about 550 mg, about 600 mg, about 650 mg, about 700 mg, about 750 mg, about 800 mg, about 850 mg, or about 900 mg, e.g., in a buffered solution.

The pharmaceutical compositions can be, e.g., formulated to be compatible with any intended route of administration (e.g., intravenous).

Also provided herein are kits including any of the pharmaceutical compositions described herein. In some embodiments, a kit can include a solid composition (e.g., a lyophilized composition including any of the proteins described herein) and a liquid for solubilizing the lyophilized composition.

In some embodiments, a kit can include a pre-loaded syringe including any of the pharmaceutical compositions described herein. In some embodiments, the kit includes a vial comprising any of the pharmaceutical compositions described herein (e.g., formulated as an aqueous pharmaceutical composition). In some embodiments, the kits can include instructions for performing any of the methods described herein.

Also provided herein are kits including any of the proteins described herein, and instructions for performing any of the methods described herein (e.g., diagnostic methods). In some embodiments, a kit can include instructions for use and other necessary reagents, e.g., positive and negative control samples, negative control antibodies, any of the proteins described herein and detection reagents (e.g., antibodies that bind specifically to any of the proteins described herein and reagents necessary for detection of a tag or enzyme activity); and devices (e.g., a syringe, a finger prick) or other materials for diagnosing. In some embodiments, a kit can include a solid composition (e.g., a lyophilized composition) of the proteins described herein and of the other kit reagents and liquid solutions for solubilizing the lyophilized components.

### EXAMPLES

### Example 1. Overview of Approach to Generate Highly Specific, Dual Antagonist Monoclonal Antibodies that Inhibit the MP and DI Domains of Human ADAM8

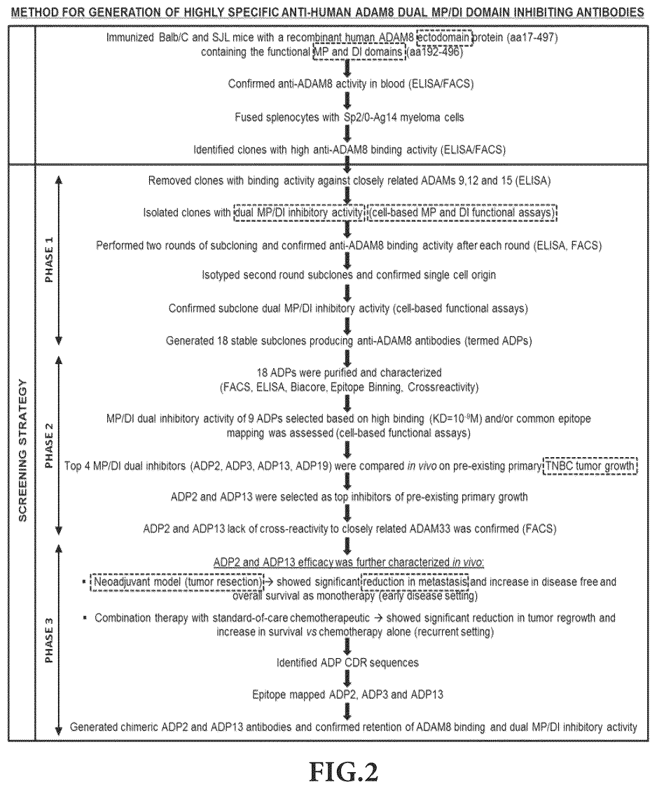

The hybridoma method (Nelson et al., Mol Pathol, 53 (3): 111-117, 2000) was used to generate antibodies against a functional human ADAM8 ectodomain fragment. These monoclonal antibodies were then subjected to a multi-stage screening strategy (FIG. 2) to first isolate a panel of dual Metalloprotease (MP) and Disintegrin (DI) domain inhibitor antibodies, termed ADPs (Phase 1). The panel of ADPs was then characterized with respect to epitope, kinetics and specificity of ADAM8 binding and certain ADPs were screened in mouse models of Triple-negative breast cancer (TNBC). Two lead therapeutic candidates (ADP2 and ADP13) were identified. A third antibody (ADP3) also showed anti-tumor effect, but to a more limited extent (Phase 2). In Phase 3, additional mouse model testing (neoadjuvant tumor resection and combination therapy), amino acid sequencing, epitope mapping, and generation and functional characterization of chimeric ADP2 and ADP13 was performed. Taken together, the results showed that anti-ADAM8 dual antagonist candidate therapeutics, ADP2 and ADP13, bound to sequences within the ADAM8 DI domain, leading to reduced primary tumor growth, reduced risk of recurrence and reduced frequency and extent of metastasis, significantly improving overall survival.

### Example 2. Generation of Hybridomas Producing Highly Specific, Monoclonal Antibodies Against Functional ADAM8

Recombinant human ADAM8 (rHuADAM8) ectodomain protein (aa 17-497) produced in mammalian HEK293 cells, containing both MP and DI domains and capable of autocatalytically activating itself, was purchased from ACRO Biosystems (AD8-H5223) for use as an immunogen. The activity of this recombinant protein was confirmed using functional MP and DI assays for: (a) MP domain activity: activation of a quenched fluorescent CD23 peptide; and (b) DI domain activity: binding of Chinese Hamster Ovary (CHO) cells ectopically expressing α9β1 integrin to recombinant human ADAM8 coated plates.

Balb/c and SJL mice were selected for immunization in order to give the broadest range of immune response. Ten mice of each strain were pre-bled and then each injected with 50-100 μg rHuADAM8 with complete Freund's adjuvant on Day 0. On Days 14 and 35, mice were boosted with 25-50 μg rHuADAM8 with incomplete Freund's adjuvant and then bled 7 days later. These test bleed sera were evaluated for binding to rHuADAM8 using Enzyme-linked immunosorbent assays (ELISA), and for binding to native cell-surface expressed ADAM8 using HEK293-ADAM8 cells in Fluorescence-activated cell sorting (FACS) analysis to confirm a high level of anti-ADAM8 activity. Mice were given one additional boost (Day 56) prior to fusion of lymphocytes from the best responders to the Sp2/0-Ag14 myeloma cell line.

Three fusions were performed with B lymphocytes from 6 mice. Cell supernatants from resulting hybridomas, containing 0.25 to 10 μg IgG/ml, were tested to identify clones with high anti-ADAM8 activity by ELISA and FACS. Then, a novel 3 phase screening strategy (FIG. 2) was performed to isolate dual antagonist monoclonal antibodies (termed ADPs) that inhibit the two critical MP and DI domain functions of ADAM8 using cell-based assays. Four ADPs were selected and further screened against primary TNBC growth in mice, resulting in identification of ADP2 and ADP13 as lead antibodies. Extensive additional preclinical mouse testing of ADP2 and ADP13 confirmed that these dual ADAM8 inhibitory antibodies had the necessary functional characteristics, as described in U.S. Patent Publication No. 2016/0130365, to ultimately be successful therapeutic antibodies.

### Example 3. Screening of Anti-ADAM8 ADP Antibodies

In Phase 1 screening, hybridomas making antibodies cross-reacting with recombinant human ADAM9, ADAM12 and ADAM15 proteins, which are closely related to ADAM8 (Takeda, Toxins, 8 (5). pii: E155, 2016), were identified in ELISA assays and excluded from consideration. Supernatants from the remaining hybridoma clones, grown in hybridoma-serum free medium, i.e., in the absence of fetal bovine serum (FBS), to enhance antibody production, were then tested for simultaneous antagonist activity against ADAM8 MP and DI domains using cell-based assays. Hybridomas demonstrating dual antagonist activity were subcloned in two rounds by serial dilution. Following each round of subcloning, ADAM8 binding activity was confirmed in ELISA and FACS experiments. Second round subclones were isotyped, which also confirmed single clone origin, and subjected to MP/DI activity assays using cell-based functional assays. Eighteen stable subclones producing anti-human ADAM8 antibodies (termed ADPs) with dual MP and DI domain antagonist activity were identified.

In Phase 2 screening, purified ADPs were further characterized with respect to their ADAM8 binding properties [using FACS, ELISA, Biacore, epitope binning and cross-reactivity (specificity) assays], as well as their ability to inhibit the MP and DI domains of ADAM8 (in cell-based MP/DI functional assays) and to slow tumor growth in mice (using in vivo models of pre-existing TNBC cell line-derived tumors). ADP2, ADP3 and ADP13 all showed anti-tumor growth inhibitory activity in vivo. However, ADP2 and ADP13 were selected for further testing as the two most effective antibodies.

In Phase 3 screening, additional testing in vivo was performed to test the ability of the lead ADPs to reduce metastasis and improve survival (using a neoadjuvant TNBC tumor resection model followed by tissue imaging). ADP2 and ADP13 were then tested for their ability to work in combination with the chemotherapeutic agent Nanoparticle Albumin-Bound Paclitaxel (NPAC), which is a standard-of-care treatment for patients with recurrent TNBC. The results from these TNBC animal models that closely mimic patient treatment protocols validated the ability of ADP2 and ADP13, to significantly improve disease outcome when administered as monotherapies or in combination with chemotherapy.

Amino acid sequencing analysis identified the Complementarity-Determining Regions (CDRs) of ADP2, ADP13, and ADP3. With the exception of CDR L2 and CDR H1 of ADP2 and ADP13, which bear some similarity to each other, the other CDRs of the light and heavy chains of these ADPs were different. FACS analysis using cells that express ADAM8 MP and DI domains vs DI domain alone, showed that both ADP2 and ADP13 bound to the DI domain. Epitope mapping at the peptide level using hydrogen deuterium exchange (HDX) mass spectrometry peptide analysis confirmed ADP2 and ADP13 bind to the ADAM8 DI domain. Chimeras of the light chain variable domain (VL) and heavy chain variable domain (VH) of ADP2 and ADP13 with the C region of human IgG1 (chADP2 and chADP13, respectively) were generated to test for the ability of the mouse V regions to function in the context of human C regions. Binding to ADAM8 and dual MP/DI antagonist activity were retained by the two chimeras. Finally, ADP2 and ADP13 were epitope mapped at the amino acid level using shotgun mutagenesis.

### Example 4. Maintenance of Hybridoma Clones and Monoclonal Antibody Preparation

Anti-ADAM8 antibody producing hybridoma lines and control mouse hybridomas producing isotype-matched IgGs were grown in HyClone™ CCM1 media (GE Healthcare). Control hybridomas expressed bromodeoxyuridine (BrdU) IgG1 (clone G3G4, Developmental Studies Hybridoma Bank, U. Iowa City) and anti-Manduca sexta ecdysone IgG2b (clone 10F1, Developmental Studies Hybridoma Bank). All lines were confirmed mycoplasma-free using a polymerase chain reaction (PCR)-based test (Venor™MGeM Mycoplasma Detection Kit, Sigma). Antibodies were purified from supernatants using recombinant Protein A affinity purification. Sterile filtered purified antibodies in 0.02 M potassium phosphate, 0.15 M sodium chloride (pH 7.2-7.4) buffer had low endotoxin levels (<2EU/mg) and displayed >95% purity as determined by sodium dodecyl sulfate polyacrylamide gel electrophoresis (SDS-PAGE) analysis.

### Example 5. ELISA Assays of ADP Binding to Recombinant Human ADAM8 (rHuADAM8)

ELISAs were performed to assess the anti-ADAM8 antibodies for binding to rHuADAM8 during preparation and screening stages, using mouse sera, hybridoma supernatants and purified mouse and chimeric ADPs. ELISA plates (96-well) were coated with 1 μg/ml rHuADAM8 (Acro Biosystems, AD8-H5223) overnight at 4° C. Plates were washed three times with phosphate buffered saline (PBS) containing 0.05% Tween 20 (PBST) and blocked with 1% bovine serum albumin (BSA) in PBST at 37° C. for 1 hr. Plates were then exposed to sera, supernatants or purified antibodies for 1 hour at 37° C.

For analysis of antibodies in the serum of mice, pre-bleed and test bleed samples in 6 dilutions ranging from 1:100 to 1:10,000,000 were tested. For analysis of antibodies in hybridoma clone and subclone supernatants, plates were subjected to 50 μl of supernatants. A test bleed sample (1:1000) and hybridoma culture medium were used as positive and negative controls, respectively. For purified antibody characterization, coated plates were incubated with eight increasing concentrations of each specific ADP ranging from 10⁻⁵ to 10³ nM. Normal mouse IgG (1 μg/ml) was used as a negative control to indicate the level of general non-specific binding and a test bleed sample (1:100) was used as a positive control. Samples were washed three times with PBST and incubated for 30 minutes at 37° C. with a secondary goat anti-mouse IgG (Fc specific)-HRP antibody (Sigma A0168, 1:5000). After washing three times with PBST, the signal was developed with addition of 100 μl of the horseradish peroxidase substrate 3,3′,5,5′-Tetramethylbenzidine (TMB) for 10 minutes at room temperature, followed by quenching with 50 μl 1N HCl. Signal was read in a 96-well spectrophotometer at an optical density (OD) of 450 nm.

### Example 6. FACS Analysis of ADP Binding to Native ADAM8

FACS analysis was used to assess mouse sera, hybridoma supernatants and purified mouse or chimeric ADPs for binding to native ADAM8. Variations of the same basic protocol were used. For these experiments, the human embryonic kidney (HEK) cell line 293 (HEK293), which does not naturally express ADAM8, was purchased from the American Type Culture Collection (ATCC) and maintained in medium recommended by ATCC. Stable cell lines HEK293-full-length-ADAM8 (termed HEK293-ADAM8 or Full-length), HEK293-remnant-ADAM8 (termed Remnant) and HEK293-Empty Vector (EV) were generated by transfection of HEK293 cells with full-length human ADAM8 cDNA (MGC: 134985; Genbank: BC115404.1), the remnant form ADAM8 cDNA and control pCDNA3.1 Version B DNA (Invitrogen), respectively, using Lipofectamine® 2000 (Invitrogen, 11668), according to the manufacturer's directions, and selection in 500 μg/ml geneticin (G418, Teknova). ADAM8 cDNA constructs were as described previously (Romagnoli et al., EMBO Mol Med, 6 (2): 278-294, 2014; Das et al., Breast Cancer Res, 18 (1): 40-58, 2016; Srinivasan et al., J Biol Chem, 289 (48): 33676-33688, 2014).

For FACS analysis, cells were trypsinized and single cell suspensions generated by passing cells through a syringe with a 21 g 1½ needle. Three hundred thousand cells per sample were resuspended in 50 μL FACS Buffer (1% BSA, 0.1% sodium azide in PBS). Cells were exposed to sera, supernatants or purified antibodies for 30 minutes on ice. For analysis of antibodies in the serum of mice, pre-bleed and test bleed samples at 1:100 dilution were used. For analysis of antibodies in hybridoma clone and subclone supernatants, cells were subjected to 10 μL of supernatant samples. Purified mouse ADPs were assessed at concentrations of 10, 1 and 0.1 μg/ml. Normal mouse IgG (1 μg/ml) was used as a negative control. For a positive technical control, staining with 1 μg/ml goat anti-mouse ADAM8 antibody AF1031 (R&D Systems) vs normal goat IgG was performed. Chimeric ADPs were tested at 2 μg/ml and human IgG1 (Sigma, 15154) was used as an antibody isotype-matched control. After incubation with primary antibodies, cells were washed three times with 1 ml of FACS buffer each time and incubated in the dark in 50 μl FACS buffer with secondary antibody. Secondary antibodies were: Alexa Fluor 488 donkey anti-mouse IgG (H+L) antibody (1:1000, Life Technologies, A-21202), Alexa Fluor 488 donkey anti-goat IgG (H+L) antibody (1:1000, Life Technologies, A-11055) and Alexa Fluor 488 goat anti-human IgG (H+L) antibody (1.25 μg, Life Technologies, A-11013). After a 20 minutes incubation with secondary antibodies, cells were washed with 1 ml of FACS buffer three times, resuspended in 400 μl of FACS buffer and analyzed by flow cytometry using a BD FACSCalibur™ machine.

### Example 7. Assays for Testing of ADP Cross-Reactivity to ADAM8 Related ADAM Proteins

ELISA assays were also used to test hybridoma clones, subclones and purified ADP antibodies for specificity of ADAM8 binding, i.e., binding to rHuADAM8 (Acro Biosystems, AD8-H5223) was compared to binding to closely related recombinant human ADAM proteins: ADAM9 (R&D Systems, 939-AD-020), ADAM12 (Acro Biosystems, AD2-H5228) and ADAM15 (Sino Biological, 10517-H08H). For analysis of clones and subclones, 50 μl of cell supernatants were used. A test bleed sample (1:1000) was used as a positive control. Hybridoma culture medium or a pre-bleed serum sample (1:1000) was used as a negative control. Purified mouse ADPs were assessed at 1 μg/ml vs normal mouse IgG and with a test bleed sample as a positive control. ADP binding was detected with a goat anti-mouse IgG (Fc specific)-peroxidase antibody (1:5000, Sigma-Aldrich, A0168). All ADPs showed high ADAM8 specific binding and very low cross-reactivity to related ADAM9, ADAM12 and ADAM15 proteins.

ADP2 and ADP13 were also tested for cross-reactivity to the ADAM8 related protein ADAM33 using a variation of the above FACS protocol, which included steps for cell fixation and permeabilization. FACS was performed as an ELISA assay was precluded due to the lack of commercially available recombinant ADAM33. Cell fixation and permeabilization steps were needed as ADAM33 expression in HEK293 cells is primarily intracellular. HEK293 cells were transiently transfected over a 48-hr period with an ADAM33 construct (Clone ID HsCD00419548, Harvard Plasmid Information Database) or a control EV DNA (Plasmid #25890, Addgene) using Lipofectamine® 2000. Single cell suspensions (1×10⁶ cells/ml) were prepared in FACS buffer. Cell samples (1 ml) were centrifuged, resuspended in 50 μL cold 4% paraformaldehyde and incubated for 20 minutes at 4° C. in the dark with occasional shaking. Cells were then washed once with 1 ml FACS buffer, and then twice with 0.1% saponin FACS buffer. Samples were exposed to primary antibodies in 50 μL of 0.1% saponin FACS buffer for 30 minutes at 4° C. For ADP2, ADP13 and their respective IgG2b and IgG1 isotype matched controls, 2 μg of antibody were used. An anti-ADAM33 antibody (LifeSpan Biosciences, LS-C124915) (0.3 μg) was used as a positive control; a sample was also stained with its IgGI isotype control at the same concentration. Following primary antibody exposure, samples were washed 2× in 0.1% saponin FACS buffer and exposed to 1.25 μg secondary antibody [Alexa Fluor 488 chicken anti-mouse IgG (Life Technologies, A21200)] in 50 μL of 0.1% saponin FACS buffer for 30 minutes at 4° C. After three washes in 0.1% saponin FACS buffer, cells were resuspended in 500 μL FACS buffer and analyzed on a BD FACSCalibur™ machine.

### Example 8. ADAM8 Metalloprotease (MP) Domain Activity Assays

### Activation of a Quenched Fluorescent CD23 Peptide

MP domain activity of rHuADAM8 was confirmed prior to mouse immunization by assessing its ability to release fluorescence from a tagged/quenched peptide, derived from the ADAM8 target protein CD23. Briefly, duplicate samples (100 μl) of rHuADAM8 (1 μg) diluted in assay buffer (1 M Tris HCl, pH 8.0, 10 mM CaCl₂, 6×10⁻⁴ Brij detergent) with or without 30 mM Ethylenediaminetetraacetic acid (EDTA) were prepared. Additionally, samples (100 μl) with assay buffer plus 30 mM EDTA or assay buffer alone were prepared as controls (lacking ADAM8). EDTA is an inhibitor of ADAM8 protease enzymatic activity as it chelates the divalent cations required for MP activity. Next, 3 μl of quenched fluorescent CD23 peptide (Biozyme, PEPDAB013m001) working solution (10 mM in DMSO) was diluted (1:400) in assay buffer and 100 μl aliquots added to each experimental well. A control sample containing assay buffer alone was used to set background levels. Fluorescence was measured every hour up to 8 hr and then again at 24 hr. An increase in fluorescence was seen over time in samples with rHuADAM8, which was inhibited in the presence of the chelating agent EDTA. These results indicated that the rHuADAM8 protein had an active MP domain.

CD23 Cleavage from the Cell Surface

Cellular MP activity was measured using a modified version of the Romagnoli protocol (EMBO Mol Med, 6 (2): 278-294, 2014). Briefly, 4×10⁵ HEK293 cells/well were plated in 12-well plates. After 24 h, cells were co-transfected with 3 μg of a plasmid encoding C-terminal HA-tagged membrane isoform b of CD23, a well-known substrate of ADAM8 (Fourie et al., J Biol Chem 278 (33): 30469-30477, 2003), and 1.2 μg of either full-length ADAM8 or empty vector pCDNA3.1 DNA using Lipofectamine® 2000. After 6 hours, the transfection medium was replaced with culture medium without FBS in the presence of either: (a) concentrated and dialyzed ADPs from hybridoma supernatants, or (b) purified ADPs following generation of stable subclones. Specifically, hybridoma supernatants were concentrated ~10-fold using Amicon Ultra Centrifugal Filters (EMD Millipore) and dialyzed against PBS using Micro Float-A-Lyzer dialysis units (Spectrum Labs) to remove hybridoma media. Concentrated dialyzed ADP samples were then quantified using an Easy Titer IgG Assay kit (Thermo Fisher Scientific) and concentration confirmed by Nanodrop Lite Spectrophotometer analysis.

Activity of dialyzed ADPs (20 μg/ml) from hybridoma supernatants was compared to that of similarly processed prototype anti-ADAM8 antibody MAB1031 (R&D Systems) (used in U.S. Patent Publication No. 2016/0130365) vs its isotype-matched control mouse IgG2b, to identify hybridomas with MP domain inhibitory activity. Once stable subclones were established, the MP inhibitory activity of protein A-purified ADPs was confirmed vs MAB1031, and the appropriate isotype-matched control mouse IgGs (20 μg/ml). Control IgGs used were: BrdU IgG1 (clone G3G4, Developmental Studies Hybridoma Bank, U. Iowa City), anti-Manduca sexta ecdysone IgG2b (clone 10F1, Developmental Studies Hybridoma Bank, U. Iowa City), and IgG2c (clone 6.3, ASB-12201, Nordic Biosite). After 16 hours of exposure to either dialyzed or purified antibodies, the serum-free media was harvested and centrifuged to remove cell debris, while cells were trypsinized and counted. Conditioned cell media (volumes corresponding to 50,000 cells per each sample) were assessed for cleaved CD23 via detection of its HA-tag in Western blotting. Whole cell extracts from lysed cells were further assessed for ADAM8 levels.

MP % inhibition was calculated as a decrease in cleaved CD23 in the conditioned media of HEK293 cells co-expressing CD23 and ADAM8, following anti-ADAM8 treatment vs treatment with isotype-matched control IgG (set to 100%). EV/CD23 co-transfected HEK293 cells were used to set background staining. MP activity studies with triple-negative inflammatory breast cancer cells were performed in essentially the same way as described above but using SUM149 cells rather than HEK293 cells.

### Western Blotting

Whole-cell extracts (WCEs) from cells in culture and conditioned cell media were prepared and immunoblotted as described previously (Romagnoli et al., EMBO Mol Med, 6 (2): 278-294, 2014). Briefly, WCE were prepared using Radioimmunoprecipitation assay buffer (RIPA, 50 mM Tris pH 7.6, 150 mM NaCl, 1% NP40, 0.1% SDS, 5 mM EDTA, 1% Sodium Sarkosyl) supplemented with Halt Protease and Phosphatase Inhibitor Single-Use Cocktail (1:100, Thermo Fisher Scientific, 78442), 0.5 M EDTA (1:100) and 1 M 1,10-Phenanthroline (1:100, Sigma, 131377) to inhibit the autocatalytic activity of ADAM8. Samples (25 μg) were subjected to immunoblotting for ADAM8 with an anti-ADAM8 antibody (LifeSpan Biosciences, LS-B4068) and for β-Tubulin, as a loading control, with an anti-β-Tubulin antibody (Sigma, T6793).

For Western blot analysis of released HA-tagged CD23 in MP assays, volumes of the conditioned media corresponding to 50,000 cells per sample, prepared as described above, were subjected to immunoblotting using an anti-HA antibody (Sigma, H6908) for detection of the CD23 HA-tag. A Precision Plus Protein Dual Color Standard (Biorad, #1610374) was used as a protein size marker.

### Example 9. ADAM8 Disintegrin (DI) Domain Activity Assays

Adhesion of α9β1 integrin expressing cells to rHuADAM8

ADAM8 binding to and activation of β1 integrin on the cancer cell surface is a critical step in tumor spread mediated via the activity of a functional DI domain (Romagnoli et al., EMBO Mol Med, 6 (2): 278-294, 2014; Schlomann et al., J Biol Chem, 277:48210-48219, 2002). To mimic this process and evaluate both the presence of an active DI domain on rHuADAM8 and the ability of ADPs to inhibit this activity in vitro, adhesion of Chinese Hamster Ovary (CHO) cells expressing α9β1 integrin vs the negative control αvβ3 integrin was used (Rao et al., J Bone Miner Res, 21 (10): 1657-1665, 2006). CHO cells were maintained in DMEM high glucose (Invitrogen) supplemented with 10% FBS, 1% penicillin/streptomycin (Hyclone), L-glutamine (Gibco), non-essential amino acids (Gibco) and 100 μg/ml G418 (Teknova). Adhesion of CHO cells expressing a9B1integrin or αvβ3 integrin to plates coated with rHuADAM8 (Acro Biosystems, AD8-H5223) was assessed as follows. Briefly, 96-well plates were coated overnight with 1.5 μg of rHuADAM8 per well, blocked with 1% BSA and washed with PBS.

To test for the presence of an active DI domain on rHuADAM8, wells were then pre-treated in duplicate for 2 hours with either PBS, 10 μg/ml neutralizing rat anti-human β1 integrin antibody (BD Pharmingen, 552828) or normal rat IgG control. A single cell suspension of CHO cells expressing α9β1 integrin (1×10⁵ per well) was then added and allowed to attach for 1 hour at 37° C. CHO cells expressing αvβ3 integrin, which does not interact with ADAM8, were used as an added control. Unattached cells were washed off and attached cells counted manually on a microscope using a grid drawn on the bottom of the plate. CHO cells expressing α9β1 integrin were able to bind to rHuADAM8 and this binding was inhibited by exposure to a β1 integrin neutralizing antibody. In contrast, CHO cells expressing αvβ3 integrin were unable to attach. These data indicated that rHuADAM8 had an active DI domain.

To assess the ability of ADPs to inhibit DI activity, 96-well plates coated with rHuADAM8 as above were pre-treated in duplicate for 2 hours with either PBS, 20 μg/ml of dialyzed or protein A purified ADP, MAB1031 or control isotype-matched IgGs. Unattached cells were washed off and attached cells counted as above. DI % inhibition was calculated as a decrease in adhesion of CHO cells expressing α9β1 integrin to rHuADAM8 following ADP treatment vs treatment with an isotype-matched control IgG (set to 100%). % Cell Binding=control IgG (set to 100%)−DI % inhibition, and is given as a mean±Standard Deviation (S.D.) from 3 independent experiments.

### Transendothelial Migration

Transendothelial migration (TEM), the DI domain-mediated ability of ADAM8-expressing cells to move through a layer of endothelial cells, mimicking entry into a blood vessel, was used as an additional test for inhibition of DI activity by ADPs as described previously (Romagnoli et al., EMBO Mol Med, 6 (2): 278-294, 2014). The human triple-negative breast cancer (TNBC) cell line MDA-MB-231 and the human umbilical vein endothelial cell line HUVEC were purchased from ATCC and maintained in their recommended media. MDA-MB-231-luciferase-tagged cells were generated by lentiviral infection followed by selection in G418 (500 μg/ml) as described previously (McLaughlin et al., Cancer Cell, 24 (3): 365-278, 2013). The TNBC cell line SUM149, representative of the highly aggressive inflammatory breast cancer phenotype (Forozan et al., Br J Caner, 81 (8): 1328-1334, 1999) was maintained in Ham's F-12 medium (Mediatech), containing 5% FBS (Invitrogen), 5 μg/ml insulin (Sigma), 1 μg/ml hydrocortisone (Sigma), 100 units/ml penicillin and streptomycin (Hyclone) (Mineva et al., PLOS One, 8 (9): e73464, 2013). All lines were confirmed mycoplasma-free using a PCR-based test (Venor™MGeM Mycoplasma Detection Kit, Sigma). TNBC cell lines were authenticated using short tandem repeat analysis (Genetica DNA Laboratories).

Briefly, transwells (Costar) with 8-μm diameter pores were coated with a confluent monolayer of HUVEC cells. Single cell suspensions of 4×10⁴ SUM149 cells or 1×10⁵ MDA-MB-231 cells were pre-treated with 20 μg/ml of dialyzed or protein A purified ADP, prototype MAB1031 (R&D Systems) or their respective isotype-matched control IgGs in serum free media for 30 minutes at room temperature, layered in the upper compartment of the HUVEC-coated transwells and allowed to migrate at 37° C. After 16-24 hours incubation, cells that migrated to the lower side of the filter were quantified by crystal violet staining and OD 570 nm determination. TEM % inhibition was calculated as a decrease in transwell migration following anti-ADAM8 antibody treatment vs treatment with an isotype-matched control IgG (set to 100%).

### Endothelial Cell Adhesion

To invade through a blood vessel wall, cancer cells must first adhere to the endothelial cells lining this wall in a process specifically mediated by ADAM8 DI activity. Thus, endothelial cell adhesion was used as another test for inhibition of DI activity by ADPs as described previously (Romagnoli et al., EMBO Mol Med, 6 (2): 278-294, 2014). Briefly, 1× 10⁵ HUVEC cells were plated, in duplicate, in 48-well plates and grown for 24 hours to obtain a confluent monolayer. SUM149 cells (5×10⁴) were pre-treated with 20 μg/ml of ADP13 or isotype-matched control IgG1 for 30 minutes at room temperature. An untreated sample of SUM149 was used as an additional control. After antibody pre-treatment, SUM149 cells were washed twice with PBS and incubated for 20 minutes at 37° C. in 300 μl of EBM-2 Basal Medium (Lonza) supplemented with 1% FBS on top of the confluent HUVEC monolayer or in empty wells. Unattached SUM149 cells were removed by washing three times with PBS and attached cells counted in three random fields/well (n=6). The average percentage relative adhesion vs control samples set to 100% is presented.

### Example 10. ADP Isotyping, Binding Kinetics and Epitope Binning

An SBA Clonotyping System-HRP kit (SouthernBiotech, 5300-05) was used to determine the isotype subclass and type of light chain for each ADP antibody. Briefly, anti-mouse-Fc capture antibody (1 μg/mL) in PBS (pH 7.4) was used to coat 96-well ELISA plates overnight at 4° C. Plates were then washed three times with PBST and blocked with 1% BSA in PBS at room temperature for 1 hour. Following another 3 washes with PBST, plates were exposed to 1:50, 1:500 and 1:5000 diluted or undiluted supernatants from second round stable ADP producing hybridoma subclones in blocking buffer for 1 hour at 37° C. HRP-conjugated secondary antibodies (anti-mouse Ig, mouse IgA, mouse IgG1, mouse IgG2a, mouse IgG2b, mouse IgG3, mouse IgM, mouse k and mouse 2) at a dilution of 1:100 in blocking buffer were added to appropriate wells of the plate and incubated for 1 hour at 37° C. Plates were washed three times with PBST and signal developed with addition of 100 μl TMB for 15 minutes at room temperature. This was followed by quenching with 50 μl IN HCl. Signal was read on a plate spectrophotometer at 450 nm. Wells incubated with an unconjugated anti-mouse Ig secondary were used to determine background signal. Positive signal in a single isotype, i.e., either IgG1, IgG2b or IgG2c subclass and light chain type for each hybridoma supernatant confirmed single subclone origin of each ADP produced.

Biacore Surface Plasmon Resonance Assays for ADP Binding Kinetics to rHuADAM8

For Biacore assays, each ADP was used as the ligand in a multiple cycle kinetics method performed on a Biacore™ T200 surface plasmon resonance system (GE Healthcare Life Sciences) machine. ADP proteins were captured using anti-mouse Fc IgG attached to a dextran matrix, and rHuADAM8 (Acro Biosystems, AD8-H5223) added as the analyte at concentrations ranging from 3.75 nM to 200 nM. HBS-EP+ (10 mM HEPES, 0.15 M NaCl, 3 mM EDTA, 0.05% Surfactant P20) running buffer was passed at a flow rate of 30 μl/min and antigen injected at 30 μl/min. The association and dissociation phases were carried out for 180 s and 600 s, respectively. Surface regeneration was performed for 30 s at 30 μl/min of Glycine pH 1.5. Values for association rate constant (kₐ), dissociation rate constant (k_{d}) and equilibrium dissociation constant (KD) were calculated through the Biacore™ T200 Software.

### ADP Epitope Binning by Competitive ELISA

Each purified ADP (1 μg/ml) was individually fixed on a 96-well plate overnight at 4° C. and then blocked with 1% BSA in PBST for 1 hour at 37° C. Plates were then washed three times with PBST and interacted for 1 hour with a pre-incubated mixture of biotinylated rHuADAM8 (AD8-H5223) and excess of a second competitor ADP (ADP_{C}) or control mIgG. Washed plates were then incubated with Streptavidin-HRP (1:5000 dilution) for 30 minutes at 37° C. Plates were developed with addition of 100 μl TMB for 10 minutes at room temperature, followed by quenching with 50 μl 1N HCl. OD at 450 nm was read on a plate spectrophotometer and the extent of competition between the two ADPs determined in each case. Values for percentage of competition were calculated using the formula: (1−OD₄₅₀ ADP_{C}/OD₄₅₀ control mIg) %. If the two antibodies recognize the same region of ADAM8, the numerator OD₄₅₀ ADP_{C} will be lower, yielding a higher percentage indicative of epitope similarity. High levels of cross competition were defined as equal to or greater than 75% and used to delineate 5 ADP epitope clusters.

### Example 11. SiRNA Knockdown Analyses

Transient RNAi-mediated ADAM8 knockdown was performed as previously described with the following short interfering RNAs (siRNAs) (QIAGEN) (Romagnoli et al., EMBO Mol Med, 6 (2): 278-294, 2014): siADAM8 RNA-1 (siA8-1, Hs_ADAM8_6): 5′-CGGCACCTGCATGACAACGTA-3′ (SEQ ID NO: 101); siADAM8 RNA-2 (siA8-2, Hs_ADAM8_7): 5′-CTGCGCGAAGCTGCTGACTGA-3′ (SEQ ID NO: 102); AllStar negative control siRNA (Qiagen) was used in each experiment as a non-silencing control siRNA (siCtrl). siRNAs (10 nM) were introduced in cells using Lipofectamine RNAi Max Transfection Reagent (Invitrogen) by reverse transfection according to the manufacturer's protocol. Reduced ADAM8 levels were confirmed by Western blotting, as above. For functional assays, transfected cells were analyzed within 24 hours.

### Soft Agar Assays

Soft agar assays were performed to evaluate the role of ADAM8 in anchorage independent growth of SUM149 TNBC cells as we have previously described (Mineva et al., PLOS One, 8 (9): e73464, 2013). Briefly, 1.0×10⁵ SUM149 cells in a mix of 0.4% Bacto Agar (BD Biosciences) in complete media were plated in triplicate on six-well dishes pre-coated with a 1:1 mix of 2× Ham's F-12 medium supplemented with 10% FBS and 1.6% Bacto Agar. Cells were fed three times per week with complete Ham's F-12 medium. After 3 weeks, cells were stained overnight with 0.2 mg/ml iodonitrotetrazolium chloride (Sigma) and photographed at 40× magnification. Colonies with diameters of approximately 20 microns or greater were counted using ImageJ software (NIH).

### Matrigel Outgrowth Assays

Matrigel outgrowth assays were carried out to evaluate the role of ADAM8 in invasion of SUM149 cells through a collagenous extracellular matrix as described previously (Belguise et al., Cancer Res, 67 (12): 5763-5770, 2007). Matrigel solution (BD Biosciences, 356231) was diluted with cold serum-free Ham's F-12 medium to a working concentration of 6.3 mg/ml and kept on ice until use. Two hundred microliters of diluted Matrigel was added to each well of a 24-well dish and the dish was subsequently incubated at 37° C. for 30 minutes to allow the Matrigel to solidify. For each sample, 10 μl of a single-cell suspension containing 5.0×10³ SUM149 cells in Ham's F-12 media were then mixed with 190 μL of Matrigel at 4° C. and added to the preset Matrigel layer in 24-well plates, which was again incubated at 37° C. for 30 min. Lastly, 500 μL of complete growth SUM149 medium was layered on top of the cells. Cultures were incubated for 10 days and photographed at 20× magnification.

### Spheroid Formation Assays

Spheroid formation assays were performed to evaluate the role of ADAM8 in SUM149 cell 3D growth in suspension. Briefly, single cell suspensions of 2×10⁴ SUM149 cells in complete Ham's F-12 medium were plated, in triplicate, on ultra-low attachment 6-well dishes (Costar) and incubated at 37° C. After 5 and 7 days of culture, primary spheres were photographed at 20× magnification. Using a grid drawn on the bottom of the 6-well plate and the microscope objective ruler, spheroids with a diameter of 125 microns or greater were counted manually. Values shown are averages±S.D.

### Example 12. Xenograft Models for Evaluation of ADP Activity in TNBC

### Tumor Formation Model

The ability of ADP13 to inhibit the establishment of ADAM8-positive SUM149 TNBC cell line derived tumors was assessed in vivo using a tumor formation mouse model. Briefly, eight-week-old female Non-Obese Diabetic/Severe Combined Immunodeficient (NOD/SCID) mice were implanted with 0.5×10⁶ SUM149 cells in 30 μl of a 1:1 dilution of Matrigel (BD Biosciences) and Ham's F-12 medium, in the fourth inguinal mammary fat pad (MFP). ADP13 was administered at 1.5 mg/kg or 4.5 mg/kg vs control isotype matched IgG1 (G3G4 clone) at 4.5 mg/kg using i.p. injection 2×/week starting at the day of cell implantation into the MFP. Primary tumor growth was monitored by caliper measurement twice a week. Tumor volumes were calculated as (Length×Width²)/2 and the mean±Standard Error of the Mean (S.E.M.) presented. Mice were sacrificed when tumors in the control group approached a volume of ~1 cm³. Statistical analysis was performed using a two-tailed Student's t-test.

### Pre-Existing Primary Tumor Growth Model

ADP-mediated inhibition of established, rapidly growing, primary TNBC tumors was assessed using pre-existing cell line-derived tumor mouse models. Briefly, ten-week-old female NOD/SCID mice were implanted with 0.5×10⁶ TNBC cells (MDA-MB-231 or SUM149) in 30 μl of a 50% Matrigel (BD Biosciences) solution (1:1 Matrigel dilution+serum-free cell medium) in the fourth inguinal MFP. Tumors were allowed to form and grow undisturbed initially. When tumors reached ~50-75 mm³, mice were randomly distributed into groups and treated with ADP antibody or isotype-matched control IgG in doses ranging from 1 to 30 mg/kg, as indicated. Antibody was administered 3×/week using i.p. injection. Tumor volume was measured 3×/week using calipers and calculated, as above. Mice were sacrificed when average tumor growth in the control group approached 1 cm³. Significance was determined using a two-tailed Student's t-test.

### Neoadjuvant Treatment Protocol

To test whether ADP monotherapy can decrease metastasis and improve outcome, a neoadjuvant treatment, surgical resection protocol was performed. MDA-MB-231-luciferase tagged cells, which preferentially metastasize to the bone, were injected in the MFP of ten-week old female NOD/SCID mice as above. Once tumors reached a volume of ~50-75 mm³, mice were treated with 10 mg/kg ADP2, ADP13 or their control IgGs in i.p injection 3×/week. Tumors in all treatment groups were surgically removed when a volume of ~200 mm³ was reached in the control IgG treated group (about a week after treatment initiation). Following surgical removal of tumors, antibody treatment was continued for 12 weeks. Mouse health was assessed 3×/week and recurrence of a tumor at the primary site detected using palpation. Mice were sacrificed when recurrent tumors reached 0.9 cm³. Kaplan-Meier curves for disease-free survival and overall survival were generated using Prism software. Statistical significance was determined using a Log rank test.

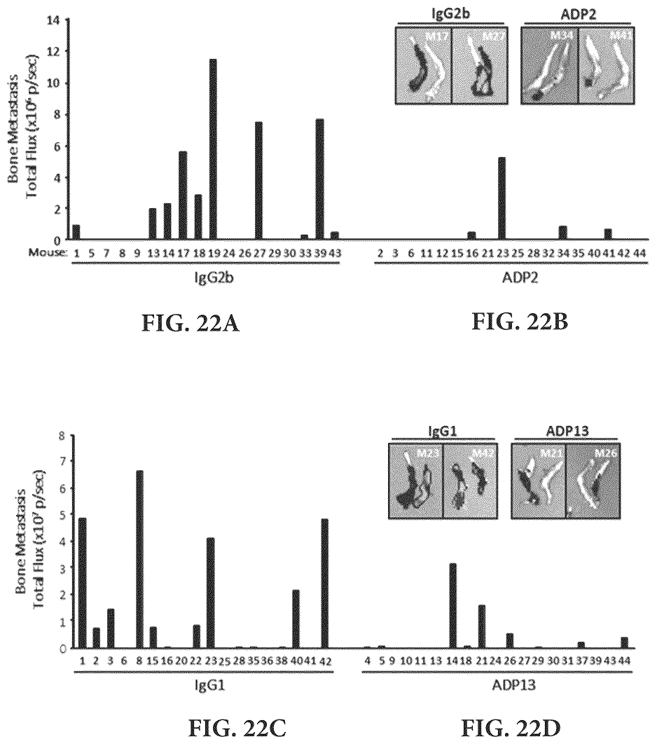

At sacrifice, either due to the presence of a large recurrent tumor or at the end of the experiment (Day 88 post-resection), mice were also assessed for metastases using biophotonic imaging of dissected bones on a Xenogen IVIS-200 machine for detection of activity from the luciferase tag expressed in MDA-MB-231 cells. Total flux indicates the presence and extent of metastasis in dissected bones. Representative images of hind leg bone metastases are shown, e.g., in FIGS. 22B, 22D, 28C, 28D, 31C and 31D. A grey color on the bone indicates a small to medium metastasis. A black color on the bone corresponds to a large metastatic lesion. A white color on the bone indicates no metastasis.

### Combinatorial Treatment Protocol

In the clinical setting, new therapies are tested initially in combination with standard of care chemotherapy so that patients are not put in a position of having to choose between a proven, while not necessarily efficient, therapy and a new experimental drug. In the preclinical setting, this strategy is mimicked by testing combination regimens in tumor regression and regrowth mouse models, where full disease regression is driven by multiple cycles of chemotherapy and regrowth delayed by the new agent being studied (Volk et al., Neoplasia, 10 (6): 613-623, 2008).

To test ADP2 and ADP13 therapy under these conditions, a combinatorial regimen with the TNBC standard-of-care chemotherapeutic Nanoparticle Albumin-Bound Paclitaxel (NPAC, brand name Abraxane®) was selected. NPAC is more stable than unmodified paclitaxel and has demonstrated greater efficacy in clinical trials compared to both paclitaxel or docetaxel (Gradishar et al., J Clin Oncol, 23 (31): 7794-7803, 2005; Gradishar et al., J Clin Oncol, 27 (22): 3611-3619, 2009).

To determine an appropriate NPAC dose, NOD/SCID mice bearing MDA-MB-231-luciferase tagged cell line derived tumors, obtained as above, were treated with 5 consecutive i.v. doses (1 cycle) ranging up to 30 mg/kg NPAC (in carrier saline). The maximum effective dose of NPAC with no substantial adverse effects was 10 mg/kg in these mice [Tumor Growth Inhibition (TGI)=78%;]. Thus, to fully regress primary tumors, for the combinatorial studies 2 consecutive cycles of NPAC were performed.

Female NOD/SCID mice were injected with MDA-MB-231-luciferase-tagged cells as described above and tumor growth followed. On Day 19 after cell implantation, mice bearing well-established, rapidly growing ~150 mm³ tumors were divided into 4 groups. Treatment was initiated on Day 20 to the groups as follows: a) Isotype-matched control IgG+Saline, b) ADP+Saline, c) IgG+NPAC and d) ADP+NPAC. NPAC was administered in 2 cycles of 5 consecutive i.v. treatments of 10 mg/kg NPAC with one week of rest in between; an equivalent volume of vehicle saline was also given. ADP2, ADP13 or their respective isotype-matched controls (IgG2b and IgG1) were administered i.p. 3×/week. Antibodies were administered using the dosing regimen proposed from Pharmacokinetic (PK) studies to achieve steady state concentrations in the blood of mice (see below). A first loading dose of 20 mg/kg was followed by maintenance doses of 10 mg/kg 3×/week. Antibody treatment was started concurrently with the first NPAC cycle and continued throughout the time course. Tumor volume (Mean±S.E.M.) over time is presented. The endpoint for evaluation of TGI was an average tumor volume approaching 1 cm³ in the IgG+Saline vs ADP+Saline groups, and in the IgG+NPAC vs ADP+NPAC groups. Percentages indicate level of inhibition of tumor growth vs corresponding control group. Statistical significance was determined using a Student's t-test.

When individual mice in the experiment reached humane endpoint (the IACUC protocol allowed tumor volume of ~1.5 cm³ or a size of ~2.0 cm in any one direction for this model), survival lengths (in days) were plotted into Kaplan-Meier curves comparing IgG+Saline vs ADP+Saline and IgG+NPAC vs ADP+NPAC. Statistical significance was determined using a Log-rank test. At sacrifice, all bones were dissected and examined for metastases using biophotonic imaging for luciferase activity. IgG+Saline vs ADP+Saline and IgG+NPAC vs ADP+NPAC groups were analyzed as above.

### Example 13. PK Studies of ADP2 and ADP13

PK studies of ADP2 and ADP13 in mice were performed using ELISA assays as described below. First, to establish a relationship between optical density (OD) reading and antibody concentration, ADP2 and ADP13 standard curves were established. Blood samples from untreated NOD/SCID mice (n=6) were collected by submandibular puncture according to IACUC guidelines. Blood samples (100 μl) were collected from each mouse in Eppendorf tubes containing 40 μl of EDTA (0.5M) and pooled together. Tubes were then centrifuged at 1,300 g for 15 minutes and the resulting top layer of clear plasma was removed, aliquoted and frozen at −80° C. until time of analysis. Next, 96-well ELISA plates (Medisorp) were coated overnight at 4° C. with 100 μl of rHuADAM8 or BSA at 1.0 μg/ml in reagent diluent (10% FBS in 1×PBS, pH 7.4). Plates were washed three times with wash buffer (1×PBS with 0.05% Tween 20) and blocked with 100 μl of Blocking solution (1% BSA in 1×PBS with 0.05% Tween 20) for 1 hour at 37° C. Then, 100 μl of plasma samples (1:5000 dilution in reagent diluent) spiked with either ADP2, ADP13 antibodies or their isotype matched IgGs (8 concentrations ranging from 0.021 nM to 0.250 nM), or left untreated, were added to wells. Microplates were incubated for 2 hours at 37° C. Bound primary antibodies were detected with a goat anti-mouse IgG (whole molecule)-horseradish peroxidase conjugate (1:2500 in reagent diluent) for 30 minutes at 37° C. Signal was developed using a 1-Step Ultra TMB-ELISA Substrate Solution (Thermo Fisher Scientific) as per manufacturer's recommendations. Briefly, 100 μl were added to each well for 20 minutes in the dark. The reaction was stopped with 50 μl/well 2N sulfuric acid, and OD read at 450 nm on a microplate reader. Specific binding was defined by subtraction of the binding observed in wells with plasma from untreated mice (unspecific binding) from that observed in the wells with spiked samples. Control IgG samples were negative for binding as expected.

Three independent ELISA assays for each antibody were performed and analyzed separately before the results were averaged to establish robust concentration standard curves for ADP2 and ADP13. These assays followed current FDA bioanalytical recommendations. Specifically, over 6 concentrations of each ADP were used and intra- and inter-assay precision with less than 20% coefficient of variation and accuracy within 20% of the nominal concentration were confirmed.

Generation of these concentration standard curves enabled PK analysis of ADP2 and ADP13 following injection in mice. Female NOD/SCID mice (n=63) were weighed the day before injection. Each animal received a single i.p. injection of ADP2 or ADP13 at a dose of 10 mg/kg. Following sacrifice using CO2 asphyxiation and a secondary method of cervical dislocation, blood samples (n=3 mice per time point) were collected by intra-cardiac puncture at 2 hours, 4 hours, 6 hours (only for ADP2), 8 hours, 24 hours, 48 hours, 96 hours, 7 days, 14 days, and 21 days. Approximatively 500 μl of blood was collected in an Eppendorf tube containing 70 μl of EDTA (0.5 M, pH 8.0) and plasma was isolated as described above. The specific concentration values Mean (nM)+/−S.D. of ADP2 or ADP13 protein in plasma were determined in three independent ELISA runs using the previously established ADP standard curves. These values were then used to determine the PK profiles for both antibodies using SAS software. Ln (concentration) vs time for ADP2 and ADP13 was plotted. Based on visual evaluation, the best estimate on what points constitute the terminal beta phase was made.

For ADP2, this was 96 hours while for ADP13 it was 48 hr. Linear regression was applied on the plots to determine the elimination rate constant (Ke), which was then used to calculate the T_{1/2} in hours and days. The area under curve (AUC) from the origin out to the last observed quantifiable concentration (Cₗₐₛₜ=504 hr) was determined using the linear trapezoidal method. The extrapolated area from 504 to infinity was calculated as Cₗₐₛₜ/Ke, which was added to the truncated AUC to yield the total AUC. The clearance (CI), which is determined physiologically by blood flow to the organ that metabolizes or clears the drug, was calculated as the ratio Dose/(total AUC) converted to ml/min. Volume of distribution (Vd) was not calculated as the absolute systemic availability with the extravascular dosage route is unknown. The decay pattern was bi-exponential, and a “non-compartmental” approach was used to calculate the above values.

Based on the PK parameters obtained for the two antibodies, a treatment regimen composed of a loading dose of 20 mg/kg ADP followed by maintenance doses of 10 mg/kg 3× per week was proposed to establish steady state concentrations needed for long term treatment experiments. To confirm that the proposed dosing regimen results in the desired steady state, female NOD/SCID mice (n=30) received an i.p. injection of either 20 mg/kg ADP2 or ADP13 followed by injections of 10 mg/kg on days 2, 4 and 7. Blood samples (n=3 mice per time point) were collected by intra-cardiac puncture 2 hours after each antibody injection. On day 7, blood samples were also collected from 3 mice before the time of next dosing. Plasma was isolated and analyzed as above for antibody concentration over time, which confirmed the effectiveness of the regimen.

### Example 14. ADP2 and ADP13 Cloning and Sequencing/CDR Determination and Chimera Synthesis

RNA was extracted from hybridoma lines and subjected to 5′ cDNA synthesis, and 5′ RACE using primers specific for amplifying mouse V_{L} and V_{H} chain DNA. The products were analyzed by agarose gel electrophoresis. Bands running in the correct positions, i.e., between 500-700 base pairs, were visualized for each hybridoma, and cloned using TOPO (Thermo Fisher Scientific). These cloned DNAs were PCR-amplified, and purified by gel electrophoresis. Individual cloned DNAs were recovered from the gels and subjected to DNA sequencing. Analysis of multiple samples per V_{L} and V_{H} chain DNA confirmed the findings. CDR analysis of the sequencing data was performed using VBASE2 (vbase2.org). The DNA and resulting amino acid sequence for the V_{L} and V_{H} chains are presented.

Chimeras of the V_{L} and V_{H} chain DNAs were made with germline human IgG1 C_{L} and C_{H} regions, respectively using DNA ligation. Resulting chimeric chADP2-IgG1 (SEQ ID NO: 82 and SEQ ID NO: 83) and chADP13-IgG1 (SEQ ID NO: 84 and SEQ ID NO: 85) proteins were synthesized in CHO cells, and affinity purified using Protein A. Purified chimeric proteins were tested for their ability to bind ADAM8 (using ELISA and FACS) and to inhibit ADAM8 MP and DI domain activity (in CD23 cleavage and transendothelial migration assays), as described above.

### Example 15. Epitope Mapping of ADP2 and ADP13 Binding to Human ADAM8

### Mapping to Native ADAM8 Using Deletion Construct Analysis

To delineate the epitope binding regions of ADP2 and ADP13, FACS analysis was performed with HEK293 cells stably expressing either EV DNA, full-length ADAM8 or remnant ADAM8 (which lacks the pro- and MP domains). Cells were prepared using the basic FACS protocol described above, with 2 μg of ADP2 or ADP13 as the primary antibodies and 1.25 μg of Alexa Fluor 488 donkey anti-mouse IgG (H+L) (Life Technologies, A-21202) as the secondary antibody, and analyzed on a BD FACSCalibur™ machine. ADP2 and ADP13 were found to bind to both full-length and remnant ADAM8 expressing cells. Given the immunogen used to generate these antibodies contained only the MP and DI domains, and the DI domain is the only common region between the two expression constructs used in this analysis, the data point to the DI domain as the broad epitope region for ADP2 and ADP13 binding.

### Mapping by Hydrogen/Deuterium Exchange (HDX)

To confirm the region of ADP binding and map it at the peptide level HDX analysis was performed. First, pepsin/protease XIII digestion and liquid chromatography-mass spectrometry (LC-MS) with rHuADAM8 were used to generate peptides and assess the extent of protein coverage provided by this method. Recombinant human ADAM8 (8 μg, 1031-AD-020, R&D Systems) in 130 μl control buffer (50 mM phosphate, 100 mM sodium chloride at pH 7.4) was denatured by adding 130 μl of 4 M guanidine hydrochloride, 0.85 M TCEP buffer (final pH 2.5) and incubating the mixture for 3 minutes at 10° C. The mixture was then subjected to pepsin/protease XIII digestion using a pepsin/protease XIII (w/w, 1:1) column. The resultant peptides were analyzed using an ultra-performance (UP) LC-MS system comprised of a Waters Acquity UPLC coupled to a Q Exactive plus Hybrid Quadrupole-Orbitrap Mass Spectrometer (Thermo). The peptides were separated on a 50 mm×1 mm C8 column with a 16.5 min gradient from 2-30% solvent B (0.2% formic acid in acetonitrile). Solvent A was 0.2% formic acid in water. The injection valve, enzyme column and their related connecting tubing were inside a cooling box maintained at 20° C. The second switching valve, C8 column and their related connecting stainless steel tubing were inside a chilled circulating box maintained at −6° C. Peptide identification was done through searching MS/MS data against the human ADAM8 sequence with Mascot. The mass tolerance for the precursor and product ions were 7 ppm and 0.02 Da, respectively. Sequence coverage of 69.7% to 74% was achieved for rHuADAM8, primarily due to autocatalytic clipping of the prodomain, as expected. Next, HDX with pepsin/protease XIII digestion was carried out.

Human ADAM8 (8 μg in 20 μl) or 20 μl ADAM8 mixed with ADP2, ADP13 or ADP3 (8 μg: 24 μg) was incubated with 110 μl deuterium oxide labeling buffer (50 mM sodium phosphate, 100 mM sodium chloride at pH 7.4) for 0 s, 60 s, 600 s, or 3600 s at 10° C. Hydrogen/deuterium exchange was quenched by adding 130 μl of 4 M guanidine hydrochloride, 0.85 M TCEP buffer (final pH 2.5). Subsequently, the quenched samples were subjected to on column pepsin/protease XIII digestion and LC-MS analysis as described above. The mass spectra were recorded in MS only mode. Raw MS data was processed using HDX WorkBench software for the analysis of hydrogen/deuterium exchange MS data (Pascal et al., J. Am. Soc. Mass Spectrom. 23 (9), 1512-1521, 2012). The deuterium levels were calculated using the average mass difference between the deuterated peptide and its native form (t0). The deuterium levels at the various peptides were monitored from the mass shift on LC-MS. Deuterium buildup curves over exchange time for all peptides were plotted and ADAM8 sequences with reduction in deuterium uptake levels upon binding to antibody identified.

### Mapping by Shotgun Mutagenesis

The specific amino acid residues that mediate the interaction between ADP2 or ADP13 and ADAM8 were identified using shotgun mutagenesis paired with high-throughput flow cytometry. This was performed as described in Davidson and Doranz (Immunology 143:13-20, 2014). Specifically, a library of human ADAM8 expression constructs was prepared with single alanine mutations introduced into each amino acid residue from 192 to 497 except when alanine was the original amino acid—in which case, it was mutated to serine. This mutation library comprises the MP and DI domains of ADAM8 and was generated by high-throughput, site-directed mutagenesis. The constructs were transfected into HEK293 human embryonic kidney cells and arrayed in 384-well microplates. Only one mutated ADAM8 protein was expressed, in its native configuration, in an individual clone. These individual clones were grown in culture and their expression on the surface of HEK293 cells was confirmed using flow cytometry with a positive control anti-ADAM8 antibody (Control Ab) whose binding is not affected by the amino acid changes. Clones were then screened for binding to either ADP2 or ADP13 as test antibodies to determine which amino acid changes affect test antibody binding to ADAM8. Additionally, HEK293 cells were transfected with a wild-type (WT) ADAM8 construct or empty vector DNA, as a positive and negative control, respectively. A variety of experimental parameters were optimized for high-throughput flow cytometry including blocking buffer, and primary and secondary antibody concentrations, to identify the optimal conditions for screening. Through these studies ADP2 and ADP13 were determined to be high affinity binding antibodies. Notably, for antibodies that bind with high affinity, it is harder to identify critical amino acid binding residues unless stringency is increased.

Thus, for these studies antigen-binding fragments (Fabs) of ADP2 and ADP13, which bind with lower affinity than the full antibody, were generated. In addition, higher binding stringency conditions were tested, such as increased pH, salinity, and temperature, and/or increased washing, to weaken binding sufficiently to allow identification of critical binding residues in flow cytometry. The final experimental flow cytometry conditions were: primary antibody incubation with ADP2 Fab (0.50 μg/ml), ADP13 Fab (5.00 μg/ml) and Control Ab (0.16 μg/ml) for 60 minutes in flow cytometry buffer containing 10% goat serum in PBS (Ca²⁺ and Mg²⁺ free). Secondary antibody incubation was performed for 30 minutes with an Alexa Fluor 488 AffiniPure F(ab′) 2 fragment specific goat anti-mouse IgG (1:200, Jackson ImmunoResearch 115-546-006) for detection of ADP Fab binding and Alexa Fluor 488 AffiniPure goat anti-mouse IgG (H+L) [1:400, Jackson ImmunoResearch 115-545-003] for Control Ab binding. Samples were washed three times after primary antibody incubation and twice after secondary antibody incubation with PBS (Ca²⁺ and Mg²⁺ free). Next, HEK293 cells expressing WT or mutated ADAM8 proteins or EV DNA were incubated with ADP2 or ADP13 Fabs or Control Ab at their optimal concentrations. Fab binding was detected using Alexa Fluor 488-conjugated secondary antibodies and mean cellular fluorescence determined using the Intellicyt iQue flow cytometry platform. Mutated residues were identified as being critical to ADP2 or ADP13 epitope if they did not support the reactivity of the test Fabs but did support the reactivity of the reference Control Ab. This counter-screen strategy facilitates the exclusion of mutants that are locally misfolded or that have an expression defect. Binding of each test Fab to each mutant clone in the alanine scanning library was determined, in duplicate, by high-throughput flow cytometry. For each point, background fluorescence was subtracted from the raw data and antibody reactivity normalized to WT ADAM8.

To identify primary critical clones/residues, a threshold of Control Ab binding >70% of WT and test antibody binding <20% of WT binding was applied. Residues that did not meet the latter requirement but that still demonstrated substantially reduced test Fab binding and proximity to critical residues were identified as secondary important residues of the epitope. Lastly, the positions of the ADP2 and ADP13 critical and secondary binding residues were mapped on an ADAM8 crystal structure model that is based on the structure of vascular apoptosis-inducing protein-1 (PDB ID #2ERP, Takeda et al., EMBO J. 25:2388-2396, 2006).

### Example 16. Preparation and Isolation of Highly Specific Anti-ADAM8 Dual MP/DI Inhibitory Antibodies

### Hybridoma Method of Antibody Preparation

Highly specific, mouse monoclonal antibodies (mAbs) were prepared against human ADAM8 using a traditional hybridoma method combined with a unique three-phase screening approach (as outlined in FIG. 2). To make the anti-ADAM8 hybridomas, purified recombinant human ADAM8 ectodomain protein (aa17-497) (rHuADAM8), containing biologically active MP and DI domains as present on the outside of cancer cells, was injected into Balb/c and SJL mice, in order to give the broadest range of immune response. Anti-ADAM8 activity was confirmed in the blood of injected mice by ELISA [binding to rHuADAM8] and by FACS (binding to HEK293 cells ectopically expressing native human ADAM8). Following fusion of mouse splenocytes to Sp2/0-Ag14 myeloma cells, supernatants from the resulting hybridoma cells were screened by ELISA and FACS and clones expressing antibodies with high anti-ADAM8 binding activity identified (FIG. 2).

### Example 17. Multi-Phase Screening Strategy: Overview of Phases 1-3

### Phase 1

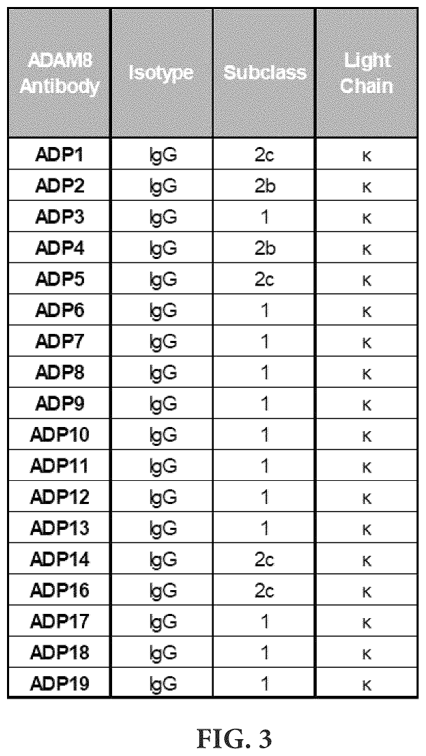

In Phase 1 of the screening strategy, hybridoma clones making antibodies cross-reacting with human ADAM9, ADAM12, and ADAM15 proteins, which have high homology with ADAM8, were identified by ELISA using recombinant ADAM proteins. Since high specificity for ADAM8 is critical for patient treatment, clones showing cross-reactivity to any of the related ADAM8 proteins were excluded from further consideration. Hybridoma supernatants were next tested in cell-based assays. Clones demonstrating dual antagonist MP and DI activity were identified and subcloned in two rounds by serial dilution. Retention of high ADAM8 binding was confirmed in ELISA and FACS experiments following each round of subcloning. Second round (final) subclones were isotyped to confirm single clone origin and dual MP/DI activity was confirmed in cell-based functional assays. Eighteen stable subclones were generated that produce dual antagonist antibodies (termed ADPs). ADPs are of either the IgG1, IgG2b, or IgG2c subclass and contain the more common k light chain (FIG. 3).

### Phase 2

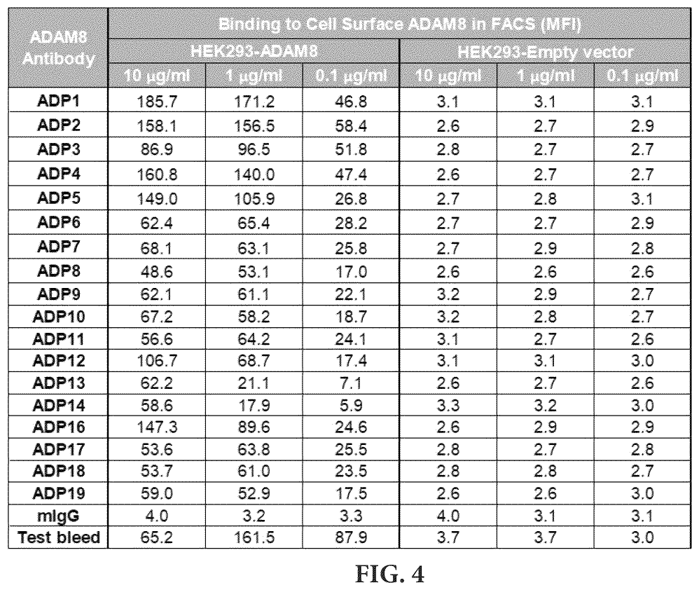

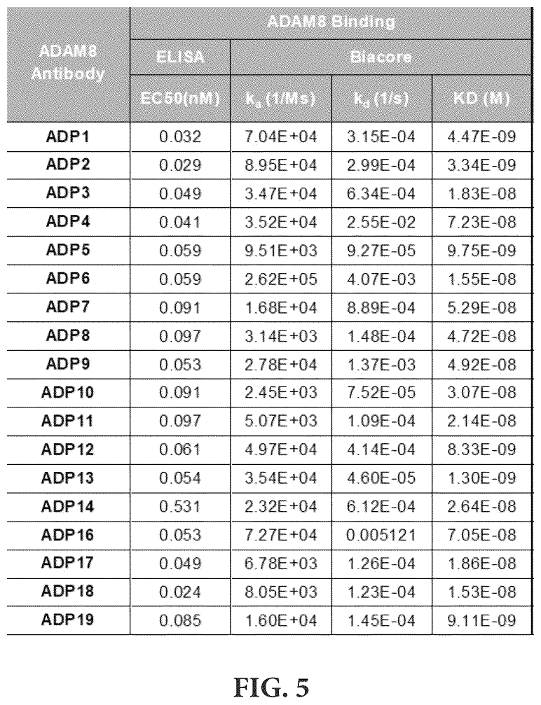

In Phase 2 of the screening procedure, purified ADPs were extensively characterized as outlined in FIG. 2. Specificity and binding activity of the antibodies to native ADAM8 was first characterized using FACS analysis with HEK293 cells, which normally do not express ADAM8, but that were transfected with either a full-length human ADAM8 expression vector or an empty vector (EV) DNA. FACS analysis, using an antibody dose-response curve, demonstrated all 18 of the ADPs have the ability to bind to ADAM8 specifically (FIG. 4). As negative and positive controls normal mouse IgG (mIgG) and a test bleed sample from a mouse injected with rHuADAM8 were used, which confirmed the specificity of the binding. An ELISA assay confirmed effective binding of purified ADPs to rHuADAM8 (FIG. 5). A Biacore binding kinetics assay was performed on each of the 18 mAbs. The association rate constant (kₐ) and dissociation rate constant (k_{d}) indicate that the ADPs bind ADAM8 with low equilibrium dissociation constants (K_{D}) [1.3×10⁻⁹ M and 7.23×10⁻⁸ M] (FIG. 5).

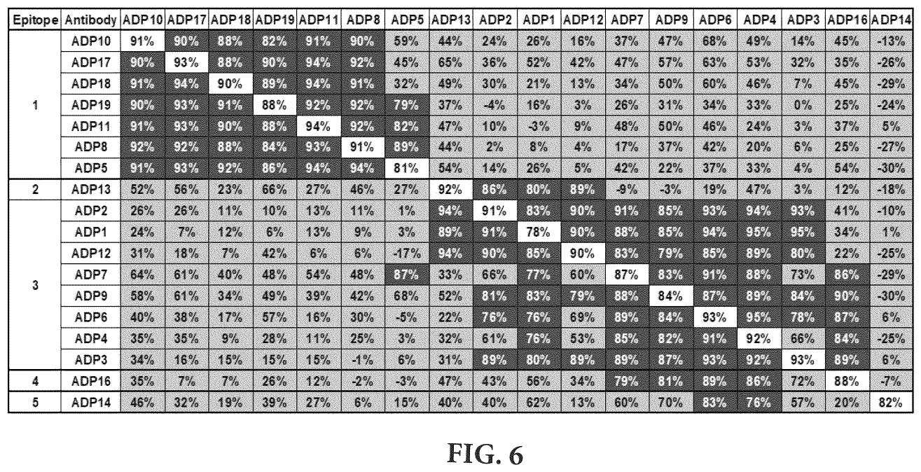

For epitope binning of these mAbs, a competition analysis was performed to determine whether they bind to overlapping or to distinct regions of the ADAM8 protein. Specifically, binding of an ADP to rHuADAM8 was challenged with excess of a second “competitor” ADP (ADP_{C}). Values for percentage of competition were calculated as follows: (1-OD₄₅₀ ADP_{C}/OD₄₅₀ control mIgG) % (FIG. 6). Thus, a higher extent of cross-competition for ADAM8 indicates a greater epitope similarity or identity.

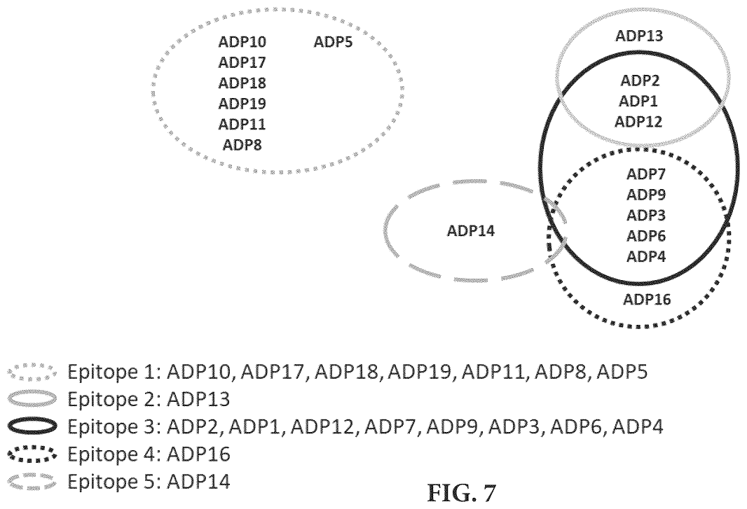

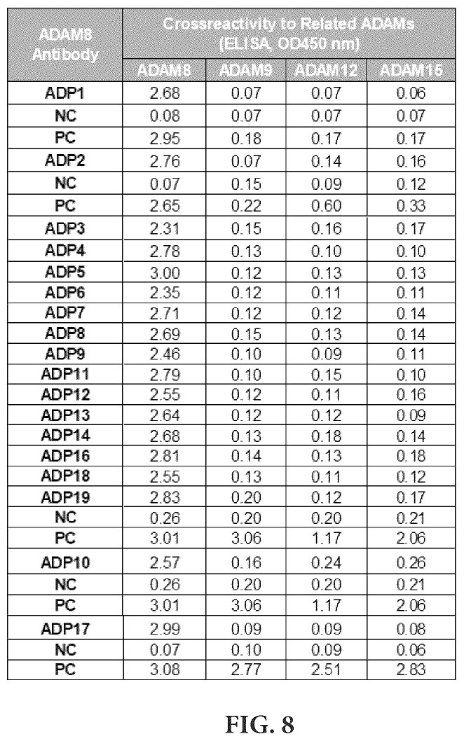

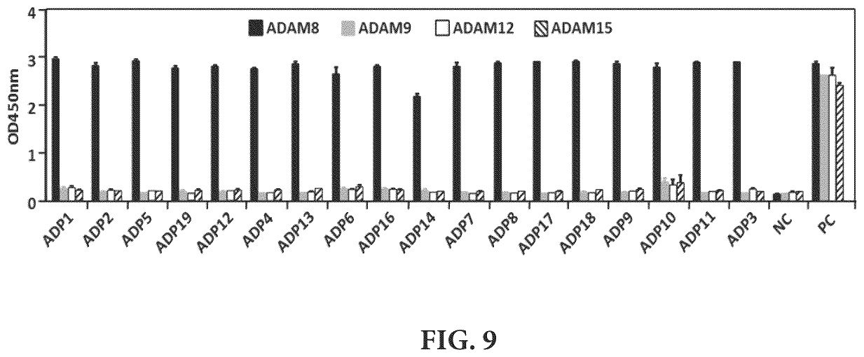

Using a cross-competition level of equal to or greater than 75% (marked in black, FIG. 6), the ADPs could be epitope binned into 5 groups, of which 4 were partially overlapping as indicated by their respective extents of competition (FIG. 7). The lack of cross-reactivity of each of the ADPs to closely related recombinant ADAM9, ADAM12 and ADP15 protein in ELISA assays (FIG. 8) confirms the specificity of ADP binding to ADAM8, which is shown in bar graph form in FIG. 9.

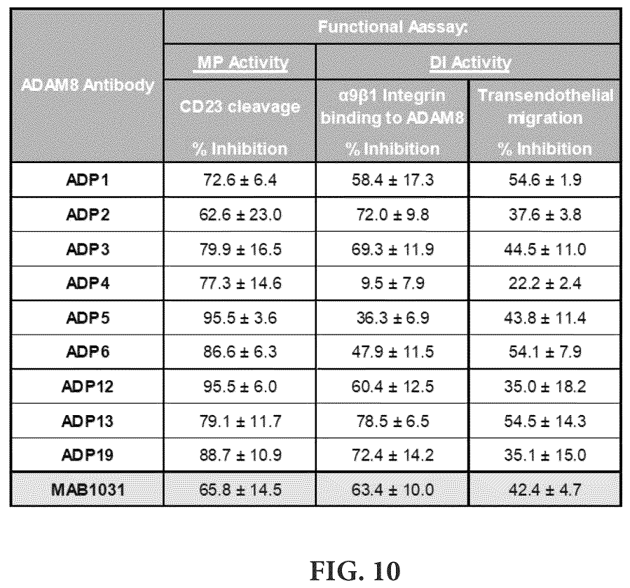

Nine ADPs with high ADAM8 binding activity (KD=1.3×10⁻⁹ to 8.3×10⁻⁸) and/or in the more abundant epitope groups (Epitopes 1, 2 and 3) were selected for further characterization. The MP and DI inhibitory activities of these ADPs were compared to the dual antagonist MAB1031 antibody (R&D Systems) (used in U.S. Patent Publication No. 2016/0130365) in cell-based assays (FIG. 10). The top four antibodies were then compared for their ability to inhibit pre-existing TNBC primary growth in mice. ADP2 and ADP13 were identified as lead tumor inhibitors. ADP3 and ADP19 showed incrementally lower activity under the tested conditions.

### Phase 3

In Phase 3, the efficacy of antibodies ADP2 and ADP13 was further characterized in vivo in neoadjuvant and chemotherapy combinatorial mouse models (FIG. 2). Amino acid sequence analysis was performed to determine the distinctiveness of each antibody in terms of CDR sequences and binding epitope mapped for ADP2, ADP13, and ADP3. Finally, chimeric ADP2 and ADP13 antibodies were constructed and their ADAM8 binding and dual inhibitory activity tested.

### Example 18. Phase 2: MP Inhibitory Activity

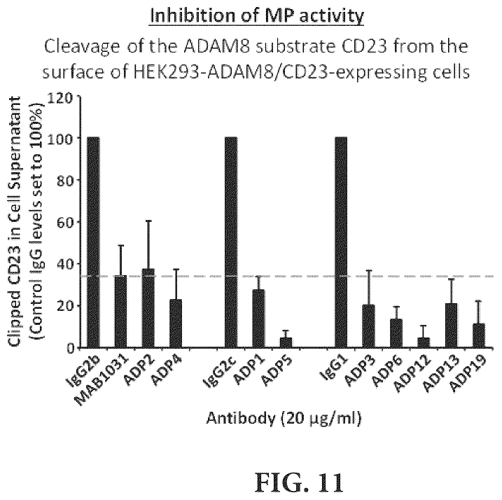

To rank the ADPs that have the highest binding affinity and belong to the more common epitopes (i.e., Epitopes 1, 2, and 3, FIG. 7), we selected 9 candidates for functional cell-based assays. MP activity was assessed first using a CD23 cleavage assay. CD23 is a well-known target of ADAM8 MP activity (Fourie et al., J Biol Chem, 278 (33): 30469-30477, 2003). ADAM8 digestion results in the release specifically of a 29 kDa CD23 fragment into the media from the surface of HEK293 cells transiently expressing a C-terminal HA-tagged CD23 and full-length ADAM8 protein that can be detected using Western blot analysis (Romagnoli et al., EMBO Mol Med, 6 (2): 278-294, 2014; Srinivasan et al., J Biol Chem, 289 (48): 33676-22688, 2014). Addition of an ADP antibody that inhibits the ADAM8 MP activity causes a reduction in release of cell-associated CD23 (Romagnoli et al., EMBO Mol Med, 6 (2): 278-294, 2014; Srinivasan et al., J Biol Chem, 289 (48): 33676-22688, 2014) (FIG. 10 and FIG. 11). All 9 of the ADP antibodies tested inhibited MP-mediated release of the 29 kDa CD23 band to an equal extent or better than the MAB1031 antibody. Thus, all 9 of the tested ADPs have potent anti-MP activity.

### Example 19. Phase 2: DI Inhibitory Activity

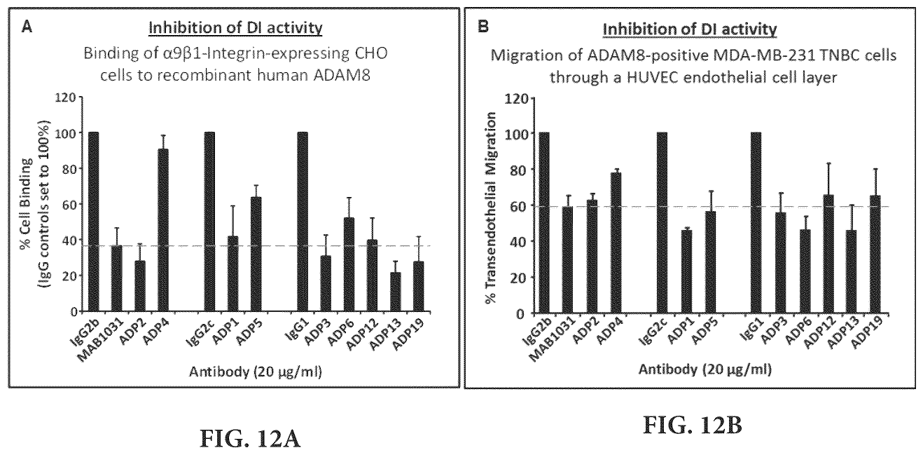

Two assays were employed to rank the 9 candidate ADPs for their DI antagonist activity. Since active ADAM8 DI domain binds to and modifies β1-integrin on the surface of cells (Romagnoli et al., EMBO Mol Med, 6 (2): 278-294, 2014), the first assay tested the effects of addition of an ADP on binding of CHO cells expressing α9β1 integrin to rHuADAM8. Six of the ADPs were either equal to or more effective than MAB1031 at inhibiting cell binding and therefore DI activity, with ADP2 and ADP13 showing the most robust inhibition (FIG. 10 and FIG. 12A). ADP5 and ADP6 displayed somewhat reduced activity compared to MAB1031, while ADP4 showed minimal DI inhibitory activity in this assay.

To further assess the ability of the ADPs to inhibit DI activity, they were next tested in transendothelial migration (TEM) assays (FIG. 10 and FIG. 12B). The modification of β1-integrin by the DI domain of ADAM8 is essential for the ability of a cell to bind to and move through an endothelial layer as mimicked in vitro by human umbilical vein endothelial cells (HUVEC). All ADPs, except ADP4, showed a level of inhibition of TEM that was comparable to or more effective than MAB1031. While ADP4 reduced TNBC cell migration across the endothelial layer to a measurable extent, it was only modestly effective.

Thus, eight of the tested 9 ADPs (all K Light chain, IgGs) had potent dual antagonist activity comparable to or better than MAB1031 with respect to either MP, DI or both domains. ADP2, ADP3, ADP13, and ADP19 were selected for in vivo testing in an orthotopic MFP mouse model with pre-existing tumors based on high KD values [ADP2, K_{D}=3.3×10⁻⁹; ADP3, K_{D}=1.8×10⁻⁸; ADP13, K_{D}=1.3×10⁻⁹; ADP19, K_{D}=9.1×10⁻⁹] and significant dual inhibitory activity in cell-based assays.

### Example 20. Phase 2: Growth Inhibitory Activity on Pre-Existing Orthotopic TNBC Tumors in Mice

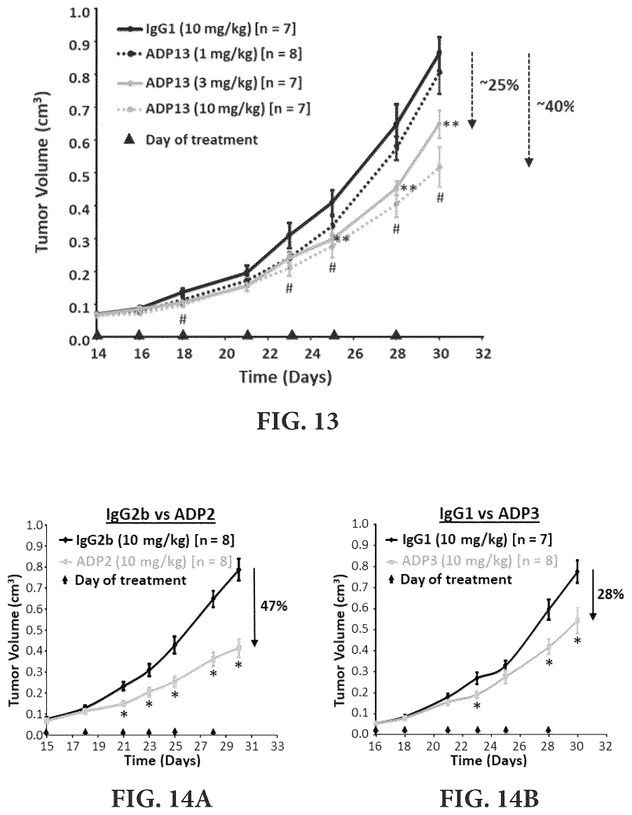

An optimal ADP treatment dose was first determined using ADP13 as the test antibody. Female NOD/SCID mice were implanted with luciferase-labeled MDA-MB-231 (MDA-MB-231-luc) TNBC cells in the fourth inguinal MFP. Once tumors reached ~50-75 mm³, mice were treated with 1, 3 or 10 mg/kg ADP13 vs 10 mg/kg control IgG1. Tumor size was measured using calipers 3×/week. Tumor volume was calculated as (Length×Width²)/2. Mice were sacrificed when tumor growth in the control group approached the 1 cm³ limit of the IACUC protocol. ADP13 inhibited tumor growth in a dose-dependent manner such that 10 mg/kg was the maximum effective dose (FIG. 13); further escalation to 30 mg/kg had no additional effect. Thus, a 10 mg/kg mAb dose was chosen for the efficacy comparison of the 4 selected ADPs.

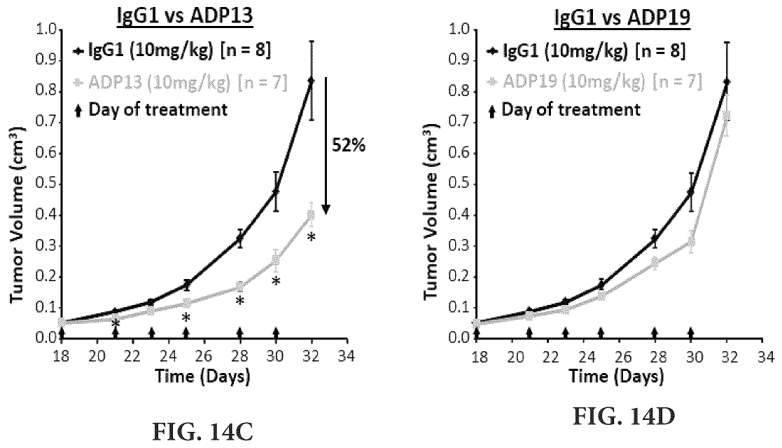

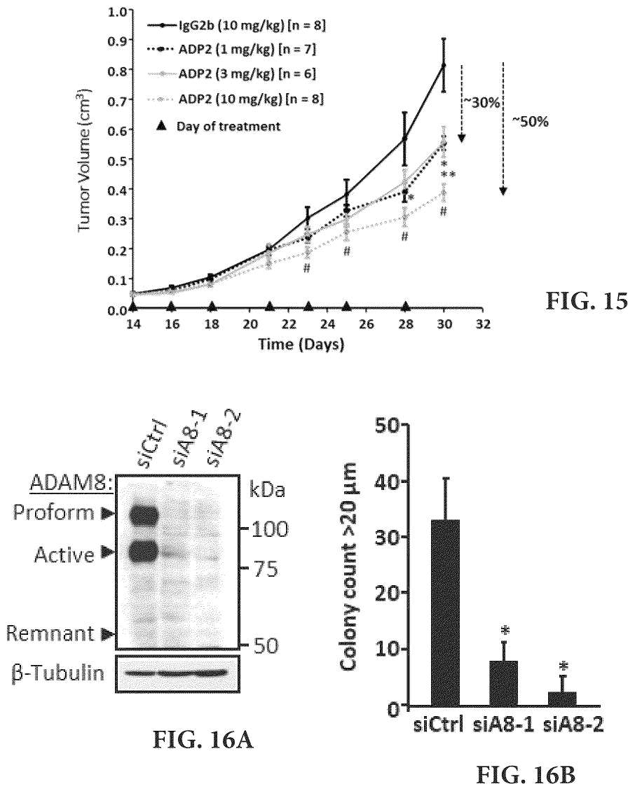

A single-dose (10 mg/kg) comparison of the ability of ADP2, ADP3, ADP13, and ADP19 to inhibit tumor growth was carried out. As controls, their isotype-matched control IgGs were used. Tumor Growth Inhibition (TGI) was: 47% for ADP2 (P=0.0001), 28% with ADP3 (P=0.0155), and 52% with ADP13 (P=0.0089). ADP19 showed no tumor growth inhibition under the tested conditions (FIGS. 14A-D). A dose-response curve up to 30 mg/kg was next performed with ADP2 vs IgG2B and 10 mg/kg was again the optimal inhibitory dose (TGI=50%, P=0.0004) (FIG. 15).

### Example 21. Phase 2: ADP13 Inhibits Growth of Tumors Derived from a Second TNBC Line

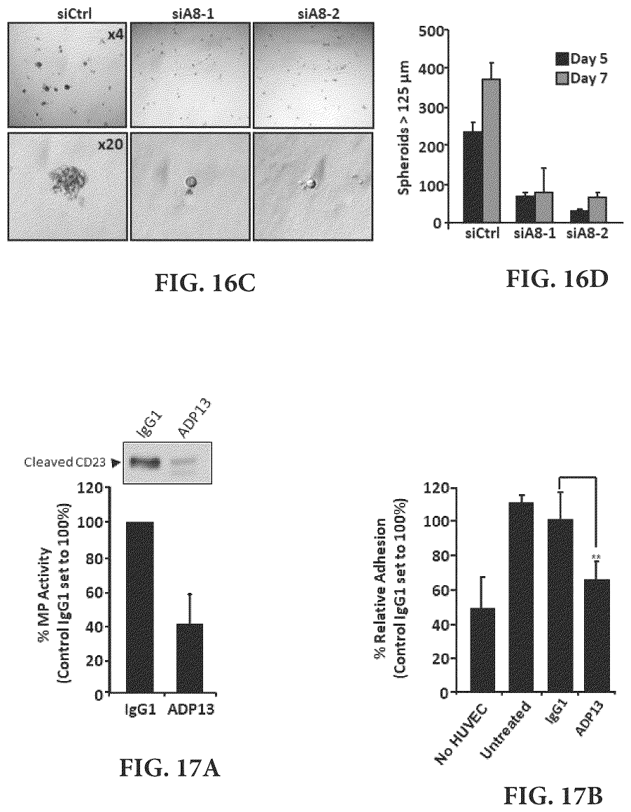

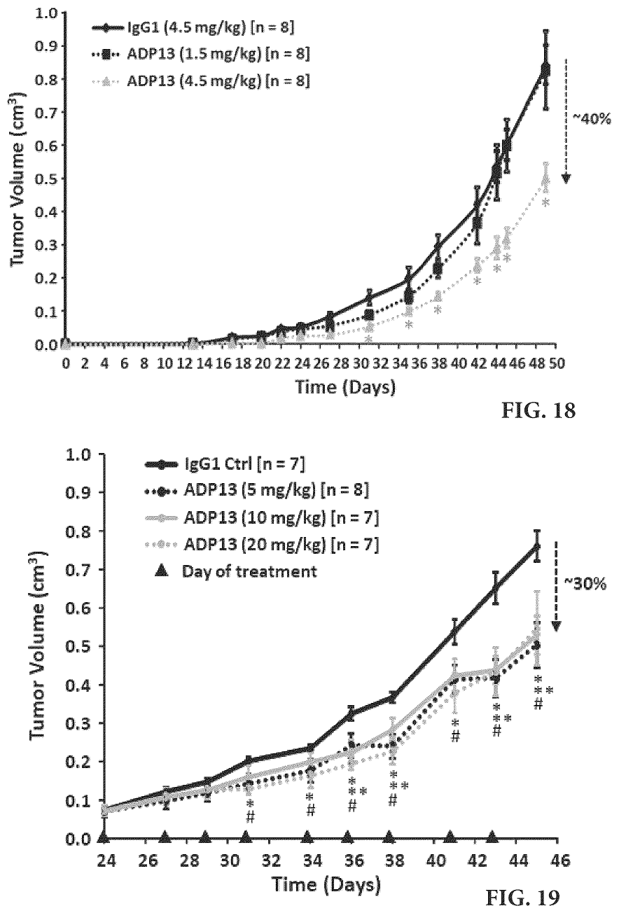

To test whether the inhibition of ADAM8 activity can reduce growth of a second ADAM8-driven TNBC cell line, the SUM149 line was selected. SUM149 TNBC cells are representative of the highly aggressive inflammatory breast cancer (IBC) subtype. SUM149 cells express a very high level of ADAM8. Knockdown of ADAM8 using siRNA technology (FIG. 16A) demonstrated that this protein mediates the ability of SUM149 cells to grow in an anchorage independent fashion in agarose (FIG. 16B), to invade through Matrigel (FIG. 16C) and to form spheroids in suspension culture (FIG. 16D). Treatment with ADP13 inhibited the MP and DI activities of ADAM8 in SUM149 cells, as judged by reduced cleavage of co-expressed CD23 (FIG. 17A) and decreased ability to bind to a monolayer of endothelial cells (FIG. 17B), respectively. Notably, treatment of mice with 4.5 mg/kg ADP13 initiated at the time of SUM149 cell implantation into the MFP led to significant inhibition of tumor growth (TGI=40%, P=0.0004) (FIG. 18). Treatment of pre-existing SUM149 cell-derived TNBC tumors also significantly reduced growth, i.e., treatment with 5 mg/kg ADP13 results in TGI of 30% (P=0.0307) (FIG. 19). Thus, ADP treatment reduces growth of a second ADAM8-positive TNBC cell line, although, not-surprisingly to a reduced degree given its highly aggressive IBC phenotype.

### Example 22. Phase 2: Lack of Cross-Reactivity to Closely Related ADAM33 Protein

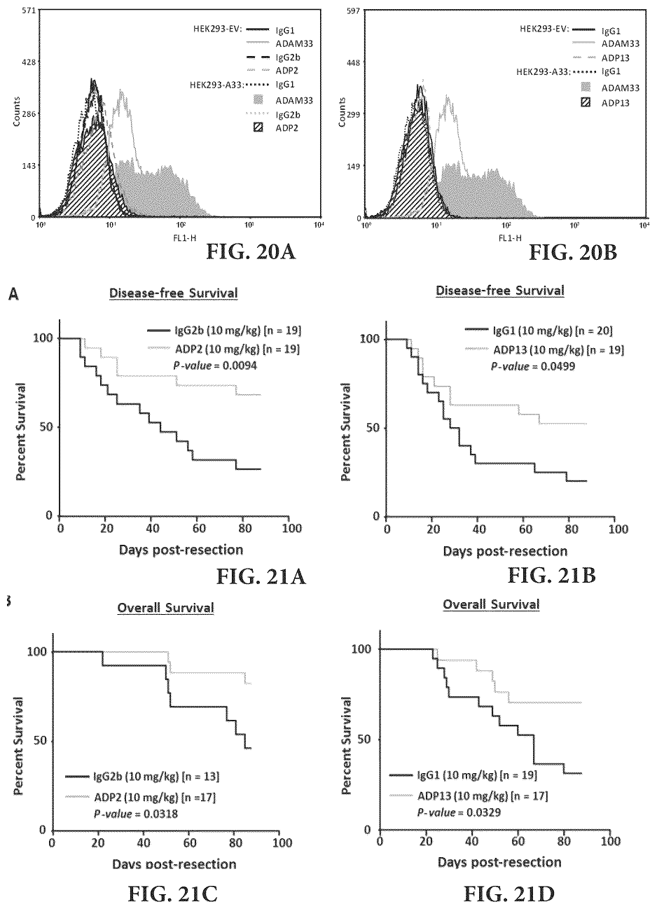

The specificity of the ADP2 and ADP13 antibodies for ADAM8 was further tested with analysis of their binding to ADAM33, another closely related ADAM protein (Takeda, Toxins, 8 (5). pii: E155, 2016). Both ADP2 and ADP13 failed to bind to HEK293 cells that were transfected to express ADAM33, termed HEK293-A33 cells, thus further confirming the specificity of these antibodies for ADAM8 (FIGS. 20A-B).

These studies demonstrate that the anti-ADAM8 MP/DI dual antagonist ADP2 and ADP13 mAbs are highly specific and display an ability to inhibit the growth of tumors derived from aggressive TNBC cells. Thus, ADP2 and ADP13 were identified as lead antibodies.

### Example 23. Phase 3: Dual Antagonist ADP2 and ADP13 Antibodies Inhibit Metastases of Pre-Existing Orthotopic TNBC Tumors and Improve Survival in a Neoadjuvant Model

To test whether ADP antibodies could decrease metastasis and improve outcome, a neoadjuvant protocol was performed, as is currently done in patients to better assess tumor response to therapy. MDA-MB-231-luc cells, which preferentially metastasize to the bone, were used. Once tumors reached 50-75 mm³, mice were treated with ADP2, ADP13 or their control IgGs, as described above. Tumors (~200 mm³) were then surgically removed and the mAb treatment continued for 12 weeks. The resulting Kaplan-Meier (KM) curves indicate ADP2 and ADP13 increase disease-free and overall survival of mice when the primary tumor has been surgically removed (FIGS. 21A-D).

Metastases are responsible for 90% of all breast cancer deaths (World Health Organization, WHO). Thus, mice from the above survival experiment were assessed for metastases to the bone. Imaging of dissected organs was performed when mice were sacrificed either due to the presence of a large recurrent tumor or at the end of the experiment. Treatment with either ADP2 or ADP13 resulted in a robust reduction in bone metastasis with respect to both their frequency and size (FIGS. 22A-D).

Thus, these studies indicated anti-ADAM8 antagonist ADP2 and ADP13 mAbs inhibited the dissemination of tumors derived from aggressive TNBC cells and improved outcome for the tumor-bearing mice.

### Example 24. Phase 3: Pharmacokinetic Profiles of ADP2 and ADP13 in NOD/SCID Mice Indicate Administration of a Bolus Initial Dose Leads to a More Constant Steady-State Antibody Level

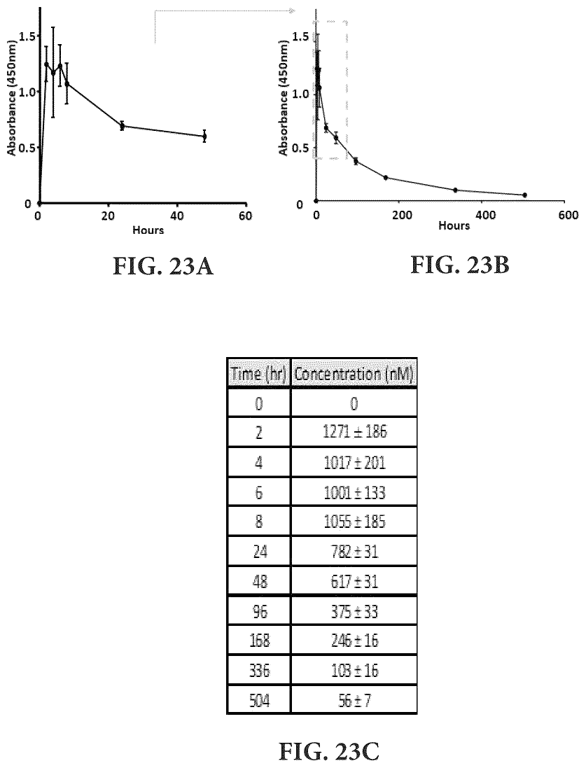

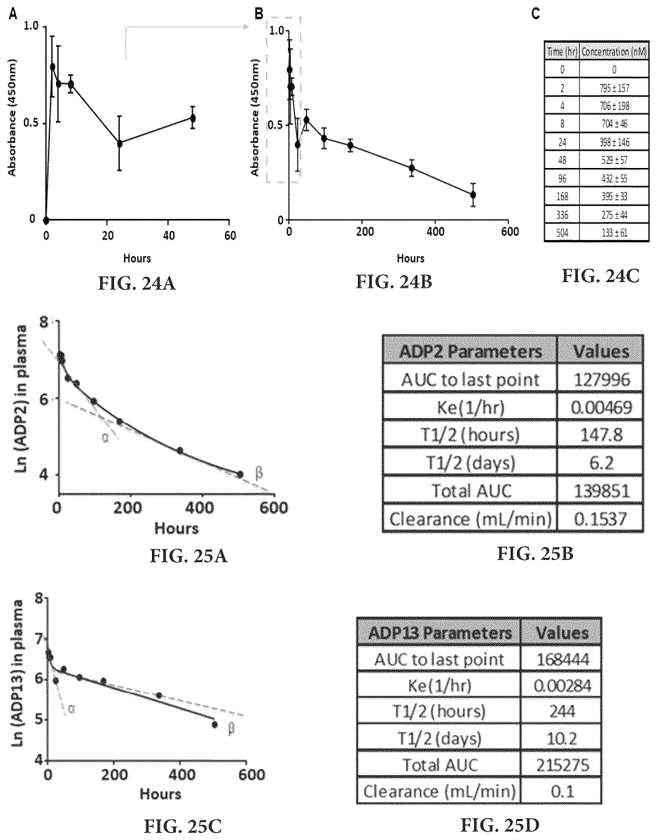

Pharmacokinetic (PK) analysis was performed to elucidate the levels of ADP2 or ADP13 in the blood over time in antibody treated NOD/SCID mice. Following a single i.p. injection with either ADP2 (FIGS. 23A-C) or ADP13 (FIGS. 24A-C), plasma concentrations of antibody were determined over a 21-day time course (n=3 mice per time point) using an ELISA assay that detects anti-ADAM8 mouse antibodies.

Based on the PK curve for ADP2, the maximum plasma concentration of the antibody over the dosing (Cmax) was ~1270 nM at 2 hours (FIG. 23C). The concentration of ADP2 was stable between 4 and 8 hours, but started to decrease thereafter, such that, the antibody was fully eliminated by 21 days. The plasma concentration curve was biphasic (FIG. 25A). Thus, a conservative “non-compartmental” approach was used to calculate the PK parameters: the area under curve (AUC) was 127996 nM·hr/mL and the clearance rate was 0.1537 mL/min (FIG. 25B). Thus, the decay pattern of ADP2 was bi-exponential and nonlinear.

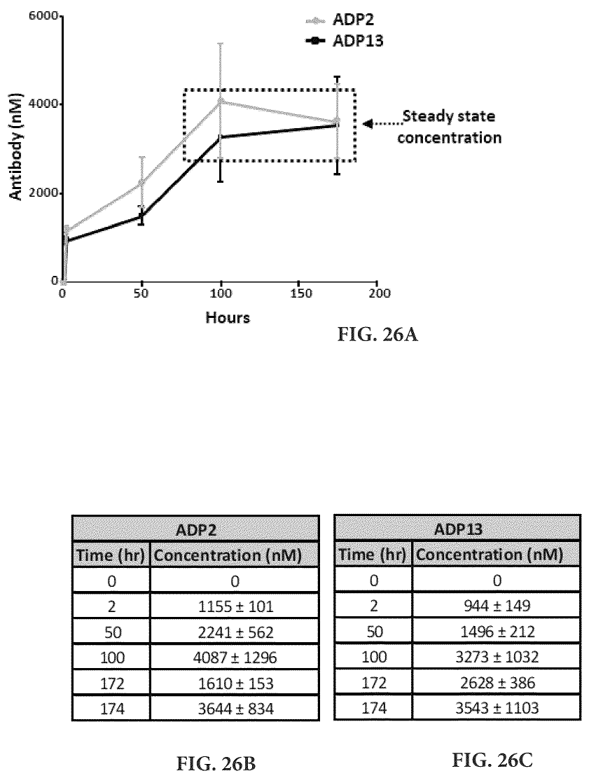

This pattern fits a two-compartment model composed of a distribution phase [alpha (α)-phase] followed by an elimination phase [beta(β)-phase]. The elimination half-life (T½) calculated using the formula (Ln2/Ke) is ~6.2 days. Similar findings were made for ADP13. The Cₘₐₓ (~795 nM) was reached at 2 hours (FIG. 24C). The concentration of ADP13 was stable for 8 hours, followed by a rapid decrease to ~398 nM after 24 hours. Thus, it appeared that once ADP13 equilibrated between the different body compartments and the plasma after 2 days (~529 nM), a linear decline was observed until 21 days. For ADP13, the AUC was 168444 nM·hr/mL, the clearance rate was 0.1 mL/min and a T½ of ~10.2 days was calculated (FIGS. 25C and 25D). These PK profiles were used to determine a modified treatment regimen that would achieve a more constant steady-state ADP2 or ADP13 antibody concentration in the mice: a loading bolus dose of 20 mg/kg followed by 3×/week maintenance doses of 10 mg/kg. The effectiveness of this new regimen was confirmed in a second series of mouse studies (FIGS. 26A-C), and thus has been used in subsequent preclinical testing.

### Example 25. Phase 3: ADP-Mediated Inhibition of ADAM8 Enhances NPAC-Mediated TNBC Tumor Growth Inhibition in a Tumor Regrowth Model

Recurrence and metastatic dissemination occur frequently in TNBC patients treated with the current chemotherapy options (Dent et al., Clin Cancer Res, 13 (15): 4429-4434, 2007; Pogoda et al., Med Oncol, 30:388-395, 2013). Here the hypothesis that addition of ADP2 or ADP13 to a current Standard-of-Care (SoC) chemotherapy for TNBC will either enhance tumor killing, tumor growth inhibition or inhibit life-threatening metastases, thereby improving outcome was tested. SoC chemotherapies for metastatic TNBC include taxols, which are mitotic inhibitors that lead to tumor killing. NPAC was selected as it is more stable and more effectively taken up by the cancer cell than unmodified paclitaxel (PAC) and has demonstrated greater efficacy in clinical trials compared to paclitaxel or docetaxel (Gradishar et al., J Clin Oncol, 23 (31): 7794-7803, 2005; Gradishar et al., J Clin Oncol, 27 (22): 3611-3619, 2009).

NOD/SCID mice bearing well-established, rapidly growing MDA-MB-231-luc tumors were divided into 4 treatment groups: a) isotype-matched control IgG+Saline, b)

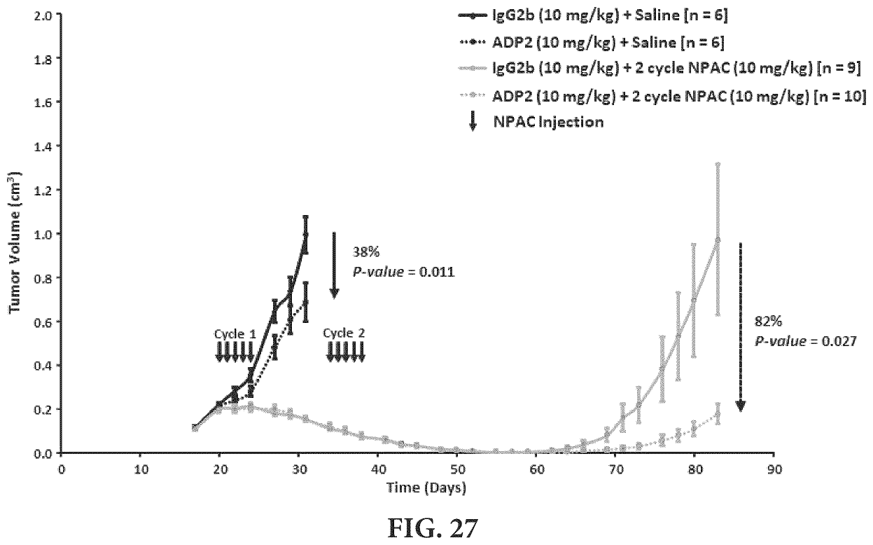

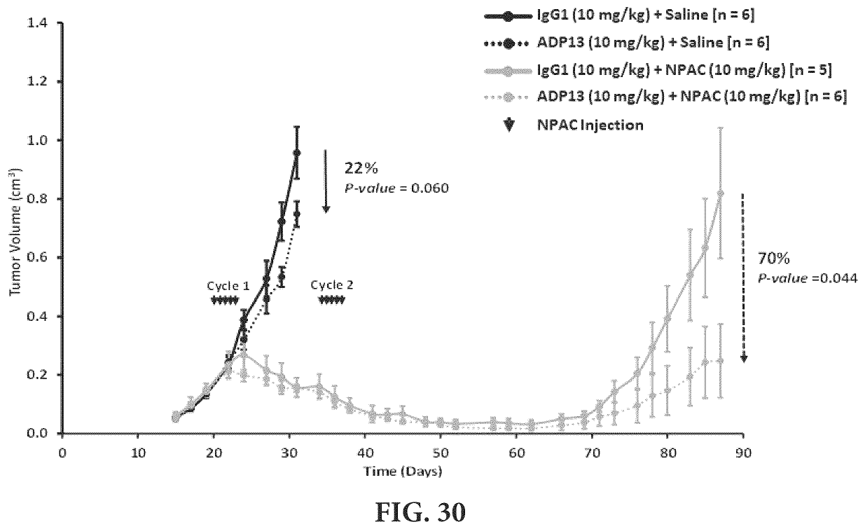

ADP2 or ADP13+Saline, c) isotype-matched control IgG+NPAC and d) Combinatorial regimen of ADP2 or ADP13+NPAC. To treat with NPAC, mice were given one cycle of 5 consecutive i.v. treatments of 10 mg/kg NPAC (dissolved in saline), rested for one week and then given a second cycle (FIG. 27 and FIG. 30); alternatively, an equivalent volume of vehicle saline was administered. ADP or control IgGs were given starting with a loading dose of 20 mg/kg initiated concurrently with the first chemotherapy cycle, followed by maintenance doses of 10 mg/kg given 3×/week throughout the time course. The endpoint for evaluation of Tumor Growth Inhibition (TGI, percent reduction in mean tumor volume) was an average tumor volume approaching 1 cm³ in the appropriate control group (IgG+NPAC vs ADP+NPAC and IgG+Saline vs ADP+Saline). NPAC treatment led to dramatic disease regression such that tumors were barely palpable by Day 55-62 in both IgG+NPAC and ADP+NPAC groups (FIG. 27 and FIG. 30). However, over time tumors regrew. Strikingly, tumor regrowth in the combinatorial groups with ADP2 or ADP13+NPAC was profoundly inhibited compared to animals treated with IgG+NPAC [ADP2: TGI=82%, P-value=0.027; ADP13: TGI=70%, P-value=0.044).

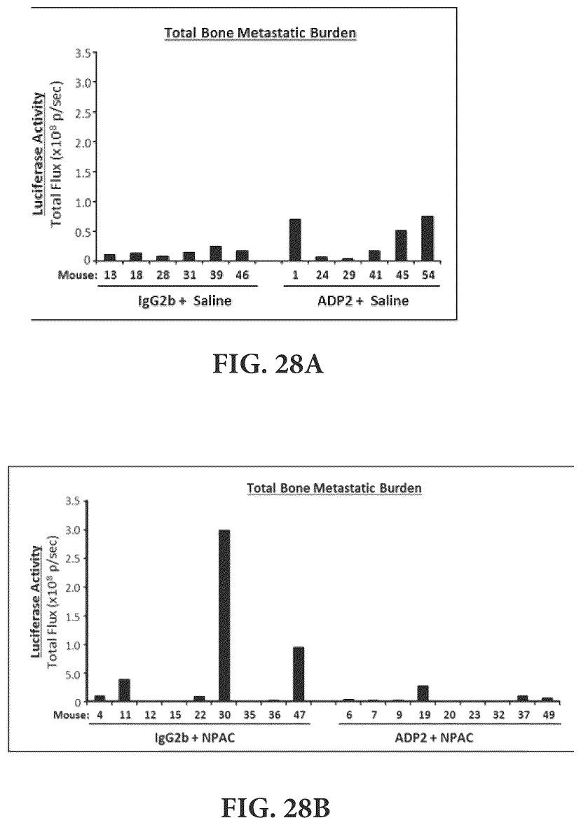

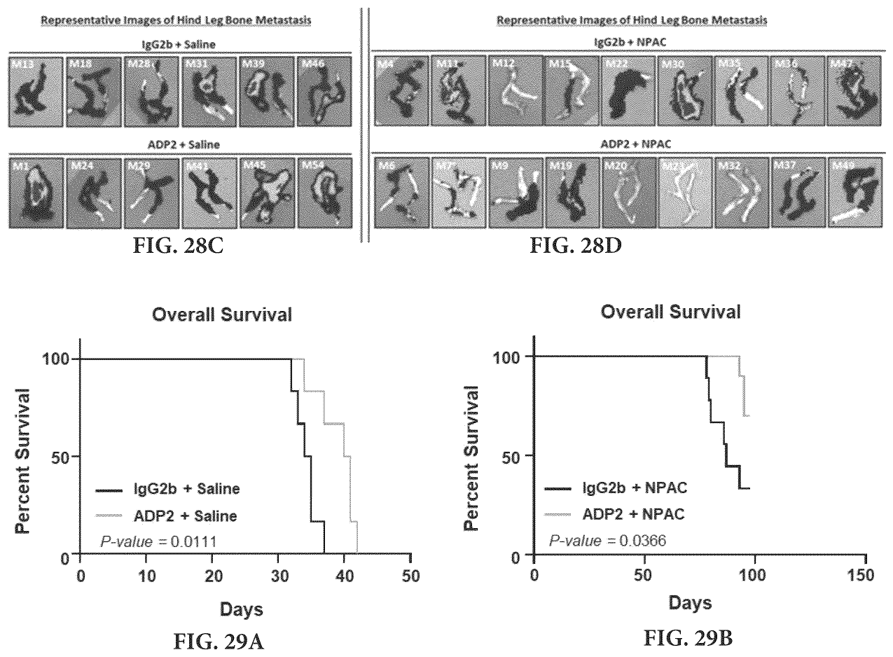

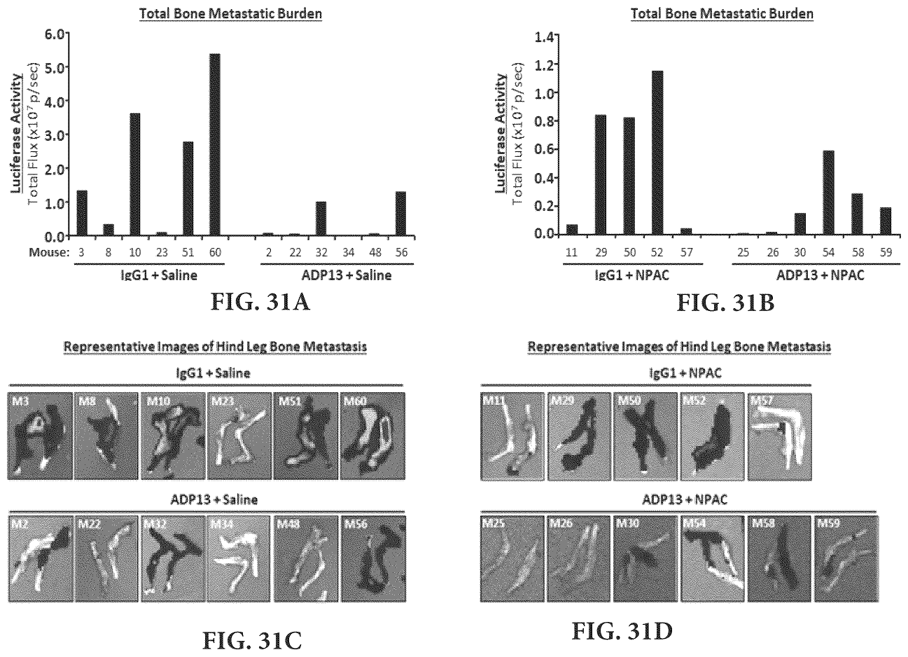

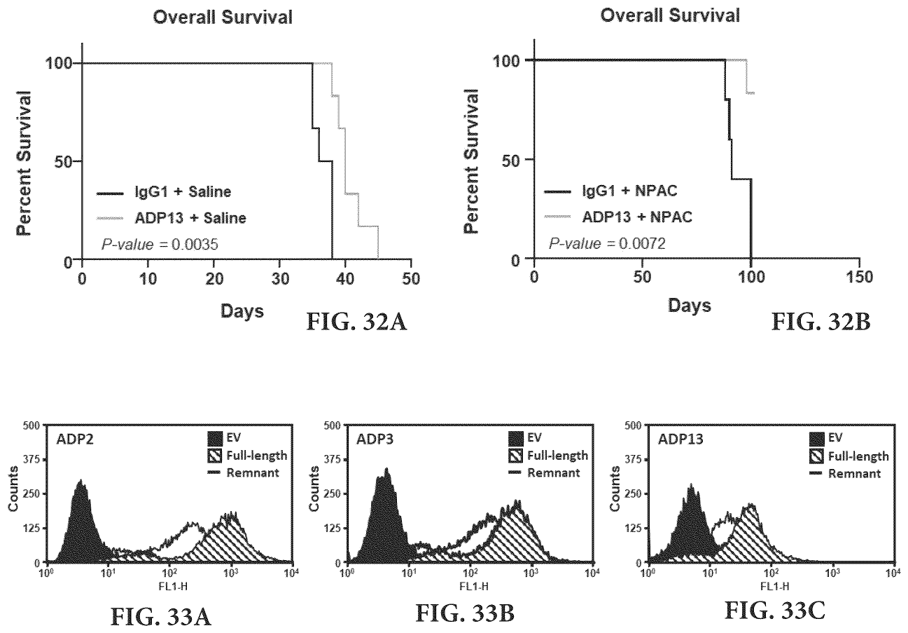

Furthermore, both the frequency and extent of metastases to the bone was decreased with the combinatorial regimen (FIGS. 28A-D and FIGS. 31A-D). Consistently, a significant survival benefit was seen with ADP2 or ADP13+NPAC vs IgG2b or IgG1+NPAC (FIGS. 29A-B and FIGS. 32A-B). As monotherapies, ADP2 (TGI=38%, P-value=0.011) and ADP13 (TGI=22%, P-value=0.06) led to a significant and modest benefit in primary TGI, respectively, under these more advanced disease conditions (larger primary tumor size at start of treatment). Thus, the combinatorial regimen of ADP2 or ADP13 and NPAC worked synergistically to reduce tumor regrowth and dissemination, and to improve survival vs NPAC or ADP treatment alone.

These studies demonstrated that the anti-ADAM8 MP/DI dual antagonist ADP2 and ADP13 mAbs were highly active against TNBC tumors both as monotherapies and in combination with SoC chemotherapy.

### Example 26. The CDR Sequence of ADP H and L Chain V-Regions

The Heavy (H) and Light (L) chain Variable (V) regions of ADP1, ADP2, ADP3, ADP4, ADP12, ADP13, ADP17 and ADP19 were subcloned and sequenced. The three complementarity-determining regions (CDRs) of the light and heavy chains and the germline genes were identified. With the exception of CDR L2 and CDR H1 of ADP2 and ADP13, which bear some similarity to each other, the other CDRs of the light and heavy chains of ADP2, ADP3 and ADP13 were quite different, indicating these were distinct antibodies. Notably CDR H3 of ADP13 was longer than its counterparts in ADP2 or ADP3. Moreover, the similarities in H and L chain CDRs amongst the 8 sequenced ADPs are consistent with their epitope binning (FIG. 7).

### Example 27. Epitope Mapping ADP-ADAM8 Interactions Using FACS Analysis and Hydrogen Deuterium Exchange (HDX) Mass Spectrometry

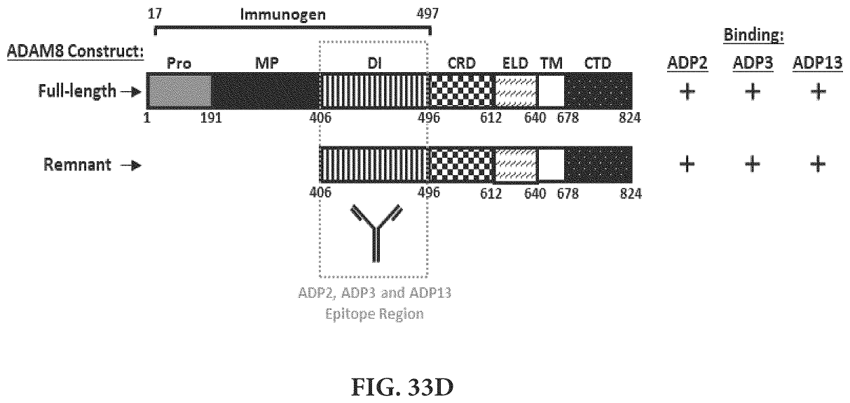

To begin to map the epitope of ADAM8 that interacts with ADP antibodies to inhibit tumor formation in vivo, ADP2, ADP13 and ADP3 were selected for analysis. FACS was performed using HEK293 cells expressing either full-length ADAM8, which includes both the MP and DI domains, or the remnant form, which lacks the MP domain but still contains the DI domain (FIGS. 1A-B and FIG. 33A-D). The ability of ADP2, ADP3, and ADP13 to bind to both the expressed full-length and remnant forms of ADAM8 suggested that the epitopes for these antibodies were contained within the DI domain.

HDX mass spectrometry of recombinant human ADAM8 was next used to identify the ADP2, ADP3, and ADP13 epitopes at the peptide level. For this analysis, ADAM8 was incubated with deuterium oxide for 60 s, 600 s, or 3600 s in either the absence or presence of ADP2, ADP3 or ADP13 and then subjected to pepsin/protease XIII digestion. The effects of antibody binding on the amount of deuterium in the resulting peptides was determined using liquid chromatography-mass spectrometry (LC-MS) (FIG. 34). ADAM8 showed specific reduction in deuterium uptake at the following sequences within the DI domain going from the amino to carboxy termini:

ADP3:
(SEQ ID NO: 88)

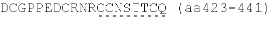

ADP2:
(SEQ ID NO: 86)

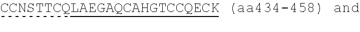

ADP13:
(SEQ ID NO: 87)
LAEGAQCAHGTCCQECKVKPAGELCRPKKDMCDLEEFCDGRHPECPEDAF
(aa442-491).

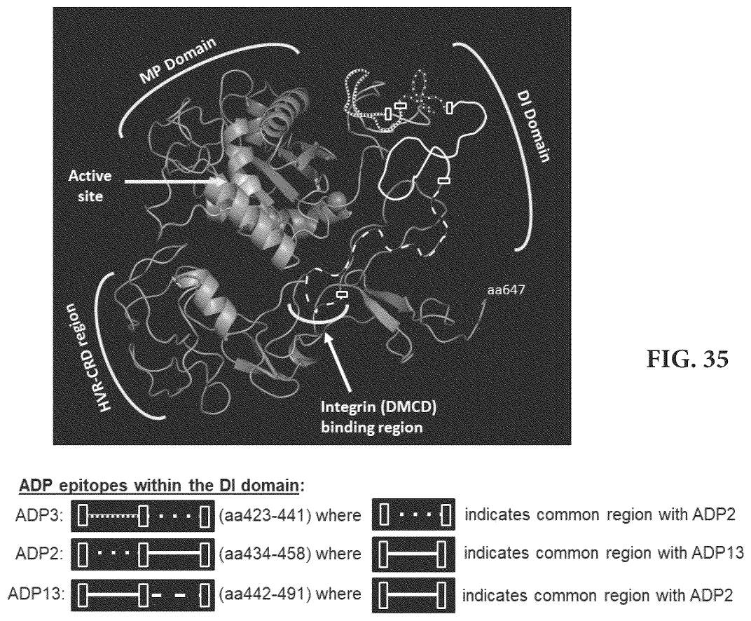

The three antibodies bound to sequences, which were partially overlapping (FIG. 34 and FIG. 35). The overlapping regions between (i) ADP3 and ADP2 and (ii) between ADP2 and ADP13 are indicated above by a dashed line, and a solid line, respectively. In their entirety the sequences bound by these three antibodies define SEQ ID NO: 1.

These findings were consistent with the binning described in FIG. 6, which showed no significant epitope overlap between ADP3 and ADP13, whereas the epitopes for (i) ADP3 and ADP2 and (ii) ADP2 and ADP13 substantially overlapped (~90%). Together these results indicate that ADP13, ADP2 and ADP3, define a family of ADPs, with dual MP/DI inhibitory activity that bind to SEQ ID NO: 1 within the DI domain and display significant in vivo functionality.

Competition binning analysis indicated that, within the panel of antibodies, this family includes Epitope 2 antibody ADP13, and Epitope 3 antibodies ADP2, ADP3, ADP1, ADP12, ADP4, ADP6, ADP7 and ADP9 (FIG. 7). The KD for ADP2 was 3.34E-09. The KD for ADP13 was 1.3E-09. The KD for ADP3 was 1.83E-08.

These findings showed that binding to the DI region inhibited the activities of both the MP and DI domains required for tumor growth and metastasis. ADP binding to the DI region likely disrupted the ability of ADAM8 to interact with integrins and other ECM components via multiple mechanisms.

For ADP13, whose site overlaps with the integrin binding site (DMCD) and the consensus Ca²⁺ binding site III (aa473-489 (CDLEEFCDGRHPECPED (SEQ ID NO: 103)), inhibition of DI activity may result via direct competition with integrins and other ECM components for interaction with ADAM8 (FIG. 34). For antibodies within the Epitope 3 group, additional mechanisms may be involved. These mechanisms include: 1) altering/destroying the tight structural constraints of the C-shaped DI/CRD structure; 2) reducing structural stability by preventing binding of the required Ca²⁺ at sites II and III; 3) blocking access to the integrin binding motif.

Notably, binding to the DI region has profound inhibitory effects on MP activity. This inhibition may also occur via several potential mechanisms. ADP binding at the DI domain may: 1) prevent substrate access to the MP domain or the HVR of the CRD domain; 2) change the orientation of the MP domain relative to the DI/CDR regions by disturbing the sequence linking the MP and DI domains; 3) change the configuration of the MP domain active site; 4) reduce MP function by preventing binding of the required Ca²⁺ at site I; 5) disrupt the tight structural constraints of the C-shaped DI/CRD structure, which would impact the HVR loops at the end of the CRD domain that are critical for substrate recognition by the ADAM8 metalloproteinase activity.

### Example 28. Mouse V-Region/Human IgG1 Constant (C)-Region Chimeras are Functional

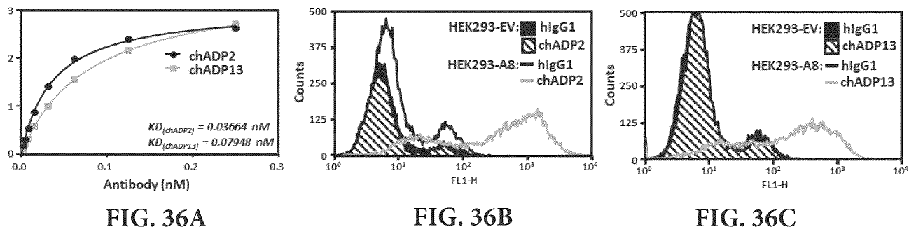

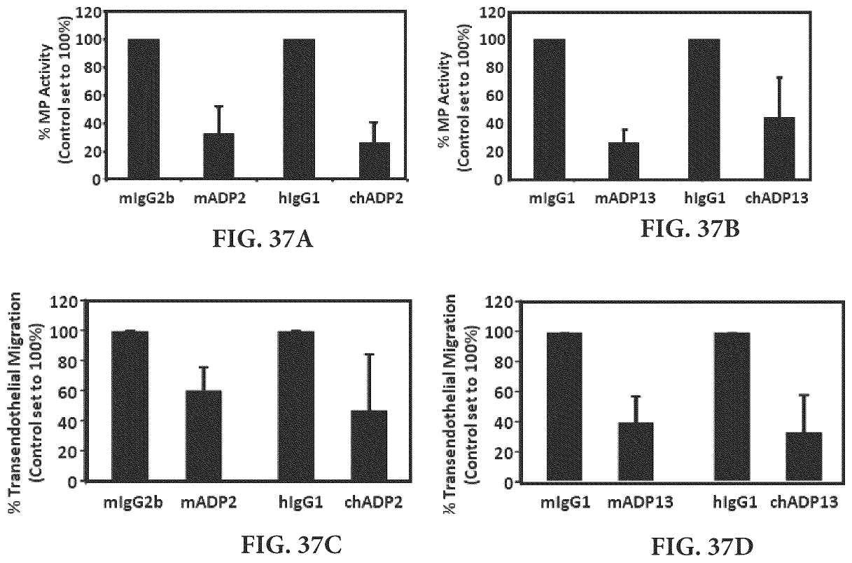

Vectors expressing chimeric ADP2 and ADP13 proteins with their V_{L} and V_{H} regions linked to the C region of human IgG1, a commonly used Fc region in therapeutic antibodies, were prepared. The chimeric proteins have been termed chADP2-IgG1 and chADP13-IgG1. A mutation had to be introduced into ADP13 V_{L} chain to avoid the creation of a new site of glycosylation. This change made the V_{L} more germline. Chimeric proteins were synthesized in CHO cells, purified and characterized for binding to ADAM8 and for dual MP and DI domain antagonist activity. The ability of chADP2-IgG1 and chADP13-IgGI to bind to ADAM8 was confirmed using both ELISA assays with recombinant protein (FIG. 36A) and FACS analysis of HEK293 cells stably expressing full-length ADAM8 vs control empty vector (EV) DNA (FIGS. 36B-C). Functionally, in cell-based assays, the two chimeras inhibited the MP and DI activities equal to or better than mouse ADP2 and ADP13 (FIGS. 37A-D).

### Example 29. Epitope Mapping Interactions of ADP2 and ADP13 with ADAM8 Via Shotgun Mutagenesis

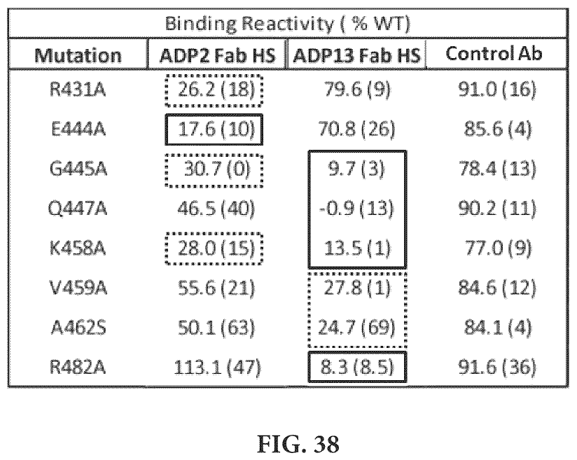

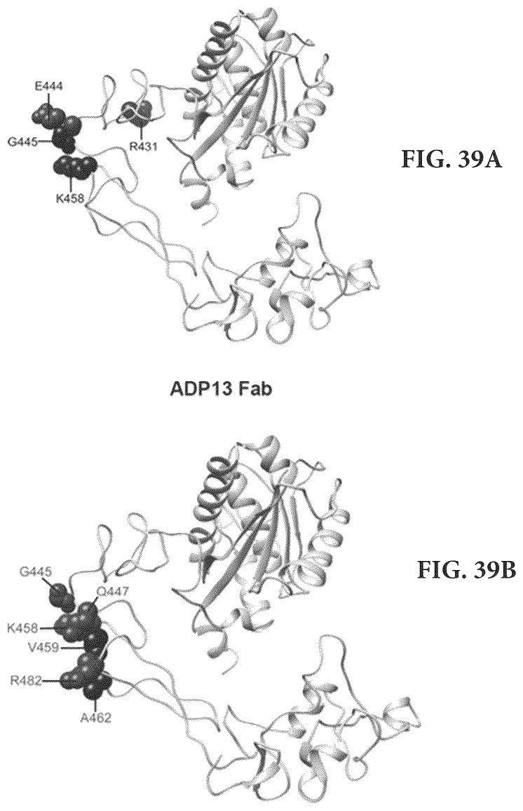

Shotgun mutagenesis paired with high-throughput flow cytometry was next used to identify specific amino acid residues that mediate the interaction of ADP2 and ADP13 with ADAM8. Both the MP and DI domains were assessed to confirm the data obtained by HDX mass spectrometry. For this analysis, high-throughput, site-directed mutagenesis was used to generate a library of human ADAM8 expression constructs with single alanine mutations introduced into each amino acid residue between 192 to 497 (covering the MP and DI domains) except when alanine was the original amino acid—in which case it was mutated to serine. Following transfection into HEK293 cells and subcloning, the library was screened by high-throughput flow cytometry under high stringency (HS) conditions using antigen-binding fragments (Fabs) of ADP2 and ADP13, or a positive control ADAM8 antibody (Control Ab). One amino acid in ADP2 (E444) and four amino acids (G445, Q447, K458, and R482) in ADP13 reached the threshold guidelines for critical binding residues (i.e., control antibody binding >70% of WT and test antibody binding <20% of WT binding) (FIG. 38). Mutations in these amino acid positions resulted in a significant reduction of ADP2 and ADP13 Fab binding, respectively but no reduction of control antibody binding. Three amino acids in ADP2 (R431, G445, and K458) and two amino acids in ADP13 (V459 and A462) are residues of secondary importance, i.e., that did not reach the <20% of WT binding criterion for critical residues, but still led to a substantial reduction in ADP2 and ADP13 Fab binding activity (FIG. 38). Mutations in these amino acid positions resulted in a significant reduction of ADP2 and ADP13 Fab binding, but no reduction of control antibody binding. This change in binding in combination with their proximity to critical residues indicates that they are part of the antibody epitope. The positions of the ADP2 and ADP13 critical and secondary binding residues are indicated in an ADAM8 crystal structure model that is based on the structure of vascular apoptosis-inducing protein-1 (PDB ID #2ERP, Takeda et al. EMBO J. 25, 2388-2396, 2006) (FIG. 39A and FIG. 39B, respectively).

Overall, there is tremendous concordance between the amino acid residues identified by shotgun mutagenesis and HDX mass spectrometry, i.e., all of the critical amino acid residues mapped by mutagenesis fall within the peptide regions of ADP2 and ADP13 mapped by HDX mass spectrometry, and only the ADAM8 R431 amino acid of secondary importance for ADP2 binding maps three amino acids upstream, thus extending the region mediating binding to ADP2 to RNRCCNSTTCQLAEGAQCAHGTCCQECK (aa431-458) (SEQ ID NO: 104).

These findings demonstrate that antibodies binding within SEQ ID NO: 1 of the DI domain function to inhibit both the MP and DI activities of ADAM8.

### Example 30. Overview of Approach Used to Identify ADP2 as a Lead Diagnostic Antibody for IHC-Based Detection of ADAM8 and to Generate a Breast Control Cell Line Microarray (CCM) Scoring System

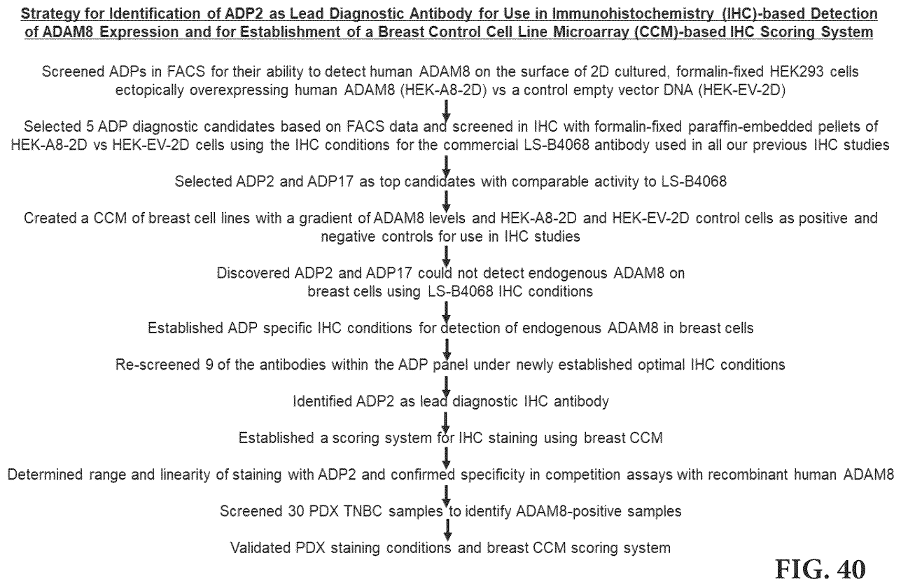

To identify diagnostic antibodies for use in IHC-based detection of ADAM8-positive tumors, the multistage strategy outlined in FIG. 40 was performed. Briefly, the ADP panel of antibodies was first screened using FACS analysis of fixed cells with ectopic (i.e., exogenous) ADAM8 expression. ADPs with good binding activity under fixed conditions were then tested in IHC with formalin-fixed paraffin-embedded (FFPE) pellets of cells with both exogenous ADAM8 expression (HEK293-ADAM8 cells) and endogenous ADAM8 expression (untransformed breast and breast cancer cells), and the staining conditions for the RUO ADAM8 LS-B4068 antibody, previously used in our studies of patient biopsies (Romagnoli et al., EMBO Mol Med, 6 (2): 278-294, 2014). The breast cancer studies led us to determine that the LS-B4068 conditions were not appropriate for use with the ADP panel of antibodies. ADP-specific IHC staining conditions were then identified. Using these newly optimized conditions, multiple ADPs were found capable of detecting ADAM8 in IHC. ADP2 was selected as lead diagnostic antibody and ADP17 as a backup. Furthermore, by comparing Western blotting for active ADAM8 protein levels with IHC staining of FFPE breast cell pellets, a control cell line microarray (CCM) with low, medium and high ADAM8 levels was established for an IHC 1+, 2+, 3+ scoring system. Finally, ADP2 IHC staining and the CCM scoring system were validated using TNBC patient-derived xenograft (PDX) samples.

### Example 31. Identification of ADP Candidate Diagnostic Antibodies Using FACS Analysis of Fixed HEK293-ADAM8 Cells

To determine whether the antibodies within the ADP panel were able to detect ADAM8 under fixed conditions, a critical requirement of all IHC antibodies, 12 antibodies were selected, based on high binding affinity (ELISA and Biacore) and production, for screening in FACS analysis of fixed 2D cultured HEK293-ADAM8 (HEK-A8-2D) cells. As controls, 2D grown HEK293-Empty Vector cells (HEK-EV-2D) that did not express ADAM8 and isotype-matched control IgGs were used. Mouse IgG2b (anti-Manduca sexta ecdysone clone 10F1, Developmental Studies Hybridoma Bank, U. Iowa City) was the isotype matched control IgG for ADP2 and ADP4, while mouse IgG1 (anti-BrdU clone G3G4, Developmental Studies Hybridoma Bank, U. Iowa City) was the isotype matched control IgG for ADP3, ADP6, ADP7, ADP9, ADP10, ADP11, ADP13, ADP17, ADP18 and ADP19. Additionally, unfixed samples of these cells were used to confirm the FACS activity of these antibodies under native conditions, thus establishing if activity under fixed conditions is retained, improved or lost.

Briefly, HEK293-ADAM8 and HEK293-EV cells were trypsinized and single cell suspensions generated by passing cells through a syringe with a 21-gauge 1.5-inch needle. For unfixed samples, three hundred thousand cells per sample were resuspended in 50 μL FACS Buffer (1% BSA, 0.1% sodium azide in PBS) and exposed to 2 μg primary ADP or IgG control for 30 min on ice. After incubation, cells were washed twice in FACS buffer and then exposed to 1.25 μg of secondary antibody Alexa Fluor 488 donkey anti-mouse IgG (H+L) (Life Technologies, A-21202) for 30 minutes on ice. Cells were finally washed 3 times with FACS buffer, resuspended in 400 μL of fresh buffer and analyzed by flow cytometry using a BD FACSCalibur. Analysis of fixed cells was performed as above except, after a single cell suspension of cells was generated, samples containing three hundred thousand cells were resuspended in 50 μL of 4% paraformaldehyde and incubated on ice for 20 min before proceeding to primary antibody staining.

### Example 32. Generation of Breast Specific Control Cell Line Microarray (CCM) and ADAM8 Scoring System

A CCM containing breast cell lines was created with a gradient of endogenous ADAM8 levels and the HEK293-ADAM8 and HEK293-EV cells, as positive and negative controls, respectively, for use in optimization of ADP IHC conditions and as an ADAM8 IHC scoring system for evaluation of tissue samples. Briefly, to select appropriate lines for the CCM, ADAM8 protein levels were assessed in untransformed MCF-10A breast epithelial cells, and TNBC SUM149, MDA-MB-231 and MDA-MB-231-LUC cells using Western blotting. Cultures were grown either under 2D or 3D conditions for 48 hours. Whole cell extracts (WCE) were prepared and subjected to Western blotting for ADAM8. Briefly, WCE were prepared using Radioimmunoprecipitation assay buffer (RIPA, 50 mM Tris pH 7.6, 150 mM NaCl, 1% NP40, 0.1% SDS, 5 mM EDTA, 1% Sodium Sarkosyl) supplemented with Halt Protease and Phosphatase Inhibitor Single-Use Cocktail (1:100, Thermo Fisher Scientific, 78442), 0.5 M EDTA (1:100) and 1 M 1,10-Phenanthroline (1:100, Sigma, 131377) to inhibit the autocatalytic activity of ADAM8. Samples (30 μg) were subjected to Western blotting for ADAM8 with the LS-B4068 anti-ADAM8 antibody (LifeSpan BioSciences). Sample blotting with a β-actin antibody (Sigma) was used as a control for loading. A Precision Plus Protein Dual Color Standard (Biorad, #1610374) was used as a protein size marker.

Based on protein expression data from this Western blotting analysis, MCF10A-2D, MDA-MB-231-2D, MDA-MB-231-3D, HEK293-Empty Vector-2D and HEK293-ADAM8-2D were selected as appropriate cells with a broad range of ADAM8 levels. Next, the CCM was established. Briefly, for 2D cultured cells, confluent 100-mm plates were dissociated using Accutase (Gibco/StemPro A1110501) and subcultured at dilutions of 1:3 (MCF10A-2D), 1:2 (MDA-MB-231-2D) and 1:5 (HEK293-ADAM8-2D and HEK293-EV-2D) onto tissue culture treated 100-mm plates and grown to confluency (48-72 hours). For 3D culture, confluent 100-mm plates of 2D grown MDA-MB-231 cells were dissociated with Accutase and the entire single cell suspension per plate transferred to a 100-mm low attachment plate (Corning, 3262) for 48 hours to induce ADAM8 levels. Cultures of 30×10⁶-50×10⁶ cells for each of these five lines were then collected by dissociation with Accutase and/or centrifugation and washed with 1×PBS. Cells were then resuspended in formalin, pelleted for 5 minutes at 1,000 rpm and allowed to fix overnight at 4° C. Pellets were washed once with 70% ethanol, without disrupting the pellet, centrifuged at 1,000 rpm for 5 minutes, to ensure a compact pellet is formed, and then paraffin-embedded together in a single block to create the microarray. Finally, sections (4-5 μM) were cut from this block onto slides for IHC analysis.

For quantification of the levels of active ADAM8 in the CCM breast cell lines, Western blot analyses of protein extracts were performed with LS-B4068 as above, except extracts from each of the five cell lines were loaded at various concentrations (2-120 μg) to ensure that blots displayed bands with levels within the linear range. Gels were quantified using densitometry and compared to staining from ADP2 IHC analyses of the CCM slides. Experiments were repeated with a second anti-ADAM8 antibody (LS-C20181, LifeSpan BioSciences) to confirm results. MCF10A-2D, MDA-MB-231-2D and MDA-MB-231-3D displayed a stepwise ~5-7-fold increase in relative active ADAM8 levels in Western blotting, and commensurately, a low, medium and high percent cell culture staining positivity in IHC and were thus defined as having a simple 1+, 2+ and 3+ADAM8 IHC staining score. This scoring system was used to evaluate IHC results of TNBC PDX samples. PDX staining scores were determined by direct visual comparison to the scores established for the breast lines within the CCM.

### Example 33. Method for IHC Analysis

The CCM, tissue microarrays (TMAs), and single section slides of formalin fixed paraffin embedded (FFPE) cell lines and TNBC patient derived xenograft (PDX) samples were analyzed by IHC in a Ventana BenchMark ULTRA automated clinical diagnostic slide stainer at the Tufts Medical Center Histopathology Laboratory, on a fee-for-service basis.

### Staining Conditions for LS-B4068 ADAM8 Antibody

For IHC analysis with LS-B4068 (LifeSpan BioSciences), following the standard automated deparaffinization protocol (medium temperature 72° C.), slides were subjected to Heat-Induced Epitope Retrieval (HIER) using the Ventana standard Cell Conditioning (CC) 1 Tris-EDTA-based basic buffer for 64 minutes. This incubation can disrupt the covalent bonds formed by formalin in tissue. Removing these bonds allows renaturation of protein molecules and increases antibody accessibility thereby improving binding and signal to noise ratio. Slides were then incubated with primary antibody LS-B4068, or its isotype matched control rabbit polyclonal IgG (Abcam, ab37415) at dilutions of 1:50 to 1:20,000 for 32 minutes. For detection, a Ventana iVIEW DAB kit (760-091) was used. This kit uses biotin-bound secondary antibodies and HRP-tagged streptavidin. The interaction of these reagents at the site of primary antibody binding promotes the conversion of hydrogen peroxide substrate and the 3,3′-diaminobenzidine tetrahydro-chloride (DAB) chromogen into a brown precipitate that can be easily observed using light microscopy. Immunostained slides were counterstained with hematoxylin for visualization of cell nuclei. ADP antibodies were initially used in IHC with these conditions before optimal conditions for their use were identified.

### Staining Conditions for ADP Antibodies

As ADPs were found to detect only exogenously expressed ADAM8 in HEK293 cells and not endogenous protein in breast cells under LS-B4068 IHC staining conditions, a variety of parameters were modified to identify ideal conditions for use of the ADP antibodies in IHC. First, the antigen retrieval method was modified to HIER with CC2 citrate-based acidic buffer instead of the CC1 Tris-EDTA-based basic buffer. This resulted in even less staining and thus more focus was placed on the CC1 buffer and trying to optimize staining conditions with it. Next, the time of retrieval with CCI was varied from the standard 64 minutes of incubation to a shorter (20 minute) and a longer (98 minute) period. This led to the discovery that shorter incubation is better and the realization that HIER conditioning even with CC1 in fact inhibits ADP staining. Thus, IHC was performed without epitope retrieval. This improved staining but the relative extent of staining of HEK293-ADAM8-2D vs MDA-MB-231-2D and MDA-MB-231-3D cells was still much lower than expected based on Western blot analysis. To try to improve the accessibility of the ADAM8 protein to antibody staining, a Proteolytic-Induced Epitope Retrieval (PIER) method with Protease 2 (P2), an alkaline endopeptidase of the serine protease family, was used. A further improvement of ADAM8 staining was seen following 4 minutes of this PIER protocol, but the relative staining in the breast cancer vs HEK293-ADAM8-2D cells still appeared weaker than expected.

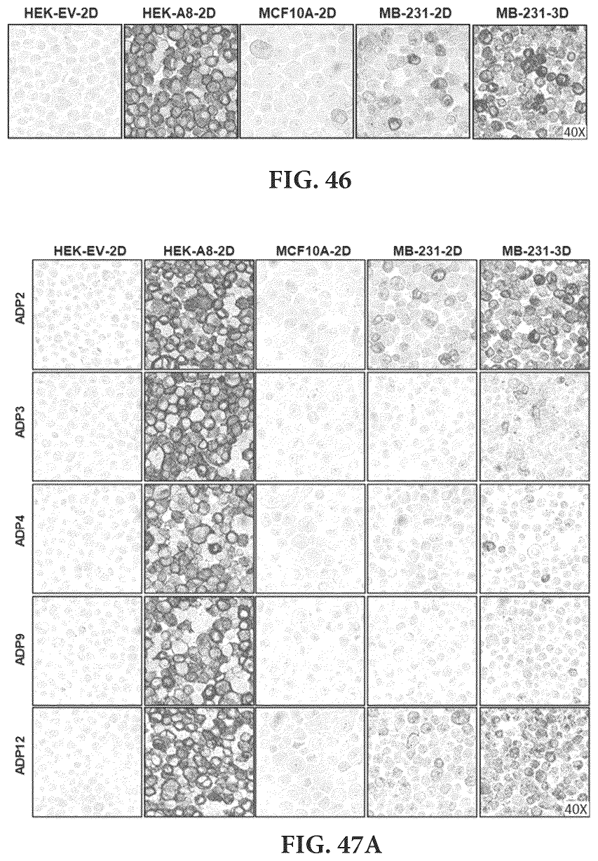

To enhance the level of staining, an amplification step was added using the Ventana Amplification Kit (760-080), which relies on anti-mouse IgG heavy and light chains and mouse anti-rabbit IgG heavy chains that bind to the primary antibody on the tissue, increasing the total number of antibodies bound at the site of antigen to amplify the signal. A robust increase in staining was observed (FIG. 46), such that ADAM8 detection levels in the breast vs HEK293-ADAM8-2D cells appeared consistent with the Western blot analysis (FIG. 44). Thus, PIER method with P2 plus an amplification step was identified as the optimal protocol for use of ADP antibodies in IHC-based detection of ADAM8.

ADPs were tested at dilutions which ranged from 1:50 to 1:120,000. Isotype matched controls were mouse IgG1 (Abcam, ab18443) and IgG2b (ab18428 and ab18457, Abcam). To identify ADAM8-positive samples, two TMAs containing a total of 30 TNBC PDX samples were assessed using either LS-B4068 or ADP2, at a dilution of 1:100 and with the appropriate optimal staining conditions for each antibody. Three samples with substantial staining were identified: PDX 5998, PDX 3561, and PDX 4849.

To validate ADP2 staining in tissues and the CCM as a scoring system, two sets of single section slides for each of these 3 TNBC PDX tumors (4 consecutively cut slides/PDX/set) together with CCM slides were subjected on different days to IHC using ADP2 at dilutions of 1:50, 1:100 and 1:500 vs the isotype-matched control IgG2b at a 1:50 dilution. PDX samples were scored as described above.

### Example 34. Screening of ADP Panel by FACS for Identification of Diagnostic Candidates that Detect Fixed ADAM8

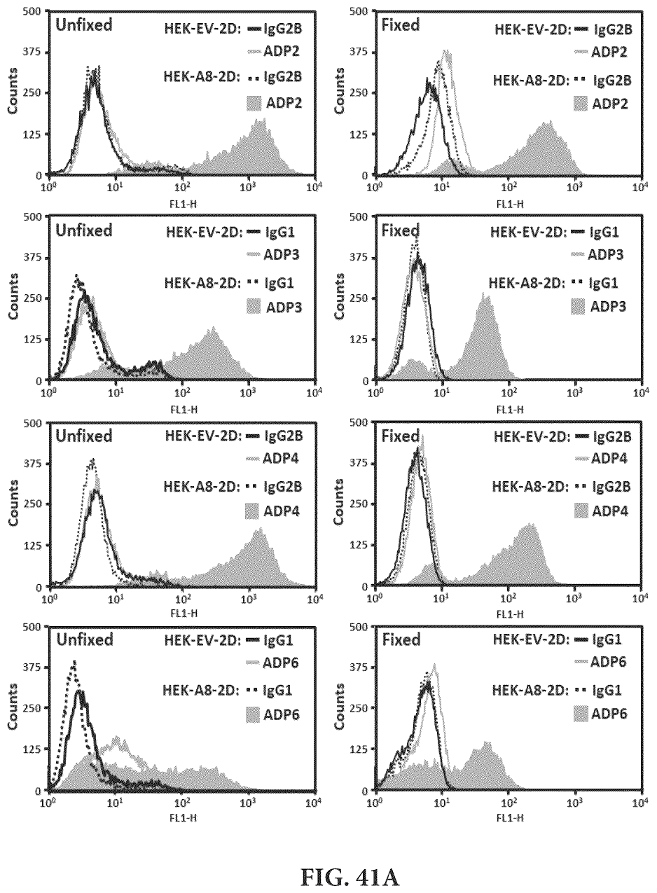

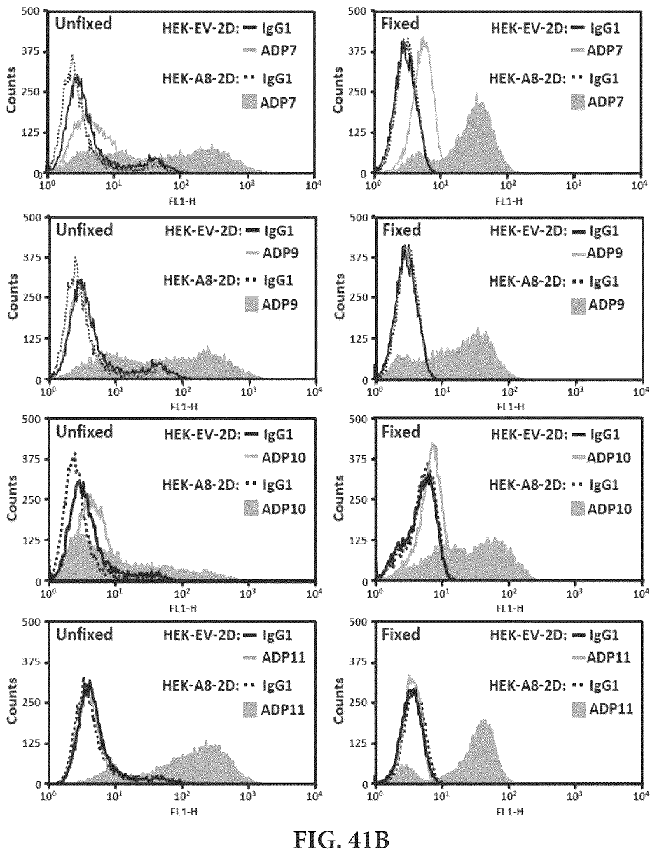

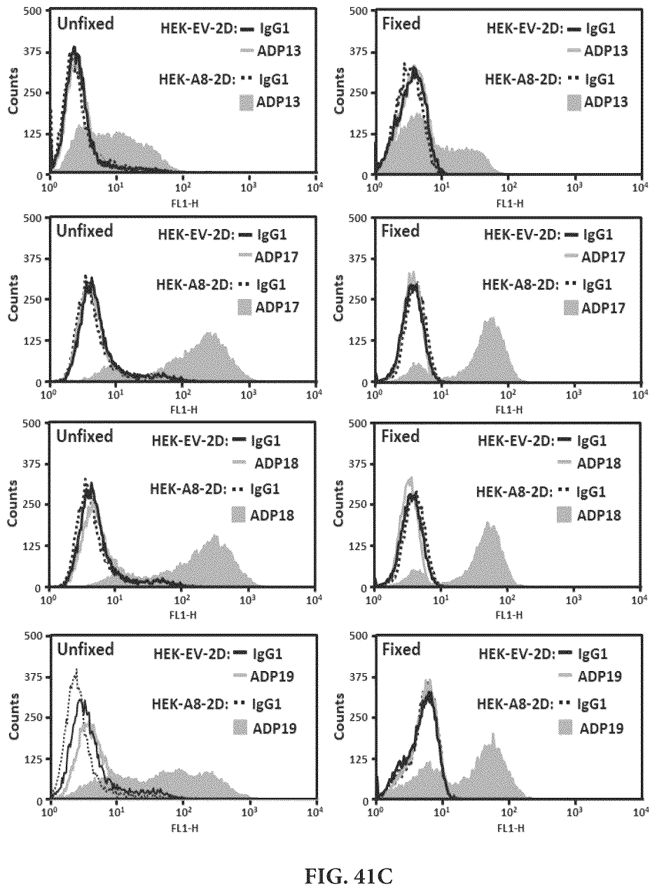

FACS was used first to screen the panel of ADP antibodies to test their ability to detect exogenously expressed ADAM8 on the surface of fixed HEK293-ADAM8 cells that had been grown as adherent cultures on tissue culture treated plates (2D) (FIGS. 41A-C). As controls, HEK293-Empty Vector cells that did not express ADAM8 and isotype-matched control IgGs were used. Additionally, unfixed samples of these cells were used to demonstrate the FACS activity of these antibodies under native conditions. HEK293-ADAM8 and HEK293-Empty Vector cells cultured on tissue culture plates (2D) are abbreviated as HEK-A8-2D and HEK-EV-2D, respectively in FIGS. 40-51. Five ADPs (ADP2, ADP3, ADP4, ADP13, and ADP17) were found to detect ADAM8 exceptionally well in FACS analysis of fixed HEK293-ADAM8 vs HEK293-Empty Vector cells and selected for further testing in IHC.

### Example 35. Testing of ADP Antibodies in IHC with Research Use Only (RUO) LS-B4068 Antibody Staining Conditions

Conditions optimized for the RUO LS-B4068 anti-ADAM8 IHC antibody (LifeSpan BioSciences), described in Example 33 (Romagnoli et al., EMBO Mol Med, 6 (2): 278-294, 2014), and slides cut from FFPE pellets of HEK293-Empty Vector-2D and HEK293-ADAM8-2D cells were used. IHC analysis was performed in a Ventana BenchMark ULTRA automated clinical diagnostic slide stainer. Following the standard automated deparaffinization protocol (medium temperature 72° C.), slides were subjected to Heat-Induced Epitope Retrieval (HIER) using the Ventana standard Cell Conditioning (CC) 1 Tris-EDTA-based basic buffer for 64 minutes.

Slides were then incubated with primary antibodies (ADPs, LS-B4068, or their appropriate control IgGs) at dilutions of 1:50 to 1:20,000 for 32 minutes. For detection, a Ventana iVIEW DAB kit (760-091) was used. This kit employed biotin-bound secondary antibodies and HRP-tagged streptavidin. The interaction of these reagents at the site of primary antibody binding promoted the conversion of hydrogen peroxide substrate and the 3,3′-diaminobenzidine tetrahydro-chloride (DAB) chromogen into a brown precipitate that could be easily observed using light microscopy. Immunostained slides were counterstained with hematoxylin.

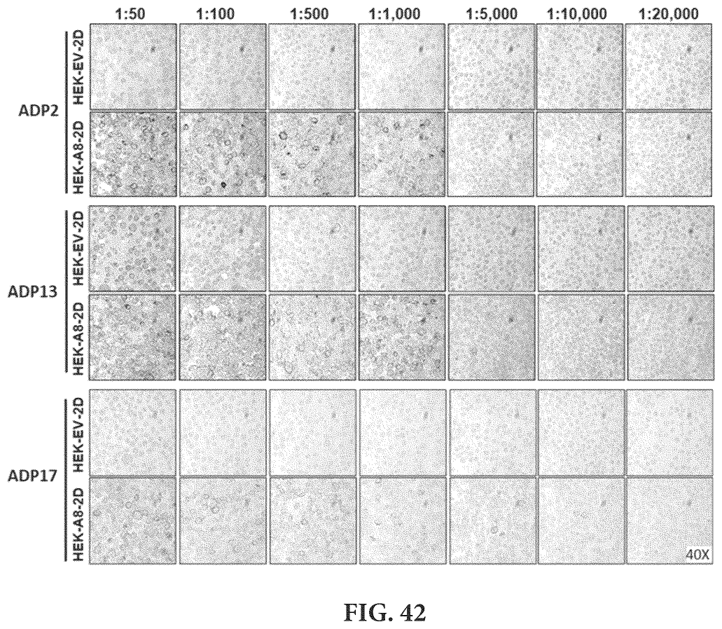

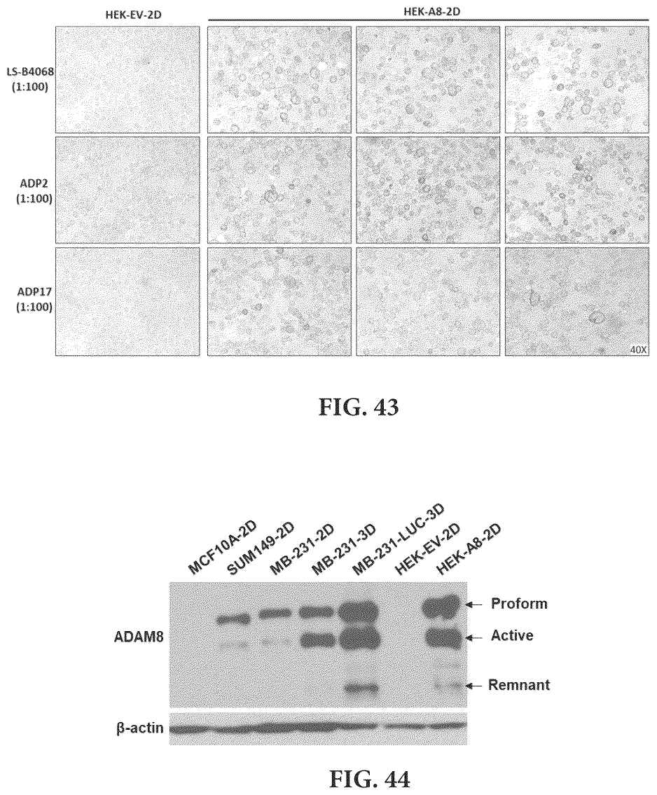

ADP2 and ADP17 demonstrated strong ADAM8 staining, which was dose-dependent in a range from 1:50 to 1:1,000 (FIG. 42). ADP13 also displayed dose-dependent HEK293-ADAM8-2D positive staining, but the background was substantially higher (FIG. 42); whereas, ADP3 and ADP4 had no activity in IHC with HEK293-ADAM8-2D cells (data not shown). No staining was observed with the ADAM8-negative HEK293-Empty Vector-2D cells. Importantly, analysis of ADP2 and ADP17 vs LS-B4068 revealed comparable staining of HEK293-ADAM8-2D cells (FIG. 43).

### Example 36. Selection of Cell Lines for Generation of Breast CCM

To extend IHC studies to analysis of endogenous ADAM8 levels, first a variety of cell lines were assessed for ADAM8 protein expression using Western blotting. Untransformed MCF-10A had very low ADAM8 levels compared to SUM149 or MDA-MB-231 TNBC cells grown in 2D (Romagnoli et al., EMBO Mol Med, 6 (2): 278-294, 2014). Moreover, growth of TNBC MDA-MB-231 cells in suspension culture (3D), e.g., in low attachment plates, resulted in a substantial induction in the overall level of ADAM8 and specifically the amount of active form (Romagnoli et al., EMBO Mol Med, 6 (2): 278-294, 2014). To compare the levels of ADAM8 in the various breast epithelial and cancer cell lines, Western blotting with the LS-B4068 anti-ADAM8 antibody was performed. Cultures were grown either under 2D or 3D conditions for 48 hours. Whole cell extracts were prepared (Romagnoli et al., EMBO Mol Med, 6 (2): 278-294, 2014) and subjected to Western blotting for ADAM8 (FIG. 44).

A gradient of ADAM8 expression was observed. MCF10A-2D breast epithelial cells express barely detectable ADAM8 levels, which were visible only when very long film exposures were performed (data not shown), whereas, SUM149-2D and MDA-MB-231-2D cells express moderate levels. Growth in 3D of MDA-MB-231 cells and of its more aggressive derivative MDA-MB-231-LUC induces extremely high endogenous ADAM8 levels, which are comparable to those seen in HEK293-ADAM8-2D cells, with exogenous ADAM8 expression (FIG. 44). Based on this protein expression data, MCF10A-2D, MDA-MB-231-2D, MDA-MB-231-3D, HEK293-Empty Vector-2D and HEK293-ADAM8-2D were selected to create a breast CCM with a gradient of low, medium and high ADAM8 levels for further ADP IHC optimization and assessment of their use in an early ADAM8 scoring system. Cells were grown in 2D or 3D conditions, fixed in formalin and paraffin-embedded in a single block to create a microarray containing these five samples. The block was sectioned onto slides which were then subjected to IHC.

### Example 37. IHC Conditions for the RUO LS-B4068 ADAM8 Antibody are Inappropriate for ADP Recognition of Endogenous ADAM8 in the CCM

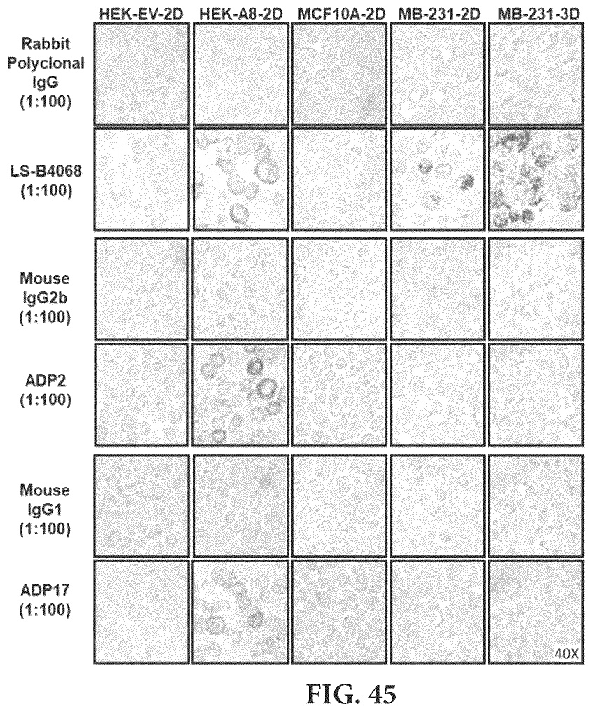

CCM slides were subjected to IHC analysis under the conditions optimized for the LS-B4068 antibody with LS-B4068, ADP2 and ADP17 vs their respective control isotype matched IgGs (FIG. 45).

ADP2 and ADP17 detected ADAM8 only in HEK293-ADAM8-2D but not in MDA-MB-231-2D or MDA-MB-231-3D cells (FIG. 45); whereas, LS-B4068 detected ADAM8 in all three. As expected, neither antibody displayed staining in control HEK293-Empty Vector-2D or in the MCF10A-2D cells. These findings indicated that the conditions identified for LS-B4068 are not optimal for IHC use of ADPs, which more likely recognize ADAM8 in its native configuration.

### Example 38. Established IHC Conditions for Detection of Endogeneous ADAM8 by ADP Antibodies

As ADPs were found to detect only exogenously expressed ADAM8 in HEK293 cells and not endogenous protein in breast cells under LS-B4068 IHC staining conditions, a variety of parameters were modified to identify ideal conditions for use of the ADP antibodies in IHC as detailed in Example 33. ADP2 was used as the prototype antibody for the ADP panel in these studies. Optimal conditions were established using Proteolytic-Induced Epitope

Retrieval (PIER) treatment with Protease 2 (P2), an alkaline endopeptidase of the serine protease family, followed by an amplification step using the Ventana Amplification Kit (760-080). This step, which relies on anti-mouse IgG heavy and light chains and mouse anti-rabbit IgG heavy chains that bind to the primary antibody on the tissue, increase the total number of antibodies bound at the site of antigen to amplify the signal. Using this protocol, a robust increase in staining was observed (FIG. 46), such that ADAM8 detection levels in the breast vs HEK293-ADAM8-2D cells appeared consistent with the Western blot analysis (FIG. 44). Thus, PIER method with P2 plus an amplification step was identified as the optimal protocol for use of ADP antibodies in IHC-based detection of ADAM8.

### Example 39. Selection of ADP2 as lead diagnostic antibody

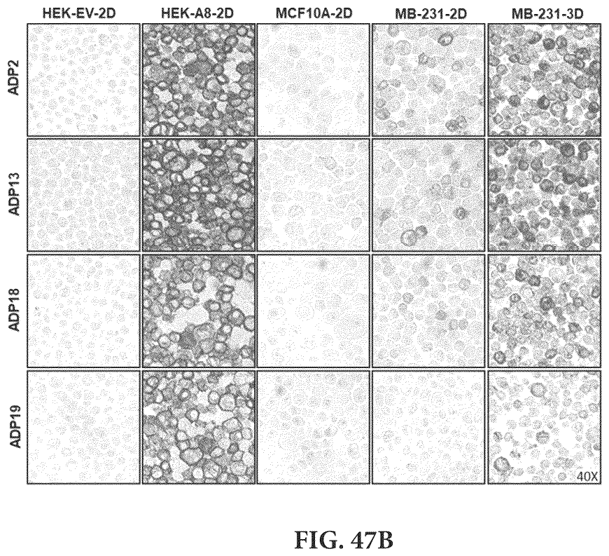

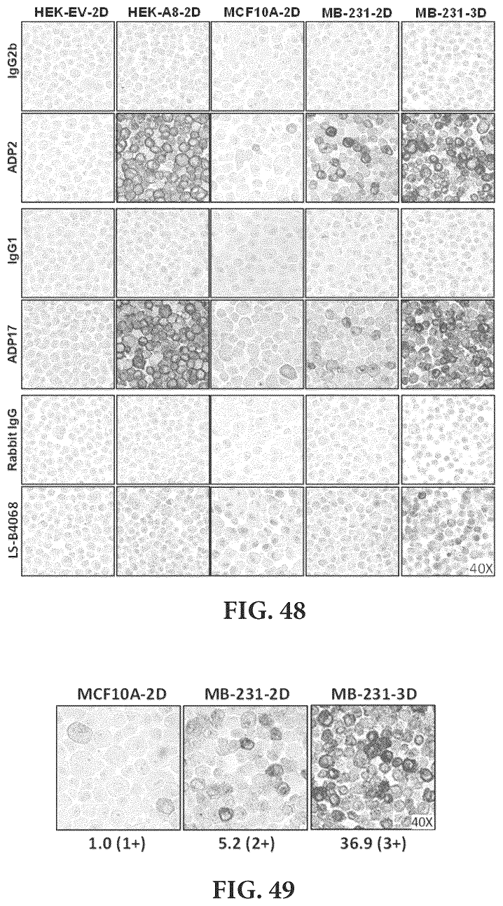

Using the newly optimized ADP-specific IHC conditions, the ADP panel of antibodies was then re-assessed for their activity in IHC, focusing on the original 5 top binders identified in FACS and an additional 4 antibodies. ADP2, ADP13 and ADP17 showed comparable staining (FIGS. 47A, 47B and 48). ADP2 displayed the highest signal to background ratio, followed by ADP17 and then ADP13. ADP12 and ADP18 demonstrated substantial staining of ADAM8, but this was below the levels seen with ADP2; while, staining with ADP3, ADP4, ADP9 and ADP19 was barely detectable. Notably, only very poor staining was seen with LS-B4068 under the conditions optimized for ADP staining (FIG. 48). Thus, ADP2 was selected as the lead diagnostic antibody and ADP17 as a backup.

### Example 40. Establishment of CCM Scoring System

To quantify the levels of active ADAM8 in the CCM breast cell lines, Western blot analyses were performed of protein extracts that were loaded at various concentrations to ensure that levels were within the linear range. Gels were quantified using densitometry and compared to staining from ADP2 IHC analyses of CCM slides. Relative levels of active ADAM8 were determined by densitometry of multiple Western blot images. The means are presented relative to the MCF-10A-2D cell line, which was set to 1.0 (FIG. 49).

MCF10A-2D, MDA-MB-231-2D and MDA-MB-231-3D cells displayed a stepwise ~5-7-fold increase in relative active ADAM8 levels in Western blotting, and commensurately, a low, medium and high percent cell culture staining positivity in IHC and were thus defined as having a simple 1+, 2+ and 3+ADAM8 IHC staining score, respectively (FIG. 49). Based on the findings of all of these studies, MCF-10A-2D, MDA-MB-231-2D, and MDA-MB-231-3D were determined to be appropriate cells with a gradient of ADAM8 levels for a breast focused CCM scoring system to be used for evaluation of IHC stained tissue samples. HEK293-ADAM8-2D and HEK293-Empty Vector-2D were included in the CCM as positive and negative controls of staining.

### Example 41. Validation of ADP2 Staining and CCM

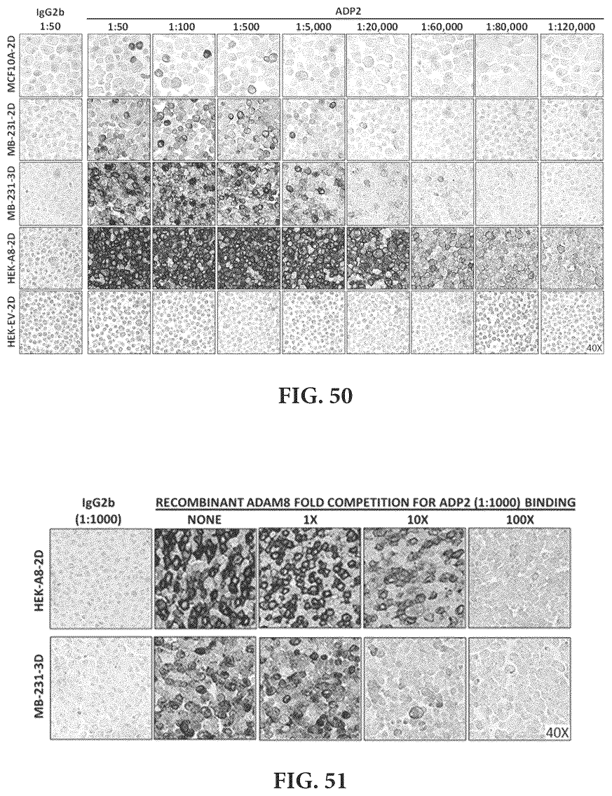

To examine the range and linearity of ADP2 ADAM8 staining using the CCM, dilutions of ADP2 ranging from 1:50 to 1:120,000 were used (FIG. 50). A low level of ADAM8 was detected at 1:50 dilution in MCF10A-2D cells, which displayed a low level of ADAM8 in longer exposure of Western blots. This staining decreased with dilution to 1:5,000 where very little staining was observed; no staining was detected with further dilution to 1:20,000. With MDA-MB-231-2D, and MDA-MB-231-3D, which express an increasing gradient of ADAM8 levels (FIGS. 44 and 46), staining was detected up to 1:20,000 and 1:80,000, respectively (FIG. 50). Staining with HEK293-ADAM8-2D cells was still visible although greatly reduced even after a 1:120,000 dilution. No staining was observed in HEK293-Empty Vector-2D cells even at the highest dose of ADP2, consistent with lack of ADAM8 expression in the parental HEK293 cells (Romagnoli et al., EMBO Mol Med, 6 (2): 278-294, 2014). Thus, dose-dependent ADAM8 staining was observed in all of the lines examined, with ADP2 being able to detect both very low and high levels of its target protein, demonstrating that this antibody had an excellent range and linearity of staining, appropriate for a diagnostic antibody and the analysis of patient samples with a wide range of protein levels.

As a critical test of ADP2 binding specificity, a competition assay was performed using increasing doses of the purified recombinant human ADAM8 (rhADAM8) protein (AA17-497, ACRO Biosystems AD8-H5223) that was the immunogen for generation of the ADP antibodies in mice. ADP2 was incubated overnight at 4° C. in the absence or presence of 1×, 10× or 100× molar equivalents of rhADAM8 and used in IHC of HEK293-ADAM8-2D and MDA-MB-231-3D cells (FIG. 51). A dose-dependent decrease in staining of both lines was seen with addition of rhADAM8. Competition using a rhADAM8 (AA1-497) from a second source (R&D Systems, 1031-AD) and HEK293-ADAM8-2D demonstrated a similar dose-dependent decrease in staining (data not shown). These data confirm the specificity of the ADP2 staining for ADAM8.

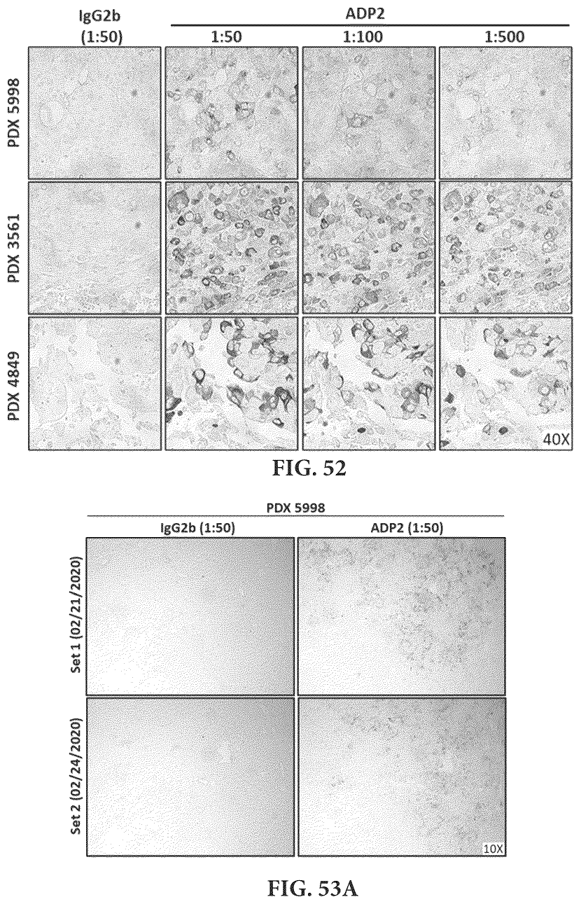

Patient-Derived Xenograft (PDX) samples were maintained in mice and displayed a stable phenotype across multiple transplant generations, as judged by histologic, transcriptomic, proteomic and genomic analyses (Zhang et al., Cancer Res, 73 (15): 4885-4897, 2013; Zhang et al., Curr Protoc Mouse Biol, 3 (1): 21-29, 2013). In order to further validate the IHC conditions and the CCM, two sets of TMAs with 30 TNBC PDX samples total were assessed using either LS-B4068 or ADP2, at a dilution of 1:100 and with the appropriate optimal staining conditions for each antibody. ADP2 staining demonstrated superior detection of ADAM8 in PDX samples vs LS-B4068 (data not shown). Three samples with substantial staining were selected for further dose-response and reproducibility analysis: PDX 5998, PDX 3561, and PDX 4849. Next, two sets of single section slides for each of these three TNBC PDX tumors (four consecutively cut slides/PDX/set) were subjected on different days to IHC using ADP2 at dilutions of 1:50, 1:100 and 1:500 vs the isotype-matched control IgG2b at 1:50. All three PDX samples displayed strong staining at the 1:50 dilution of ADP2 (FIG. 52). Decreased staining was seen at the 1:100 and 1:500 dilutions, whereas the isotype control was negative. Thus, the 1:50 dilution of ADP2 was selected as optimal for analysis of tissues.

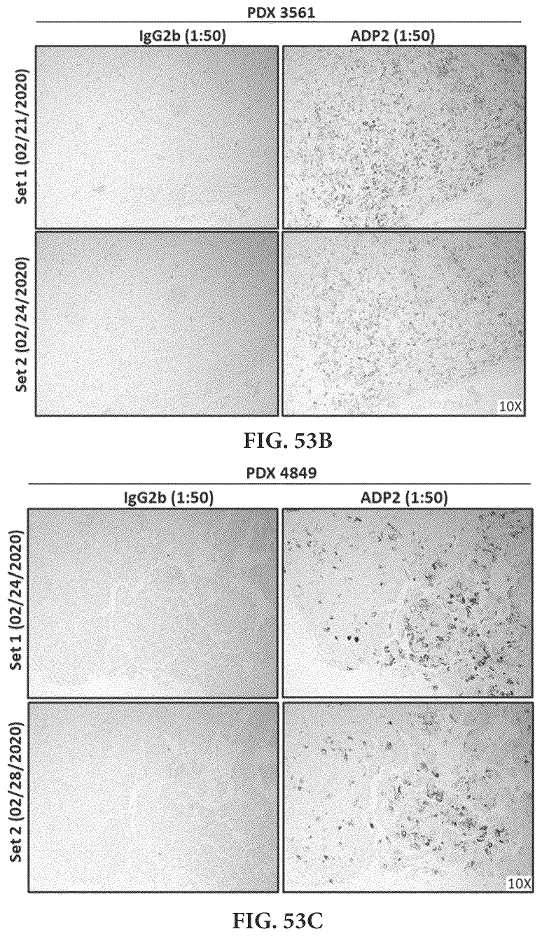

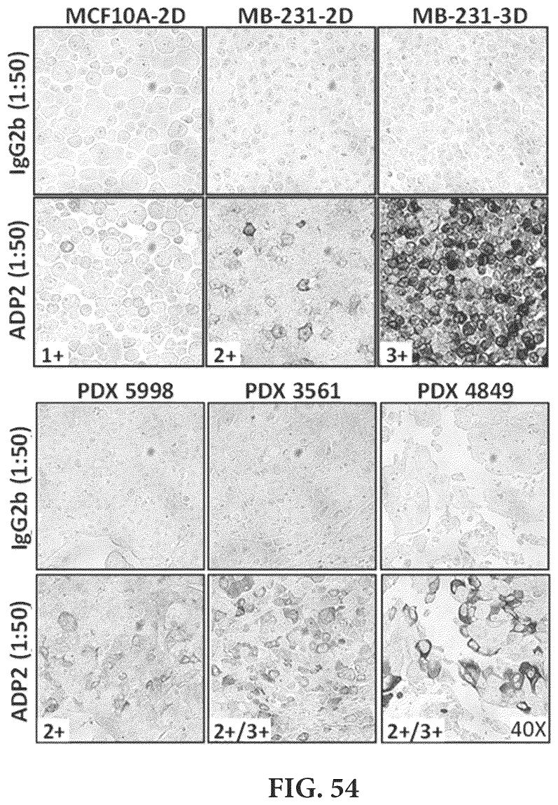

Comparable staining was seen of the two sets performed on different days for each PDX sample, demonstrating the reproducibility of the assay (FIGS. 53A-C). Importantly, the PDX samples had IHC scores between 2+ and 3+, which were within the range of the breast lines of the CCM, confirming appropriate cells have been selected for scoring of tumor samples (FIG. 54).

At present, there are no marketed diagnostics for ADAM8-positive cancers. Commercially available RUO IHC antibodies which have been used in publications to define ADAM8 levels in different cancers cannot be used in clinical diagnostics as they lack the necessary characterization (e.g., demonstration of target specificity-no IHC recombinant ADAM8 competition studies or cross-reactivity studies against related ADAM family proteins). In addition, many of them, including LS-B4068 used in the preliminary assessment of ADAM8 in breast cancer samples, are polyclonal rabbit antibodies, which are difficult to reproduce long-term from rabbit to rabbit. One such antibody in particular, the MilliporeSigma rabbit anti-ADAM8 antibody (HPA064637) has been widely used by the Human Protein Atlas to characterize ADAM8 expression in normal human tissues (proteinatlas.org website). Of note, in addition to being a rabbit antibody, it was raised against a peptide within the ADAM8 prodomain, and therefore detects only inactive protein.

Taken together these observations demonstrate that ADAM8 clinical diagnostic-grade antibodies must be developed specifically for patient biopsy characterization and informed treatment against active ADAM8 and that having any antibody that simply binds this target, without extensive biological, specificity and manufacturing considerations, is not sufficient. In addition to being highly specific, and demonstrating excellent IHC staining range, linearity and reproducibility, ADP2 has the advantage of being a mouse monoclonal antibody, that is, it can be reliably supplied long term and it was also raised against functionally active forms of ADAM8.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. Section headings and any descriptions of materials, methods, and examples are illustrative only and not intended to be limiting.

What is claimed is:

1. A method of treating an ADAM8-associated breast cancer in a subject, the method comprising administering to a subject identified as having ADAM8-associated breast cancer an antibody that comprises an antigen-binding domain comprising:
(a) heavy chain variable domain CDRs of GFSFPDYY (SEQ ID NO: 2),
IRDSANGYTT (SEQ ID NO: 3), and ARYSRYYGMDY (SEQ ID NO: 4), and light chain
variable domain CDRs of QTVNYD (SEQ ID NO: 5), FAS (SEQ ID NO: 6), and
QQDYSAPWT (SEQ ID NO: 7);
(b) heavy chain variable domain CDRs of GYTFTDYY (SEQ ID NO: 12),
ISPNIGGA (SEQ ID NO: 13), and TRGGSSYPYFYAMDY (SEQ ID NO: 14), and light
chain variable domain CDRs of QSLLYSSNQKKY (SEQ ID NO: 15), WAS (SEQ ID NO:
16), and QQFYSYPYT (SEQ ID NO: 17);
(c) heavy chain variable domain CDRs of GFTFSDAW (SEQ ID NO: 22),
IRGKVNNLAT (SEQ ID NO: 23), and LGRYDATYAMDY (SEQ ID NO: 24), and light
chain variable domain CDRs of QSLVHSDGNTY (SEQ ID NO: 25), KLS (SEQ ID NO: 26),
and SQSTHVPWT (SEQ ID NO: 27);
(d) heavy chain variable domain CDRs of GFSFTDYY (SEQ ID NO: 32),
IRDSANGYTA (SEQ ID NO: 33), and ARYSRYYAMDY (SEQ ID NO: 34), and light
chain variable domain CDRs of QSVNYD (SEQ ID NO: 35), FAS (SEQ ID NO: 36), and
QQDYSSPWT (SEQ ID NO: 37);
(e) heavy chain variable domain CDRs of GYTFTDYN (SEQ ID NO: 42),
INPNNGGT (SEQ ID NO: 43), and ARKRGLGQAWLAY (SEQ ID NO: 44), and light
chain variable domain CDRs of QSLLYSGNQKNY (SEQ ID NO: 45), GAS (SEQ ID NO:
46), and QNDHSYPLT (SEQ ID NO: 47); or
(f) heavy chain variable domain CDRs of GFTFSYAW (SEQ ID NO: 52),
IRSKANNYAT (SEQ ID NO: 53), and MGRYDAAYGMDY (SEQ ID NO: 54), and light
chain variable domain CDRs of QSLVHSNGITY (SEQ ID NO: 55), KVS (SEQ ID NO: 56),
and SQSTHVPWT (SEQ ID NO: 57)

2. The method of claim 1, wherein the antibody is an IgG antibody.

3. The method of claim 1, wherein the antibody further comprises a conjugated toxin or therapeutic agent.

4. The method of claim 1, wherein the ADAM8-associated breast cancer is triple negative breast cancer.

5. The method of claim 1, wherein the ADAM8-associated breast cancer is estrogen receptor (ER)-negative breast cancer.

6. The method of claim 1, wherein the ADAM8-associated breast cancer is progesterone receptor (PR)-negative breast cancer.

7. The method of claim 1, wherein the ADAM8-associated breast cancer is human epidermal growth factor receptor 2 (HER2)-negative.

8. The method of claim 1, wherein the antigen-binding domain comprises heavy chain variable domain CDRs of SEQ ID NO: 2, SEQ ID NO: 3, and SEQ ID NO: 4, and light chain variable domain CDRs of SEQ ID NO: 5, SEQ ID NO: 6, and SEQ ID NO: 7.

9. The method of claim 8, wherein the antigen-binding domain comprises a light chain variable domain sequence of SEQ ID NO: 8 and heavy chain variable domain sequence of SEQ ID NO: 10.

10. The method of claim 1, wherein the antigen-binding domain comprises heavy chain variable domain CDRs of SEQ ID NO: 12, SEQ ID NO: 13, and SEQ ID NO: 14, and light chain variable domain CDRs of SEQ ID NO: 15, SEQ ID NO: 16, and SEQ ID NO: 17.

11. The method of claim 10, wherein the antigen-binding domain comprises a light chain variable domain sequence of SEQ ID NO: 18 and a heavy chain variable domain sequence of SEQ ID NO: 20.

12. The method of claim 1, wherein the antigen-binding domain comprises heavy chain variable domain CDRs of SEQ ID NO: 22, SEQ ID NO: 23, and SEQ ID NO: 24, and light chain variable domain CDRs of SEQ ID NO: 25, SEQ ID NO: 26, and SEQ ID NO: 27.

13. The method of claim 12, wherein the antigen-binding domain comprises a light chain variable domain sequence of SEQ ID NO: 28 and a heavy chain variable domain sequence of SEQ ID NO: 30.

14. The method of claim 1, wherein the antigen-binding domain comprises heavy chain variable domain CDRs of SEQ ID NO: 32, SEQ ID NO: 33, and SEQ ID NO: 34, and light chain variable domain CDRs of SEQ ID NO: 35, SEQ ID NO: 36, and SEQ ID NO: 37.

15. The method of claim 14, wherein the antigen-binding domain comprises a light chain variable domain sequence of SEQ ID NO: 38 and a heavy chain variable domain sequence of SEQ ID NO: 40.

16. The method of claim 1, wherein the antigen-binding domain comprises heavy chain variable domain CDRs of SEQ ID NO: 42, SEQ ID NO: 43, and SEQ ID NO: 44, and light chain variable domain CDRs of SEQ ID NO: 45, SEQ ID NO: 46, and SEQ ID NO: 47.

17. The method of claim 16, wherein the antigen-binding domain comprises a light chain variable domain sequence of SEQ ID NO: 48 and a heavy chain variable domain sequence of SEQ ID NO: 50.

18. The method of claim 1, wherein the antigen-binding domain comprises heavy chain variable domain CDRs of SEQ ID NO: 52, SEQ ID NO: 53, and SEQ ID NO: 54, and light chain variable domain CDRs of SEQ ID NO: 55, SEQ ID NO: 56, and SEQ ID NO: 57.

19. The method of claim 18, wherein the antigen-binding domain comprises a light chain variable domain sequence of SEQ ID NO: 58, and a heavy chain variable domain sequence of SEQ ID NO: 60.

20. The method of claim 1, wherein the method further comprises administering to the subject a therapeutically effective amount of: a chemotherapeutic agent, a targeted therapy, or an immunotherapy.


SEQUENCE LISTING (104 sequences)

SEQ ID NO: 1 - protein, 69 aa, Homo sapiens
DCGPPEDCRNRCCNSTTCQLAEGAQCAHGTCCQECKVKPAGELCRPKKDMCDLEEFCDGRHPECPEDAF

SEQ ID NO: 2 - protein, 8 aa, synthetic construct
GFSFPDYY

SEQ ID NO: 3 - protein, 10 aa, synthetic construct
IRDSANGYTT

SEQ ID NO: 4 - protein, 11 aa, synthetic construct
ARYSRYYGMDY

SEQ ID NO: 5 - protein, 6 aa, synthetic construct
QTVNYD

SEQ ID NO: 6 - skipped

SEQ ID NO: 7 - protein, 9 aa, synthetic construct
QQDYSAPWT

SEQ ID NO: 8 - protein, 107 aa, synthetic construct
SIVMTQTPKILLVSAGDRVTITCKASQTVNYDVAWYQQKPGQSPKPVIYFASNRYTGVPDRFTGSGFGTDFTFTISTVQAEDLAVYFCQQDYSAPWTFGGGTKLEIK

SEQ ID NO: 9 - DNA, 321 bp, synthetic construct
  source 1..321: mol_type = other DNA
agtattgtgatgacccagactcccaaaatcctgcttgtgtcagcaggagacagggttaccataacctgcaaggccagtcagactgtgaattatgatgtagcttggtaccaacagaagccagggcagtctcctaaaccggtgatatactttgcatccaatcgctacactggggtccctgatcgcttcactggcagtggatttgggacggatttcactttcaccatcagcactgtgcaggctgaagacctggcagtttatttctgtcagcaggattatagtgctccgtggacgttcggtggaggcaccaagctggaaatcaaa

SEQ ID NO: 10 - protein, 120 aa, synthetic construct
EVKLVESGGGLVQPGGSLSLSCAASGFSFPDYYMSWVRQPPGKALEWLGFIRDSANGYTTEYIASVKGRFTFSRDNSQSILYLQMNALRAEDSATYYCARYSRYYGMDYWGQGTSVTVSS

SEQ ID NO: 11 - DNA, 360 bp, synthetic construct
  source 1..360: mol_type = other DNA
gaggtgaagctggtggagtctggaggaggcttggtacagcctgggggttctctgagtctctcctgtgcagcttctggattctccttccctgattactacatgagctgggtccgccagcctccagggaaggcacttgagtggttgggttttattagagacagtgctaatggttacacaacagagtacattgcatctgtcaagggtcggttcaccttctccagagataattcccaaagcatcctctatcttcaaatgaatgccctgagagctgaggacagtgccacttattactgtgcaagatatagccgatactatggtatggactattggggtcaaggaacctcagtcaccgtctcctca

SEQ ID NO: 12 - protein, 8 aa, synthetic construct
GYTFTDYY

SEQ ID NO: 13 - protein, 8 aa, synthetic construct
ISPNIGGA

SEQ ID NO: 14 - protein, 15 aa, synthetic construct
TRGGSSYPYFYAMDY

SEQ ID NO: 15 - protein, 12 aa, synthetic construct
QSLLYSSNQKKY

SEQ ID NO: 16 - skipped

SEQ ID NO: 17 - protein, 9 aa, synthetic construct
QQFYSYPYT

SEQ ID NO: 18 - protein, 114 aa, synthetic construct
DIVMSQSPSSLAVSVGEKVTMSCKSSQSLLYSSNQKKYLAWYQQKPGQSPKLLIYWASTRESGVPDRFTGSGSGTDFTLTISSVKAEDLAVYYCQQFYSYPYTFGGGTKLEINR

SEQ ID NO: 19 - DNA, 342 bp, synthetic construct
  source 1..342: mol_type = other DNA
gacattgtgatgtcacagtctccatcctccctagctgtgtcagttggagagaaggttactatgagctgcaagtccagtcagagccttttatatagtagcaatcaaaagaagtacttggcctggtaccagcagaaaccagggcagtctcctaaattgttgatttactgggcatccactagggaatctggggtccctgatcgcttcacaggcagtggatctgggacagatttcactctcaccatcagcagtgtgaaggctgaagacctggcagtttattactgtcagcaattttatagctatccgtacacgttcggaggggggaccaagctggaaataaaccgg

SEQ ID NO: 20 - protein, 122 aa, synthetic construct
EVQLQQSGPEMVKPGTSVKISCKASGYTFTDYYINWVKQSHGKSLEWIGDISPNIGGATYNPKFKGKAILTVDKSARTAYMELRSLTSEDSAVYCCTRGGSSYPYFYAMDYWGQGTSVTVSS

SEQ ID NO: 21 - DNA, 366 bp, synthetic construct
  source 1..366: mol_type = other DNA
gaggtccagctgcaacaatctggacctgaaatggtgaagcctgggacttcagtgaagatatcgtgtaaggcttctggatacacgttcactgactactacataaactgggtgaagcagagccatggaaagagccttgagtggattggagatattagtcctaacattggtggtgcgacctacaacccgaagttcaagggcaaggccatattgactgtagacaagtccgccagaacagcctacatggagctccgcagcctgacatctgaggactctgcagtctattgctgtacaagaggcggtagtagctacccctatttctatgctatggactactggggtcaaggaacctcagtcaccgtctcctca

SEQ ID NO: 22 - protein, 8 aa, synthetic construct
GFTFSDAW

SEQ ID NO: 23 - protein, 10 aa, synthetic construct
IRGKVNNLAT

SEQ ID NO: 24 - protein, 12 aa, synthetic construct
LGRYDATYAMDY

SEQ ID NO: 25 - protein, 11 aa, synthetic construct
QSLVHSDGNTY

SEQ ID NO: 26 - skipped

SEQ ID NO: 27 - protein, 9 aa, synthetic construct
SQSTHVPWT

SEQ ID NO: 28 - protein, 112 aa, synthetic construct
DVVMTQTPLSLPVSLGDQASISCRSSQSLVHSDGNTYLHWYLQKPGQSPKLLIYKLSNRFSGVPDRFSGSGSGTDFTLKISRVEAEDLGVYFCSQSTHVPWTFGGGTKLEIK

SEQ ID NO: 29 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacacagtgatggaaacacctatttacattggtacctgcagaagccaggccagtctccaaagctcctgatctacaaactttccaaccgattttctggggtcccagacaggttcagtggcagtggatcagggacagatttcacactcaagatcagcagagtggaggctgaggatctgggagtttatttctgctctcaaagtacacatgttccgtggacgttcggaggggggaccaagctggaaataaaa

SEQ ID NO: 30 - protein, 121 aa, synthetic construct
EVKLEESGGGLVQPGGSMKLSCAASGFTFSDAWMDWVRQSPEKGLEWVAEIRGKVNNLATYYVESVKGRFTISRDDSKSSVYLQMNSLRAEDTGIYYCLGRYDATYAMDYWGQGTSVTVSS

SEQ ID NO: 31 - DNA, 363 bp, synthetic construct
  source 1..363: mol_type = other DNA
gaagtgaagcttgaggagtctggaggaggcttggtgcaacctggaggatccatgaaactctcttgtgctgcctctggattcacttttagtgacgcctggatggactgggtccgccagtctccagagaagggacttgagtgggttgctgaaattagaggcaaagttaataatcttgcaacgtactatgttgagtctgtgaaagggaggttcaccatctcaagagatgattccaaaagtagtgtctacctgcaaatgaacagcttaagagctgaagacactggcatttattactgtttggggaggtacgacgcgacatatgctatggactactggggtcaaggaacctcagtcaccgtctcctca

SEQ ID NO: 32 - protein, 8 aa, synthetic construct
GFSFTDYY

SEQ ID NO: 33 - protein, 10 aa, synthetic construct
IRDSANGYTA

SEQ ID NO: 34 - protein, 11 aa, synthetic construct
ARYSRYYAMDY

SEQ ID NO: 35 - protein, 6 aa, synthetic construct
QSVNYD

SEQ ID NO: 36 - skipped

SEQ ID NO: 37 - protein, 9 aa, synthetic construct
QQDYSSPWT

SEQ ID NO: 38 - protein, 107 aa, synthetic construct
FIVMTQTPKILLVSAGDRITITCKASQSVNYDVAWYQQKPGQSPKPVIYFASNRYTGVPDRFTGSGFGTDFTFTISTVQAEDLAVYFCQQDYSSPWTFGGGTKLEIK

SEQ ID NO: 39 - DNA, 321 bp, synthetic construct
  source 1..321: mol_type = other DNA
tttattgtgatgacccagactcccaaaatcctgcttgtatcagcaggagacaggatcaccataacctgcaaggccagtcagagtgtgaattatgatgtcgcttggtaccaacagaagccagggcagtctcctaaaccggtgatatactttgcatccaatcgctacactggagtccctgatcgcttcactggcagtggatttgggacggatttcactttcaccatcagcactgtgcaggctgaagacctggcagtttatttctgtcagcaggattatagctctccgtggacgttcggtggaggcaccaagctggaaatcaaa

SEQ ID NO: 40 - protein, 120 aa, synthetic construct
EVKLVESGGGLVQPGGSLSLSCETSGFSFTDYYMIWVRQPPGKALEWLGFIRDSANGYTAEYIASVKGRFTFSRDNSQSILYLQMNALRAEDSATYYCARYSRYYAMDYWGQGTSVTVAP

SEQ ID NO: 41 - DNA, 360 bp, synthetic construct
  source 1..360: mol_type = other DNA
gaggtgaagctggtggagtctggaggaggcttggtacagcctgggggttctctgagtctctcctgtgaaacttctggattctccttcactgattactatatgatctgggtccgccagcctccagggaaggcacttgagtggttgggttttatcagagacagtgctaatggttacacagcagagtacattgcatctgtgaagggtcggttcaccttctccagagataattcccaaagcatcctctatcttcaaatgaatgccctgagagctgaggacagtgccacttattactgtgcaagatatagccgatactatgctatggactattggggtcagggaacctcagtcaccgtcgcccca

SEQ ID NO: 42 - protein, 8 aa, synthetic construct
GYTFTDYN

SEQ ID NO: 43 - protein, 8 aa, synthetic construct
INPNNGGT

SEQ ID NO: 44 - protein, 13 aa, synthetic construct
ARKRGLGQAWLAY

SEQ ID NO: 45 - protein, 12 aa, synthetic construct
QSLLYSGNQKNY

SEQ ID NO: 46 - skipped

SEQ ID NO: 47 - protein, 9 aa, synthetic construct
QNDHSYPLT

SEQ ID NO: 48 - protein, 113 aa, synthetic construct
DIVMTQSPSSRSVSAGEKVTMSCKSSQSLLYSGNQKNYLAWYQQKPGQPPKLLIYGASTRESGVPDRFTGSGSGTDFTLTISSVQAEDLAVYYCQNDHSYPLTFGAGTKLELK

SEQ ID NO: 49 - DNA, 339 bp, synthetic construct
  source 1..339: mol_type = other DNA
gacattgtgatgacacagtctccatcctcccggagtgtgtcagcaggagagaaggtcactatgagctgcaagtccagccagagtctgttatacagtggaaatcaaaagaactacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccgctcacgttcggtgctgggaccaagctggagctgaaa

SEQ ID NO: 50 - protein, 120 aa, synthetic construct
EVQLQQSGPELVKPGASVKIPCKASGYTFTDYNMDWVKQSHGKSLDWIGDINPNNGGTIYNQKFKGKATLTVDKSSSTAYMELRSLTSEDTAVYYCARKRGLGQAWLAYWGQGTLVTVSA

SEQ ID NO: 51 - DNA, 360 bp, synthetic construct
  source 1..360: mol_type = other DNA
gaggtccagctgcaacagtctggacctgagctggtgaagcctggggcttcagtgaagataccctgcaaggcttctggatacacattcactgactacaacatggactgggtgaagcagagccatggaaagagccttgactggattggagatattaatccaaacaatggtggtactatctacaaccagaagttcaagggcaaggccacattgactgtagacaagtcctccagcacagcctacatggagctccgcagcctgacatctgaggacactgcagtctattactgtgcaagaaagagaggcctgggacaggcctggttagcttactggggccaagggactctggtcactgtctctgca

SEQ ID NO: 52 - protein, 8 aa, synthetic construct
GFTFSYAW

SEQ ID NO: 53 - protein, 10 aa, synthetic construct
IRSKANNYAT

SEQ ID NO: 54 - protein, 12 aa, synthetic construct
MGRYDAAYGMDY

SEQ ID NO: 55 - protein, 11 aa, synthetic construct
QSLVHSNGITY

SEQ ID NO: 56 - skipped

SEQ ID NO: 57 - protein, 9 aa, synthetic construct
SQSTHVPWT

SEQ ID NO: 58 - protein, 112 aa, synthetic construct
DVVMTQTPLSLPVSLGYQASISCRSSQSLVHSNGITYLHWYLQKPGQSPKLLIYKVSNRFSGVPDRFSGSGSGTDFTLKISRVEAEDLGVYFCSQSTHVPWTFGGGTKLEIK

SEQ ID NO: 59 - DNA, 336 bp, synthetic construct
  source 1..336: mol_type = other DNA
gatgttgtgatgacccaaactccactctccctgcctgtcagtcttggatatcaagcctccatctcttgcagatctagtcagagccttgttcacagcaatggaatcacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 60 - protein, 121 aa, synthetic construct
EVKLEESGGGLVQPGGSMKLSCAASGFTFSYAWMDWVRQSPEKGLEWVAEIRSKANNYATYYAESVKGRFTISRNDSKSSVYLQMNSLRIEDTGIYYCMGRYDAAYGMDYWGQGTSVTVSS

SEQ ID NO: 61 - DNA, 363 bp, synthetic construct
  source 1..363: mol_type = other DNA
gaagtgaagcttgaggagtctggaggaggcttggtgcaacctggaggatccatgaaactctcttgtgctgcctctggattcacttttagttacgcctggatggactgggtccgccagtctcccgagaagggacttgagtgggttgctgaaattagaagcaaagctaataattatgcaacatactatgctgagtctgtgaaagggaggttcaccatctcaagaaatgattccaaaagtagtgtctacctacaaatgaacagcttgagaattgaagacactggcatttattactgtatggggaggtacgacgcggcctatggtatggactactggggtcaaggaacctcagtcaccgtctcctca

SEQ ID NO: 62 - protein, 8 aa, synthetic construct
GFTFSDAW

SEQ ID NO: 63 - protein, 10 aa, synthetic construct
IRNKANSHAT

SEQ ID NO: 64 - protein, 12 aa, synthetic construct
TRDGGYYAWFAY

SEQ ID NO: 65 - protein, 11 aa, synthetic construct
QSIVHSDGNTY

SEQ ID NO: 66 - skipped

SEQ ID NO: 67 - protein, 9 aa, synthetic construct
FHGSHIPLT

SEQ ID NO: 68 - protein, 112 aa, synthetic construct
GALLTQTPLSLPVSLGDQVSISCRSSQSIVHSDGNTYLEWYLQKPGQSPKLLIYRVSNRFSGVPDRFSGSGSGTDFTLKISRVEAEDLGIYYCFHGSHIPLTFGAGTKLELK

SEQ ID NO: 69 - DNA, 336 bp, synthetic construct
  source 1..336: mol_type = other DNA
ggtgctttgctgacccaaactccactctccctgcctgtcagtcttggagatcaagtctccatctcttgcaggtctagtcagagcattgtacatagtgatggaaacacctatttagaatggtacctgcagaagccaggccagtctccaaagctcctgatttacagagtttccaaccgattttctggggtcccagacaggttcagtggcagtggatcagggacagacttcacactcaagatcagcagagtggaggctgaggatctgggaatttattactgctttcacggttcacatattccgctcacgttcggtgctgggaccaagctggagctgaaa

SEQ ID NO: 70 - protein, 121 aa, synthetic construct
EVKFEESGGGLVQPGGSMKLSCAASGFTFSDAWMDWVRQSPEKGLEWVAEIRNKANSHATYYAESVKGRFTISRDDSKSTVYLQMNSLRAEDTGIYYCTRDGGYYAWFAYWGQGTLVTVSA

SEQ ID NO: 71 - DNA, 363 bp, synthetic construct
  source 1..363: mol_type = other DNA
gaagtgaagtttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaaacaaagctaatagtcatgcaacatactatgctgagtctgtgaaagggaggttcaccatctcaagagatgattccaaaagtactgtctacctgcaaatgaacagcttaagagctgaagacactggcatttattactgtaccagggatggtggttactacgcctggtttgcttactggggccaagggactctggtcactgtctctgca

SEQ ID NO: 72 - protein, 8 aa, synthetic construct
GFTFSDAW

SEQ ID NO: 73 - protein, 10 aa, synthetic construct
IRNKANNHAT

SEQ ID NO: 74 - protein, 12 aa, synthetic construct
TRDGGYYAWFAY

SEQ ID NO: 75 - protein, 11 aa, synthetic construct
QSIVHSDGNTY

SEQ ID NO: 76 - skipped

SEQ ID NO: 77 - protein, 9 aa, synthetic construct
FHGSHIPLT

SEQ ID NO: 78 - protein, 112 aa, synthetic construct
GVLMTQTPLSLPVSLGDQASISCRSSQSIVHSDGNTYLEWYLQKPGQSPKLLLYKVSNRFSGVPDRFSGSGSGTDFTLKISRVEAEDLGVYYCFHGSHIPLTFGAGTKLELK

SEQ ID NO: 79 - DNA, 336 bp, synthetic construct
  source 1..336: mol_type = other DNA
ggtgttttgatgacccaaactccactctccctgcctgtcagtcttggagatcaagcctccatctcttgcaggtctagtcagagcattgtacatagtgatggaaacacctatttagaatggtacctgcagaagccaggccagtctccaaagctcctgctctacaaagtttccaaccgattttctggggtcccagacaggttcagtggcagtggatcagggacagatttcacactcaagatcagcagagtggaggctgaggatctgggagtttattactgctttcatggttcacatattccgctcacgttcggtgctgggaccaagctggagctgaaa

SEQ ID NO: 80 - protein, 121 aa, synthetic construct
EVKFEESGGGLVQPGGSMKLSCAASGFTFSDAWMDWVRQSPEKGLEWVAEIRNKANNHATYYAESVKGRFTISRDDSKSSVYLQMNSLRAEDTGIYYCTRDGGYYAWFAYWGQGTLVTVSA

SEQ ID NO: 81 - DNA, 363 bp, synthetic construct
  source 1..363: mol_type = other DNA
gaagtgaagtttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaaacaaagctaataatcatgcaacatactatgctgagtctgtgaaagggaggttcaccatctcaagagatgattccaaaagtagtgtctacctgcaaatgaacagtttaagagctgaagacactggcatttattactgtaccagggatggtggttactacgcctggtttgcttactggggccaagggactctggtcactgtctctgca

SEQ ID NO: 82 - protein, 449 aa, synthetic construct
EVKLVESGGGLVQPGGSLSLSCAASGFSFPDYYMSWVRQPPGKALEWLGFIRDSANGYTTEYIASVKGRFTFSRDNSQSILYLQMNALRAEDSATYYCARYSRYYGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 214 aa, synthetic construct
SIVMTQTPKILLVSAGDRVTITCKASQTVNYDVAWYQQKPGQSPKPVIYFASNRYTGVPDRFTGSGFGTDFTFTISTVQAEDLAVYFCQQDYSAPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 451 aa, synthetic construct
EVQLQQSGPEMVKPGTSVKISCKASGYTFTDYYINWVKQSHGKSLEWIGDISPNIGGATYNPKFKGKAILTVDKSARTAYMELRSLTSEDSAVYCCTRGGSSYPYFYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 220 aa, synthetic construct
DIVMSQSPSSLAVSVGEKVTMSCKSSQSLLYSSNQKKYLAWYQQKPGQSPKLLIYWASTRESGVPDRFTGSGSGTDFTLTISSVKAEDLAVYYCQQFYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 25 aa, synthetic construct
CCNSTTCQLAEGAQCAHGTCCQECK

SEQ ID NO: 87 - protein, 50 aa, synthetic construct
LAEGAQCAHGTCCQECKVKPAGELCRPKKDMCDLEEFCDGRHPECPEDAF

SEQ ID NO: 88 - protein, 19 aa, Homo sapiens
DCGPPEDCRNRCCNSTTCQ

SEQ ID NO: 89 - protein, 9 aa, Homo sapiens
CRPKKDMCD

SEQ ID NO: 90 - protein, 11 aa, synthetic construct
HEXXHXXGXXH

SEQ ID NO: 91 - protein, 824 aa, Homo sapiens
MRGLGLWLLGAMMLPAIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 92 - DNA, 2475 bp, Homo sapiens
  source 1..2475: mol_type = genomic DNA
atgcgcggcctcgggctctggctgctgggcgcgatgatgctgcctgcgattgcccccagccggccctgggccctcatggagcagtatgaggtcgtgttgccgtggcgtctgccaggcccccgagtccgccgagctctgccctcccacttgggcctgcacccagagagggtgagctacgtccttggggccacagggcacaacttcaccctccacctgcggaagaacagggacctgctgggctccggctacacagagacctatacggctgccaatggctccgaggtgacggagcagcctcgcgggcaggaccactgcttctaccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcccgagagacccgctacgtggagctgtatgtggtcgtggacaatgcagagttccagatgctggggagcgaagcagccgtgcgtcatcgggtgctggaggtggtgaatcacgtggacaagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgggtgtcgacttcaccgggactaccgtggggtttgccagggtgtccgccatgtgctcccacagctcaggggctgtgaaccaggaccacagcaagaaccccgtgggcgtggcctgtaccatggcccatgagatgggccacaacctgggcatggaccatgatgagaacgtccagggctgccgctgccaggaacgcttcgaggccggccgctgcatcatggcgggcagcattggctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggactgccggaaccgctgctgcaactctaccacctgccagctggctgagggggcccagtgtgcgcacggtacctgctgccaggagtgcaaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgggcaggctgccgaggagtcctgcttctcctatgacatcctaccaggctgcaaggccagccggtacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtttgctggaaaggacgttgccaggacttacacgtttacagatccagcaactgctctgcccagtgccacaaccatggggtgtgcaaccacaagcaggagtgccactgccacgcgggctgggccccgccccactgcgcgaagctgctgactgaggtgcacgcagcgtccgggagcctccccgtcttcgtggtggtggttctggtgctcctggcagttgtgctggtcaccctggcaggcatcatcgtctaccgcaaagcccggagccgcatcctgagcaggaacgtggctcccaagaccacaatggggcgctccaaccccctgttccaccaggctgccagccgcgtgccggccaagggcggggctccagccccatccaggggcccccaagagctggtccccaccacccacccgggccagcccgcccgacacccggcctcctcggtggctctgaagaggccgccccctgctcctccggtcactgtgtccagcccacccttcccagttcctgtctacacccggcaggcaccaaagcaggtcatcaagccaacgttcgcacccccagtgcccccagtcaaacccggggctggtgcggccaaccctggtccagctgagggtgctgttggcccaaaggttgccctgaagccccccatccagaggaagcaaggagccggagctcccacagcaccctag

SEQ ID NO: 93 - protein, 742 aa, Homo sapiens
MRGLGLWLLGAMMLPAIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAGCQPRAGQGRGSSPIQGPPRAGPHHPPGPARPTPGLLGGSEEAAPCSSGHCVQPTLPSSCLHPAGTKAGHQANVRTPSAPSQTRGWCGQPWSS

SEQ ID NO: 94 - DNA, 2229 bp, Homo sapiens
  source 1..2229: mol_type = genomic DNA
atgcgcggcctcgggctctggctgctgggcgcgatgatgctgcctgcgattgcccccagccggccctgggccctcatggagcagtatgaggtcgtgttgccgtggcgtctgccaggcccccgagtccgccgagctctgccctcccacttgggcctgcacccagagagggtgagctacgtccttggggccacagggcacaacttcaccctccacctgcggaagaacagggacctgctgggctccggctacacagagacctatacggctgccaatggctccgaggtgacggagcagcctcgcgggcaggaccactgcttctaccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcccgagagacccgctacgtggagctgtatgtggtcgtggacaatgcagagttccagatgctggggagcgaagcagccgtgcgtcatcgggtgctggaggtggtgaatcacgtggacaagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgggtgtcgacttcaccgggactaccgtggggtttgccagggtgtccgccatgtgctcccacagctcaggggctgtgaaccaggaccacagcaagaaccccgtgggcgtggcctgtaccatggcccatgagatgggccacaacctgggcatggaccatgatgagaacgtccagggctgccgctgccaggaacgcttcgaggccggccgctgcatcatggcgggcagcattggctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggactgccggaaccgctgctgcaactctaccacctgccagctggctgagggggcccagtgtgcgcacggtacctgctgccaggagtgcaaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgggcaggctgccgaggagtcctgcttctcctatgacatcctaccaggctgcaaggccagccggtacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtttgctggaaaggacgttgccaggacttacacgtttacagatccagcaactgctctgcccagtgccacaaccatggggtgtgcaaccacaagcaggagtgccactgccacgcgggctgggccccgccccactgcgcgaagctgctgactgaggtgcacgcaggctgccagccgcgtgccggccaagggcggggctccagccccatccaggggcccccaagagctggtccccaccacccacccgggccagcccgcccgacacccggcctcctcggtggctctgaagaggccgccccctgctcctccggtcactgtgtccagcccacccttcccagttcctgtctacacccggcaggcaccaaagcaggtcatcaagccaacgttcgcacccccagtgcccccagtcaaacccggggctggtgcggccaaccctggtccagctga

SEQ ID NO: 95 - protein, 733 aa, Homo sapiens
MRGLGLWLLGAMMLPGPAPREGELRPWGHRAQLHPPPAEEQGPAGLRLHRDLYGCQWLRGDGAASRAGPLLLPGPRRGVPGLSRQPQHLCRPQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQGAVGPKVALKPPIQRKQGAGAPTAP

SEQ ID NO: 96 - DNA, 2202 bp, Homo sapiens
  source 1..2202: mol_type = genomic DNA
atgcgcggcctcgggctctggctgctgggcgcgatgatgctgcctgggcctgcacccagagagggtgagctacgtccttggggccacagggcacaacttcaccctccacctgcggaagaacagggacctgctgggctccggctacacagagacctatacggctgccaatggctccgaggtgacggagcagcctcgcgggcaggaccactgcttctaccagggccacgtagaggggtacccggactcagccgccagcctcagcacctgtgccggcctcaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcccgagagacccgctacgtggagctgtatgtggtcgtggacaatgcagagttccagatgctggggagcgaagcagccgtgcgtcatcgggtgctggaggtggtgaatcacgtggacaagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgggtgtcgacttcaccgggactaccgtggggtttgccagggtgtccgccatgtgctcccacagctcaggggctgtgaaccaggaccacagcaagaaccccgtgggcgtggcctgtaccatggcccatgagatgggccacaacctgggcatggaccatgatgagaacgtccagggctgccgctgccaggaacgcttcgaggccggccgctgcatcatggcgggcagcattggctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggactgccggaaccgctgctgcaactctaccacctgccagctggctgagggggcccagtgtgcgcacggtacctgctgccaggagtgcaaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgggcaggctgccgaggagtcctgcttctcctatgacatcctaccaggctgcaaggccagccggtacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtgtgcaaccacaagcaggagtgccactgccacgcgggctgggccccgccccactgcgcgaagctgctgactgaggtgcacgcagcgtccgggagcctccccgtcttcgtggtggtggttctggtgctcctggcagttgtgctggtcaccctggcaggcatcatcgtctaccgcaaagcccggagccgcatcctgagcaggaacgtggctcccaagaccacaatggggcgctccaaccccctgttccaccaggctgccagccgcgtgccggccaagggcggggctccagccccatccaggggcccccaagagctggtccccaccacccacccgggccagcccgcccgacacccggcctcctcggtggctctgaagaggccgccccctgctcctccggtcactgtgtccagcccacccttcccagttcctgtctacacccggcaggcaccaaagcagggtgctgttggcccaaaggttgccctgaagccccccatccagaggaagcaaggagccggagctcccacagcaccctag

SEQ ID NO: 97 - protein, 826 aa, Mus sp.
MLGLWLLSVLWTPAVAPGPPLPHVKQYEVVWPRRLAASRSRRALPSHWGQYPESLSYALGTSGHVFTLHLRKNRDLLGSSYTETYSAANGSEVTEQLQEQDHCLYQGHVEGYEGSAASISTCAGLRGFFRVGSTVHLIEPLDADEEGQHAMYQAKHLQQKAGTCGVKDTNLNDLGPRALEIYRAQPRNWLIPRETRYVELYVVADSQEFQKLGSREAVRQRVLEVVNHVDKLYQELSFRVVLVGLEIWNKDKFYISRYANVTLENFLSWREQNLQGQHPHDNVQLITGVDFIGSTVGLAKVSALCSRHSGAVNQDHSKNSIGVASTMAHELGHNLGMSHDEDIPGCYCPEPREGGGCIMTESIGSKFPRIFSRCSKIDLESFVTKPQTGCLTNVPDVNRFVGGPVCGNLFVEHGEQCDCGTPQDCQNPCCNATTCQLVKGAECASGTCCHECKVKPAGEVCRLSKDKCDLEEFCDGRKPTCPEDAFQQNGTPCPGGYCFDGSCPTLAQQCRDLWGPGARVAADSCYTFSIPPGCNGRMYSGRINRCGALYCEGGQKPLERSFCTFSSNHGVCHALGTGSNIDTFELVLQGTKCEEGKVCMDGSCQDLRVYRSENCSAKCNNHGVCNHKRECHCHKGWAPPNCVQRLADVSDEQAASTSLPVSVVVVLVILVAAMVIVAGIVIYRKAPRQIQRRSVAPKPISGLSNPLFYTRDSSLPAKNRPPDPSETVSTNQPPRPIVKPKRPPPAPPGAVSSSPLPVPVYAPKIPNQFRPDPPTKPLPELKPKQVKPTFAPPTPPVKPGTGGTVPGATQGAGEPKVALKVPIQKR

SEQ ID NO: 98 - DNA, 2481 bp, Mus sp.
  source 1..2481: mol_type = genomic DNA
atgcttggcctctggctgctcagcgtcttatggacaccagcagtagcccctggacctcctttgccccatgtgaaacagtatgaagtggtttggcctcggcgcctagctgcatcccgctcccgcagagccctgccctcccactggggccagtacccagagagtctgagctatgctcttgggaccagcgggcacgttttcaccctgcaccttcgaaagaacagggacctgctgggctcaagctacacagagacctactcagctgccaatggctctgaggtgacagagcaactgcaggagcaggaccattgcctctaccaaggccatgtggaagggtacgagggctcagctgccagtattagcacctgtgctggcctcaggggctttttccgagttgggtccactgtccacttgattgagcctctggatgctgatgaagaggggcaacatgcgatgtatcaggcaaagcatctgcaacagaaggctgggacctgtggggtcaaagataccaacctgaatgacctagggcctcgggcattagaaatatacagggctcagccacggaactggctgatacccagagaaacccgctatgtggagttgtatgtggttgcagacagccaagagttccagaagttggggagcagagaggccgtgcgccagcgagtgctggaggttgtaaaccacgtggacaagctttatcaggaactcagttttcgagttgtcctggtgggcctggagatctggaacaaggacaaattctacatcagccgctatgccaacgtgacactggagaacttcttgtcctggagggaacagaacttgcaagggcagcacccacatgacaacgtgcaacttatcacgggggttgatttcattgggagcactgttggactggctaaggtgtctgccctgtgttcccgtcactccggagctgtgaatcaggaccactccaagaactccattggtgtagcctccaccatggcccatgagctgggccacaacctgggcatgagccatgatgaggacattccaggatgctactgtcctgaaccacgggagggtggtggctgcatcatgaccgaaagcatcggctccaagttccccaggatattcagcaggtgtagcaagattgacctagagtcattcgtgacaaaaccccagacaggctgcctgaccaatgttccagatgtcaaccggttcgtgggtggccctgtgtgtggaaacctgtttgtggagcatggagagcagtgtgactgtggcacacctcaggactgtcaaaacccctgctgcaatgccaccacttgccagctggtcaagggggcagagtgtgccagtggtacctgttgtcatgaatgcaaggtgaagccagctggagaggtgtgtcgtctcagtaaggacaaatgtgacctggaggagttctgtgatggccggaagccaacatgtcccgaagatgccttccaacagaatggcactccctgcccagggggctactgctttgatgggagctgtcccaccctggcacagcagtgccgggatctgtgggggccaggtgctcgggtagcagccgactcctgctatacctttagcatccctccgggctgcaatgggaggatgtactctggcaggatcaaccggtgtggagcgctgtactgtgagggaggccagaagccccttgaacgctccttctgcactttctcctccaaccatggagtctgccatgctcttggcacaggcagcaacattgacacctttgagctggtattgcagggcaccaagtgcgaggagggaaaggtttgcatggatggaagctgccaggacctccgtgtatacagatctgaaaactgctctgctaaatgcaacaaccatggggtatgcaaccacaagagggagtgccactgtcacaagggctgggcaccacccaactgtgtacagcggctggcagatgtatcagatgaacaagcagcgtctacgagcctcccagtcagtgtggttgtggtcttggtgatcctggtggctgcgatggtcatcgtggcaggcatcgtcatctaccgaaaggctccgagacaaatccagaggaggagtgtggcacccaagcctatctcggggctctccaaccccctattctacacaagggacagcagcctgccagctaagaacaggcctccagacccttctgagacagtttctaccaaccagcccccaagacccatagtgaaaccaaagaggcctccccctgcacctccaggtgctgtgtccagttcaccactcccagttcctgtttatgccccaaagataccaaatcagtttagacctgatcctcccaccaagcccctcccagagctgaaacccaagcaggtcaagccaacctttgcacccccgacaccaccagtcaagcccgggactggagggacggtgcctggagcaactcagggagctggtgagccaaaggttgctctgaaggtccccatccagaagaggtga

SEQ ID NO: 99 - protein, 825 aa, Mus sp.
MLGLWLLSVLWTPVAPGPPLPHVKQYEVVWPRRLAASRSRRALPSHWGQYPESLSYALGTSGHVFTLHLRKNRDLLGSSYTETYSAANGSEVTEQLQEQDHCLYQGHVEGYEGSAASISTCAGLRGFFRVGSTVHLIEPLDADEEGQHAMYQAKHLQQKAGTCGVKDTNLNDLGPRALEIYRAQPRNWLIPRETRYVELYVVADSQEFQKLGSREAVRQRVLEVVNHVDKLYQELSFRVVLVGLEIWNKDKFYISRYANVTLENFLSWREQNLQGQHPHDNVQLITGVDFIGSTVGLAKVSALCSRHSGAVNQDHSKNSIGVASTMAHELGHNLGMSHDEDIPGCYCPEPREGGGCIMTESIGSKFPRIFSRCSKIDLESFVTKPQTGCLTNVPDVNRFVGGPVCGNLFVEHGEQCDCGTPQDCQNPCCNATTCQLVKGAECASGTCCHECKVKPAGEVCRLSKDKCDLEEFCDGRKPTCPEDAFQQNGTPCPGGYCFDGSCPTLAQQCRDLWGPGARVAADSCYTFSIPPGCNGRMYSGRINRCGALYCEGGQKPLERSFCTFSSNHGVCHALGTGSNIDTFELVLQGTKCEEGKVCMDGSCQDLRVYRSENCSAKCNNHGVCNHKRECHCHKGWAPPNCVQRLADVSDEQAASTSLPVSVVVVLVILVAAMVIVAGIVIYRKAPRQIQRRSVAPKPISGLSNPLFYTRDSSLPAKNRPPDPSETVSTNQPPRPIVKPKRPPPAPPGAVSSSPLPVPVYAPKIPNQFRPDPPTKPLPELKPKQVKPTFAPPTPPVKPGTGGTVPGATQGAGEPKVALKVPIQKR

SEQ ID NO: 100 - DNA, 2478 bp, Mus sp.
  source 1..2478: mol_type = genomic DNA
atgcttggcctctggctgctcagcgtcttatggacaccagtagcccctggacctcctttgccccatgtgaaacagtatgaagtggtttggcctcggcgcctagctgcatcccgctcccgcagagccctgccctcccactggggccagtacccagagagtctgagctatgctcttgggaccagcgggcacgttttcaccctgcaccttcgaaagaacagggacctgctgggctcaagctacacagagacctactcagctgccaatggctctgaggtgacagagcaactgcaggagcaggaccattgcctctaccaaggccatgtggaagggtacgagggctcagctgccagtattagcacctgtgctggcctcaggggctttttccgagttgggtccactgtccacttgattgagcctctggatgctgatgaagaggggcaacatgcgatgtatcaggcaaagcatctgcaacagaaggctgggacctgtggggtcaaagataccaacctgaatgacctagggcctcgggcattagaaatatacagggctcagccacggaactggctgatacccagagaaacccgctatgtggagttgtatgtggttgcagacagccaagagttccagaagttggggagcagagaggccgtgcgccagcgagtgctggaggttgtaaaccacgtggacaagctttatcaggaactcagttttcgagttgtcctggtgggcctggagatctggaacaaggacaaattctacatcagccgctatgccaacgtgacactggagaacttcttgtcctggagggaacagaacttgcaagggcagcacccacatgacaacgtgcaacttatcacgggggttgatttcattgggagcactgttggactggctaaggtgtctgccctgtgttcccgtcactccggagctgtgaatcaggaccactccaagaactccattggtgtagcctccaccatggcccatgagctgggccacaacctgggcatgagccatgatgaggacattccaggatgctactgtcctgaaccacgggagggtggtggctgcatcatgaccgaaagcatcggctccaagttccccaggatattcagcaggtgtagcaagattgacctagagtcattcgtgacaaaaccccagacaggctgcctgaccaatgttccagatgtcaaccggttcgtgggtggccctgtgtgtggaaacctgtttgtggagcatggagagcagtgtgactgtggcacacctcaggactgtcaaaacccctgctgcaatgccaccacttgccagctggtcaagggggcagagtgtgccagtggtacctgttgtcatgaatgcaaggtgaagccagctggagaggtgtgtcgtctcagtaaggacaaatgtgacctggaggagttctgtgatggccggaagccaacatgtcccgaagatgccttccaacagaatggcactccctgcccagggggctactgctttgatgggagctgtcccaccctggcacagcagtgccgggatctgtgggggccaggtgctcgggtagcagccgactcctgctatacctttagcatccctccgggctgcaatgggaggatgtactctggcaggatcaaccggtgtggagcgctgtactgtgagggaggccagaagccccttgaacgctccttctgcactttctcctccaaccatggagtctgccatgctcttggcacaggcagcaacattgacacctttgagctggtattgcagggcaccaagtgcgaggagggaaaggtttgcatggatggaagctgccaggacctccgtgtatacagatctgaaaactgctctgctaaatgcaacaaccatggggtatgcaaccacaagagggagtgccactgtcacaagggctgggcaccacccaactgtgtacagcggctggcagatgtatcagatgaacaagcagcgtctacgagcctcccagtcagtgtggttgtggtcttggtgatcctggtggctgcgatggtcatcgtggcaggcatcgtcatctaccgaaaggctccgagacaaatccagaggaggagtgtggcacccaagcctatctcggggctctccaaccccctattctacacaagggacagcagcctgccagctaagaacaggcctccagacccttctgagacagtttctaccaaccagcccccaagacccatagtgaaaccaaagaggcctccccctgcacctccaggtgctgtgtccagttcaccactcccagttcctgtttatgccccaaagataccaaatcagtttagacctgatcctcccaccaagcccctcccagagctgaaacccaagcaggtcaagccaacctttgcacccccgacaccaccagtcaagcccgggactggagggacggtgcctggagcaactcagggagctggtgagccaaaggttgctctgaaggtccccatccagaagaggtga

SEQ ID NO: 101 - DNA, 21 bp, synthetic construct
  source 1..21: mol_type = other DNA
cggcacctgcatgacaacgta

SEQ ID NO: 102 - DNA, 21 bp, synthetic construct
  source 1..21: mol_type = other DNA
ctgcgcgaagctgctgactga

SEQ ID NO: 103 - protein, 17 aa, Homo sapiens
CDLEEFCDGRHPECPED

SEQ ID NO: 104 - protein, 28 aa, synthetic construct
RNRCCNSTTCQLAEGAQCAHGTCCQECK